(19) United States
(12) Patent Application Publication
(10) Pub. No.: US 2007/0244388 A1
(43) Pub. Date: Oct. 18, 2007

(54) Position Detection System, Guidance System, Position Detection Method, Medical Device, and Medical Magnetic-Induction and Position-Detection System

(76) Inventors: Ryoji Sato, Tokyo (JP); Akio Uchiyama, Kanagawa (JP); Atsushi Kimura, Tokyo (JP); Hironao Kawano, Tokyo (JP)

Correspondence Address: SCULLY, SCOTT, MURPHY & PRESSER, P.C., 400 GARDEN CITY PLAZA, SUITE 300, GARDEN CITY, NY, 11530, US

(21) Appl. No.: 11/629,340

(22) PCT Filed: Dec. 16, 2005
(86) PCT No.: PCT/JP05/23550

§ 371 Date: Feb. 12, 2007

(30) Foreign Application Priority Data
Dec. 17, 2004 (JP) 2004-366665
Mar. 28, 2005 (JP) 2005-092033
Aug. 8, 2005 (JP) 2005-229474
Sep. 22, 2005 (JP) 2005-275105

(51) Int. Cl. A61B 5/06

(52) U.S. Cl. 600/424; 600/550; 606/130

(57) ABSTRACT

There are provided a position detection system, a guidance system, and a position detection method which obviate the need for frequency adjustment of an alternating magnetic field used in position detection of a device and which allow the device to be made more compact and less expensive. There are included a device (capsule endoscope 20) provided with a magnetic induction coil, a drive coil 51 for generating an alternating magnetic field, a plurality of magnetic sensors 52 for detecting an induced magnetic field, a frequency determining section 50B for determining a position calculating frequency based on a resonance frequency of the magnetic induction coil, and a position analyzing unit 50A for calculating, at the position calculating frequency, at least one of the position and the orientation of the device 20 based on the difference between outputs from the magnetic sensors 52 when only the alternating magnetic field is applied and outputs from the magnetic sensors 52 when the alternating magnetic field and the induced magnetic field are applied; and at least one of a frequency range of the alternating magnetic field and an output frequency range of the magnetic field sensors is limited based on the position calculating frequency.

### TECHNICAL FIELD

The present invention relates to a position detection system, a guidance system, a position detection method, a medical device, and a medical magnetic-induction and position detection system.

### BACKGROUND ART

Recently, there has been research and development of swallowable capsule medical devices, as represented by capsule endoscopes and the like, that are swallowed by a subject to enter the subject's body, where they traverse a passage in the body cavity to capture images of a target site inside the passage in the body cavity. The capsule endoscopes described above have a configuration in which an imaging device that can perform the medical procedure described above, for example, a CCD (Charge Coupled Device) that can acquire images or the like, is provided and perform image acquisition at the target site inside the passage in the body cavity.

However, the capsule medical device described above simply moves in the digestive tract by means of peristalsis, and it is not possible to control the position and orientation of the capsule medical device. In order for this capsule medical device to reliably reach the target site in the passage of the body cavity or to indwell at the target site to perform detailed examination or the like, which requires some time, it is necessary to perform guidance control of the capsule medical device rather than relying on peristalsis of the passage in the body cavity. Thus, one solution that has been proposed to guide the capsule medical device is to control the position and so forth of this device by installing a magnet inside the device and applying a magnetic field from the outside. Furthermore, a technique for driving the capsule medical device inside the passage in the body cavity has also been proposed (for example, see Japanese Unexamined Patent Application Publication No. 2002-187100 (hereinafter referred to as Document 1)).

In order to facilitate diagnosis with the capsule medical device, it is necessary for guiding this capsule medical device to detect where in the passage inside the body cavity the capsule medical device is located; therefore, a technique has been proposed for detecting the position of the capsule medical device when it has been guided to a location (such as inside the passage in the body cavity) where its position cannot be visually confirmed (see, for example, International Publication No. 2004/014225 Pamphlet (hereinafter referred to as Document 2), Japanese Patent No. 3321235 (hereinafter referred to as Document 3), Japanese Unexamined Patent Application Publication No. 2004-229922 (hereinafter referred to as Document 4), and Japanese Unexamined Patent Application Publication No. 2001-179700 (hereinafter referred to as Document 5)). A magnetic position detection method is also a known method for detecting the position of the medical device. As one method for magnetically detecting the position, there is a known technique for identifying the position of an object to be detected by applying an external magnetic field to the object to be detected, in which a coil is installed, and detecting the magnetic field generated due to the induced electromotive force thereof (see, for example, Japanese Unexamined Patent Application Publication No: HEI-6-285044 (hereinafter referred to as Document 6), and Tokunaga, Hashi, Yabukami, Kouno, Toyoda, Ozawa, Okazaki, and Arai, “High-resolution position detection system using LC resonant magnetic marker”, Magnetics Society of Japan, 2005, 29, p. 153-156 (hereinafter referred to as Document 7)).

Document 2 described above discloses a technique for detecting the position of a capsule medical device by detecting, using a plurality of external detectors, the electromagnetism issuing from the capsule medical device, which is provided with a magnetic-field generating circuit in which an AC power supply is connected to an LC resonant circuit.

However, the frequency characteristics of a coil used in the LC resonant circuit described above exhibit variations within a predetermined range due to variations occurring when manufacturing the coil. In addition, the frequency characteristics of the LC resonant circuit are also affected by variations in the characteristics of the coil and capacitors, resulting in the problem of variations occurring within a predetermined range.

One known solution to the problems described above is a technique using capacitors whose capacitance can be adjusted (variable capacitors), coils whose frequency characteristics can be adjusted (coils in which the position of the core of the coil can be adjusted), and so forth.

However, because an adjustment mechanism is provided for elements such as these adjustable capacitors and coils, there is a problem in that it is difficult to reduce the size of the capsule medical device.

Furthermore, a technique in which variations in the frequency characteristics of the LC resonant circuit can be suppressed by selecting capacitors with different capacitances to match the coil characteristics is also known.

However, if the capacitances of the capacitors are selected according to the individual LC resonant circuit, the number of manufacturing steps of the LC resonant circuit increases, resulting in the problem of increased manufacturing costs of the capsule medical device.

Moreover, it is difficult to reduce the size of the capsule because it is necessary to use a power supply inside the capsule and because it is necessary to increase the power supply capacity. In addition, there is also the problem of reduced operating time of the capsule.

### DISCLOSURE OF INVENTION

The present invention has been conceived to overcome the problems described above, and an object thereof is to provide a position detection system, a guidance system, and a position detection method that do not require frequency adjustment of an alternating magnetic field used in position detection of a device such as a capsule medical device or the like and that can reduce the size and cost of the device.

In order to achieve the object described above, the present invention provides the following solutions.

A first aspect of the present invention is position detection system comprising a device equipped with a magnetic induction coil; a drive coil for generating an alternating magnetic field; a plurality of magnetic field sensors for detecting an induced magnetic field generated when the magnetic induction coil receives the alternating magnetic field; a frequency determining section for determining a position calculating frequency which is based on a resonance frequency of the magnetic induction coil; and a position analyzing unit for calculating, at the position calculating frequency, at least one of the position and the orientation of the device based on the difference between an output of the magnetic field sensor when only the alternating magnetic field is applied and an output of the magnetic field sensor when the alternating magnetic field and the induced magnetic field are applied, wherein, based on the position calculating frequency, at least one of a frequency range of the alternating magnetic field and an output frequency range of the magnetic sensor is limited.

According to this aspect, because it is possible to determine the frequency characteristic (the resonance frequency is one such frequency characteristic) of the magnetic induction coil by detecting the induced magnetic field, even if the frequency characteristic of individual magnetic induction coil varies, the frequency determining section can determine a position calculating frequency based on those varying frequency characteristics. Accordingly, the position detection system of this aspect can always calculate the position and orientation of the device based on the position calculating frequency, even if the frequency characteristics of the magnetic induction coils vary.

As a result, there is no need to install elements for adjusting the frequency characteristic of the magnetic induction coil or the like, which allows the device to be reduced in size. More specifically, to adjust the resonance frequency, it is not necessary to select or adjust elements such as capacitors constituting the resonant circuit together with the magnetic induction coil, which can prevent the manufacturing cost of the device from increasing.

Because only an alternating magnetic field at the position calculating frequency is used in calculating the position and orientation of the device, the time required for calculating the position and orientation can be reduced compared to a method in which, for example, the frequency of the alternating magnetic field is swept over a predetermined range.

Furthermore, an example of a case in which the resonance frequency of the magnetic induction coil changes is a case where, in a configuration for controlling the motion of the device, by building a magnet into the device and applying an external magnetic field to control the movement of the built-in magnet, the resonance frequency of the magnetic induction coil changes due to the effect of the built-in magnet.

In this case too, because the frequency determining section can determine the position calculating frequency based on the resonance frequency affected by the built-in magnet, it is possible to calculate the position and orientation of the device without using elements for adjusting the resonance frequency and so forth.

In the first aspect of the invention described above, preferably, the frequency determining section determines the position calculating frequency based on the output from the magnetic field sensor when the induced magnetic field is applied.

According to this configuration, the resonance frequency of the magnetic induction coil is determined based on the output from the magnetic field sensor due to the induced magnetic field, and the position calculating frequency is determined based on that resonance frequency. Accordingly, it is possible to use an appropriate position calculating frequency to calculate the position and orientation of individual device. As a result, a reduction in calculation accuracy of the position and orientation of the device can be prevented, and the time required for calculation can be prevented from increasing.

Furthermore, the first aspect described above preferably further includes a magnetic-field-frequency varying section for periodically varying the frequency of the alternating magnetic field, wherein the frequency determining section determines the position calculating frequency based on the outputs from the magnetic field sensors when applying the induced magnetic field generated by receiving the alternating magnetic field whose frequency is time varying.

According to this configuration, because the alternating magnetic field whose frequency is time varying is used to determine the resonance frequency of the magnetic induction coil, it is possible to determine the resonance frequency even if the variation in resonance frequencies of the magnetic induction coils is large. Accordingly, it is possible to use appropriate position calculating frequencies to calculate the position and orientation of individual device, which enables a reduction in calculation accuracy of the position and orientation of the devices to be prevented and an increase in the time required for calculation to be prevented.

The first aspect described above preferably further includes an impulse-magnetic-field generating section for applying an impulse drive voltage to the drive coil to generate an impulse magnetic field, wherein the frequency determining section determines the position calculating frequency based on the outputs from the magnetic field sensors when applying the induced magnetic field generated by receiving the impulse magnetic field.

According to this configuration, because the impulse magnetic field has many frequency components, it is possible to determine the frequency characteristic of the magnetic induction coil in a shorter period of time compared to a method in which, for example, the frequency of the magnetic field is swept, and in addition, it is possible to determine the resonance frequency over a wider frequency range. As a result, it is possible to use an appropriate position calculating frequency to calculate the position and orientation of individual device, which allows a reduction in calculation accuracy of the position and orientation of the devices to be prevented and allows the time required for calculation to be prevented from increasing.

The first aspect described above preferably further includes a mixed-magnetic-field generating section for generating an alternating magnetic field in which a plurality of different frequencies are mixed; and a variable band limiting section for limiting the output frequency range of the magnetic field sensor and for changing the range of limitation, wherein the frequency determining section determines the position calculating frequency based on output which is acquired, through the variable band limiting section, from the outputs of the magnetic field sensors when applying the induced magnetic field generated by receiving the alternating magnetic field in which a plurality of different frequencies are mixed.

According to this configuration, because an alternating magnetic field having a mixture of a plurality of different frequencies is used to determine the resonance frequency of the magnetic induction coil, it is possible to more easily determine the resonance frequency compared to a case in which an alternating magnetic field with a time varying predetermined frequency is used, even if the variation in resonance frequencies of the magnetic induction coils is large.

Also, by using the variable band limiting section, it is possible to determine the position calculating frequency based on the output in a predetermined frequency range from among the outputs of the magnetic field sensors when applying thereto the induced magnetic field that is generated by receiving the alternating magnetic field described above.

The first aspect described above preferably further includes a memory section for storing information concerning the resonance frequency of the magnetic induction coil, wherein the frequency determining section receives the information and determines the position calculating frequency based on the information.

According to this configuration, by determining the position calculating frequency based on information concerning the resonance frequency of the magnetic induction coil, held in the memory section, it is possible to reduce the time required to calculate the position and orientation of the device compared to a method in which the resonance frequency is measured each time position detection of the device is carried out to determine the position calculating frequency.

The first aspect described above may further include a drive-coil control section for controlling the drive coil based on the position calculating frequency.

According to this configuration, because the drive coil can be controlled based on the position calculating frequency, it is possible to control the frequency of the alternating magnetic field generated by the drive coil.

In the first aspect described above, the position detection system preferably further includes a band limiting section for controlling the output frequency band of the magnetic field sensor based on the position calculating frequency.

According to this configuration, it is possible to control the output frequency band of the induced magnetic field and the like that the magnetic field sensor detects, based on the position calculating frequency. Accordingly, it is possible to obtain a magnetic field sensor output in a frequency range including the position calculating frequency, with low noise, and it is possible to calculate the position and orientation of the device based on this.

In the first aspect described above, the band limiting section preferably uses a Fourier transform.

According to this configuration, the use of a Fourier transform by the band limiting section enables more effective elimination of noise.

In the first aspect described above, the plurality of magnetic field sensors are preferably disposed at a plurality of orientations facing an operating region of the device.

According to this configuration, regardless of the position of the device, an induced magnetic field with a detectable intensity acts on the magnetic field sensor disposed in at least one direction from among the magnetic field sensors disposed in the plurality of directions described above.

The intensity of the induced magnetic field acting on the magnetic field sensor affected by the distance between the device and the magnetic field sensor and the distance between the device and the drive coil. Accordingly, even if the device is at a position where the induced magnetic field acting on the magnetic field sensor disposed in one direction is weak, in the magnetic field sensors disposed in the other directions, the induced magnetic field acting thereat is not weak.

As a result, regardless of the position of the device, the magnetic field sensor can always detect the induced magnetic field.

Since the number of pieces of magnetic field information obtained is the same as the number of magnetic field sensors disposed at different positions, it is possible to obtain position information and so forth of the device according to the number of pieces of magnetic field information.

For example, the information obtained about the device contains a total of six pieces of information, namely, the X, Y, and Z coordinates of the device, rotational phases φ and θ about two axes that are orthogonal to the central axis of the built-in coil and that are also orthogonal to each other, and the intensity of the induced magnetic field. Accordingly, if six or more pieces of magnetic field information are obtained, the six pieces of position information described above can be determined, and it is possible to determine the position and orientation of the device, as well as the intensity of the induced magnetic field.

The first aspect described above preferably further includes a magnetic-field-sensor selecting unit for selecting a magnetic field sensor whose signal output is strong from among the output signals of the plurality of magnetic field sensors.

According to this configuration, because a signal output having few noise components relative to the signal strength can be obtained by selecting the magnetic field sensor having a strong signal output, it is possible to reduce the amount of information to be computationally processed, which enables the computational load to be reduced. Also, since the computational load is reduced, the time required for calculation can be shortened.

In the first aspect described above, the drive coil and the magnetic field sensors are preferably disposed at opposing positions on either side of the operating region of the device.

According to this configuration, since the drive coils and the magnetic field sensors are disposed at opposing positions on either side of the operating region described above, it is possible to position the drive coils and the magnetic field sensors such that they do not structurally interfere.

The first aspect described above may further include a relative-position measuring unit for measuring a relative position between the drive coil and the magnetic field sensors; an information storing section for storing, in association with each other, a reference value, which is an output value from the magnetic field sensor when only the alternating magnetic field is applied, and an output from the relative-position measuring unit at that time; and a current-reference-value generating section for generating, as a current reference value, a current output value of the magnetic field sensor when only the alternating magnetic field is applied, based on the output of the relative-position measuring unit and the information in the information storing section.

According to this configuration, even through the drive coils and the magnetic field sensors can be shifted relatively, it is possible to determine the position and orientation of the device.

Since reference values and the relative positions of the drive coils are stored, there is no need to re-measure the reference values and so forth, even if the relative positions of the drive coils and the magnetic field sensors differ when detecting the position of the device.

In the first aspect described above, the current-reference-value generating section preferably generates, as the current reference value, the reference value which is associated with the relative position closest to the current output of the relative-position measuring unit.

According to this configuration, because the reference value associated with the relative position closest to the output of the relative-position measuring unit is defined as the current reference value, the time required to generate the current reference value can be reduced.

In the first aspect described above, the current-reference-value generating section preferably determines a predetermined approximate equation which relates the relative position and the reference value and generates the current reference value based on the predetermined approximate equation and the current output from the relative-position measuring unit.

According to this configuration, since the current reference value is generated based on a predetermined approximate equation, a more accurate current reference value can be generated compared to a method in which, for example, the reference value directly defines the current reference value.

In the first aspect described above, the device is preferably employed as a capsule medical device.

Furthermore, a second aspect of the present invention is a guidance system including a position detection system according to the first aspect described above; a guidance magnet installed in the device; a guidance-magnetic-field generating unit for generating a guidance magnetic field to be applied to the guidance magnet; and a guidance-magnetic-field-direction control unit for controlling the direction of the guidance magnetic field.

According to the second aspect of the present invention, by controlling the direction of the magnetic field applied to the guidance magnet built into the device, it is possible to control the direction of the force exerted on the guidance magnet, and it is possible to control the direction of motion of the device.

Also, at the same time, it is possible to detect the position of the device and to guide the device to a predetermined position.

In the second aspect described above, preferably, the guidance-magnetic-field generating unit includes three pairs of frame-shaped electromagnets disposed to oppose each other in mutually orthogonal directions; a space in which a subject can be disposed is provided at the inner sides of the electromagnets; and the drive coil and the magnetic field sensors are disposed around the space in which the subject can be disposed.

According to this configuration, by controlling the respective magnetic field intensities generated from the three pairs of frame-shaped electromagnets that are disposed to oppose in mutually orthogonal directions, it is possible to control the direction of the parallel magnetic field produced at the inner sides of the electromagnets in a predetermined direction. Accordingly, a magnetic field in a predetermined direction can be applied to the device, which allows the device to be moved in a predetermined direction.

Also, in a case where the device is a capsule medical device, the space at the inner sides of the electromagnets is a space where a subject can be positioned, and the drive coils and the magnetic field sensors are disposed around this space; therefore, it is possible to guide the device (capsule medical device) to a predetermined location within the body of the subject.

In the second aspect described above, a helical part for converting a rotary force around the longitudinal axis of the device into propulsion force in direction of the longitudinal axis is preferably provided on an outer surface of the device.

According to this configuration, when a rotary force about the longitudinal axis is applied to the device, a force that propels the device in the longitudinal direction thereof is generated by the action of the helical part. Since the helical part generates a propulsion force, by controlling the rotation direction about the longitudinal axis, it is possible to control the direction of the propulsion force acting on the device.

In the second aspect described above, if the device is a capsule medical device, the guidance system preferably further includes an image-capturing unit, in the device (capsule medical device), having an optical axis along the longitudinal axis of the device; a display unit for displaying images captured by the image-capturing unit; and an image control unit for rotating the images captured by the image-capturing unit in the opposite direction, based on rotation information about the longitudinal axis of the device, by means of a guidance-magnetic-field-direction control unit, and for displaying them on the display unit.

According to this configuration, since the above-described acquired images are subjected to processing for rotating them in the direction opposite to the rotation direction of the device (capsule medical device) based on the rotation information (rotational phase information about the longitudinal axis), it is possible to always display them on the display unit as if they were images acquired with a predetermined rotational phase, regardless of the rotational phase of the device.

For example, when guiding the capsule medical device while the operator views the images displayed on the display unit, converting the displayed images to images having a predetermined rotational phase, as described above, makes it easier to guide the capsule medical device to a predetermined position compared to the case where the displayed images rotate together with the rotation of the capsule medical device.

A third aspect of the present invention is a position detection method for a device, comprising a step of obtaining a characteristic of a magnetic induction coil installed in the device; a step of determining a position calculating frequency from the characteristic; a step of limiting at least one of a frequency range of an alternating magnetic field and a frequency range of a magnetic sensor based on the position calculating frequency; a step of generating the alternating magnetic field, which includes a position calculating frequency component; a measuring step for obtaining an output from the magnetic field sensor; and a position calculating step of determining at least one of the position and the orientation of the magnetic induction coil.

According to the third aspect described above, it is not necessary to provide elements and the like for adjusting the resonance frequency of the magnetic induction coil, which allows the device to be reduced in size. More specifically, it is not necessary to select or adjust elements such as capacitors and the like constituting the resonant circuit together with the magnetic induction coil in order to adjust the resonance frequency, which prevents the manufacturing costs of the device from increasing.

Since only an alternating magnetic field at the position calculating frequency is used to calculate the position and direction of the device, the time required for calculating the position and orientation can be reduced compared to a method in which, for example, the frequency of the alternating magnetic field is swept over a predetermined range each time position detection of the device is carried out.

Furthermore, according to the third aspect described above, because it is possible to determine the characteristics of the magnetic induction coil by, for example, detecting the induced magnetic field, even if there is some variation in the characteristics of the magnetic induction coils, it is possible to determine the position calculating frequency based on the characteristics having such a variation. Accordingly, even if the characteristics of the magnetic induction coil vary, it is always possible to calculate the position and orientation of the device based on the position calculating frequency.

Furthermore, according to the third aspect described above, it is possible to determine the position calculating frequency based on, for example, characteristics of the magnetic induction coil stored in advance in the device. Accordingly, it is possible to shorten the time required for calculating the position and orientation of the device compared to a method in which the characteristics are obtained each time position detection of the device is carried out to determine the position calculating frequency.

In the third aspect described above, the measuring step and the position calculating step are preferably repeated.

According to this configuration, by repeating the measuring step and the position calculating step, at least one of the position and orientation of the magnetic induction coil can be repeatedly determined.

According to the position detection system, the guidance system, and the device position detection method of the present invention described in the above-described first to third aspects, since the frequency determining section can determine the calculating frequency based on the varying resonance frequency thereof and can calculate the position and orientation of the device based on the calculating frequency, an advantage is afforded in that it is possible to eliminate the need for frequency adjustment of the alternating magnetic field or the like used in position detection of the device.

Thus, it is not necessary to provide elements or the like for adjusting the resonance frequency of the magnetic induction coil, which is advantageous in that the device can be reduced in size. More specifically, to adjust the resonance frequency, it is not necessary to select or adjust elements such as capacitors and the like constituting the resonant circuit together with the magnetic induction coil, thus providing an advantage in that it is possible to reduce the manufacturing costs of the device.

A fourth aspect of the present invention is a medical magnetic-induction and position detection system comprising a medical device that is inserted into the body of a subject and that has at least one magnet and a circuit including a built-in coil; a first magnetic-field generating section for generating a first magnetic field; a magnetic-field detecting section for detecting an induced magnetic field induced in the built-in coil by the first magnetic field; and one or more sets of opposing coils for generating a second magnetic field to be applied to the magnet, wherein the two coils constituting the opposing coils are driven individually.

According to the fourth aspect, by individually driving the two respective coils constituting the opposing coils, even under conditions where mutual induction with respect to the first magnetic field is induced in one of the coils of the opposing coils, it is possible to prevent an electrical current caused by the electromotive force due to the mutual induction from flowing from the one coil to the other coil. Accordingly, the other coil does not generate a magnetic field that is in-phase with the mutual-induction magnetic field, which is in anti-phase with the first magnetic field, and generates only the second magnetic field.

As a result, since it is possible to prevent the generation of a magnetic field that cancels out the first magnetic field from the other coil, the formation of a region where the first magnetic field becomes substantially zero can be prevented, which allows the formation of a region where no induced magnetic field is generated in the built-in coil to be avoided.

A fifth aspect of the present invention is a medical magnetic-induction and position detection system comprising a medical device that is inserted into the body of a subject and that has at least one magnet and a circuit including a built-in coil; a first magnetic-field generating section for generating a first magnetic field; a magnetic-field detecting section for detecting an induced magnetic field induced in the built-in coil by the first magnetic field; one or more sets of opposing coils for generating a second magnetic field to be applied to the magnet; and a switching section for electrically connecting to the opposing coils, wherein the switching section enters a disconnected state only while the magnetic-field detecting section detects the position of the built-in coil.

According to the fifth aspect described above, by disconnecting the switching section only while the magnetic-field detecting section is detecting the position of the built-in coil, it is possible to prevent the formation of a mutual-induction magnetic field, even under conditions where mutual induction with respect to the first magnetic field is induced in the opposing coils. On the other hand, by connecting the switching section while the magnetic-field detecting section is not detecting the position of the built-in coil, it is possible to generate a second magnetic field in the opposing coils.

A sixth aspect of the present invention is a medical magnetic-induction and position detection system comprising a medical device that is inserted into the body of a subject and that has at least one magnet and a circuit including a built-in coil; a first magnetic-field generating section for generating a first magnetic field; a magnetic-field detecting section for detecting an induced magnetic field induced in the built-in coil by the first magnetic field; and one or more set of opposing coils for generating a second magnetic field to be applied to the magnet, wherein the two coils constituting the opposing coils are driven in parallel.

According to the sixth aspect described above, by driving the two coils constituting the opposing coils in parallel, even under conditions where mutual inductance with respect to the first magnetic field is induced in one of the two coils, it is possible to prevent an electrical current caused by an electromotive force due to the mutual inductance from flowing from one coil to the other coil. Accordingly, the other coil does not generate a magnetic field that is in-phase with the mutual-inductance magnetic field, which is in anti-phase with the first magnetic field, and generates only a second magnetic field.

Since it is possible, as a result, to prevent the generation of a magnetic field that cancels out the first magnetic field from the other coil, the formation of a region where the first magnetic field becomes substantially zero can be prevented, and the formation of a region where no induced magnetic field is generated in the built-in coil can be prevented.

In the fourth aspect to the sixth aspect described above, preferably, at least three sets of the opposing coils are provided around a region where the magnet is disposed; the first magnetic-field generating section includes a magnetic-field generating coil disposed close to one of the coils in the at least one set of opposing coils; the magnetic-field detecting section includes a magnetic field sensor disposed close to the other coil in the at least one set of opposing coils; and, from among the at least three sets of opposing coils, the direction of a central axis of at least one set of opposing coils is arranged to be a direction intersecting a surface formed from the central axes of the two other sets of opposing coils.

According to this aspect, the magnetic-field generating coil generates a first magnetic field which induces an induced magnetic field in the built-in coil included in the medical device. The induced magnetic field generated from the built-in coil is detected by the magnetic field sensor and is used to detect the position or orientation of the medical device having the built-in coil. Also, the second magnetic field generated in the at least three sets of opposing coils is applied to the magnet included in the medical device to control the position and orientation of the medical device. Therefore, since the direction of the central axis of the at least one set of opposing coils is disposed so as to correspond to a direction intersecting the surface formed from the central axes of the other two sets of opposing coils, the magnetic force lines of the second magnetic field can be oriented three-dimensionally in any direction. Thus, it is possible to three-dimensionally control the position and orientation of the medical device having the magnet.

In addition, by means of the first magnetic field generated from the magnetic-field generating coil disposed close to one of the coils of the opposing coils, even under conditions where mutual inductance is induced in the one of the opposing coils, at least the other coil does not generate a magnetic field that is in-phase with the mutual-inductance magnetic field, which is in anti-phase with the first magnetic field, and generates only a second magnetic field. Since it is possible, as a result, to prevent the generation of a magnetic field that cancels out the first magnetic field from the other coil of the opposing coils, the formation of a region where the first magnetic field becomes substantially zero can be prevented.

With the medical magnetic-induction and position detection systems according to the fourth aspect to the sixth aspect of the present invention described above, even under conditions where mutual inductance is induced in one of the coils of the two coils constituting the opposing coils, since it is possible to prevent the generation of a mutual-inductance magnetic field in at least the other coil, the formation of a region where the first magnetic field is canceled out and the intensity of the magnetic field becomes substantially zero can be prevented, which affords an advantage in that it is possible to prevent the magnetic field intensity used for position detection from decreasing.

A seventh aspect of the present invention is a medical device comprising at least one magnet and a circuit including a built-in coil having a core formed of a magnetic material, wherein the position of the built-in coil is detected by a magnetic position detection unit disposed outside the body of a subject, and wherein the core is disposed at a position where there is no magnetic saturation by the magnetic field that the magnet produces.

According to the seventh aspect described above, by using the core made from a magnetic material in the built-in coil, it is possible to improve the performance of the built-in coil, and the occurrence of problems during position detection of the medical device can thus be prevented.

For example, when applying an external magnetic field (for example, an alternating magnetic field) for position detection to the built-in coil, the intensity of the magnetic field that the built-in coil produces is stronger compared to a case where a core made from magnetic material is not used in the built-in coil. Accordingly, the position detection unit can more easily detect the magnetic field that the built-in coil produces, which prevents the occurrence of problems when detecting the position of the medical device.

Furthermore, because the core is disposed at a position where the magnetic flux density inside the core due to the magnetic field that the magnet produces is not magnetically saturated, it is possible to prevent the performance of the built-in coil from degrading.

For example, when applying an alternating magnetic field for position detection and a steady magnetic field for position control to the built-in coil, the amount of change in the intensity of the magnetic field that the built-in coil produces in response to a change in the intensity of the alternating magnetic field is larger than in a case where the core is disposed at a position where the internal magnetic flux density is magnetically saturated. Accordingly, the position detection unit can more easily detect the amount of change in the magnetic field intensity mentioned above, and it is possible to prevent the occurrence of problems when detecting the position of the medical device.

In the seventh aspect described above, preferably, the core has a shape for which the demagnetizing factor in the core for the central axis direction of the built-in coil is smaller than the demagnetizing factor for other directions, and the direction of the magnetic field that the magnet produces at the core position is a direction intersecting the central axis direction.

According to this configuration, since the core has a shape for which the demagnetizing factor for the central axis direction of the built-in coil is smaller than the demagnetizing factor for other directions and the magnetic field direction of the magnet at the core position intersects the central axis direction, it is possible to improve the performance of the built-in coil further.

More specifically, because the magnetic field of the magnet impinges on the core from a direction other than the direction in which the demagnetizing factor is minimized, it is possible to increase the magnetic field intensity required to magnetically saturate the core. Accordingly, even if an external magnetic field is applied to the built-in coil, it is possible to prevent the core from magnetically saturating.

In the seventh aspect described above, preferably, the direction of the magnetic field that the magnet produces at the position of the built-in coil and the direction for which the demagnetizing factor in the core is minimized are different.

According to this configuration, because the magnetic field direction of the magnet at the position of the built-in coil and the direction in which the demagnetizing factor in the core is minimized are different, the magnetic field of the magnet impinges on the core from a direction other than the direction in which the demagnetizing factor is minimized. Accordingly, it is possible to increase the magnetic field intensity required for the core to magnetically saturate. Thus, even if an external magnetic field is applied to the built-in coil, it is possible to prevent the core from magnetically saturating.

In the seventh aspect described above, it is particularly preferable that the angle formed between the direction of the magnetic field that the magnet produces at the position of the built-in coil and the direction for which the demagnetizing factor in the core is minimized be about 90 degrees.

According to this configuration, because the magnetic field direction of the magnet at the position of the built-in coil and the direction in which the demagnetizing factor in the core is minimized form an angle of substantially 90 degrees, the magnetic field of the magnet impinges on the core from a direction other than the direction in which the demagnetizing factor is minimized.

For example, when the shape of the core is plate-like or rod-like, because the magnetic field of the magnet impinges on the core from a direction in which the demagnetizing factor is maximized, it is possible to maximize the demagnetizing field produced inside the core. Accordingly, the effective magnetic field inside the core can be minimized, and the core can be prevented from magnetically saturating.

In the seventh aspect described above, it is preferable that the core be positioned so that the demagnetizing factor for the central axis direction is smaller than the demagnetizing factors for other directions, and that the direction of the magnetic field that the magnet produces at the position of the built-in coil and the central axis direction be substantially orthogonal.

According to this configuration, because the core is disposed so that the demagnetizing factor for the central axis direction is smaller than the demagnetizing factors for other directions and because the magnetic field direction of the magnet is substantially orthogonal to the central axis direction, the magnetic field of the magnet impinges on the core from a direction other than the direction in which the demagnetizing factor is minimized. Accordingly, it is possible to prevent the demagnetizing field produced inside the core from being minimized and to prevent the effective magnetic field inside the core from being maximized, which enables magnetic saturation of the core to be prevented.

Preferably, the magnet is disposed at the position described above so that the center of gravity is located on the central axis, and the magnetization direction of the magnet is substantially orthogonal to the central axis.

According to this configuration, because the center of gravity of the magnet is located on the central axis and the magnetization direction of the magnet is substantially orthogonal to the central axis, the magnetic field direction of the magnet at the position of the core is substantially orthogonal to the central axis.

In the seventh aspect described above, it is preferable that the built-in coil be disposed at a position where the magnetic flux density inside the core produced by the magnetic field of the magnet becomes ½ or less the saturated magnetic flux density in the core.

According to this configuration, since the built-in coil is disposed at a position where the magnetic flux density formed by the magnetic field of the magnet inside the core is half or less the saturation magnetic flux density in the core, it is possible to suppress a reduction in the reversible magnetic susceptibility in the core. Accordingly, for the other magnetic field of the magnet, even if an alternating magnetic field used in position detection of the built-in coil is formed at the position of the core, it is possible to prevent the magnetic flux density formed inside the core from exceeding the saturation magnetic flux density, and a degradation in performance of the built-in coil can be prevented.

In the seventh aspect described above, the circuit is preferably a resonant circuit.

According to this aspect, by using, for example, an alternating magnetic field with a frequency equal to the resonance frequency of the resonance circuit in position detection of the built-in coil, it is possible to increase the intensity of the magnetic field generated from the built-in coil and so on. More specifically, it is possible to reduce the electrical power consumption of the circuit.

In the seventh aspect described above, the built-in coil may have a hollow structure, the core may be formed to be substantially C-shaped in the cross-section perpendicular to the central axis direction, and the core may be disposed inside the hollow structure.

According to this configuration, by disposing the core inside the hollow structure of the built-in coil, the intensity of the magnetic field generated in the built-in coil can be increased compared to a case where the magnetic field is not applied. More specifically, a magnetic field having weaker intensity can be received by the built-in coil.

Moreover, by forming the cross-sectional shape of the core substantially in the form of a letter C, it is possible to prevent the generation of shielding currents (eddy currents) flowing substantially in the form of loops in the cross-section of the core. Accordingly, shielding of the magnetic field by the shielding currents can be prevented, and it is possible to prevent the generation of a magnetic field in the built-in coil or suppressed reception of the magnetic field.

Since the core is substantially C-shaped in cross-section, the volume of magnetic material used can be reduced compared to a core whose cross-sectional shape is solid.

Other components can be disposed inside the core, which allows the medical device to be reduced in size.

For example, by reducing the thickness in the radial direction in the substantially C-shaped cross-section of the core to form thin layers, it is possible to suppress the generation of eddy currents flowing in the thickness direction of the layers. Or even if they do occur, they can be suppressed to such a level that they have no effect on the position detection of the built-in coil.

For example, when the direction of the magnet's magnetic field impinging on the core is in the thickness direction in the substantially C-shaped cross-section of the core, because the demagnetizing factor for the thickness direction of the core is large, the demagnetizing field formed inside the core is maximized. Accordingly, the effective magnetic field inside the core can be minimized, and the core can be prevented from magnetically saturating.

In the seventh aspect described above, in a configuration where the built-in coil is disposed at a position where the magnetic flux density inside the core produced by the magnetic field of the magnet is half or less the saturated magnetic flux density in the core, the medical device may include a biological-information acquiring unit for acquiring information about the inside of the body of the subject, the magnet may have a hollow structure, and at least part of the biological-information acquiring unit may be disposed inside the hollow structure.

According to this configuration, since the biological-information acquiring unit is disposed inside the hollow structure of the magnet, the medical device can be reduced in size.

In the seventh aspect described above, preferably, the magnet is formed of an assembly of plural magnet pieces, and insulators are disposed between the plurality of magnet pieces.

According to this configuration, because insulators are disposed between the plurality of magnet pieces, it is possible to make it difficult for shielding currents to flow in the magnet formed of an assembly of plural magnet pieces. Accordingly, it is possible to prevent the magnetic field that the built-in coil generates or receives from being shielded by shielding currents flowing in the magnet. More specifically, it is possible to reduce the effect of the shielding currents on the built-in coil, which allows a degradation in performance of the built-in coil to be prevented.

In the seventh aspect described above, the plurality of magnets are preferably formed substantially in the shape of plates.

According to this configuration, because the plurality of magnet pieces are formed in the shape of plates, it is possible to easily form an assembly thereof by laminating the plurality of magnet pieces. In addition, because they are formed in the shape of plates, it is possible to easily sandwich insulators between the magnet pieces.

In the seventh aspect described above, the plurality of magnet pieces formed substantially in the shape of plates may be polarized in the thickness directions thereof.

According to this configuration, by polarizing the plurality of magnet pieces in the thickness directions thereof, it is easier to laminate the plurality of magnet pieces since the magnet pieces are attracted together, and it is easy to construct a magnet which is an assembly thereof.

In the seventh aspect described above, the plurality of magnet pieces formed substantially in the shape of plates may be polarized in directions along the surfaces thereof.

According to this configuration, since the plurality of magnet pieces are polarized in directions along the surfaces thereof, it is possible to intensify the magnetic force of the plurality of magnet pieces compared to the case where they are polarized in the thickness directions thereof, and it is possible to intensify the magnetic force of the magnet which is an assembly thereof.

In the seventh aspect described above, the magnet which is an assembly of the plural magnet pieces is preferably formed to be substantially cylindrical.

According to this configuration, for example, it is possible to dispose other constituent elements of the medical device inside the substantially cylindrical magnet described above, which allows the medical device to be reduced in size.

In the seventh aspect described above, two of the built-in coils may be provided and the two built-in coils may be positioned so that their respective central axes are aligned, and in addition, they may be positioned so as to be separated in the direction of their central axes and the magnet may be positioned between the two built-in coils.

According to this configuration, since the magnet is disposed close to the center of the medical device, when, for example, the magnet is used in driving control of the medical device, driving of the medical device can be facilitated compared to a case where the magnet is disposed towards one end of the medical device.

In the above, two magnets may be provided, the two magnets may be positioned so as to be separated in the direction of the central axis of the built-in coil, and the built-in coil may be positioned between the two magnets.

According to this configuration, since the built-in coil can be disposed close to the center of the medical device, it is possible to more accurately detect the position of the medical device compared to a case where the built-in coil is disposed towards one end of the medical device.

In the seventh aspect described above, preferably, the medical device is a capsule medical device that is put into the body of a subject and has a biological-information acquiring unit for acquiring information about the interior of the body of the subject.

According to this configuration, because the medical device has a biological-information acquiring unit and is put into the body of a subject, this medical device can obtain information about the interior of the body of the subject.

In the seventh aspect described above, in the case where the medical device is a capsule medical device, the built-in coil may have a hollow structure, and at least part of the biological-information acquiring unit may be disposed inside the hollow structure.

According to this configuration, because at least part of the biological-information acquiring unit is disposed inside the hollow structure of the built-in coil, the medical device can be reduced in size and can more easily be inserted inside the body of the subject.

In the seventh aspect described above, in the case where the medical device is a capsule medical device, a power supply unit for driving at least one of the circuit and the biological-information acquiring unit may be provided, the built-in coil may have a hollow structure, and the power-supply unit may be disposed inside the hollow structure.

According to this configuration, because the power supply unit is disposed inside the hollow structure of the built-in coil, the medical device can be reduced in size.

In the seventh aspect described above, in the case where the medical device is a capsule medical device, a power supply unit for driving at least one of the circuit and the biological-information acquiring unit may be provided, the magnet may have a hollow structure, and the power supply unit may be disposed inside the hollow structure.

According to this configuration, because the power supply unit is disposed inside the hollow structure of the magnet, the medical device can be reduce in size.

An eighth aspect of the present invention is a medical magnetic-induction and position-detection system comprising a medical device according to the seventh aspect described above; and a position detection unit including a driving section for generating an induced magnetic field in the built-in coil and a magnetic-field detecting section for detecting the induced magnetic field generated by the built-in coil, wherein the circuit is a magnetic-field generating unit for generating a magnetic field directed from the built-in coil to the position detection unit.

According to the eighth aspect of the present invention, the position detection unit can detect the position of the built-in coil based on the induced magnetic field which the driving section generates in the built-in coil.

More specifically, detecting the generated magnetic field with the magnetic-field detecting section provided in the position detection unit allows the position of the built-in coil to be estimated based on information about the detected magnetic field and so forth.

In the eighth aspect described above, preferably, the driving section of the position detection unit forms a magnetic field in the region where the built-in coil is disposed, and the magnetic-field generating unit receives, by means of the built-in coil, the magnetic field that the position detection unit produces and generates an induced magnetic field from the built-in coil.

According to this configuration, the position detection unit can detect the position of the built-in coil based on the induced magnetic field generated from the built-in coil of the magnetic-field generating unit.

More specifically, the position of the built-in coil can be estimated by detecting the induced magnetic field generated in the built-in coil with the magnetic-field detecting section of the position detection unit.

In the eighth aspect described above, the position detection unit preferably includes a plurality of the magnetic-field detecting sections and a calculating apparatus for calculating at least one of the position and orientation of the built-in coil based on the outputs of the plurality of magnetic-field detecting sections.

According to this configuration, because the calculating apparatus calculates at least one of the position and orientation of the built-in coil based on the outputs of the plurality of magnetic-field detecting sections, at least one of the position and orientation of the built-in coil can be estimated.

Since there are a plurality of magnetic-field detecting sections, a plurality of outputs are also used in calculating the position and orientation of the built-in coil. For example, by selecting the output used in the calculation in the calculating apparatus, it is possible to increase the accuracy of the calculation result of the position and orientation of the built-in coil.

A ninth aspect of the present invention is a medical magnetic-induction and position-detection system comprising a medical device according to the seventh aspect described above; and a position detection unit including a driving section for forming magnetic fields, from a plurality of directions, in a region where the built-in coil is disposed, wherein the circuit includes an internal magnetic-field detecting section for receiving the plurality of magnetic fields that the position detection unit forms and a position-information transmitting unit for transmitting information on the plurality of received magnetic fields to the position detection unit.

According to the ninth aspect of the present invention, the position detection unit can detect the position of the built-in coil based on a plurality of pieces of magnetic field information transmitted from the position-information transmission unit.

More specifically, the internal magnetic-field detecting section receives the magnetic fields formed from a plurality of directions by the driving section, and the plurality of pieces of magnetic field information output from the internal magnetic-field detecting section are transmitted to the position detection unit by the position-information transmitting unit. The position detection unit can estimate the position of the built-in coil based on the plurality of pieces of magnetic field information.

In the ninth aspect described above, the position detection unit preferably includes a calculating apparatus for calculating at least one of the position and orientation of the built-in coil based on the information about the plurality of received magnetic fields at the internal magnetic-field detecting section.

According to this configuration, since the calculating apparatus can calculate at least one of the position and orientation of the built-in coil based on the magnetic field information detected by the internal magnetic-field detecting section, at least one of the position and orientation of the built-in coil can be estimated.

Since there are plurality of pieces of magnetic field information, it is possible to increase the accuracy of the calculation result of the position and orientation of the built-in coil by, for example, selecting the magnetic field information to be used in the calculation in the calculating apparatus.

In either the above-described eighth aspect or the above-described ninth aspect which has the calculating apparatus, preferably, the medical magnetic-induction and position-detection system includes a guidance-magnetic-field generating unit, disposed outside the operating region of the medical device, for generating a guidance magnetic field to be applied to the magnets; and a magnetic-field-direction control unit for controlling the direction of the guidance magnetic field by controlling the guidance-magnetic-field generating unit.

According to this configuration, by providing the guidance-magnetic-field generating unit and the magnetic-field-direction control unit, the medical magnetic-induction and position detection system can generate a guidance magnetic field and can control the direction of the guidance magnetic field. Accordingly, the medical device including the magnet, which is controlled by the guidance magnetic field, can be guided to a predetermined position.

According to the medical device and the medical magnetic-induction and position detection system of the seventh to ninth aspects of the present invention described above, the performance of the built-in coil can be improved by using a core made from a magnetic material in the built-in coil. Accordingly, an advantage is afforded in that the magnetic position detection system can operate more effectively and problems can be prevented from occurring during position detection of the medical device.

Furthermore, since the core is disposed at a position where the magnetic flux density inside the core due to the magnetic field that the magnet produces is not magnetically saturated, an advantage is afforded in that the magnetic position detection system can operate more effectively, and a reduction in performance of the built-in coil can be prevented.

### BRIEF DESCRIPTION OF DRAWINGS

FIG. 1 is a schematic diagram of a medical magnetic-induction and position-detection system according to a first embodiment of the present invention.

FIG. 2 is a perspective view of the medical magnetic-induction and position-detection system in FIG. 1.

FIG. 3 is a schematic diagram showing a cross-section of the medical magnetic-induction and position-detection system in FIG. 1.

FIG. 4 is a schematic diagram showing the circuit configuration of a sense-coil receiving circuit in FIG. 1.

FIG. 5 is a schematic diagram showing the configuration of a capsule endoscope in FIG. 1.

FIG. 6 is a flowchart showing how to determine a calculating frequency and a procedure for detecting the position and orientation of the capsule endoscope according to the present embodiment.

FIG. 7 is a flowchart showing how to determine a calculating frequency and a procedure for detecting the position and orientation of the capsule endoscope according to the present embodiment.

FIG. 8 is a graph showing a frequency characteristic of a resonant circuit.

FIG. 9 is a diagram showing another positional relationship of drive coils and sense coils.

FIG. 10 is a diagram showing another positional relationship of the drive coils and the sense coils.

FIG. 11 is a diagram showing the positional relationship of a drive coil and a magnetic induction coil.

FIG. 12 is a diagram showing the positional relationship between the drive coils and the sense coils.

FIG. 13A is a diagram depicting an impulse drive voltage applied to the drive coils. FIG. 13B is a diagram depicting an impulse magnetic field.

FIG. 14 is a schematic diagram of a medical magnetic-induction and position-detection system according to a second embodiment of the present invention.

FIG. 15 is a schematic diagram showing the configuration of a capsule endoscope in FIG. 14.

FIG. 16 is a flowchart showing a procedure for determining a frequency characteristic of the magnetic induction coil, up to the point of storage in a memory section 134A.

FIG. 17 is a flowchart showing a procedure for detecting the position and orientation of the capsule endoscope.

FIG. 18 is a flowchart showing a procedure for detecting the position and orientation of the capsule endoscope.

FIG. 19 is a diagram showing the positional relationship of drive coils and sense coils according to a third embodiment of the present invention.

FIG. 20 is a schematic diagram showing a cross-section of the medical magnetic-induction and position-detection system.

FIG. 21 shows drive coils and sense coils according to a fourth embodiment of the present invention.

FIG. 22 is a diagram showing the positional relationship between drive coils and sense coils according to a modification of the fourth embodiment of the present invention.

FIG. 23 shows an outline view of a medical magnetic-induction and position-detection system according to a fifth embodiment of the present invention.

FIG. 24 is a diagram showing the positional relationship between a drive coil unit, sense coils, and so forth in FIG. 23.

FIG. 25 shows an outline view of the configuration of the drive coil unit in FIG. 24.

FIG. 26 is a flowchart showing a procedure for detecting the position and orientation of the capsule endoscope according to the present embodiment.

FIG. 27 is a flowchart showing a procedure for detecting the position and orientation of the capsule endoscope according to the present embodiment.

FIG. 28 is a flowchart showing a procedure for detecting the position and orientation of the capsule endoscope according to the present embodiment.

FIG. 29 is an outline view of a position detection system of the capsule endoscope according to the present invention.

FIG. 30 is a diagram schematically showing the configuration of a medical magnetic-induction and position-detection system according to a first modification of the present invention.

FIG. 31 is a connection diagram depicting the configuration of guidance-magnetic-field generating coils in FIG. 30.

FIG. 32 is a diagram showing another modification of the medical magnetic-induction and position-detection system in FIG. 30.

FIG. 33 is a diagram for explaining the magnetic field intensity formed in the medical magnetic-induction and position-detection system in FIG. 30.

FIG. 34 is a diagram schematically showing the configuration of a medical magnetic-induction and position-detection system according to a second modification of the present invention.

FIG. 35 is a connection diagram showing the configuration of guidance-magnetic-field generating coils in FIG. 34.

FIG. 36 is a diagram showing another modification of the medical magnetic-induction and position-detection system in FIG. 34.

FIG. 37 is a diagram schematically showing a medical magnetic-induction and position-detection system according to a third modification of the present invention.

FIG. 38 is a connection diagram for explaining the configuration of guidance-magnetic-field generating coils in FIG. 37.

FIG. 39 is a diagram showing another modification of the medical magnetic-induction and position-detection system in FIG. 37.

FIG. 40 is a diagram schematically showing the configuration of a medical magnetic-induction and position-detection system according to a fourth modification of the present invention.

FIG. 41 is a block diagram schematically depicting the configuration of guidance-magnetic-field generating coils in FIG. 40.

FIG. 42 is a diagram depicting the magnetic field intensity formed in a conventional medical magnetic-induction and position-detection system.

FIG. 43 is a schematic diagram of a medical magnetic-induction and position-detection system according to a sixth embodiment of the present invention.

FIG. 44 is a perspective view of a medical magnetic-induction and position-detection system.

FIG. 45 is a schematic diagram showing a cross-section of a medical magnetic-induction and position-detection system.

FIG. 46 is a schematic diagram showing the circuit configuration of a sense-coil receiving circuit in FIG. 43.

FIG. 47 is a schematic diagram showing the configuration of a capsule endoscope in FIG. 43.

FIG. 48A is a diagram as viewed from the tip of a guidance magnet in the capsule endoscope in FIG. 47. FIG. 48B is a diagram as viewed from the side face of the guidance magnet.

FIG. 49 is a diagram depicting an induced-magnetic-field generating section in the capsule endoscope in FIG. 47.

FIG. 50 is a graph showing a frequency characteristic of the induced-magnetic-field generating section in the capsule endoscope in FIG. 47.

FIG. 51 is a diagram showing the positional relationship of a drive coil and a magnetic induction coil.

FIG. 52 is a diagram showing the positional relationship of drive coils and sense coils.

FIG. 53 is a diagram showing another positional relationship of drive coils and sense coils.

FIG. 54 is a diagram showing another positional relationship of drive coils and sense coils.

FIG. 55 is a diagram depicting the outline of an experimental apparatus used in practice.

FIG. 56A is a diagram depicting the positional relationship of a magnetic induction coil and a battery. FIG. 56B is a diagram depicting the positional relationship of a magnetic induction coil, a battery, and a guidance magnet.

FIG. 57 is a diagram showing the relationship between the gain change of the sense coils and phase change in the experimental apparatus in FIG. 55.

FIG. 58 is a diagram showing the relationship between the gain change of the sense coils and phase change in the experimental apparatus in FIG. 55.

FIG. 59 is a diagram showing the positional relationship of a magnetic induction coil and a guidance magnet in the experimental apparatus in FIG. 55.

FIG. 60A is an elevational view depicting the configuration of a solid-core guidance magnet used in the experimental apparatus in FIG. 55. FIG. 60B is a side view depicting the configuration of the solid-core guidance magnet used in the experimental apparatus in FIG. 55.

FIG. 61A is a side view depicting the configuration of a hollow guidance magnet used in the experimental apparatus in FIG. 55. FIG. 61B is a side view of a large hollow guidance magnet.

FIG. 62 is a diagram showing a frequency characteristic of a sense coil in a guidance magnet formed of five individual magnet pieces.

FIG. 63 is a diagram showing a frequency characteristic of a sense coil in a case where the guidance magnet is formed of five individual magnet pieces and insulators are sandwiched between the individual magnet pieces.

FIG. 64 is a diagram showing a frequency characteristic of a sense coil in a case where the guidance magnet is formed of three individual magnet pieces and insulators are sandwiched between the individual magnet pieces.

FIG. 65 is a diagram showing a frequency characteristic of a sense coil in a case where the guidance magnet is formed of a single magnet piece.

FIG. 66 is a diagram showing a frequency characteristic of a sense coil in a case where the distance between the guidance magnet and the magnetic induction coil is 0 mm.

FIG. 67 is a diagram showing a frequency characteristic of a sense coil in a case where the distance between the guidance magnet and the magnetic induction coil is 5 mm.

FIG. 68 is a diagram showing a frequency characteristic of a sense coil in a case where the distance between the guidance magnet and the magnetic induction coil is 10 mm.

FIG. 69 is a diagram showing a frequency characteristic of a sense coil in a hollow guidance magnet.

FIG. 70 is a diagram showing a frequency characteristic of a sense coil in a large hollow guidance magnet.

FIG. 71 is a diagram showing the relationship between the distance between the guidance magnet and the magnetic induction coil and the magnitude of the output oscillation of the magnetic induction coil.

FIG. 72 is a diagram showing an outline view of an apparatus for measuring the magnetic field intensity that the guidance magnet produces.

FIG. 73 is a diagram showing the relationship between the intensity of the magnetic field produced by the guidance magnet in the center of the magnetic induction coil and the intensity of the output oscillation of the magnetic induction coil.

FIG. 74 is a diagram showing a hysteresis curve for a permalloy layer in FIG. 49.

FIG. 75 is a graph showing the reversible magnetic susceptibility in the permalloy layer in FIG. 49.

FIG. 76 is a schematic diagram depicting the intensity of an effective magnetic field in the permalloy layer.

FIG. 77 is a schematic diagram depicting the intensity of the demagnetizing factor in the permalloy layer.

FIG. 78 is a diagram showing the configuration of a capsule endoscope according to a second embodiment of the present invention.

FIG. 79A is an elevational diagram showing the configuration of a guidance magnet in the capsule endoscope shown in FIG. 78. FIG. 79B is a side view showing the configuration of the guidance magnet.

FIG. 80 is a diagram showing the configuration of a capsule endoscope according to an eighth embodiment of the present invention.

FIG. 81 is a diagram showing the configuration of a capsule endoscope according to a ninth embodiment of the present invention.

FIG. 82 is a diagram showing the configuration of a capsule endoscope according to a tenth embodiment of the present invention.

FIG. 83A is an elevational diagram showing the configuration of a guidance magnet in the capsule endoscope shown in FIG. 82. FIG. 83B is a side view showing the configuration of the guidance magnet.

FIG. 84 is a diagram showing the configuration of a capsule endoscope according to an eleventh embodiment of the present invention.

FIG. 85 is a schematic diagram showing the positions of drive coils and sense coils in a position detection unit according to a twelfth embodiment of the present invention.

FIG. 86 is a schematic diagram showing the cross-section of a medical magnetic-induction and position-detection system.

FIG. 87 is a diagram showing the positional relationship of drive coils and sense coils in a position detection unit according to a thirteenth embodiment of the present invention.

FIG. 88 is a diagram showing the positional relationship of drive coils and sense coils in a position detection unit according to a modification of the thirteenth embodiment of the present invention.

FIG. 89 is a schematic diagram of a medical magnetic-induction and position-detection system according to a fourteenth embodiment of the present invention.

FIG. 90 is a schematic diagram of a medical magnetic-induction and position-detection system according to a fifteenth embodiment of the present invention.

FIG. 91 is a diagram showing the configuration of an electromagnet system serving as a magnetic-field generating unit.

### BEST MODE FOR CARRYING OUT THE INVENTION

### First to Fifth Embodiments

### (Medical Magnetic-Induction and Position-Detection System)

### First Embodiment

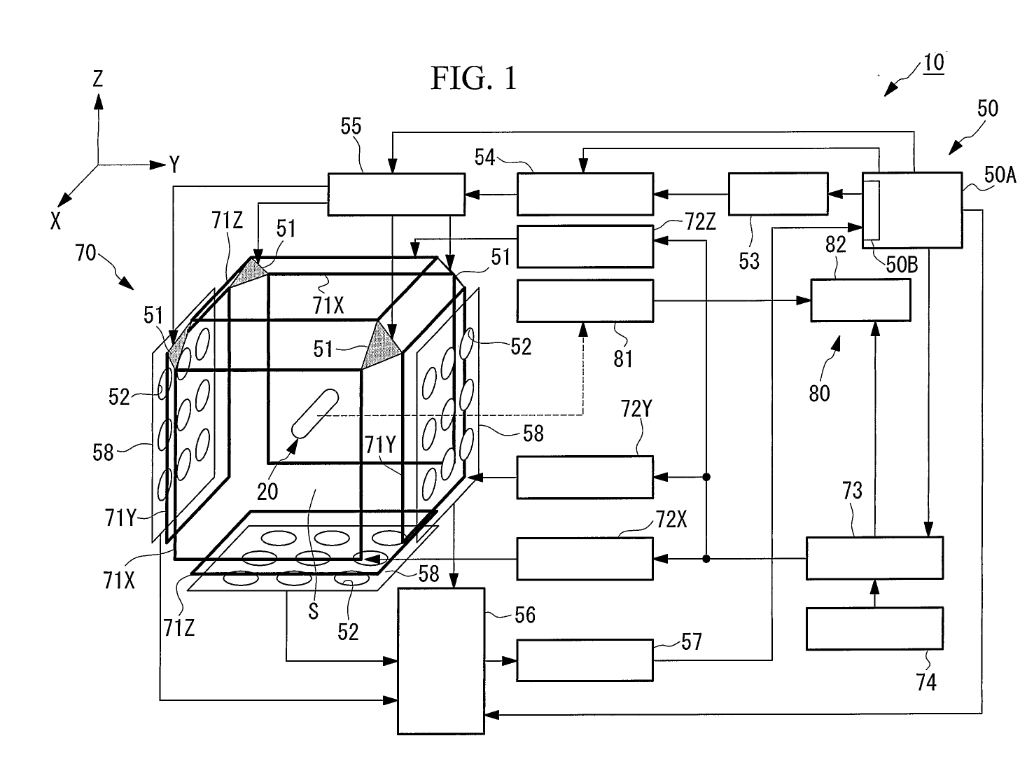

A first embodiment of a medical magnetic-induction and position-detection system according to the present invention will now be described with reference to FIGS. 1 to 13B.

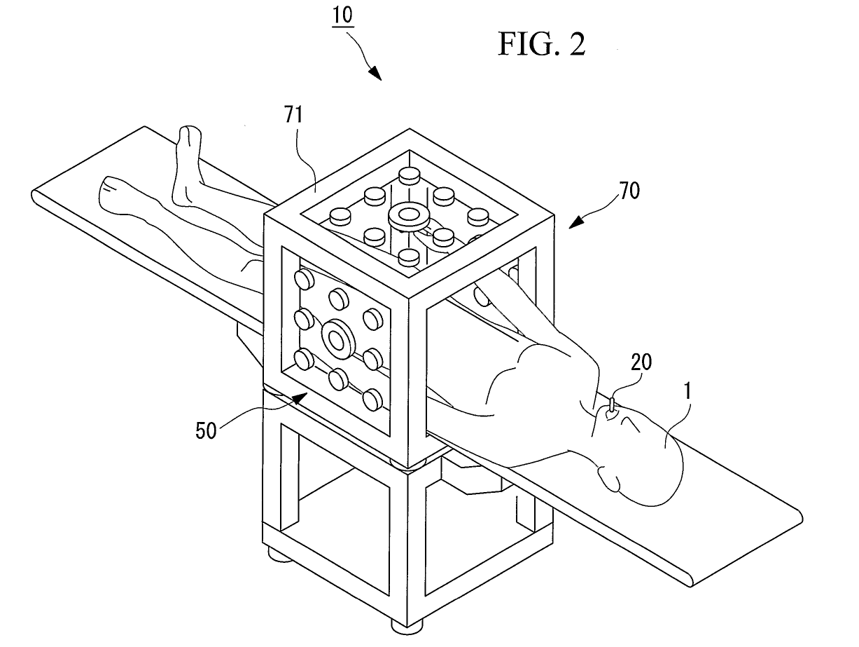

FIG. 1 is a diagram schematically showing a medical magnetic-induction and position-detection system according to this embodiment. FIG. 2 is a perspective view of the medical magnetic-induction and position-detection system.

As shown in FIGS. 1 and 2, a medical magnetic-induction and position-detection system 10 is mainly formed of a capsule endoscope (medical device) 20 that is introduced into a body cavity of a subject 1, per oral or per anus, to optically image an internal surface of a passage in the body cavity and wirelessly transmit an image signal; a position detection unit (position detection system, position detector, calculating apparatus) 50 that detects the position of the capsule endoscope 20; a magnetic induction apparatus 70 that guides the capsule endoscope 20 based on the detected position of the capsule endoscope 20 and instructions from an operator; and an image display apparatus 80 that displays the image signal transmitted from the capsule endoscope 20.

As shown in FIG. 1, the magnetic induction apparatus 70 is mainly formed of a three-axis guidance-magnetic-field generating unit (guidance-magnetic-field generating unit, electromagnet) 71 that produces parallel magnetic fields for driving the capsule endoscope 20; a Helmholtz-coil driver 72 that controls the gain of currents supplied to the three-axis guidance-magnetic-field generating unit 71; a rotation-magnetic-field control circuit (magnetic-field-orientation control unit) 73 that controls the directions of the parallel magnetic fields for driving the capsule endoscope 20; and an input device 74 that outputs to the rotation-magnetic-field control circuit 73 the direction of movement of the capsule endoscope 20 that the operator inputs.

Although the three-axis guidance-magnetic-field generating unit 71 is employed assuming that Helmholtz-coil conditions are satisfied in this embodiment, it is not necessary that the three-axis guidance-magnetic-field generating unit 71 strictly satisfies Helmholtz-coil conditions. For example, the coils may be substantially rectangular, as shown in FIG. 1, instead of circular. Furthermore, it is acceptable that the gaps between opposing coils do not satisfy Helmholtz-coil conditions as long as the function of this embodiment is achieved.

As shown in FIGS. 1 and 2, the three-axis guidance-magnetic-field generating unit 71 is formed in a substantially rectangular shape. The three-axis guidance-magnetic-field generating unit 71 includes three-pairs of mutually opposing Helmholtz coils (electromagnets, opposed coils) 71X, 71Y, and 71Z, and each pair of Helmholtz coils 71X, 71Y, and 71Z is disposed so as to be substantially orthogonal to the X, Y, and Z axes in FIG. 1. The Helmholtz coils disposed substantially orthogonally with respect to the X, Y, and Z axes are denoted as the Helmholtz coils 71X, 71Y, and 71Z, respectively.

The Helmholtz coils 71X, 71Y, and 71Z are disposed so as to form a substantially rectangular space S in the interior thereof. As shown in FIG. 1, the space S serves as an operating space of the capsule endoscope 20 and, as shown in FIG. 2, is the space in which the subject 1 is placed.

The Helmholtz-coil driver 72 includes Helmholtz-coil drivers 72X, 72Y, and 72Z for controlling the Helmholtz coils 71X, 71Y, and 71Z, respectively.

Direction-of-movement instructions for the capsule endoscope 20, which the operator inputs from the input device 74, are input to the rotation-magnetic-field control circuit 73, together with data from the position detection apparatus, to be described later, indicating the direction in which the capsule endoscope 20 is currently pointing (the direction of a rotation axis (longitudinal axis) R of the capsule endoscope 20). Then, signals for controlling the Helmholtz-coil drivers 72X, 72Y, and 72Z are output from the rotation-magnetic-field control circuit 73, and rotational phase data of the capsule endoscope 20 is output to the image display apparatus 80.

An input device for specifying the direction of movement of the capsule endoscope 20 by moving a joystick is used as the input device 74.

As mentioned above, the input device 74 may use a joystick-type device, or another type of input device may be used, such as an input device that specifies the direction of movement by pushing direction-of-movement buttons.

As shown in FIG. 1, the position detection unit 50 is mainly formed of drive coils (driving coils) 51 that generate induced magnetic fields in a magnetic induction coil (described later) in the capsule endoscope 20; sense coils (magnetic field sensors, magnetic-field detection sections) 52 that detect the induced magnetic fields generated in the magnetic induction coil; and a position detection apparatus (position analyzing unit, magnetic-field-frequency varying section, drive-coil control section) 50A that computes the position of the capsule endoscope 20 based on the induced magnetic fields that the sense coils 52 detect and that controls the alternating magnetic fields formed by the drive coils 51.

The position detection apparatus 50A is provided with a calculating-frequency determining section (frequency determining section) 50B to receive signals from a sense-coil receiving circuit to be described later.

Between the position detection apparatus 50A and the drive coils 51 there are provided a signal generating circuit 53 that generates an AC current based on the output from the position detection apparatus 50A; a drive-coil driver 54 that amplifies the AC current input from the signal generating circuit 53 based on the output from the position detection apparatus 50A; and a drive-coil selector 55 that supplies the AC current to a drive coil 51 selected on the basis of the output from the position detection apparatus 50A.

Between the sense coils 52 and the position detection apparatus 50A there are provided a sense-coil selector (magnetic-field-sensor selecting unit) 56 that selects from the sense coils 52 AC current that includes position information of the capsule endoscope 20 and so on, based on the output from the position detection apparatus 50A; and a sense-coil receiving circuit 57 that extracts an amplitude value from the AC current passing through the sense-coil selector 56 and outputs it to the position detection apparatus 50A.

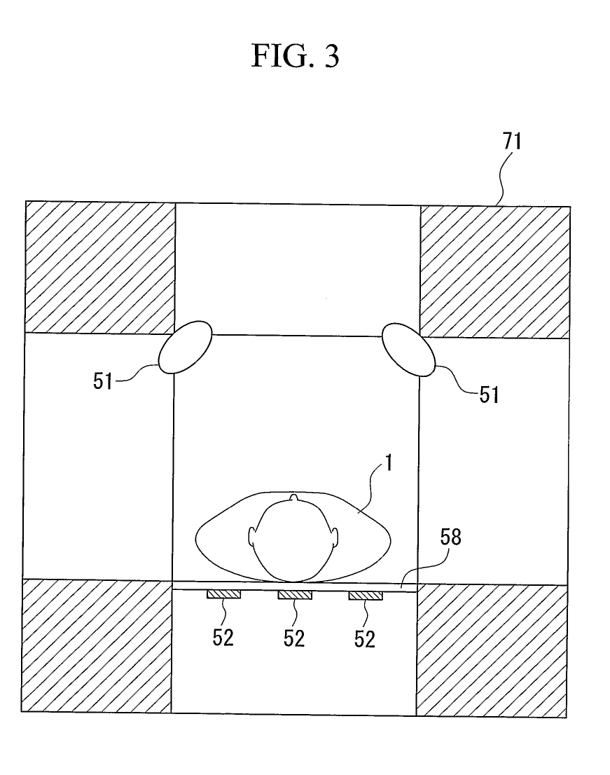

FIG. 3 is a schematic diagram showing a cross-section of the medical magnetic-induction and position-detection system.

Here, as shown in FIGS. 1 and 3, the drive coils 51 are positioned at an angle at the four upper (in the positive direction of the Z-axis) corners of the substantially rectangular operating space formed by the Helmholtz coils 71X, 71Y, and 71Z. The drive coils 51 form substantially triangular coils that connect the corners of the square-shaped Helmholtz coils 71X, 71Y, and 71Z. By disposing the drive coils 51 at the top in this way, it is possible to prevent interference between the drive coils 51 and the subject 1.

The drive coils 51 may be substantially triangular coils, as mentioned above, or it is possible to use coils of various shapes, such as circular coils, etc.

The sense coils 52 are formed as air-core coils, and are supported, at the inner side of the Helmholtz coils 71X, 71Y, and 71Z, by three planar coil-supporting parts 58 that are disposed at positions facing the drive coils 51 and at positions mutually opposing each other in the Y-axis direction, with the operating space of the capsule endoscope 20 being disposed therebetween. Nine of the sense coils 52 are arranged in the form of a matrix in each coil-supporting part 58, and thus a total of 27 sense coils 52 are provided in the position detection unit 50.

The sense coils 52 can be arranged freely. For example, the sense coils 52 may be arranged on the same surfaces as those of the Helmholtz coils 71X, 71Y, and 71Z or may be arranged outside the Helmholtz coils 71X, 71Y, and 71Z.

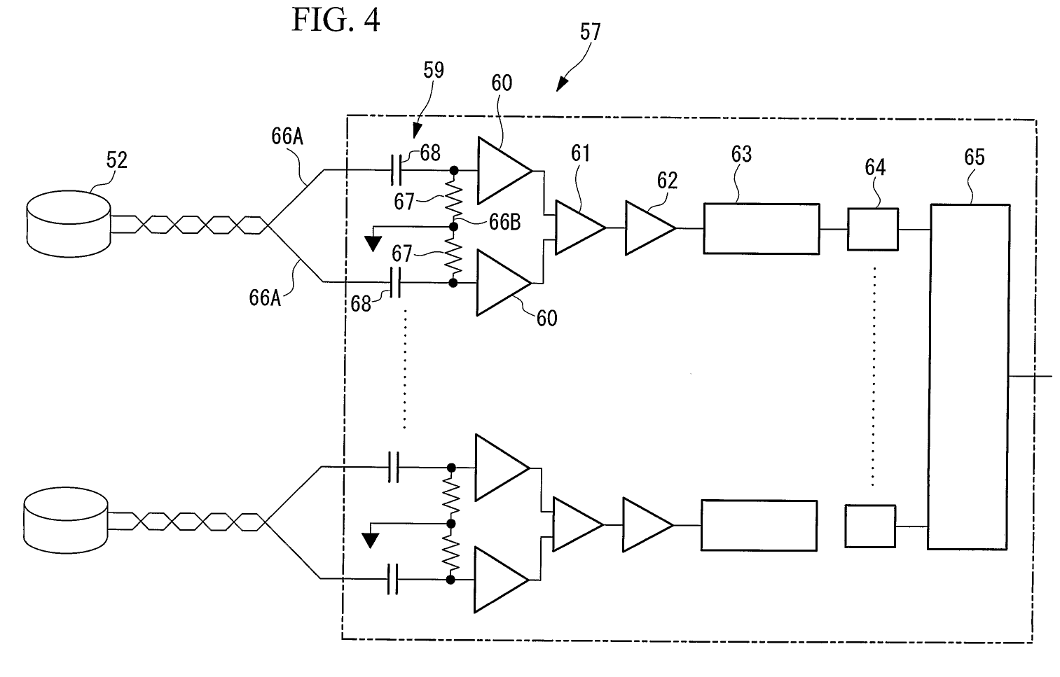

FIG. 4 is a schematic diagram showing the circuit configuration of the sense-coil receiving circuit 57.

As shown in FIG. 4, the sense-coil receiving circuit 57 is formed of a high-pass filter (HPF) 59 that removes low-frequency components of input AC voltages including the position information of the capsule endoscope 20; pre-amplifiers 60 that amplify the AC voltages; a band-pass filter (BPF, band limiting section) 61 that removes high frequencies included in the amplified AC voltages; an amplifier (AMP) 62 that amplifies the AC voltage from which the high frequencies have been removed; a root-mean-square detection circuit (True RMS converter) 63 that detects the amplitude of the AC voltage and that extracts and outputs an amplitude value; an A/D converter 64 that converts the amplitude value to a digital signal; and a memory 65 for temporarily storing the digitized amplitude value.

Here, the high-pass filter (HPF) 59 also serves to eliminate low-frequency signals which have been induced by rotating magnetic fields occurring in the Helmholtz coils 71X, 71Y, and 71Z and have been detected by the sense coils 52. By doing so, the position detection unit 50 can be operated normally while the magnetic induction apparatus 70 is being operated.

The high-pass filter 59 is formed of a pair of capacitors 68 disposed in a pair of wires 66A extending from the sense coil 52; a wire 66B that is connected to the pair of wires 66A and that is grounded substantially at the center thereof; and resistors 67 disposed opposite each other in the wire 66B, with the grounding point therebetween. The pre-amplifiers 60 are disposed in the pair of wires 66A, respectively, and the AC voltages output from the pre-amplifiers 60 are input to the single band-pass filter 61. The memory 65 temporarily stores the amplitude values obtained from the nine sense coils 52 and outputs the stored amplitude values to the position detection apparatus 50A.

In addition to the above-described components, a common-mode filter capable of removing common-mode noise may be provided.

The band-pass filter 61 may be to remove high-frequency components of the AC voltages, as mentioned above; however, the band limiting section may be a section which performs a Fourier transform.

The root-mean-square detection circuit 63 may be used to extract the amplitude value of the AC voltage, as mentioned above, the amplitude value may be detected by smoothing the magnetic field information using a rectifying circuit and detecting the voltage, or the amplitude value may be detected using a peak detecting circuit that detects a peak in the AC voltage.

Regarding the waveform of the detected AC voltage, the phase with respect to a waveform applied to the drive coil 51 changes depending on the presence and the position of a magnetic induction coil 42. This phase change may be detected with a lock-in amplifier or the like.

As shown in FIG. 1, the image display apparatus 80 is formed of an image receiving circuit 81 that receives the image transmitted from the capsule endoscope 20 and a display section (display unit, image control unit) 82 that displays the image based on the received image signal and a signal from the rotation-magnetic-field control circuit 73.

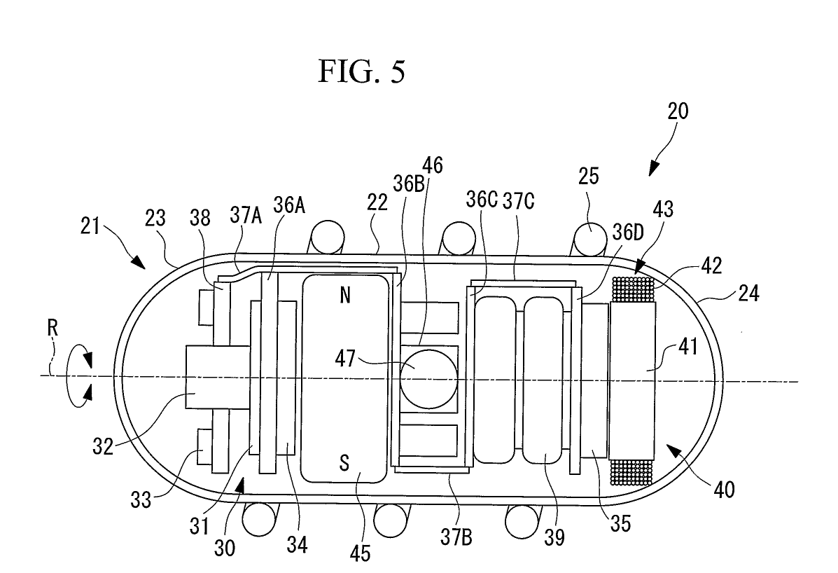

FIG. 5 is a schematic diagram showing the configuration of the capsule endoscope.

As shown in FIG. 5, the capsule endoscope 20 is mainly formed of an outer casing 21 that accommodates various devices in the interior thereof; an imaging section (biological-information acquiring unit) 30 that images an internal surface of a passage in the body cavity of the subject; a battery 39 for driving the imaging section 30; an induced-magnetic-field generating section 40 that generates induced magnetic fields by means of the drive coils 51 described above; and a guidance magnet (permanent magnet) 45 that drives the capsule endoscope 20 by receiving magnetic fields occurring in the magnetic induction apparatus 70.

The outer casing 21 is formed of an infrared-transmitting cylindrical capsule main body (hereinafter abbreviated simply as main body) 22 whose central axis defines a rotation axis (longitudinal axis) R of the capsule endoscope 20, a transparent hemispherical front end portion 23 that covers the front end of the main body 22, and a hemispherical rear end portion 24 that covers the rear end of the main body, to form a sealed capsule container with a watertight construction.

A helical part (helical mechanism) 25 in which a wire having a circular cross-section is wound in the form of a helix about the rotation axis R is provided on the outer circumferential surface of the main body of the outer casing 21.

When the guidance magnet rotates upon receiving rotating magnetic fields generated in the magnetic induction apparatus 70, this helical part also rotates to guide the capsule endoscope 20 in the direction of the rotation axis R in the passage in the body cavity of the subject.

The imaging section 30 is mainly formed of a board 36A positioned substantially orthogonal to the rotation axis R; an image sensor 31 disposed on the surface at the front end portion 23 side of the board 36A; a lens group 32 that forms an image of the internal surface of the passage inside the body cavity of the subject on the image sensor 31; an LED (Light Emitting Diode) 33 that illuminates the internal surface of the passage inside the body cavity; a signal processing section 34 disposed on the surface at the rear end portion 24 side of the board 36A; and a radio device 35 that transmits the image signal to the image display apparatus 80.

The signal processing section 34 is electrically connected to the battery 39 via the board 36A, boards 36B, 36C, and 36D, and flexible boards 37A, 37B, and 37C, is electrically connected to the image sensor 31 via the board 36A, and is electrically connected to the LED 33 via the board 36A, the flexible board 37A, and a support member 38. Also, the signal processing section 34 compresses the image signal that the image sensor 31 acquires, temporarily stores it (memory), and transmits the compressed image signal to the exterior from the radio device 35, and in addition, it controls the on/off state of the image sensor 31 and the LED 33 based on signals from a switch section 46 to be described later.

The image sensor 31 converts the image formed via the front end portion 23 and the lens group 32 to an electrical signal (image signal) and outputs it to the signal processing section 34. CMOS (Complementary Metal Oxide Semiconductor) devices or CCDs (Charge Coupled Devices), for example, can be used as this image sensor 31.

Moreover, a plurality of the LEDs 33 are disposed on the support member 38 positioned towards the front end portion 23 from the board 36A such that gaps are provided therebetween in the circumferential direction around the rotation axis R.

The guidance magnet 45 is disposed at the rear end portion 24 side of the signal processing section 34. The guidance magnet 45 is disposed or polarized so as to have a magnetization direction in a direction orthogonal to the rotation axis R (for example, in the vertical direction in FIG. 5).

The switch section 46, which is disposed on the board 36B, is provided at the rear end portion 24 side of the guidance magnet 45. The switch section 46 has an infrared sensor 47, is electrically connected to the signal processing section 34 via the board 36B and the flexible board 37A, and is electrically connected to the battery 39 via the boards 36B, 36C, and 36D and the flexible boards 37B and 37C.

Also, a plurality of the switch sections 46 are disposed in the circumferential direction about the rotation axis R at regular intervals, and the infrared sensor 47 is disposed so as to face the outside in the diameter direction. In this embodiment, an example has been described in which four switch sections 46 are disposed, but the number of switch sections 46 is not limited to four; any number may be provided.

At the rear end portion 24 side of the switch section 46, the battery 39 is disposed so as to be sandwiched by the boards 36C and 36D.

The radio device 35 is disposed on the surface of the board 36D at the rear end portion 24 side. The radio device 35 is electrically connected to the signal processing section 34 via the boards 36A, 36B, 36C, and 36D and the flexible boards 37A, 37B, and 37C.

The induced-magnetic-field generating section 40 is disposed at the rear end portion 24 side of the radio device 35. The induced-magnetic-field generating section 40 is formed of a core member 41 made of ferrite formed in the shape of a cylinder whose central axis is substantially the same as the rotation axis R; the magnetic induction coil 42 that is disposed at the outer circumferential part of the core member 41; and a capacitor (not shown in the drawing) that is electrically connected to the magnetic induction coil 42 and that forms a resonance circuit 43.

The capacitance of the capacitor is determined in accordance with the inductance of the magnetic induction coil 42 so that the resonance frequency of the resonance circuit 43 is close to the frequency of the alternating magnetic fields generated by the drive coils 51 of the position detection unit 50. In addition, the frequency of the alternating magnetic fields generated by the drive coils 51 may be determined in accordance with the resonance frequency of the resonance circuit 43.

In addition to ferrite, magnetic materials are suitable for the core member; iron, nickel, permalloy, cobalt or the like may be used for the core member.

Next, the operation of the medical magnetic-induction and position-detection system 10 having the above-described configuration will be described.

First, an overview of the operation of the medical magnetic-induction and position-detection system 10 will be described.

As shown in FIGS. 1 and 2, the capsule endoscope 20 is inserted, per oral or per anus, into a body cavity of a subject 1 who is lying down inside the position detection unit 50 and the magnetic induction apparatus 70. The position of the inserted capsule endoscope 20 is detected by the position detection unit 50, and it is guided to the vicinity of an affected area inside a passage in the body cavity of the subject 1 by the magnetic induction apparatus 70. The capsule endoscope 20 images the internal surface of the passage in the body cavity while being guided to the affected area and in the vicinity of the affected area. Then, data for the imaged internal surface of the passage inside the body cavity and data for the vicinity of the affected area are transmitted to the image display apparatus 80. The image display apparatus 80 displays the transmitted images on the display section 82.

A procedure for obtaining calculating frequencies used to detect the position and direction of the capsule endoscope 20 and a procedure for detecting the position and direction of the capsule endoscope 20 will now be described.

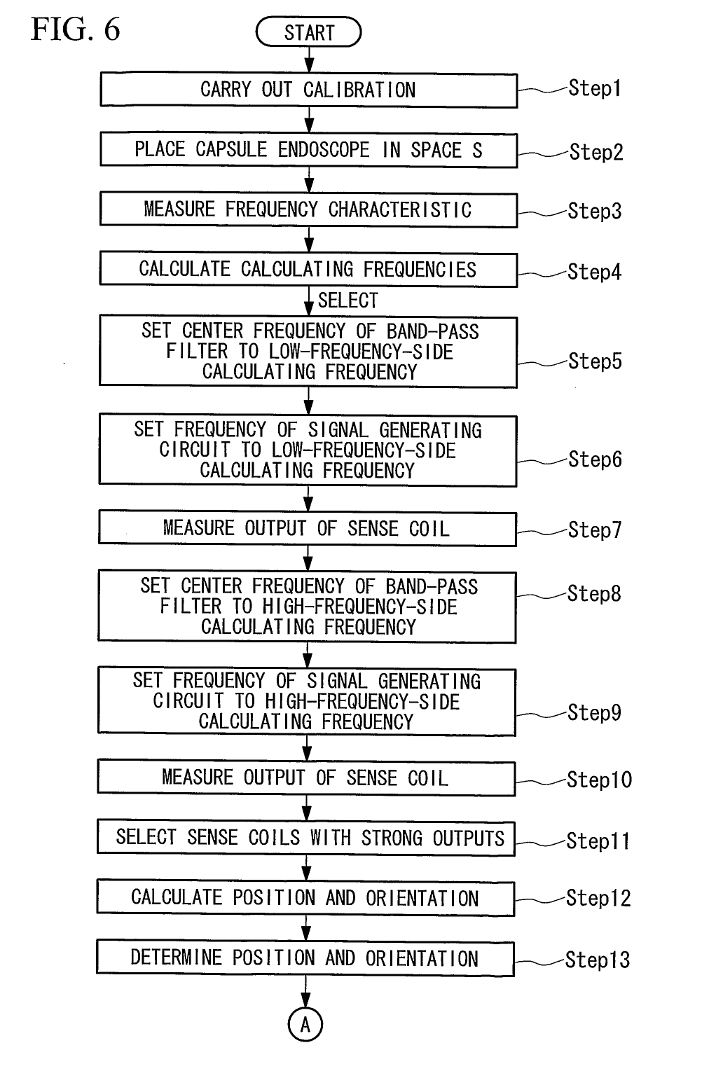

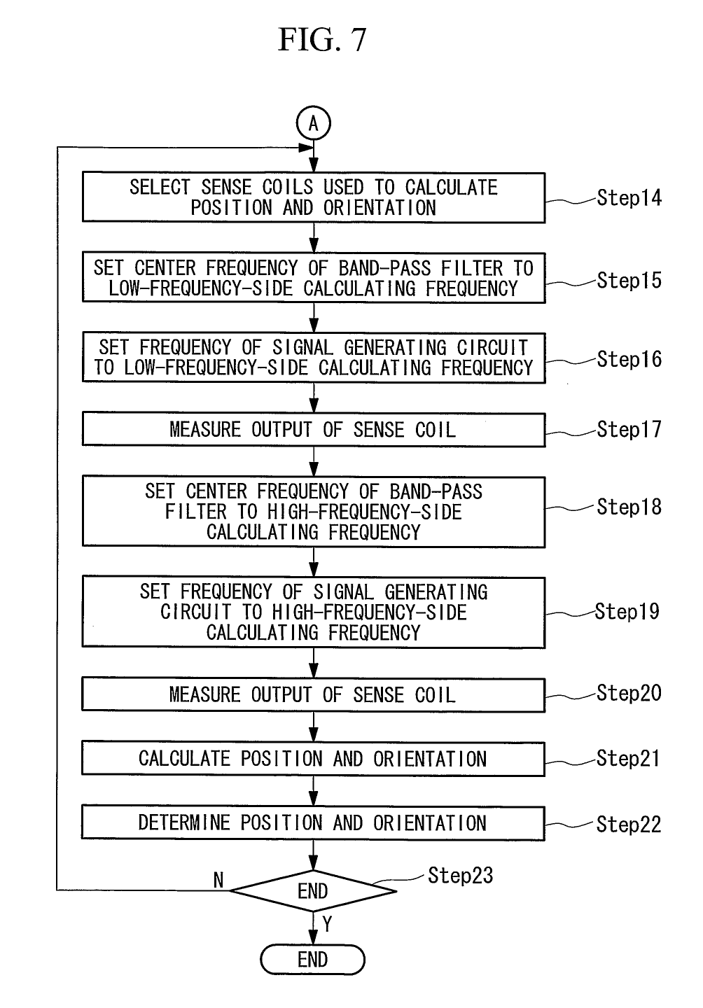

FIGS. 6 and 7 are flowcharts illustrating the procedures for obtaining calculating frequencies and for detecting the position and direction of the capsule endoscope 20.

First, as shown in FIG. 6, calibration of the position detection unit 50 is carried out (Step 1; preliminary measuring step). More specifically, the output of the sense coils 52 while the capsule endoscope 20 is not disposed in the space S, namely, the output of the sense coils 52 resulting from the operation of alternating magnetic fields formed by the drive coils 51 is measured.

A specific procedure for forming alternating magnetic fields is illustrated in FIG. 1. That is, the signal generating circuit 53 generates an AC signal, which is then output to the drive-coil driver 54. The drive-coil driver 54 power-amplifies the AC signal to supply AC current to the drive coils 51 via the drive-coil selector 55. The frequency of the generated AC current is in a frequency range from a few kHz to 100 kHz, and the frequency varies (sweeps) within the above-mentioned range over time, so as to include a resonance frequency to be described later. The resonance frequency at this stage may be obtained through estimation from the characteristic values of the magnetic induction coil 42, the capacitor, or the like. In addition, this frequency may be set to any value, as described later.

The sweep range is not limited to the range mentioned above; it may be a narrower range or it may be a wider range, and is not particularly limited.

The AC signal is amplified in the drive-coil driver 54 based on an instruction from the position detection apparatus 50A and is output to the drive-coil selector 55 as AC current. The amplified AC current is supplied to the drive coil 51 selected by the position detection apparatus 50A in the drive-coil selector 55. Then, the AC current supplied to the drive coil 51 produces an alternating magnetic field in the operating space S of the capsule endoscope 20.

As shown in FIG. 4, the formed alternating magnetic field generates an induced electromotive force in the sense coils 52 to cause an AC voltage in the sense coils 52. This AC voltage is input to the sense-coil receiving circuit 57 via the sense coil selector 56, where an amplitude value of the AC voltage is extracted.

As shown in FIG. 4, low frequency components included in the AC voltage input to the sense-coil receiving circuit 57 are first removed by the high-pass filter 59, and the AC voltage is then amplified by the pre-amplifiers 60. Thereafter, high frequencies are removed by the band-pass filter 61, and the AC voltage is amplified by the amplifier 62. The amplitude value of the AC voltage from which unwanted components have been removed in this way is extracted by the root-mean-square detection circuit 63. The extracted amplitude value is converted to a digital signal by the A/D converter 64 and is stored in the memory 65. At this time, the transmission frequency of the band-pass filter 61 is adjusted to the frequency of the alternating magnetic field for each operation.

The memory 65 stores, for example, an amplitude value corresponding to one period in which the signal generated in the signal generating circuit 53 is swept close to the resonance frequency of the resonance circuit 43, and outputs the amplitude value for one period at a time to the frequency determining section 50B of the position detection apparatus 50A. The output value at this time is expressed as Vc(f,N), where Vc is a function of f, the frequency of the alternating magnetic field, and N, the number of the sense coil.

Next, the capsule endoscope 20 is placed in the space S (Step 2). The procedure for placing the capsule endoscope 20 is not specifically limited. For example, the capsule endoscope 20 may be placed on a holder, if one is provided in the space S, to support the capsule endoscope.

Furthermore, this holder may directly support the capsule endoscope 20 or may support the capsule endoscope housed in a package (not shown in the figure). This configuration is hygienic.

Then, a frequency characteristic of the magnetic induction coil 42 installed in the capsule endoscope 20 is measured (Step 3; measuring step). More specifically, in the same manner as in Step 1, the drive coils 51 are made to produce alternating magnetic fields whose frequency changes over a predetermined band, and the output of the sense coils 52 resulting from the alternating magnetic fields and the magnetic field induced by the magnetic induction coil 42 is measured while the frequency is being changed (swept). At this time, the output is expressed as V0(f,N), where f is the frequency of the alternating magnetic field and N is the number of the sense coil 52.

Since the magnetic induction coil 42 forms the resonance circuit 43 together with the capacitor, induced current flowing in the resonance circuit 43 (magnetic induction coil 42) increases and the induced magnetic field produced becomes intense when the period of the alternating magnetic fields corresponds to the resonance frequency of the resonance circuit 43. In addition, since the core member 41 composed of dielectric ferrite is disposed in the center of the magnetic induction coil 42, the induced magnetic field is more easily concentrated in the core member 41, which causes the induced magnetic field produced to be even more intense.

Thereafter, the frequency determining section 50B calculates the difference between the output of the sense coils 52 measured in Step 1 and the output of the sense coils 52 measured in Step 3, and calculating frequencies used for the detection of the position and orientation of the capsule endoscope 20 are obtained based on the calculated difference (Step 4; frequency determination step).

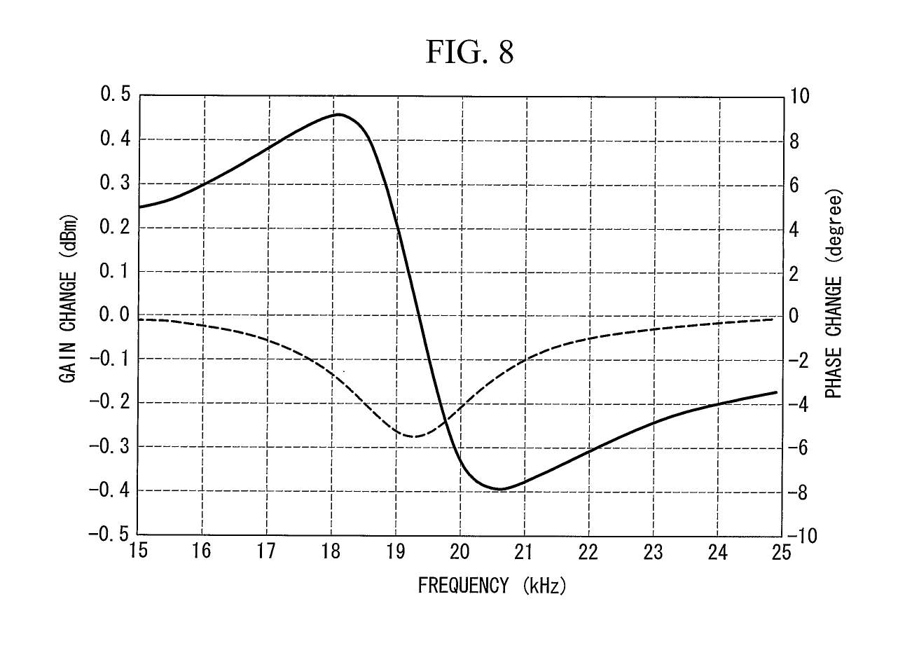

FIG. 8 is a diagram depicting the frequency characteristic of the magnetic induction coil 42, and illustrates changes in gain and phase of the output of a sense coil 52 in association with a change in the frequency of the alternating magnetic field. The gain V(f,N) of this graph is expressed as V(f,N)=V0(f,N)−Vc(f,N). That is, the gain V(f,N) is represented by the difference between the measurement in step 1 and the measurement in step 3 at each frequency.

As shown in FIG. 8, the amplitude value of the AC voltage, which is the output of the sense coil 52, greatly changes depending on the frequency characteristic of the alternating magnetic field generated by the magnetic induction coil 42, namely, the relationship with the resonance frequency of the resonance circuit 43. FIG. 8 shows the frequency of the alternating magnetic field on the horizontal axis and the variations in gain (dBm) and phase (degree) of the AC voltage flowing in the resonance circuit 43 on the vertical axes. It is shown in FIG. 8 that the variation in gain, indicated by the solid line, exhibits a maximum value at a frequency smaller than the resonance frequency, is zero at the resonance frequency, and exhibits a minimum value at a frequency higher than the resonance frequency. Also, it is shown that the variation in phase, indicated by the broken line, drops most at the resonance frequency. Here, it has been confirmed by measuring the impedance characteristic of the resonant circuit with a network analyzer, an impedance analyzer, or the like that the resonance frequency of the resonance circuit 43 corresponds to the frequency that causes the largest phase lag and to the frequency that causes the gain to cross 0.

Depending on the measurement conditions, there may be cases where the gain exhibits a minimum value at a frequency lower than the resonance frequency and exhibits a maximum value at a frequency higher than the resonance frequency, and where the phase reaches a peak at the resonance frequency.

More specifically, frequencies at which the change in gain of the above-described sense coil 52 exhibits the maximum and minimum values are obtained, and these two frequencies are used as the calculating frequencies: the lower frequency is used for the low-frequency-side calculating frequency and the higher frequency for the high-frequency-side calculating frequency. As shown in FIG. 8, the gain change exhibits its maximum and minimum values at frequencies of about 18 kHz and about 20.5 kHz, respectively. The former is the low-frequency-side calculating frequency, and the latter is the high-frequency-side calculating frequency.

In this manner, the use of the difference between the output of the sense coils 52 in Step 1 and the output of the sense coils 52 in Step 2 allows high-precision calculating frequencies to be obtained by eliminating adverse effects, such as a drift in the output value related to the temperature characteristic of the sense-coil receiving circuit 57.

Here, Vc(f_{LOW},N), Vc(f_{HIGH},N), (N: 1, 2, 3, . . . the number of the sense coils) for all sense coils are stored as reference values, where f_{LOW} represents the low-frequency-side calculating frequency and f_{HIGH} represents the high-frequency-side calculating frequency. In Step 5 and the subsequent steps, Vₛ(f_{LOW},N) and Vₛ(f_{HIGH},N) calculated based on the output of the sense coils 52 for the values used for position calculation are calculated by the following calculating formulas, where V(f_{LOW},N) (N is the number of the sense coil) represents the output of the sense coils 52 measured at the low-frequency-side calculating frequency (f_{LOW}) and V(f_{HIGH},N) (N is the number of the sense coil) represents the output of the sense coils 52 measured at the high-frequency-side calculating frequency (f_{HIGH}).
Vₛ(f_{LOW},N)=V(f_{LOW},N)−V_{c}(f_{LOW},N)
Vₛ(f_{HIGH},N)=V(f_{HIGH},N)−V_{c}(f_{HIGH},N)

Thus, in the subsequent steps, Vₛ(f_{LOW},N) and Vₛ(f_{HIGH},N) are represented as “values calculated based on the output of the sense coil 52”.

When the above-described calculating frequencies are to be obtained, the output of at least one sense coil 52 is sufficient to obtain a low-frequency-side calculating frequency and a high-frequency-side calculating frequency. More specifically, although the output frequency characteristics for all sense coils 52 are measured in step 1, it is sufficient to measure for a specific sense coil 52 in step 3 and to perform the processing of step 4 to obtain the calculating frequencies.

First, one sense coil 52 is selected. Then, alternating magnetic fields are produced from the drive coils 51 while being swept. At this time, the center frequency of the band-pass filter 61 connected to the selected sense coil 52 is swept (changed) in accordance with the frequency of the alternating magnetic fields generated by the drive coils 51. The output (output through the band-pass filter 61, amplifier 62, and True RMS converter 63) of the sense coil 52 is measured while the alternating magnetic fields generated by the drive coils 51 are being swept.

Thereafter, the capsule endoscope 20 is placed in the space S. In the same manner as described above, alternating magnetic fields are produced from the drive coils 51 while being swept, and the center frequency of the band-pass filter 61 connected to the selected sense coil 52 is swept in accordance with the frequency of the alternating magnetic fields generated from the driver coils 51 to measure the output of the sense coil 52.

Then, the difference between the measurement (output of the sense coil 52) while the capsule endoscope 20 is not placed in the space S and the measurement (output of the sense coil 52) while the capsule endoscope 20 is placed in the space S is obtained.

The result is as shown in FIG. 8 described above, and thus calculating frequencies can be obtained.

Calibration of all sense coils 52 is carried out as follows. After the calculating frequencies have been determined, the capsule endoscope 20 is removed from the space S again and the center frequency of the band-pass filter 61 is adjusted to the low-frequency-side calculating frequency. Then, the frequency of the alternating magnetic fields formed by the drive coils 51 is adjusted to the low-frequency-side calculating frequency. Alternating magnetic fields with the low-frequency-side calculating frequency are generated by the drive coils 51 and the outputs of all sense coils 52 are measured. These measurements are saved as V_{c}(f_{LOW},N).

In the subsequent step, the center frequency of the band-pass filter 61 is adjusted to the high-frequency-side calculating frequency. Then, the frequency of the alternating magnetic fields formed by the drive coils 51 is adjusted to the high-frequency-side calculating frequency, and alternating magnetic fields with the high-frequency-side calculating frequency are generated by the drive coils 51. The outputs of all sense coils 52 are measured. These values are saved as V_{c}(f_{HIGH},N).

After these calculating frequencies have been obtained, the position and direction of the capsule endoscope 20 are detected.

First, the center frequency of the band-pass filter 61 is adjusted to the low-frequency-side calculating frequency (Step 5). Furthermore, the transmission frequency range of the band-pass filter 61 is set to such a range that local extreme values of a change in gain of the sense coils 52 can be extracted.

Then, the frequency of the alternating magnetic fields formed by the drive coils 51 is adjusted to the low-frequency-side calculating frequency (Step 6). More specifically, the frequency of the alternating magnetic fields formed by the drive coils 51 is controlled by controlling the frequency of AC current generated by the signal generating circuit 53 to the low-frequency-side calculating frequency.

Then, alternating magnetic fields with the low-frequency-side calculating frequency are produced by the drive coils 51 to detect the magnetic field induced by the magnetic induction coil 42 with the sense coil 52 (Step 7; detection step) In short, the output of the sense coils 52 is measured, and Vₛ(f_{LOW},N), which is a value calculated based on the output of the sense coils 52, is obtained, where N indicates the number of the selected sense coil 52.

Next, the center frequency of the band-pass filter 61 is adjusted to the high-frequency-side calculating frequency (Step 8).

Then, the frequency of alternating magnetic fields formed by the drive coils 51 is adjusted to the high-frequency-side calculating frequency (Step 9).

Alternating magnetic fields with the high-frequency-side calculating frequency are produced by the drive coils 51 to detect the magnetic field induced by the magnetic induction coil 42 with the sense coils 52 (Step 10; detection step). In short, the output of the sense coils 52 is measured to obtain Vₛ(f_{HIGH},N), which is a value calculated based on the output of the sense coils 52, where N indicates the number of the sense coil 52.

As described above, detection with the low-frequency-side calculating frequency can be performed first, followed by detection with the high-frequency-side calculating frequency. Alternatively, detection with the high-frequency-side calculating frequency may be performed first, followed by detection with the low-frequency-side calculating frequency.

Thereafter, the position detection apparatus 50A calculates Vₛ(f_{LOW},N)−Vₛ(f_{HIGH},N), which indicates the output difference (amplitude difference) of each sense coil 52 between the low-frequency-side calculating frequency and the high-frequency-side calculating frequency, and then the sense coils 52 whose output difference is to be used to estimate the position of the capsule endoscope 20 are selected (Step 11).

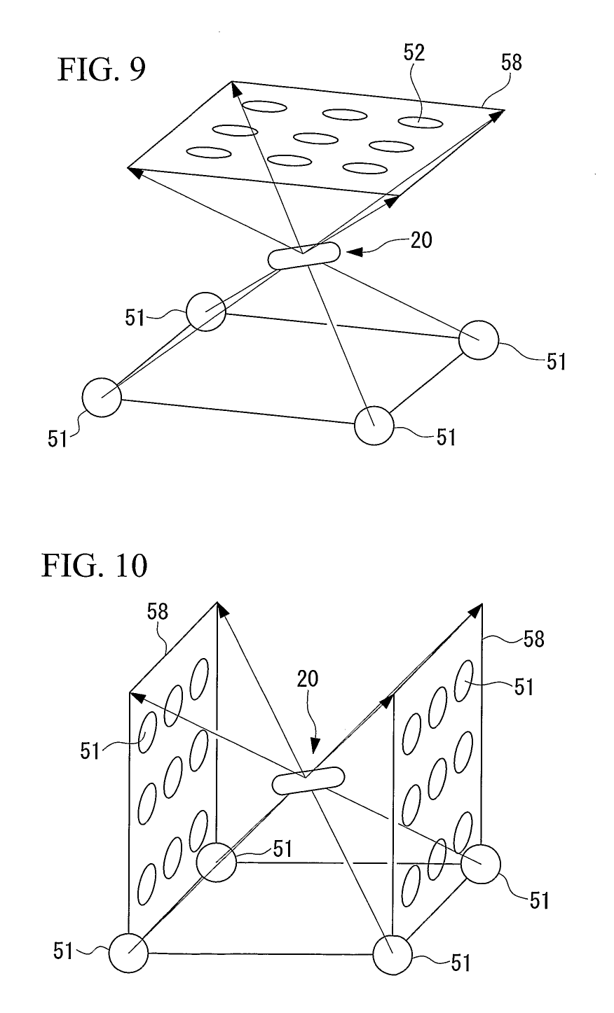

The method for selecting sense coils 52 is not limited to a particular one, as long as sense coils 52 with a large output difference can be selected. For example, sense coils 52 facing the drive coils 51 with the capsule endoscope 20 disposed therebetween may be selected, as shown in FIG. 9. Alternatively, sense coils 52 which are disposed in mutually opposing planes adjacent to the plane in which the drive coils 51 are disposed may be selected, as shown in FIG. 10.

The position detection apparatus 50A issues to the sense coil selector 56 a command for inputting the AC current from selected sense coils 52 to the sense-coil receiving circuit 57 to select the sense coils 52.

Then, the position detection apparatus 50A calculates the position and orientation of the capsule endoscope 20 based on the output difference of the selected sense coils 52 (Step 12; position calculating step) to determine the position and orientation (Step 13).

More specifically, the position detection apparatus 50A obtains the position of the capsule endoscope 20 by solving simultaneous equations involving the position, direction, and magnetic field intensity of the capsule endoscope 20 based on the amplitude difference calculated from the selected sense coils 52.

Thus, based on the output difference of the sense coils 52, it is possible to cancel changes in characteristics of the sense-coil receiving circuit due to, for example, environmental conditions (e.g., temperature), and it is therefore possible to obtain the position of the capsule endoscope 20 with a reliable degree of accuracy without being affected by environmental conditions.

The information on the position and so forth of the capsule endoscope 20 includes six pieces of information, for example, X, Y, and Z positional coordinates, directions φ and θ of the longitudinal axis (rotation axis) of the capsule endoscope 20, and the intensity of the induced magnetic field that the magnetic induction coil 42 produces.

In order to estimate these six pieces of information by calculation, the outputs of at least six sense coils 52 are necessary. Therefore, it is preferable that at least six sense coils 52 be selected in the selection of Step 11.

Then, sense coils 52 used for the subsequent control are selected as shown in FIG. 7 (Step 14).

More specifically, the position detection apparatus 50A obtains by calculation the intensity of a magnetic field produced from the magnetic induction coil 42 at the position of each sense coil 52 based on the position and orientation of the capsule endoscope 20 calculated in Step 13, and selects as many sense coils 52 as necessary disposed at positions where the magnetic field intensity is high. When the acquisition of the position and orientation of the capsule endoscope is to be repeated, sense coils 52 are selected based on the position and orientation of the capsule endoscope 20 calculated in Step 22 to be described later.

Although the number of selected sense coils 52 should be at least six in this embodiment, about ten to fifteen selected sense coils 52 are advantageous in minimizing errors in position calculation. Alternatively, sense coils 52 may be selected in such a manner that the outputs of all sense coils 52 resulting from the magnetic field produced from the magnetic induction coil 42 are calculated based on the position and orientation of the capsule endoscope 20 obtained in Step 13 (or Step 22 to be described later), and then as many sense coils 52 as necessary that have large outputs are selected.

Thereafter, the center frequency of the band-pass filter 61 is re-adjusted to the low-frequency-side calculating frequency (Step 15).

Then, the frequency of the alternating magnetic fields formed by the drive coils 51 is adjusted to the low-frequency-side calculating frequency (Step 16).

Then, alternating magnetic fields with the low-frequency-side calculating frequency are generated by the drive coils 51 to detect the magnetic field induced by the magnetic induction coil 42 with the sense coils 52 selected in Step 14 (Step 17; detection step). In the same manner as in Step 7, Vₛ(f_{LOW},N), which is a value calculated based on the output of the sense coils 52, is obtained.

Next, the center frequency of the band-pass filter 61 is adjusted to the high-frequency-side calculating frequency (Step 18).

Then, the frequency of the alternating magnetic fields formed by the drive coils 51 is adjusted to the high-frequency-side calculating frequency (Step 19).

Then, alternating magnetic fields with the high-frequency-side calculating frequency are produced by the drive coils 51 to detect the magnetic field induced by the magnetic induction coil 42 with the sense coils 52 selected in Step 13 (Step 20; detection step). Then, in the same manner as in Step 10, Vₛ(f_{HIGH},N), which is a value calculated based on the output of the sense coils 52, is obtained.

Then, the position detection apparatus 50A calculates the position and orientation of the capsule endoscope 20 based on the output difference of the sense coils 52 selected in Step 14 (Step 21; position calculating step) to determine the position and orientation (Step 22).

In Step 22, data for the calculated position and orientation of the capsule endoscope apparatus 20 may be output to another apparatus or the display section 82.

Thereafter, if detection of the position and orientation of the capsule endoscope apparatus 20 is to be continued, the flow returns to Step 14, where detection of the position and orientation is carried out.

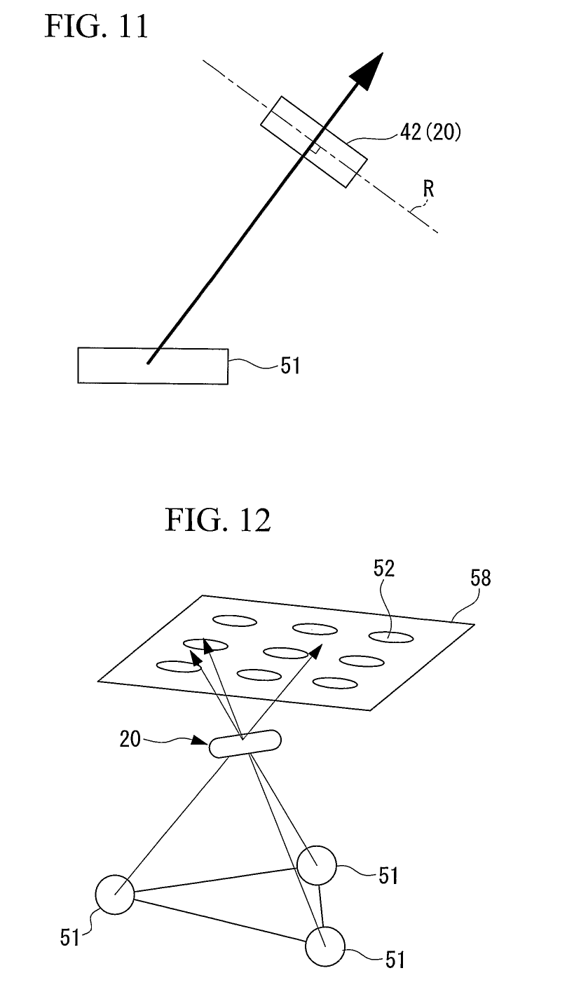

Also, in parallel with the above-described control operation, the position detection apparatus 50A selects drive coils 51 for producing magnetic fields, and outputs to the drive coil selector 55 an instruction for supplying the AC current to the selected drive coils 51. As shown in FIG. 11, in the method of selecting the drive coils 51, a drive coil 51 for which a straight line (orientation of the drive coil 51) connecting the drive coil 51 and the magnetic induction coil 42 and the central axis of the magnetic induction coil 42 (the rotation axis R of the capsule endoscope 20) are substantially orthogonal is excluded. In addition, as shown in FIG. 12, the drive coils 51 are selected so as to supply the AC current to three of the drive coils 51 in such a way that the orientations of the magnetic fields acting on the magnetic induction coil 42 are linearly independent.

A more preferable method is a method in which a drive coil 51 for which the orientation of the line of magnetic force produced by the drive coil 51 and the central axis of the magnetic induction coil 42 are substantially orthogonal is omitted.

The number of drive coils 51 forming the alternating magnetic field may be limited using the drive-coil selector 55, as described above, or the number of drive coils 51 disposed may be initially set to three without using the drive-coil selector 55.

As described above, three drive coils 51 may be selected to form the alternating magnetic field, or as shown in FIG. 9, the alternating magnetic field may be produced by all of the drive coils 51.

Switching among the drive coils 51 will now be described more specifically.

The operation of switching among the drive coils is performed as a measure against a possible problem such as, if the direction of the magnetic field produced by a drive coil 51 is orthogonal to the orientation of the magnetic induction coil 42 at the position of the capsule endoscope 20, an induced magnetic field produced by the magnetic induction coil 42 becomes small and therefore the accuracy of position detection is decreased.

The direction of the magnetic induction coil 42, namely, the direction of the capsule endoscope 20 can be recognized from an output of the position detection apparatus 50A. Furthermore, the direction of the magnetic field generated by a drive coil 51 at the position of the capsule endoscope 20 can be obtained by calculation.

Therefore, the angle between the orientation of the capsule endoscope 20 and the direction of the magnetic field produced by the drive coil 51 at the position of the capsule endoscope 20 can be obtained by calculation.

In the same manner, the directions of the magnetic fields at the position of the capsule endoscope 20, i.e., the magnetic fields generated by individual drive coils 51 disposed at different positions and orientations, can also be obtained by calculation. In the same manner, the angles between the orientation of the capsule endoscope 20 and the directions of the magnetic fields produced by the respective drive coils 51 at the position of the capsule endoscope 20 can be obtained by calculation.

By doing so, the induced magnetic field produced by the magnetic induction coil 42 can be maintained large by selecting only drive coils 51 with acute angles, at the position of the capsule endoscope 20, between the orientation of the capsule endoscope 20 and the directions of the magnetic fields produced thereby. This is advantageous in position detection.

To perform the operation of switching among the drive coils 51, the processing described below is carried out in the calibration of Step 1.

First, one drive coil 51 is selected, and an alternating magnetic field is generated by the drive coil 51 while the frequency is being changed (swept). At this time, the outputs of all sense coils 52 are measured while the center frequency of the band-pass filter 61 disposed downstream of each sense coil 52 is adjusted to the frequency of the alternating magnetic field produced by the drive coil 51 to obtain the frequency characteristics of the sense coils 52 associated with the drive coil 51.

Then, the frequency characteristics of all sense coils are stored in association with the selected drive coil 51.

Next, another drive coil 51 is selected, and an alternating magnetic field is generated by the drive coil 51 while the frequency is being changed (swept). At this time, the outputs of all sense coils 52 are measured while the center frequency of the band-pass filter 61 disposed downstream of each sense coil 52 is adjusted to the frequency of the alternating magnetic field produced by the drive coil 51 to obtain the frequency characteristics of the sense coils 52 associated with the drive coil 51.

Then, the frequency characteristics of all sense coils are stored in association with the newly selected drive coil 51.

This operation can be repeated for all drive coils to store the frequency characteristics of the sense coils 52 for all combinations of the drive coils 51 and sense coils 52.

Next, as described above, the capsule endoscope 20 is placed in the space S (STEP 2), and the frequency characteristic is measured while the capsule endoscope 20 is placed in the space S. For the measurement at this time, after any drive coil 51 and any sense coil 52 are selected, the frequency characteristic of the output of the sense coil 52 is calculated for that combination (STEP 3).

The difference between the result acquired in STEP 3 and the frequency characteristic of the sense coil 52, stored in STEP 1, for the combination of the drive coil 51 and the sense coil 52 selected in STEP 3 is obtained at each frequency component. The result is as shown in FIG. 8. Then, calculating frequencies are selected as described above.

Then, from the frequency characteristics of the sense coils 52 for all combinations of drive coils 51 and sense coils 52 obtained STEP 1, the outputs of the sense coils at the calculating frequencies for all combinations of drive coils 51 and sense coils 52 while the capsule endoscope 20 is out of the space S are extracted. Although this corresponds to the above-described V_{c}(f_{LOW},N) and V_{c}(f_{HIGH},N), denotations V_{c}(f_{LOW},N,M) and V_{c}(f_{HIGH},N,M) are used here considering associations with all drive coils, where N indicates the number of the sense coil and M indicates the number of the drive coil.

STEP 5 has already been described and thus will not be described again here.

In STEP 6, the frequency of the signal generating circuit is set to the low-frequency-side calculating frequency, and in addition, the drive-coil selector 55 is operated by the position detection apparatus 50A to select a drive coil 51 as a drive coil for output.

In STEP 7, the outputs of all sense coils are measured. The measurement at this time is carried out as described above.

Then, Vₛ(f_{LOW},N)=V(f_{LOW},N)−V_{c}(f_{LOW},N,M) which is a value calculated based on the output of the sense coils 52, is obtained, where M is the number of the drive coil selected in STEP 6. STEP 5 has already been described and thus will not be described again here.

In STEP 9, the above-described operation is carried out with the drive coil 52 selected in STEP 6 as-is.

In STEP 10, the outputs of all sense coils are measured. The measurement at this time is the same as the above-described V(f_{HIGH},N).

Then, Vₛ(f_{HIGH},N)=V(f_{HIGH},N)−V_{c}(f_{HIGH},N,M), which is a value calculated based on the output of the sense coils 52, is obtained, where M is the number of the drive coil selected in STEP 6.

STEP 11, STEP 12, and STEP 13 have already been described and thus will not be described again here.

In STEP 14, sense coils used for the subsequent position calculation are selected, and a drive coil used for the subsequent measurement is selected.

The selection of sense coils is the same as described above, and thus will not be repeated. The procedure for selecting a drive coil will now be described.

First, the direction of the magnetic field produced by a drive coil 51 at the position of the capsule endoscope 20 is obtained by calculation. Then, the angle between the orientation of the capsule endoscope 20 and the direction of the magnetic field produced by the drive coil 51 at the position of the capsule endoscope 20 is calculated.

In the same manner, the directions of the magnetic fields at the position of the capsule endoscope 20, i.e., the magnetic fields generated by individual drive coils 51 disposed at different positions and orientations, can also be obtained by calculation. In the same manner, the angles between the orientation of the capsule endoscope 20 and the directions of the magnetic fields produced by the respective drive coils 51 at the position of the capsule endoscope 20 can be obtained by calculation.

From these calculation results, the drive coil 51 with the most acute angle, at the position of the capsule endoscope 20, between the orientation of the capsule endoscope 20 and the direction of the magnetic field produced thereby is selected. By selecting drive coils 51 in this manner, the induced magnetic field produced by the magnetic induction coil 42 can be maintained large, and superior conditions for position detection are ensured.

STEP 15 has already been described and thus will not be described again here.

STEP 16, the frequency of the signal generating circuit is set to the low-frequency-side calculating frequency, and in addition, the drive-coil selector 55 is operated by the position detection apparatus 50A to select a drive coil 51 as a drive coil for output.

In STEP 17, the outputs of all sense coils 52 selected in STEP 14 are measured. This corresponds to V(f_{LOW},N). Then, the difference between the obtained V_{c}(f_{LOW},N,M), which are the outputs of the sense coils at the calculating frequencies for all combinations of drive coils 51 and sense coils 52 while the capsule endoscope 20 is outside the space S, and data representing the combination of the corresponding sense coil and drive coil is calculated as follows to obtain Vₛ(f_{LOW},N).
Vₛ(f_{LOW},N)=V(f_{LOW},N)−V_{c}(f_{LOW},N,M)

STEP 18 has already been described and thus will not be described again here.

In STEP 19, the frequency of the signal generating circuit is set to the high-frequency-side calculating frequency without switching the drive coil 55 set in STEP 16.

In STEP 20, the outputs of all sense coils 52 selected in STEP 14 are measured. This corresponds to V(f_{HIGH},N). Then, the difference between the obtained V_{c}(f_{HIGH},N,M), which are the outputs of the sense coils at the calculating frequencies for all combinations of drive coils 51 and sense coils 52 while the capsule endoscope 20 is out of the space S, and data representing the combination of the corresponding sense coil and drive coil is calculated as follows to obtain Vₛ(f_{HIGH},N)).
Vₛ(f_{HIGH},N)=V(f_{HIGH},N)−V_{c}(f_{HIGH},N,M)

In STEP 21, the position detection apparatus 50A calculates Vₛ(f_{LOW},N)−Vₛ(f_{HIGH},N), which indicates the output difference (amplitude difference) of each selected sense coil 52 between the low-frequency-side calculating frequency and the high-frequency-side calculating frequency to perform calculation for the estimation of the position and direction of the capsule endoscope 20, namely, the magnetic induction coil 42 based on the value.

STEPs 22 and 23 have already been described and thus will not be described again here.

With the above-described processing (selection of the drive coils 51 and the sense coils 52), the induced magnetic field produced by the magnetic induction coil 42 can be detected efficiently by the sense coils 52 under conditions where an induced magnetic field from the magnetic induction coil 42 that is as large as possible is ensured. This reduces the amount of data used for position calculation of the capsule endoscope 20 (magnetic induction coil 42) without sacrificing the precision. Therefore, the amount of computation can be reduced and the system can be constructed at lower cost. Other advantages are also afforded, such as the system being speeded up.

In addition, two or more drive coils 51 may be selected in selecting dive coils 51. In this case, the magnetic fields produced by all of the selected drive coils at the position of the capsule endoscope 20 (magnetic induction coil 42) are calculated, and the output of each drive coil 51 is adjusted so that the angle between the direction of the combined magnetic field and the direction of the capsule endoscope 20 (magnetic induction coil 42) is acute. The value obtained by calibration of the selected sense coils 52 may instead be calculated as a sum of the output value of the output drive coils 51 and value obtained by multiplying factor based on the outputs of the individual drive coils by V_{c}(f_{LOW},N,M), and as a sum of the output value of the output drive coils 51 and value obtained by multiplying factor based on the outputs of the individual drive coils by V_{c}(f_{HIGH},N,M), where V_{c}(f_{LOW},N,M) and V_{c}(f_{HIGH},N,M) are measurement results described above. Furthermore, some output patterns where the output ratios of drive coils have been determined may be prepared so that calibration can be performed based on those output patterns in STEP 1. In this manner, the orientation of the magnetic field at the position of the capsule endoscope 20 (magnetic induction coil 42) can be set more freely. Therefore, more correct and efficient position detection can be achieved.

In addition, the outputs of the drive coils 51 may be adjusted so that the magnetic fields at the position of the capsule endoscope 20 (magnetic induction coil 42) produced by the drive coils 51 fall within a predetermined or certain range of the magnetic field intensity. Also in this case, the value obtained by calibration of the selected sense coils 52 may instead be calculated as a sum of the output value of the output drive coils 51 and value obtained by multiplying factor based on the outputs of the individual drive coils by V_{c}(f_{LOW},N,M), and as a sum of the output value of the output drive coils 51 and value obtained by multiplying factor based on the outputs of the individual drive coils by V_{c}(f_{HIGH},N,M), where V_{c}(f_{LOW},N,M) and V_{c}(f_{HIGH},N,M) are measurement results described above.

In this manner, a more stable induced magnetic field produced by the magnetic induction coil 42 can be output. Consequently, more accurate and efficient position detection can be achieved.

Next, the operation of the magnetic induction apparatus 70 will be described.

As shown in FIG. 1, in the magnetic induction apparatus 70, first, the operator inputs a guidance direction for the capsule endoscope 20 to the rotation-magnetic-field control circuit 73 via the input device 74. In the rotation-magnetic-field control circuit 73, the orientation and rotation direction of a parallel magnetic field to be applied to the capsule endoscope 20 are determined based on the input guidance direction and the orientation (rotation axis direction) of the capsule endoscope 20 input from the position detection apparatus 50A.

Then, to produce the orientation of the parallel magnetic field, the required intensity of the magnetic fields produced by the Helmholtz coils 71X, 71Y, and 71Z is calculated, and the electrical currents required to produce these magnetic fields are calculated.

The electric current data supplied to the individual Helmholtz coils 71X, 71Y, and 71Z is output to the corresponding Helmholtz-coil drivers 72X, 72Y, and 72Z, and the Helmholtz-coil drivers 72X, 72Y, and 72Z carry out amplification control of the currents based on the input data and supply the currents to the corresponding Helmholtz coils 71X, 71Y, and 71Z.

The Helmholtz coils 71X, 71Y, and 71Z to which the currents are supplied produce magnetic fields according to the respective current values, and by combining these magnetic fields, a parallel magnetic field having the magnetic field orientation determined by the rotation-magnetic-field control circuit 73 is produced.

The guidance magnet 45 is provided in the capsule endoscope 20 and, as described later, the orientation (rotation axis direction) of the capsule endoscope 20 is controlled based on the force and torque acting on the guidance magnet 45 and the parallel magnetic field described above. Also, by controlling the rotation period of the parallel magnetic field to be about 0 Hz to a few Hz and controlling the rotation direction of the parallel magnetic field, the rotation direction about the rotation axis of the capsule endoscope 20 is controlled, and the direction of movement and the moving speed of the capsule endoscope 20 are controlled.

Next, the operation of the capsule endoscope 20 will be described.

As shown in FIG. 5, in the capsule endoscope 20, first infrared light is irradiated onto the infrared sensor 47 of the switch section 46, and the switch section 46 outputs a signal to the signal processing section 34. When the signal processing section 34 receives the signal from the switch section 46, electrical current is supplied from the battery 39 to the image sensor 31, the LEDs 33, the radio device 35, and the signal processing section 34 itself, which are built into the capsule endoscope 20, and they are turned on.

The image sensor 31 images a wall surface inside the passage in the body cavity of the subject 1, which is illuminated by the LEDs 33, converts this image into an electrical signal, and outputs it to the signal processing section 34. The signal processing section 34 compresses the input image, temporarily stores it, and outputs it to the radio device 35. The compressed image signal input to the radio device 35 is transmitted to the image display apparatus 80 as electromagnetic waves.

The capsule endoscope 20 can move towards the front end portion 23 or the rear end portion 24 by rotating about the rotation axis R by means of the helical part 25 provided on the outer circumference of the outer casing 21. The direction of motion is determined by the rotation direction about the rotation axis R and the direction of rotation of the helical part 25.

Next, the operation of the image display apparatus 80 will be described.

As shown in FIG. 1, in the image display apparatus 80, first the image receiving circuit 81 receives the compressed image signal transmitted from the capsule endoscope 20, and the image signal is output to the display section 82. The compressed image signal is reconstructed in the image receiving circuit 81 or the display section 82, and is displayed by the display section 82.

Also, the display section 82 performs rotation processing on the image signal in the opposite direction to the rotation direction of the capsule endoscope 20 based on the rotational phase data of the capsule endoscope 20, which is input from the rotation-magnetic-field control circuit 73, and displays it.

With the above-described structure, since the resonance frequency of the magnetic induction coil 42 is obtained using alternating magnetic fields whose frequency changes over time, the resonance frequency can be obtained irrespective of large variations in resonance frequency of the magnetic induction coil 42, so that calculating frequencies can be obtained based on the resonance frequency. For this reason, irrespective of variations in resonance frequency of the magnetic induction coil 42, the position and orientation of the capsule endoscope 20 can be calculated based on the calculating frequencies.

As a result, it is not necessary to provide an element and so forth for adjusting the resonance frequency of the magnetic induction coil 42, and therefore, the size of the capsule endoscope 20 can be reduced. Furthermore, it is no longer necessary to select or adjust an element such as a capacitor and so forth constituting the resonance circuit 43 together with the magnetic induction coil 42 in order to adjust the resonance frequency. This prevents an increase in the manufacturing cost of the capsule endoscope 20.

Since only alternating magnetic fields with the low-frequency-side calculating frequency and the high-frequency-side calculating frequency are used for the calculation of the position and orientation of the capsule endoscope 20, the time required to calculate the position and orientation can be reduced compared with, for example, a method for sweeping the frequency of the alternating magnetic field within a predetermined range.

Since the band-pass filter 61 can limit the band of the output frequency of the sense coils 52 based on the low-frequency-side calculating frequency and the high-frequency-side calculating frequency, the position and orientation of the capsule endoscope 20 can be calculated based on the sense coil output having frequency ranges in the vicinity of the low-frequency-side calculating frequency and the high-frequency-side calculating frequency, and therefore, the time required to calculate the position and orientation can be reduced.

Alternating magnetic fields are applied to the magnetic induction coil 42 of the capsule endoscope 20 from three or more different directions that are linearly independent. Therefore, it is possible to produce an induced magnetic field in the magnetic induction coil 42 by alternating magnetic fields from at least one direction, irrespective of the orientation of the magnetic induction coil 42.

As a result, it is always possible to produce induced magnetic fields in the magnetic induction coil 42, irrespective of the orientation (axial direction of the rotation axis R) of the capsule endoscope 20; therefore, an advantage is afforded in that it is possible to always detect the induced magnetic field by the sense coils 52, which allows the position thereof to always be detected with accuracy.

Also, since the sense coils 52 are disposed in three different directions with respect to the capsule endoscope 20, an induced magnetic field of detectable intensity acts on the sense coils 52 disposed in at least one direction of the sense coils 52 disposed in the three directions, which allows the sense coils 52 to always detect the induced magnetic field, irrespective of the position at which the capsule endoscope 20 is disposed.

Furthermore, since the number of sense coils 52 disposed in one direction is nine, as mentioned above, a sufficient number of inputs to acquire a total of six pieces of information by calculation is ensured, where the six pieces of information include the X, Y, and Z coordinates of the capsule endoscope 20, the rotational phases φ and θ about two axes orthogonal to each other and orthogonal to the rotation axis R of the capsule endoscope 20, and the intensity of the induced magnetic field.

By setting the frequency of the alternating magnetic field close to the frequency at which the resonance circuit 43 resonates (the resonance frequency), it is possible to produce an induced magnetic field with an amplitude that is large compared to a case where another frequency is used. Since the amplitude of the induced magnetic field is large, the sense coils 52 can easily detect the induced magnetic field, which makes it easy to detect the position of the capsule endoscope 20.

Also, since the frequency of the alternating magnetic field sweeps over a frequency range in the vicinity of the resonance frequency, even if the resonance frequency of the resonance circuit 43 changes due to variations in the environmental conditions (for example, the temperature conditions) or even if there is a shift in the resonance frequency due to individual differences in the resonance circuit 43, it is possible to bring about resonance in the resonance circuit 43 so long as the changed resonance frequency or the shifted resonance frequency is included in the frequency range mentioned above.

Since the position detection apparatus 50A selects the sense coils 52 that detect high-intensity induced magnetic fields by means of the sense-coil selector 56, it is possible to reduce the volume of information that the position detection apparatus 50A must calculate and process without sacrificing accuracy, which allows the computational load to be reduced. At the same time, since it is possible to simultaneously reduce the amount of computational processing, the time required for computation can be shortened.

Since the drive coils 51 and the sense coils 52 are located at positions opposing each other on either side of the operating region of the capsule endoscope 20, the drive coils 51 and the sense coils 52 can be positioned so that they do not interfere with each other in terms of their construction.

By controlling the orientation of the parallel magnetic fields acting on the guidance magnet 45 built into the capsule endoscope 20, it is possible to control the orientation of the force acting on the guidance magnet 45, which allows the direction of motion of the capsule endoscope 20 to be controlled. Since it is possible to detect the position of the capsule endoscope 20 at the same time, the capsule endoscope 20 can be guided to a predetermined position, and therefore, an advantage is afforded in that it is possible to accurately guide the capsule endoscope based on the detected position of the capsule endoscope 20.

By controlling the intensities of the magnetic fields produced by the three pairs of Helmholtz coils 71X, 71Y, and 71Z that are disposed to face each other in mutually orthogonal directions, the orientations of the parallel magnetic fields produced inside the Helmholtz coils 71X, 71Y, and 71Z can be controlled in a predetermined direction. Accordingly, a parallel magnetic field in a predetermined orientation can be applied to the capsule endoscope 20, and it is possible to move the capsule endoscope 20 in a predetermined direction.

Since the drive coils 51 and the sense coils 52 are disposed in the periphery of the space at the inner sides of the Helmholtz coils 71X, 71Y, and 71Z, which is the space in which the subject 1 can be positioned, the capsule endoscope 20 can be guided to a predetermined location in the body of the subject 1.

By rotating the capsule endoscope 20 about the rotation axis R, the helical part 25 produces a force that propels the capsule endoscope 20 in the axial direction of the rotation axis. Since the helical part 25 produces the propulsion force, it is possible to control the direction of the propulsion force acting on the capsule endoscope 20 by controlling the direction of rotation about the rotation axis R of the capsule endoscope 20.

Since the image display apparatus 80 performs the processing for rotating a display image in the rotation direction opposite to that of the capsule endoscope 20, based on information on the rotational phase about the rotational axis R of the capsule endoscope 20, it is possible to display on the display section 82 an image that is always fixed at a predetermined rotational phase, in other words, an image in which the capsule endoscope 20 appears to travel along the rotation axis R without rotating about the rotation axis R, regardless of the rotational phase of the capsule endoscope 20.

Accordingly, when the capsule endoscope 20 is guided while the operator visually observes the image displayed on the display section 82, showing the image displayed in the manner described above as a predetermined rotational phase image makes it easier for the operator to view and also makes it easier to guide the capsule endoscope 20 to a predetermined location, compared to the case where the displayed image is an image that rotates along with the rotation of the capsule endoscope 20.

The frequency of alternating magnetic fields used to obtain calculating frequencies (Step 1, Step 3) may be swept, as described above. Alternatively, an impulse magnetic field may be employed to obtain the calculating frequencies by using the position detection apparatus 50A as an impulse-magnetic-field generating section for generating an impulse magnetic field from the drive coil 51.

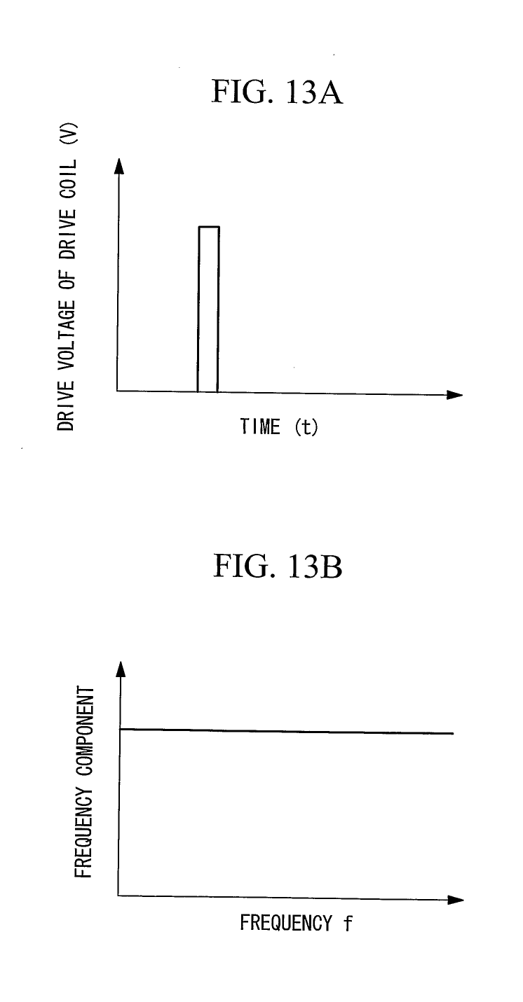

An impulse magnetic field, as shown in FIG. 13A, generated by applying an impulse drive voltage to a drive coil 51 includes many frequency components as shown in FIG. 13B. Therefore, the resonance frequency of the magnetic induction coil 42 can be obtained for a shorter period of time compared with, for example, a method for sweeping the frequency of the magnetic field, and in addition, the resonance frequency can be obtained over a much wider frequency range. In this case, by connecting a spectrum analyzer (not shown in the figure), which can perform analysis of frequency components, to the sense coil 52 connected to the sense-coil receiving circuit 57, frequency components of a signal output from the sense coil 52 when an impulse drive voltage is applied to the drive coil 51 can be detected.

Furthermore, the frequency range input to the frequency determining section 50B may be controlled by using the position detection apparatus 50A as a mixed-magnetic-field generating section which produces an alternating magnetic field containing a plurality of different frequencies by the drive coil 51 to employ an alternating magnetic field containing a plurality of different frequencies when a calculating frequency is to be obtained, and furthermore by using the band-pass filter 61 as a variable bandwidth limiting section that can change the range of transmitted frequencies.

With this structure, the resonance frequency is easier to obtain compared with a case where an alternating magnetic field with a predetermined frequency is used, despite large variations in resonance frequency of the magnetic induction coil 42.

### Second Embodiment

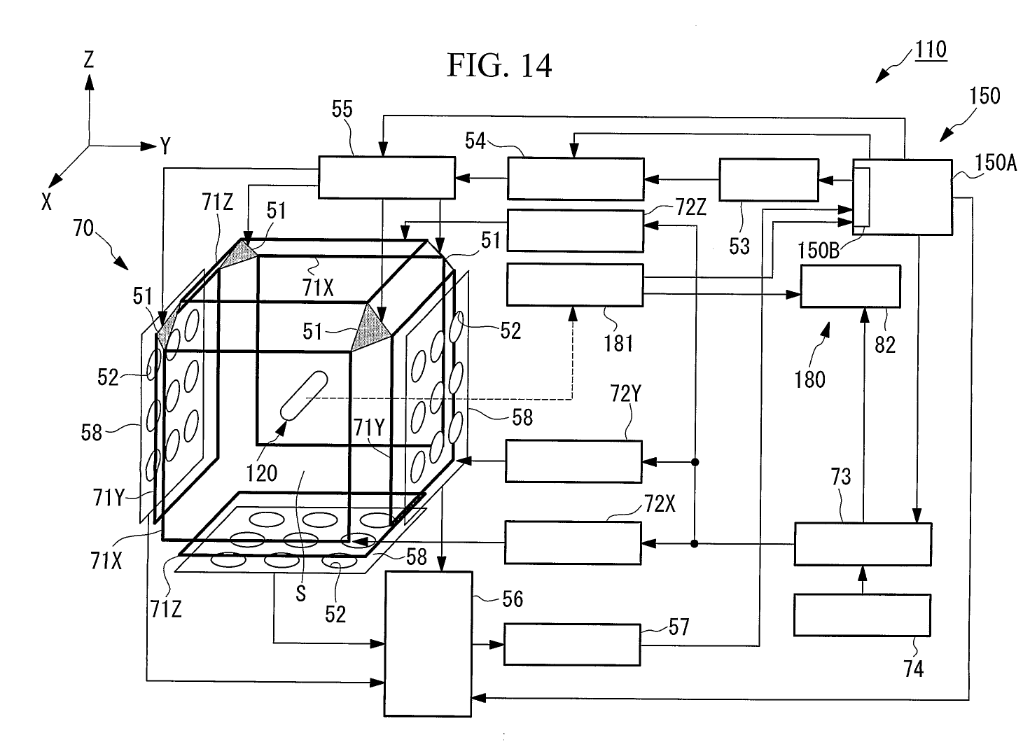

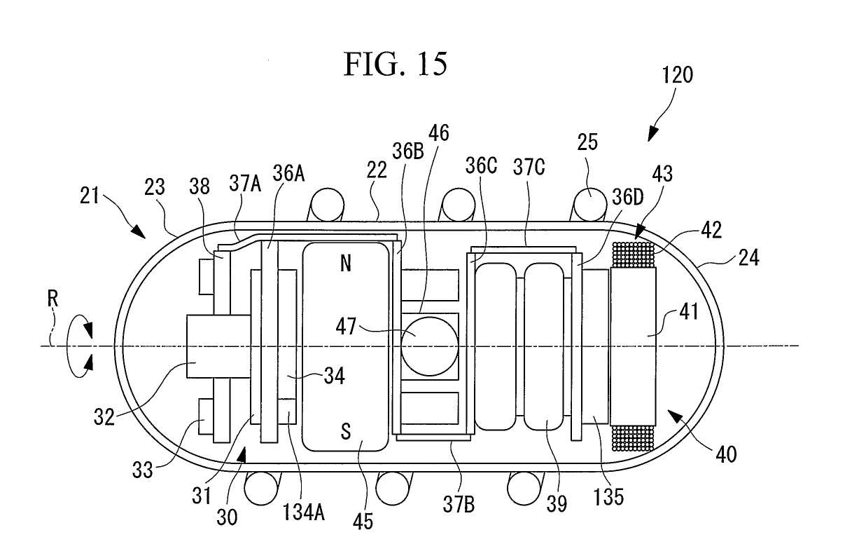

A second embodiment of the present invention will now be described with reference to FIGS. 14 and 15.

The basic configuration of the medical magnetic-induction and position-detection system according to this embodiment is the same as that in the first embodiment; however, the method of determining the calculating frequencies and the mechanism for the determination are different from those in the first embodiment. Thus, in this embodiment, only the method of determining the calculating frequencies and the mechanism for the determination shall be described with reference to FIGS. 14 and 15, and the description of the magnetic induction apparatus and so forth shall be omitted.

FIG. 14 is a diagram schematically showing a medical magnetic-induction and position-detection system according to this embodiment.

The same components as those in the first embodiment are denoted with the same reference numerals, and thus will not be described.

As shown in FIG. 14, a medical magnetic-induction and position-detection system 110 is mainly formed of a capsule endoscope (medical device) 120 that optically images an internal surface of a passage in a body cavity and wirelessly transmits an image signal; a position detection unit (position detection system, position detector, calculating apparatus) 150 that detects the position of the capsule endoscope 120; a magnetic induction apparatus 70 that guides the capsule endoscope 120 based on the detected position of the capsule endoscope 120 and instructions from an operator; and an image display apparatus 180 that displays the image signal transmitted from the capsule endoscope 120.

As shown in FIG. 14, the position detection unit 150 is mainly formed of drive coils 51 that generate induced magnetic fields in a magnetic induction coil (described later) in the capsule endoscope 120; sense coils 52 that detect the induced magnetic fields generated in the magnetic induction coil; and a position detection apparatus (position analyzing unit, magnetic-field-frequency varying section, drive-coil control section) 150A that computes the position of the capsule endoscope 120 based on the induced magnetic fields that the sense coils 52 detect and that controls the alternating magnetic fields formed by the drive coils 51.

The position detection apparatus 150A is provided with a calculating-frequency determining section (frequency determining section) 150B to receive signals from a sense-coil receiving circuit and a capsule information reception circuit to be described later.

The image display apparatus 180 is formed of a capsule information reception circuit 181 that receives the image and the values of calculating frequencies transmitted from the capsule endoscope 120 and a display section 82 that displays the image based on the received image signal and a signal from the rotation-magnetic-field control circuit 73.

FIG. 15 is a schematic diagram showing the configuration of the capsule endoscope.

As shown in FIG. 15, the capsule endoscope 120 is mainly formed of an outer casing 21 that accommodates various devices in the interior thereof; an imaging section 30 that images an internal surface of a passage in the body cavity of the subject; a battery 39 for driving the imaging section 30; an induced-magnetic-field generating section 40 that generates induced magnetic fields by means of the drive coils 51 described above; and a guidance magnet 45 that drives the capsule endoscope 120.

The imaging section 30 is mainly formed of a board 36A positioned substantially orthogonal to the rotation axis R; an image sensor 31 disposed on the surface at the front end portion 23 side of the board 36A; a lens group 32 that forms an image of the internal surface of the passage inside the body cavity of the subject on the image sensor 31; an LED (Light Emitting Diode) 33 that illuminates the internal surface of the passage inside the body cavity; a signal processing section 34 disposed on the surface at the rear end portion 24 side of the board 36A; and a radio device (communication section) 135 that transmits the image signal to the image display apparatus 80.

In the signal processing section 34, a memory section 134A for storing calculating frequencies based on the resonance frequency of the resonance circuit 43 of the induced-magnetic-field generating section 40 is also arranged. The memory section 134A is electrically connected to the radio device 135 and is constructed so as to store calculating frequencies therein and externally transmit the calculating frequencies stored therein via the radio device 135.

The operation of the medical magnetic-induction and position-detection system 110 with the above-described structure will now be described.

The outline of the operation of the medical magnetic-induction and position-detection system 110 has been described in the first embodiment, and thus will not be described again here.

A procedure for obtaining calculating frequencies used to detect the position and direction of the capsule endoscope 120 and a procedure for detecting the position and direction of the capsule endoscope 120 will now be described.

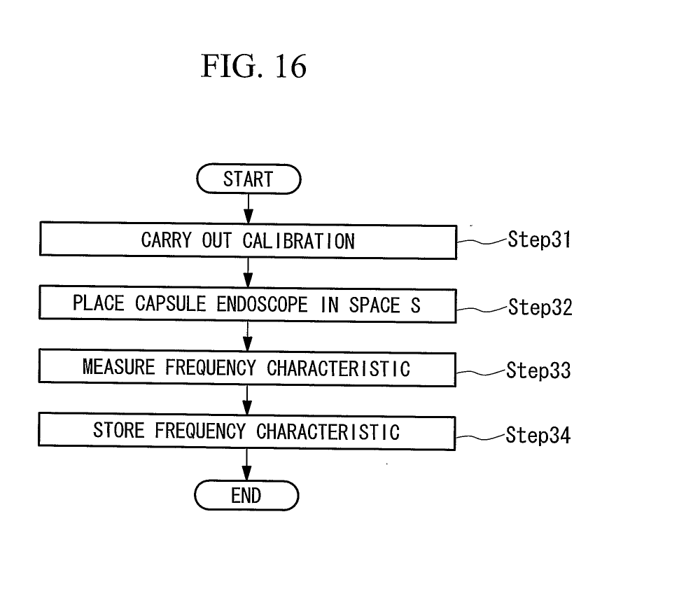

FIG. 16 is a flowchart illustrating a procedure from obtaining the frequency characteristic of the magnetic induction coil 42 to storing the obtained frequency characteristic in the memory section 134A.

First, as shown in FIG. 16, calibration of the position detection unit 150 is carried out (Step 31; preliminary measuring step). More specifically, the output of the sense coils 52 while the capsule endoscope 120 is not disposed in the space S, namely, the output of the sense coils 52 resulting from the operation of alternating magnetic fields formed by the drive coils 51, is measured.

A specific procedure for forming alternating magnetic fields and so forth has been described in the first embodiment, and thus will not be described again here.

Next, the capsule endoscope 120 is placed in the space S (Step 32).

Then, the frequency characteristic of the magnetic induction coil 42 installed in the capsule endoscope 120 is measured (Step 33; measuring step). Thereafter, in the frequency determining section 150B, the output of the sense coils 52 on which only the alternating magnetic fields are acting, namely the output measured in Step 31, is subtracted from the measured frequency characteristic of the magnetic induction coil 42 (the difference is calculated).

Thereafter, the frequency determining section 150B stores the frequency characteristic of the magnetic induction coil 42 in the memory section 134A via the radio device 135 (Step 34).

The processing for storing the above-described frequency characteristic in the memory section 134A is carried out when the capsule endoscope 120 is manufactured. For this reason, neither obtaining nor storing a frequency characteristic is required on-site where the capsule endoscope 120 is actually used.

In addition, for the processing from Step 31 to Step 34, not all components of the medical magnetic-induction and position-detection system 110 are necessary. In other words, a system capable of controlling the operation of one drive coil 51 and one sense coil 52 is sufficient.

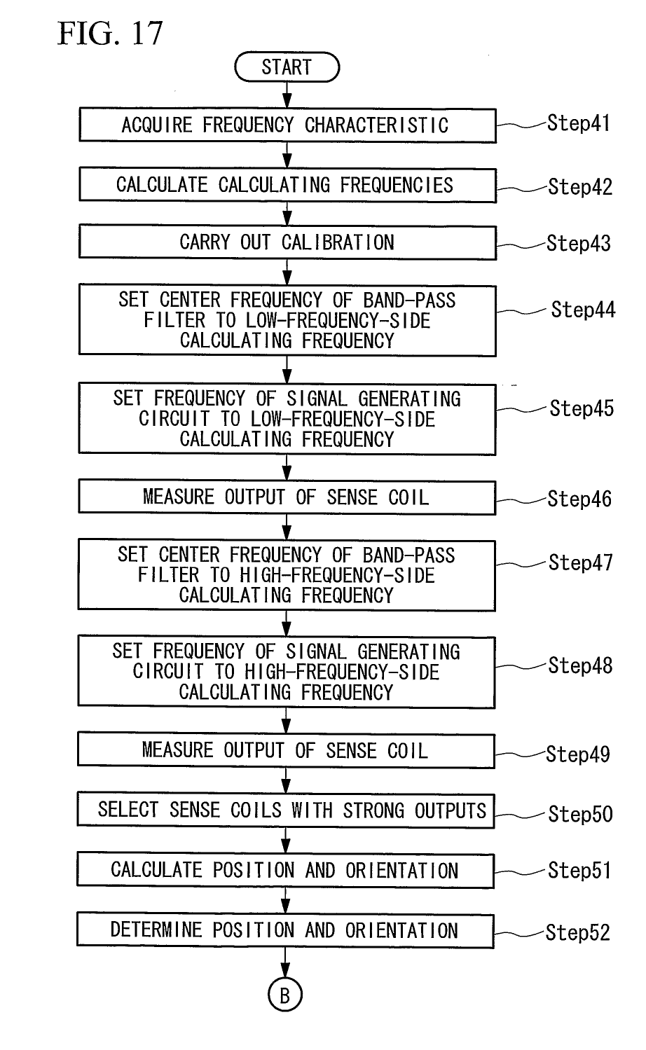

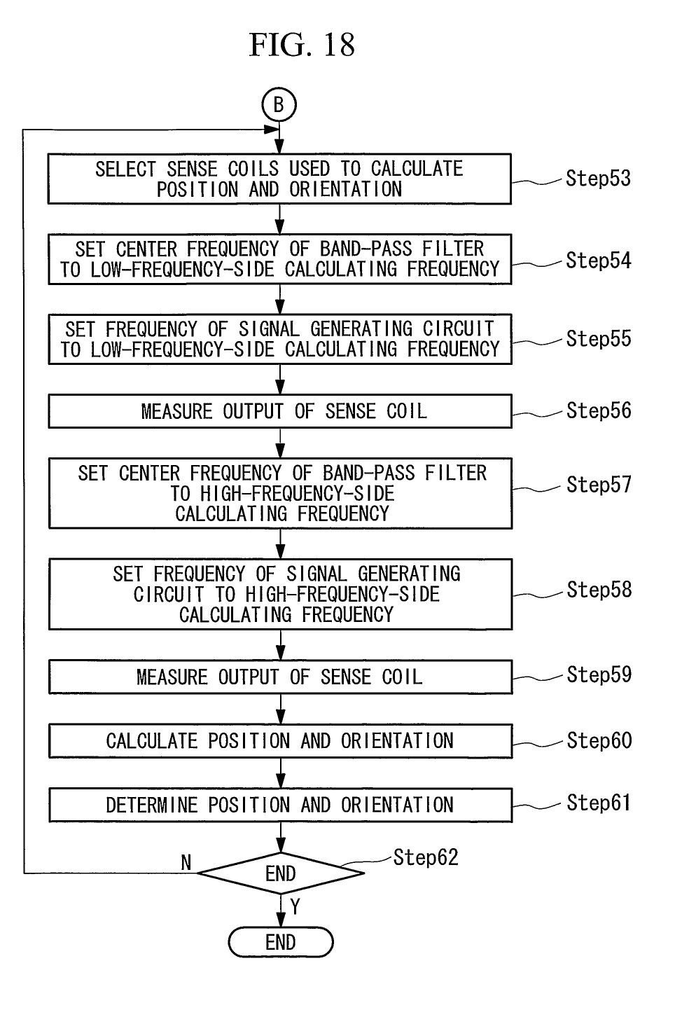

FIGS. 17 and 18 are flowcharts illustrating a procedure for acquiring the frequency characteristic stored in the memory section 134A and for detecting the position and orientation of the capsule endoscope 120.

A procedure for detecting the position and direction of the capsule endoscope 120 in which the frequency characteristic has been stored will now be described.

First, as shown in FIG. 17, when the switch of the capsule endoscope 120 is turned on, the radio device 135 externally transmits the data for the frequency characteristic stored in the memory section 134A, and the data for the transmitted frequency characteristic is received by the capsule information reception circuit 181 and is then input to the frequency determining section 150B (Step 41).

Thereafter, the frequency determining section 150B obtains calculating frequencies used to detect the position and orientation of the capsule endoscope 120 based on the acquired frequency characteristic (Step 42; frequency determination step).

As with the first embodiment, the frequencies at which a change in gain of the sense coils 52 exhibits the maximum value and the minimum value are selected for the calculating frequencies. The lower frequency is referred to as the low-frequency-side calculating frequency, and the higher frequency is referred to as the high-frequency-side calculating frequency.

Alternatively, the frequencies (low-frequency-side calculating frequency, high-frequency-side calculating frequency) used for detection of the position and direction may be stored in the memory section 134A in Step 34. In this manner, calculating frequencies can be determined merely by reading the data stored in the memory section 134A.

Then, as in Step 1 of the first embodiment, calibration of the position detection unit 150 is carried out by using alternating magnetic fields at the obtained low-frequency-side calculating frequency and high-frequency-side calculating frequency (Step 43; preliminary measuring step) to measure the outputs of all sense coils 52 when the alternating magnetic fields are applied. The measured outputs are denoted as Vc(f_{LOW},N) and Vc(f_{HIGH},N), as with the first embodiment.

Thereafter, the center frequency of the band-pass filter 61 is adjusted to the low-frequency-side calculating frequency (Step 44). Furthermore, the transmission frequency range of the band-pass filter 61 is set to such a range that local extreme values of a change in gain of the sense coils 52 can be extracted.

Then, the frequency of the alternating magnetic fields formed by the drive coils 51 is adjusted to the low-frequency-side calculating frequency (Step 45). More specifically, the frequency of the alternating magnetic fields formed by the drive coils 51 is controlled by controlling the frequency of AC current generated by the signal generating circuit 53 to the low-frequency-side calculating frequency.

Then, alternating magnetic fields with the low-frequency-side calculating frequency are produced by the drive coils 51 to detect the magnetic field induced by the magnetic induction coil 42 with the sense coil 52 (Step 46; detection step). Also here, as with the first embodiment, Vs(f_{LOW},N)=V(f_{LOW},N)−Vc(f_{LOW},N) is calculated based on the obtained V(f_{LOW},N), and Vs(f_{LOW},N) is stored as a value calculated based on the output of the sense coils 52.

Next, the center frequency of the band-pass filter 61 is adjusted to the high-frequency-side calculating frequency (Step 47).

Then, the frequency of alternating magnetic fields formed by the drive coils 51 is adjusted to the high-frequency-side calculating frequency (Step 48).

Alternating magnetic fields with the high-frequency-side calculating frequency are produced by the drive coils 51 to detect the magnetic field induced by the magnetic induction coil 42 with the sense coils 52 (Step 49; detection step). V(f_{HIGH},N) is detected at this time and, as in Step 46, Vs(f_{HIGH},N)=V(f_{HIGH},N)−Vc(f_{HIGH},N) is calculated to store Vs(f_{HIGH},N) as a value calculated based on the output of the sense coils 52.

As described above, detection with the low-frequency-side calculating frequency can be performed first, followed by detection with the high-frequency-side calculating frequency. Alternatively, detection with the high-frequency-side calculating frequency may be performed first, followed by detection with the low-frequency-side calculating frequency.

Thereafter, the position detection apparatus 150A calculates the output difference (amplitude difference) of each sense coil 52 between the low-frequency-side calculating frequency and the high-frequency-side calculating frequency, and then the sense coils 52 whose output difference is to be used to estimate the position of the capsule endoscope 120 are selected (Step 50).

The procedure for selecting the sense coils 52 has been described in the first embodiment, and thus will not be described again here.

Then, the position detection apparatus 150A calculates the position and orientation of the capsule endoscope 20 based on the output difference of the selected sense coils 52 (Step 51; position calculating step) to determine the position and orientation (Step 52).

Then, sense coils 52 used for the subsequent control are selected as shown in FIG. 18 (Step 53).

More specifically, the position detection apparatus 150A obtains by calculation the intensity of a magnetic field produced from the magnetic induction coil 42 at the position of each sense coil 52 based on the position and orientation of the capsule endoscope 120 calculated in Step 52 and selects as many sense coils 52 as necessary disposed at positions where the magnetic field intensity is high. When the acquisition of the position and orientation of the capsule endoscope 120 is to be repeated, sense coils 52 are selected based on the position and orientation of the capsule endoscope 120 calculated in Step 61 to be described later.

Although the number of selected sense coils 52 should be at least six in this embodiment, about ten to fifteen selected sense coils 52 are advantageous in minimizing errors in position calculation. Alternatively, sense coils 52 may be selected in such a manner that the outputs of all sense coils 52 resulting from the magnetic field produced from the magnetic induction coil 42 are calculated based on the position and orientation of the capsule endoscope 120 obtained in Step 52 (or Step 61 to be described later), and then as many sense coils 52 as necessary that have large outputs are selected.

Thereafter, the center frequency of the band-pass filter 61 is re-adjusted to the low-frequency-side calculating frequency (Step 54).

Then, the frequency of the alternating magnetic fields formed by the drive coils 51 is adjusted to the low-frequency-side calculating frequency (Step 55).

Then, alternating magnetic fields with the low-frequency-side calculating frequency are generated by the drive coils 51 to detect the magnetic field induced by the magnetic induction coil 42 with the selected sense coils 52 (Step 56; detection step).

Next, the center frequency of the band-pass filter 61 is adjusted to the high-frequency-side calculating frequency (Step 57).

Then, the frequency of the alternating magnetic fields formed by the drive coils 51 is adjusted to the high-frequency-side calculating frequency (Step 58).

Then, alternating magnetic fields with the high-frequency-side calculating frequency are produced by the drive coils 51 to detect the magnetic field induced by the magnetic induction coil 42 with the selected sense coils 52 (Step 59; detection step).

Then, the position detection apparatus 150A calculates the position and orientation of the capsule endoscope 120 based on the output difference of the sense coils 52 selected in Step 53 (Step 60; position calculating step) to determine the position and orientation (Step 61).

In Step 61, data for the calculated position and orientation of the capsule endoscope apparatus 120 may be output to another apparatus or the display section 82.

Thereafter, if detection of the position and orientation of the capsule endoscope apparatus 120 is to be continued, the flow returns to Step 53, where detection of the position and orientation is carried out.

With the above-described structure, when the position and orientation of the capsule endoscope 120 are to be calculated, the frequency characteristic of the magnetic induction coil 42 pre-stored in the memory section 134A is acquired to obtain a low-frequency-side calculating frequency and a high-frequency-side calculating frequency. For this reason, the time required to calculate the position and orientation of the capsule endoscope 120 can be reduced compared with a method where a resonance frequency is measured to obtain calculating frequencies each time position detection of the capsule endoscope 120 is to be carried out.

The frequency characteristic of the magnetic induction coil 42 may be stored in the memory section 134A so that the stored frequency characteristic can be automatically sent to the frequency determining section 150B via the radio device 135 and the capsule information reception circuit 181, as described above. Alternatively, the value of the frequency characteristic may be written on, for example, the outer casing 21 of the capsule endoscope apparatus 120 so that the operator can enter the value into the frequency determining section 150B. Instead of the outer casing 21, the value may be written on the enclosure of the package.

Furthermore, in the memory section 134A, the frequency characteristic of the magnetic induction coil 42 may be stored or calculating frequencies calculated based on the frequency characteristic may be stored.

In addition, the value itself of the frequency characteristic and so forth may be written on, for example, the outer casing 21, or values of frequency characteristics and so forth may be classified into several ranks so that a rank is written on, for example, the outer casing 21.

### Third Embodiment

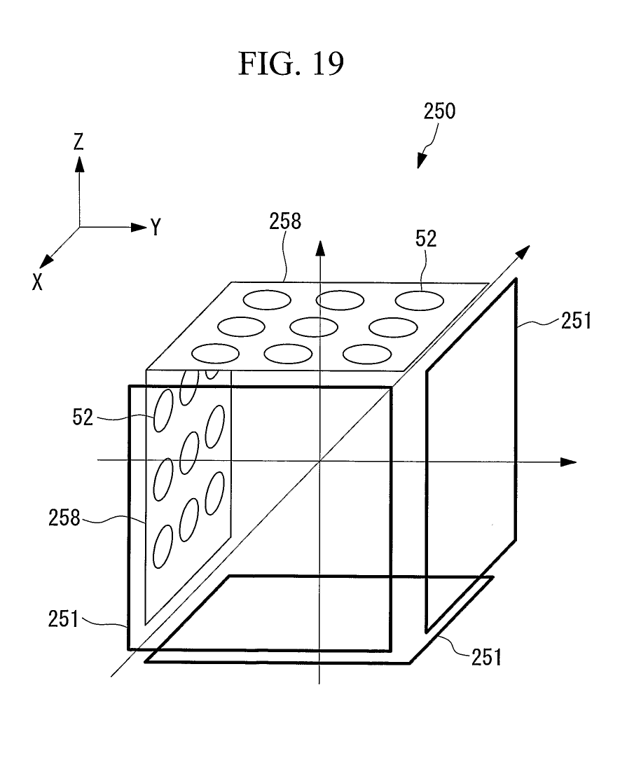

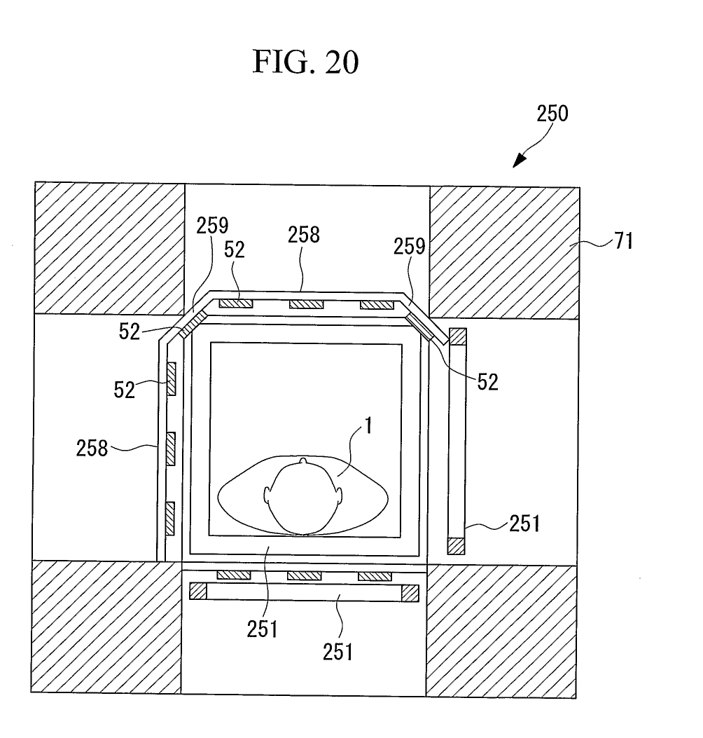

A third embodiment of the present invention will now be described with reference to FIGS. 19 and 20.

The basic configuration of the medical magnetic-induction and position-detection system according to this embodiment is the same as that in the first embodiment; however, the configuration of the position detection unit is different from that in the first embodiment. Therefore, in this embodiment, only the vicinity of the position detection unit will be described using FIGS. 19 and 20, and the description of the magnetic induction apparatus and the like will be omitted.

FIG. 19 is a schematic diagram showing the placement of drive coils and sense coils of the position detection unit.

Since the components other than the drive coils and the sense coils of the position detection unit are the same as in the first embodiment, a description thereof shall be omitted.

As shown in FIG. 19, drive coils (driving coils) 251 and sense coils 52 of the position detection unit (position detection system, position detector, calculating apparatus) 250 are arranged such that the three drive coils 251 are orthogonal to the X, Y, and Z axes, respectively, and the sense coils 52 are disposed on two planar coil-supporting parts 258 orthogonal to the Y and Z axes, respectively.

Rectangular coils as shown in the figure or Helmholtz coils may be used as the drive coils 251.

As shown in FIG. 19, in the position detection unit 250 having the configuration described above, the orientations of the alternating magnetic fields that the drive coils 251 produce are parallel to the X, Y, and Z axial directions and are linearly independent, having a mutually orthogonal relationship.

With this configuration, it is possible to apply alternating magnetic fields to the magnetic induction coil 42 in the capsule endoscope 20 from linearly independent and mutually orthogonal directions. Therefore, an induced magnetic field is easier to generate in the magnetic induction coil 42 compared to the first embodiment, regardless of the orientation of the magnetic induction coil 42.

Also, since the drive coils 151 are disposed so as to be substantially orthogonal to each other, selection of the drive coils by the drive-coil selector 55 is simplified.

The sense coils 52 may be disposed on the coil-support members 258, which are perpendicular to the Y and Z axes, as described above, or, as shown in FIG. 20, sense coils 52 may be provided on inclined coil-support members 259 disposed in the upper part of the operating region of the capsule endoscope 20.

By positioning them in this manner, the sense coils 52 can be positioned without interfering with the subject 1.

### Fourth Embodiment

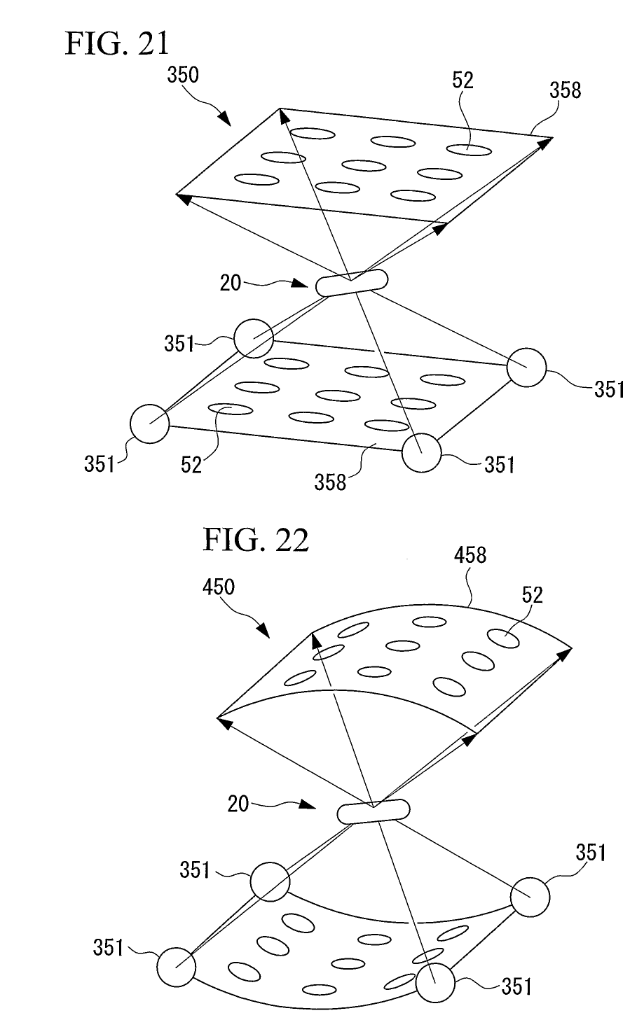

A fourth embodiment of the present invention will now be described with reference to FIG. 21.

The basic configuration of the medical magnetic-induction and position-detection system according to this embodiment is the same as that in the first embodiment; however, the configuration of the position detection unit is different from that in the first embodiment. Therefore, in this embodiment, only the vicinity of the position detection unit will be described using FIG. 21, and the description of the magnetic induction apparatus and the like will be omitted.

FIG. 21 is a schematic diagram showing the placement of drive coils and sense coils of the position detection unit.

Since the components other than the drive coils and the sense coils of the position detection unit are the same as in the first embodiment, a description thereof shall be omitted.

Regarding drive coils (driving coils) 351 and sense coils 52 of the position detection unit (position detection system, position detector, calculating apparatus) 350, as shown in FIG. 21, four drive coils 351 are disposed in the same plane, and the sense coils 52 are disposed on a planar coil-supporting member 358, which is disposed at a position opposite the position where the drive coils 351 are located, and on a planar coil-supporting member 358, which is disposed at the same side where the drive coils 351 are located, the operating region of the capsule endoscope 20 being disposed therebetween.

The drive coils 351 are arranged such that the orientations of the alternating magnetic fields that the drive coils 351 produce are linearly independent of each other, as indicated by the arrows in the figure.

According to this configuration, one of the two coil-supporting members 358 is always located close with respect to the capsule endoscope 20, regardless of whether the capsule endoscope 20 is located in a nearby region or a distant region with respect to the drive coils 351. Accordingly, a signal of sufficient intensity can be obtained from the sense coils 52 when determining the position of the capsule endoscope 20.

### Modification of Fourth Embodiment

Next, a modification of the fourth embodiment of the present invention will be described with reference to FIG. 22.

The basic configuration of the medical magnetic-induction and position-detection system of this modification is the same as that in the third embodiment; however, the configuration of the position detection unit is different from that in the third embodiment. Therefore, in this modification, only the vicinity of the position detection unit will be described using FIG. 22, and a description of the magnetic induction apparatus and the like will be omitted.

FIG. 22 is a schematic diagram showing the positioning of drive coils and sense coils of the position detection unit.

Since the components other than the drive coils and the sense coils of the position detection unit are the same as in the third embodiment, a description thereof is omitted here.

Regarding drive coils 351 and sense coils 52 of the position detection unit (position detection system, position detector, calculating apparatus) 450, as shown in FIG. 22, four drive coils 351 are disposed in the same plane, and the sense coils 52 are disposed on a curved coil-supporting member 458, which is disposed at a position opposite the position where the drive coils 351 are located, and on a curved coil-supporting member 458, which is disposed at the same side where the drive coils 351 are located, the operating region of the capsule endoscope 20 being disposed therebetween.

The coil-supporting members 458 are formed in a curved shape that is convex towards the outer side relative to the operating region of the capsule endoscope 20, and the sense coils 52 are disposed over the curved surfaces.

The shape of the coil-supporting members 458 may be curved surfaces that are convex towards the outer side with respect to the operating region, as described above, or they may be any other shape of curved surface and are not particularly limited.

With the configuration described above, since the degree of freedom of positioning the sense coils 52 is improved, it is possible to prevent the sense coils 52 from interfering with the subject 1.

### Fifth Embodiment

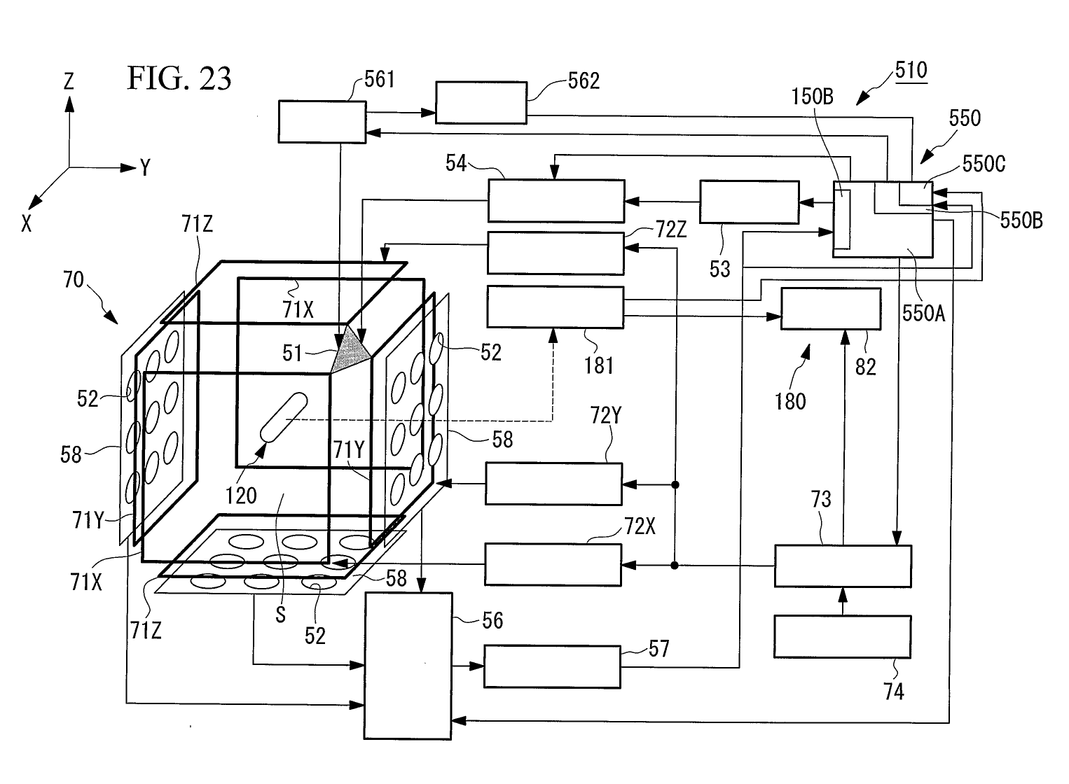

A fifth embodiment of the present invention will now be described with reference to FIGS. 23 through 28.

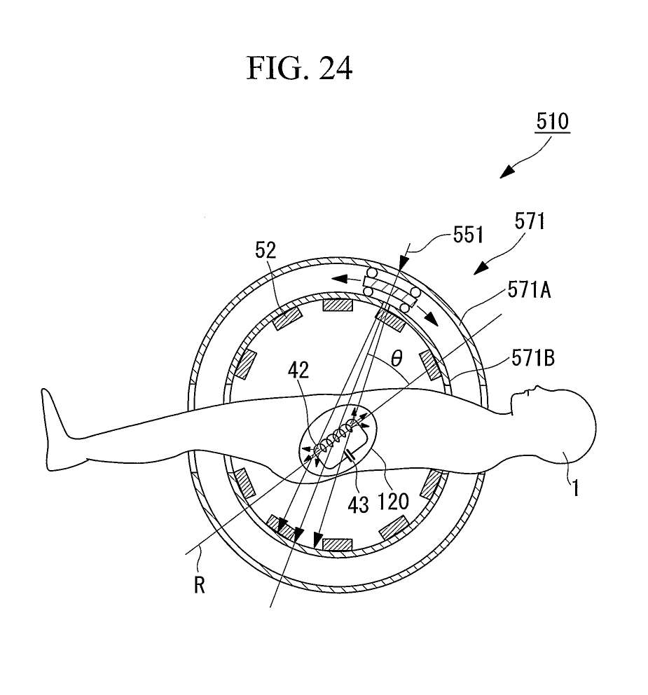

The basic configuration of the medical magnetic-induction and position-detection system according to this embodiment is the same as that in the second embodiment; however, the configuration of the position detection unit is different from that in the second embodiment. Therefore, in this embodiment, only the vicinity of the position detection unit will be described using FIGS. 23 through 24, and the description of the magnetic induction apparatus and the like will be omitted.

FIG. 23 is a diagram schematically showing a medical magnetic-induction and position-detection system according to this embodiment.

The same components as those in the second embodiment are denoted with the same reference numerals, and thus will not be described again here.

As shown in FIG. 23, a medical magnetic-induction and position-detection system 510 is mainly formed of a capsule endoscope 120 that optically images an internal surface of a passage in a body cavity and wirelessly transmits an image signal; a position detection unit (position detection system, position detector, calculating apparatus) 550 that detects the position of the capsule endoscope 120; a magnetic induction apparatus 70 that guides the capsule endoscope 120 based on the detected position of the capsule endoscope 120 and instructions from an operator; and an image display apparatus 180 that displays the image signal transmitted from the capsule endoscope 120.

As shown in FIG. 23, the position detection unit 550 is mainly formed of a drive coil 51 that generates an induced magnetic field in a magnetic induction coil (described later) in the capsule endoscope 120; sense coils 52 that detect the induced magnetic field generated in the magnetic induction coil; a relative-position changing section (relative-position changing unit) 561 for changing the relative positions of the drive coil 51 and the sense coils 52; a relative-position measuring section (relative-position measuring unit) 562 for measuring such relative positions; and a position detection apparatus (position analyzing unit, magnetic-field-frequency varying section, drive-coil control section) 550A that computes the position of the capsule endoscope 120 based on the induced magnetic field that the sense coils 52 detect and that controls the alternating magnetic field formed by the drive coil 51.

The position detection apparatus 550A is provided with a frequency determining section 150B for obtaining calculating frequencies and a current-reference-value generating section 550B for generating a reference value to receive signals from a sense-coil receiving circuit and a capsule information reception circuit to be described later. In addition, the current-reference-value generating section 550B is provided with a storage section (memory section) 550C for associating information about the relative positions of the drive coil 51 and the sense coils 52 with information about the output of the sense coils 52 to store the information therein.

Between the position detection apparatus 550A and the drive coil 51 there are provided a signal generating circuit 53 that generates an AC current based on the output from the position detection apparatus 550A; and a drive-coil driver 54 that amplifies the AC current input from the signal generating circuit 53 based on the output from the position detection apparatus 550A.

Between the position detection apparatus 550A and the drive coil 51 there is provided the relative-position changing section 561, and between the relative-position changing section 561 and the position detection apparatus 550A there is provided the relative-position measuring section 562. The output of the position detection apparatus 550A is input to a drive coil unit, to be described later, via the relative-position changing section 561. Information about the relative positions of the drive coil 51 and the sense coils 52 is acquired by the relative-position measuring section 562 from the drive coil unit via the relative-position changing section 561, and the acquired information is input to the position detection apparatus 550A.

FIG. 24 is a diagram illustrating the positional relationships between the drive coil unit provided with the drive coil 51 of FIG. 23 and the sense coils 52.

In the position detection unit 550, there are provided a frame member 571 composed of substantially spherical outer frame 571A and inner frame 571B, a drive coil unit 551 arranged movably between the outer frame 571A and the inner frame 571B, and sense coils 52 arranged on the inner surface of the inner frame 571B, as shown in FIG. 24.

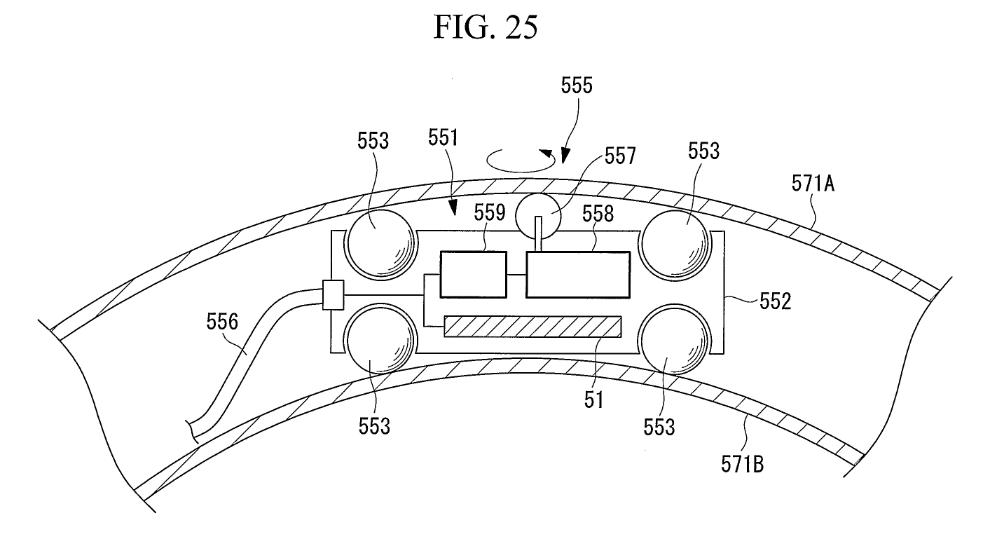

FIG. 25 is a diagram schematically showing the structure of the drive coil unit 551 of FIG. 24.

As shown in FIG. 25, the drive coil unit 551 is mainly composed of a substantially rectangular casing 552; drive sections 553 arranged in four corners of the surfaces of the casing 552, facing the outer frame 571A and the inner frame 571B; the drive coil 51; a direction changing section 555 for controlling the direction of movement of the drive coil unit 551; and a connection member 556 formed like a rope for electrically connecting the drive coil unit 551, the drive-coil driver 54, and the relative-position changing section 561.

The direction changing section 555 is mainly composed of a sphere section 557 arranged on a surface facing the outer frame 571A so as to protrude from the surface, a motor 558 for controlling the rotation of the sphere section 557, and a motor circuit 559 for controlling the driving of the motor 558.

The outline of the operation of the medical magnetic-induction and position-detection system 510 with the above-described structure is the same as that in the second embodiment, and thus a description thereof will be omitted here.

A procedure for detecting the position and orientation of the capsule endoscope 120 according to this embodiment will now be described.

The procedure for obtaining calculating frequencies used to detect the position and direction of the capsule endoscope 120, in other words, the operation up to storing the frequency characteristic of the magnetic induction coil 42 in the memory section 134A (refer to FIG. 15) is the same as that in the second embodiment, and thus the description there of will be omitted here.

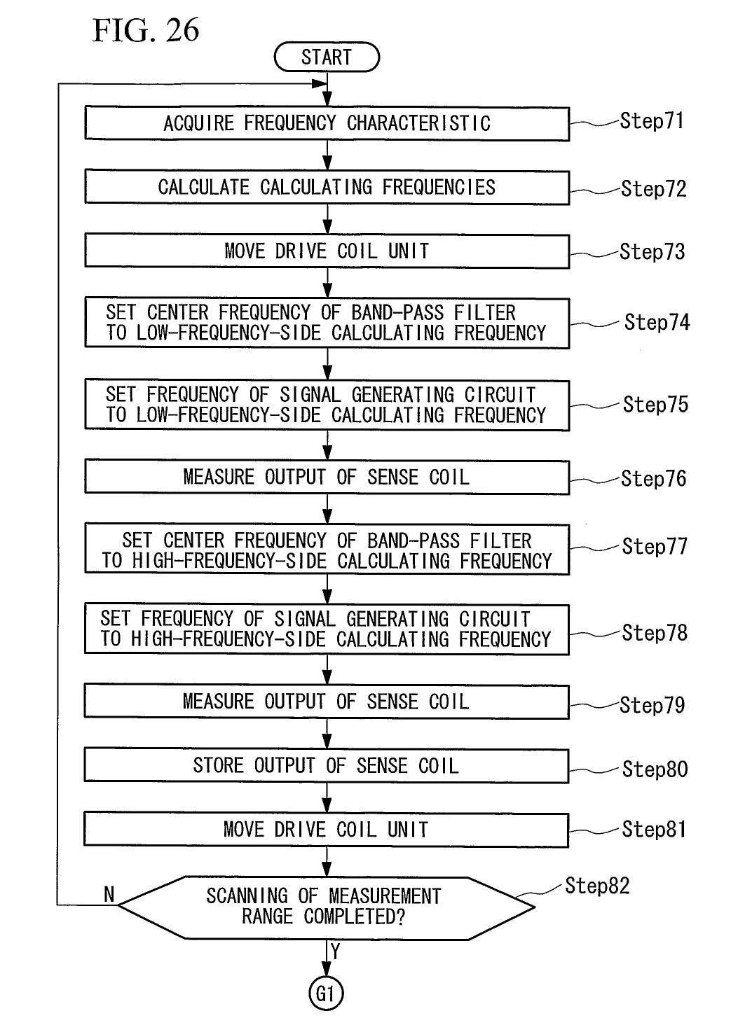

FIGS. 26, 27, and 28 are flowcharts illustrating a procedure for detecting the position and orientation of the capsule endoscope 120 according to this embodiment.

First, as shown in FIG. 26, the radio device 135 externally transmits the data for the frequency characteristic stored in the memory section 134A, and the data for the transmitted frequency characteristic is received by the capsule information reception circuit 181 and is then input to the frequency determining section 150B (Step 71).

Thereafter, the frequency determining section 150B obtains calculating frequencies used to detect the position and orientation of the capsule endoscope 120 based on the acquired frequency characteristic (Step 72; frequency determination step).

As with the first embodiment, the frequencies at which a change in gain of the sense coils 52 exhibits the maximum value and the minimum value are selected for the calculating frequencies. The lower frequency is referred to as the low-frequency-side calculating frequency, and the higher frequency is referred to as the high-frequency-side calculating frequency.

The drive coil unit 551 is moved to an end of the movable range (Step 73). More specifically, as shown in FIGS. 23 and 25, a control signal is output from the current-reference-value generating section 550B to the relative-position changing section 561, and the relative-position changing section 561 controls the driving of the drive sections 553 and the direction changing section 555 to move the drive coil unit 551.

Thereafter, as shown in FIG. 26, the center frequency of the band-pass filter 61 is adjusted to the low-frequency-side calculating frequency (Step 74). Furthermore, the transmission frequency range of the band-pass filter 61 is set to such a range that local extreme values of a change in gain of the sense coils 52 can be extracted.

Then, the frequency of the alternating magnetic field formed by the drive coil 51 is adjusted to the low-frequency-side calculating frequency (Step 75).

Then, an alternating magnetic field with the low-frequency-side calculating frequency is produced by the drive coil 51 to detect the alternating magnetic field with the sense coil 52 (Step 76).

Next, the center frequency of the band-pass filter 61 is adjusted to the high-frequency-side calculating frequency (Step 77).

Then, the frequency of an alternating magnetic field formed by the drive coil 51 is adjusted to the high-frequency-side calculating frequency (Step 78).

An alternating magnetic field with the high-frequency-side calculating frequency is produced by the drive coil 51 to detect the alternating magnetic field with the sense coils 52 (Step 79).

Thereafter, the information about relative positions of the drive coil 51 and the sense coils 52 is associated with the output of the sense coils 52 and is then stored in the storage section 550C of the current-reference-value generating section 550B as a reference value (Step 80).

Then, the drive coil unit 551 is moved to the subsequent predetermined position (Step 81). The predetermined positions are within the movable range of the drive coil unit 551 and are spaced out at predetermined intervals.

If there is a predetermined position for which a reference value is not acquired, the flow proceeds to the above-described Step 74 to repeat the acquisition of a reference value. When reference values have been acquired for all predetermined positions, the flow proceeds to the subsequent step (Step 82).

When reference values have been acquired for all predetermined positions, the capsule endoscope 120 is arranged and the drive coil unit 551 is moved to a position at which the position of the capsule endoscope 120 can be detected.

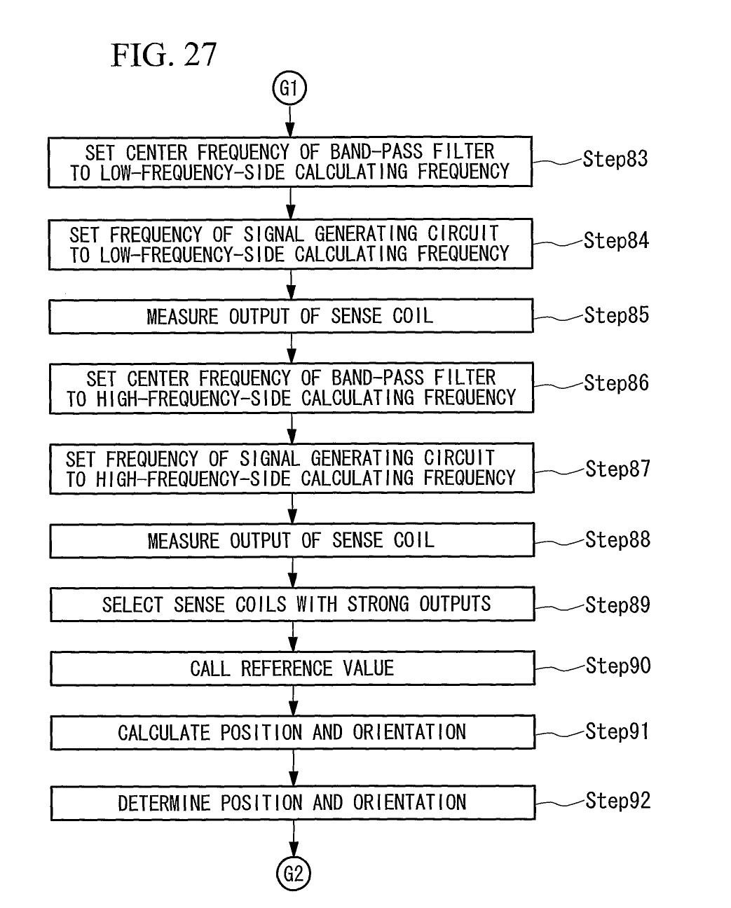

Thereafter, as shown in FIG. 27, the center frequency of the band-pass filter 61 is adjusted to the low-frequency-side calculating frequency (Step 83).

Then, the frequency of the alternating magnetic field formed by the drive coil 51 is adjusted to the low-frequency-side calculating frequency (Step 84).

Then, an alternating magnetic field with the low-frequency-side calculating frequency is produced by the drive coil 51 to detect the magnetic field induced by the magnetic induction coil 42 with the sense coils 52 (Step 85).

Next, the center frequency of the band-pass filter 61 is adjusted to the high-frequency-side calculating frequency (Step 86).

Then, the frequency of an alternating magnetic field formed by the drive coil 51 is adjusted to the high-frequency-side calculating frequency (Step 87).

An alternating magnetic field with the high-frequency-side calculating frequency is produced by the drive coil 51 to detect the magnetic field induced by the magnetic induction coil 42 with the sense coils 52 (Step 88).

As described above, detection with the low-frequency-side calculating frequency can be performed first, followed by detection with the high-frequency-side calculating frequency. Alternatively, detection with the high-frequency-side calculating frequency may be performed first, followed by detection with the low-frequency-side calculating frequency.

Thereafter, the position detection apparatus 550A calculates the output difference (amplitude difference) of each sense coil 52 between the low-frequency-side calculating frequency and the high-frequency-side calculating frequency, and then the sense coils 52 whose output difference is to be used to estimate the position of the capsule endoscope 120 are selected (Step 89).

The procedure for selecting the sense coils 52 is the same as that in the first embodiment, and a description thereof will be omitted here.

Then, the current-reference-value generating section 550B selects the reference value stored in the storage section 550C based on the current position of the drive coil 51 and sets it as the current reference value (Step 90). As a reference value to be selected, the reference value acquired for the relative positions closest to the current relative positions of the drive coil 51 and the sense coils 52 is desirable. By selecting in this manner, the time required to generate the current reference value can be reduced.

The position detection apparatus 550A calculates the position and direction of the capsule endoscope 120 based on the current reference value and the output of the sense coils 52 selected in Step 89 (Step 91) and determines the position and orientation (Step 92).

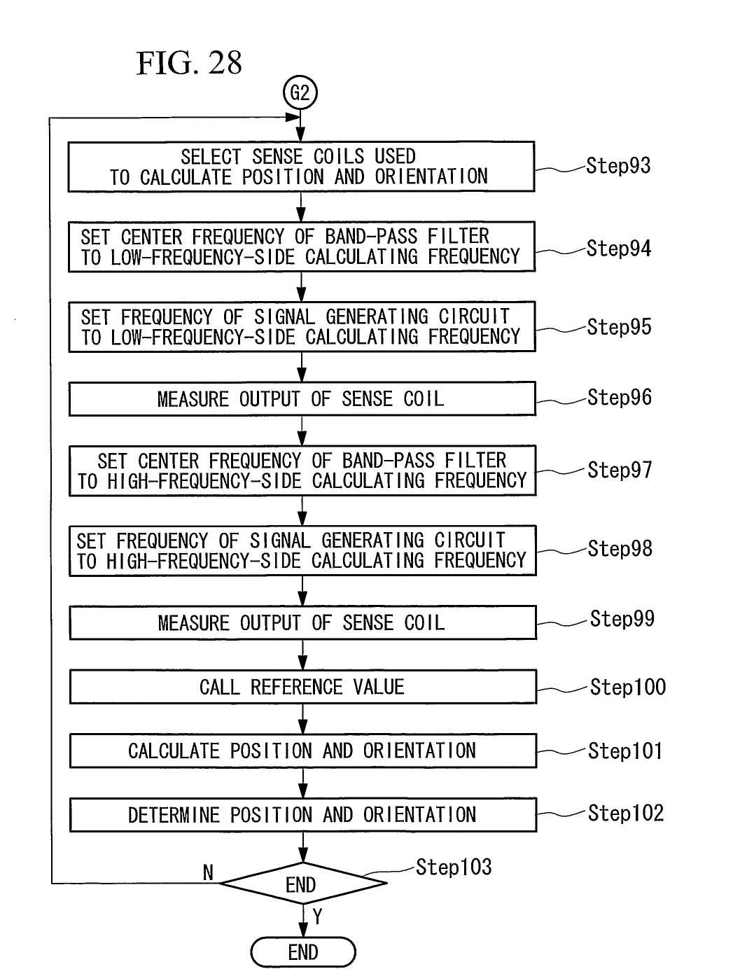

Then, sense coils 52 used for the subsequent control are selected as shown in FIG. 28 (Step 93).

More specifically, the position detection apparatus 550A estimates the direction of motion of the capsule endoscope 120 and the position and orientation after the movement of the capsule endoscope 120 based on the position and orientation of the capsule endoscope 120 determined in Step 92, and selects sense coils 52 having the largest outputs at the estimated position and orientation of the capsule endoscope 120.

Thereafter, the center frequency of the band-pass filter 61 is re-adjusted to the low-frequency-side calculating frequency (Step 94).

Then, the frequency of the alternating magnetic field formed by the drive coil 51 is adjusted to the low-frequency-side calculating frequency (Step 95).

Then, an alternating magnetic field with the low-frequency-side calculating frequency is generated by the drive coil 51 to detect the magnetic field induced by the magnetic induction coil 42 with the selected sense coils 52 (Step 96).

Next, the center frequency of the band-pass filter 61 is adjusted to the high-frequency-side calculating frequency (Step 97).

Then, the frequency of the alternating magnetic field formed by the drive coil 51 is adjusted to the high-frequency-side calculating frequency (Step 98).

Then, an alternating magnetic field with the high-frequency-side calculating frequency is produced by the drive coil 51 to detect the magnetic field induced by the magnetic induction coil 42 with the selected sense coils 52 (Step 99).

The reference value stored in the storage section 550C is selected based on the current position of the drive coil 51 and is set as the current reference value (Step 100). As a reference value to be selected, the reference value acquired for the relative positions closest to the current relative positions of the drive coil 51 and the sense coils 52 is desirable.

The position detection apparatus 550A calculates the position and orientation of the capsule endoscope 120 based on the current reference value in Step 100 and the output of the sense coils 52 selected in Step 93 (Step 101) and determines the position and orientation (Step 102).

Thereafter, if the detection of the position and orientation of the capsule endoscope apparatus 120 is continued, the flow returns to the above-described Step 93 for the detection of the position and orientation (Step 103).

With the above-described structure, even if the relative positions of the drive coil 51 and the sense coils 52 are variable, the position and orientation of the capsule endoscope 120 can be obtained.

Since the above-described reference value and the position and relative positions of the drive coil 51 are pre-stored, even if the relative positions of the drive coil 51 and the sense coils 52 when the position of the capsule endoscope 120 is detected are different, it is not necessary to re-measure the above-described reference value and so forth.

Instead of the above-described procedure for generating the current reference value, the current-reference-value generating section 550B may obtain a predetermined approximate equation that associates relative positions and reference values to generate the current reference value based on that predetermined approximate equation and the current relative positions. According to this generating method, since the current reference value is generated based on a predetermined approximate equation, a more accurate current reference value can be generated compared with, for example, a method where the reference value stored in the storage section 550C is set as the current reference value. Furthermore, the predetermined approximate equation is not particularly limited, and any known approximate equation can be used.

(Position Detection System for Capsule Endoscope)

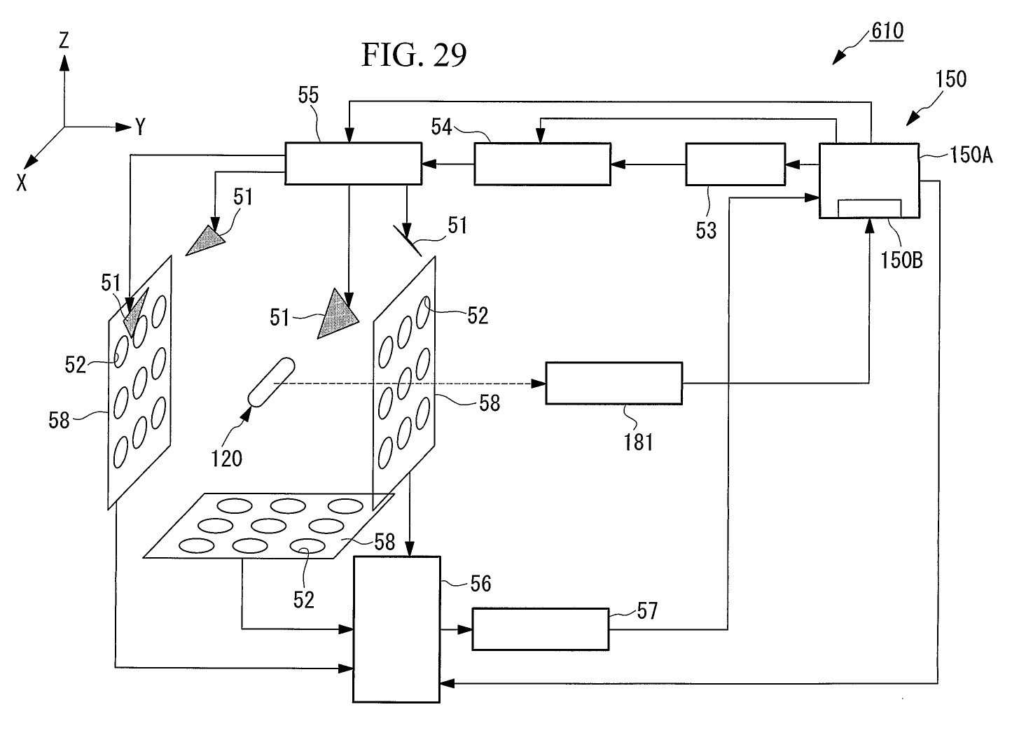

A position detection system for a capsule endoscope according to the present invention will now be described with reference to FIG. 29.

FIG. 29 is a diagram schematically showing the position detection system for a capsule endoscope according to the present invention.

A position detection system 610 for a capsule endoscope according to the present invention is composed of only the position detection unit 150 of the above-described medical magnetic-induction and position-detection system 110. Therefore, the components, operation, and advantages of the position detection system 610 for a capsule endoscope are the same as those of the medical magnetic-induction and position-detection system 110: a description thereof will be omitted and only FIG. 29 shown.

In addition, the present invention is applied to the position detection system for a capsule endoscope, the medical magnetic-induction and position-detection system, and the position detection method for a capsule medical device, as described above. However, a device that is swallowed by a subject, such as an examinee, can be used not only as a capsule endoscope but also as a capsule medical device (various types of capsule medical devices, such as a DDS capsule that holds a drug and discharges the drug at a target site in the body cavity; a sensor capsule provided with a chemical sensor, a blood sensor, a DNA probe, or the like to acquire information in the body cavity; and an indwelling capsule that is left inside a body to measure, for example, pH). Furthermore, the magnetic induction coil can be arranged in a tip catheter of an endoscope, the tip of forceps, and so forth, and the position detection system described in the present invention can also be used as a position detection system for a medical device functioning in a body cavity.

Furthermore, it is sufficient that the sense coils 52 are magnetic field sensors that can detect a magnetic field, and various sensors, such as GMR sensors, MI sensors, Hall elements, and SQUID fluxmeters, can be used.

### Other Modifications of First to Fifth Embodiments

In each of the above-described first to fifth embodiments, it is necessary to prevent the magnetic field intensity for position detection from decreasing in the operating region of the medical device.

For example, in the above-described document 6, a technique for externally arranging a substantially rectangular magnetic field source (position-detection magnetic-field generating coil) having three three-axis orthogonal magnetic-field generating coils and for arranging a magnetic field detection coil having three three-axis orthogonal magnetic-field receiving coils in a medical capsule is disclosed. According to this technique, an induction current can be generated in the magnetic field detection coil resulting from an alternating magnetic field generated by the magnetic field source to detect the position of the magnetic field detection coil, namely, the position of the medical capsule, based on the generated induction current.

On the other hand, in the above-described document 7, a position detection system including an exciting coil (position-detection magnetic-field generating coil) for generating an alternating magnetic field, an LC resonant magnetic marker for receiving the alternating magnetic field to generate an induced magnetic field, and a detection coil for detecting the induced magnetic field is disclosed. According to this position detection system, since the LC resonant magnetic marker causes resonance at a predetermined frequency due to parasitic capacitance, matching the frequency of the above-described alternating magnetic field to the above-described predetermined frequency can cause the intensity of the induced magnetic field to be dramatically higher than at other frequencies, thereby increasing the detection efficiency.

However, for the techniques disclosed in the above-described documents 6 and 7, if a technique that uses a magnetic field to guide, for example, a medical capsule is combined and a guidance-magnetic-field generating coil for generating a guidance magnetic field is arranged such that its central axis is substantially the same as that of the above-described position-detection magnetic-field generating coil, then there is danger of mutual induction occurring between the position-detection magnetic-field generating coil and the guidance-magnetic-field generating coil, depending on a change over time in the alternating magnetic field generated by the position-detection magnetic-field generating coil.

In short, there is a problem that an electromotive force generated by the above-described mutual induction in the guidance-magnetic-field generating coil causes an electric current to flow in a closed circuit formed of the guidance-magnetic-field generating coil and a guidance-coil drive apparatus and generate a magnetic field that cancels out the above-described alternating magnetic field with that electric current.

Furthermore, since the guidance-magnetic-field generating coil makes the magnetic field distribution in an induction space uniform, it is often constructed so as to provide a Helmholtz or similar function, and driving is performed typically by serially connecting two guidance-magnetic-field generating coils to the guidance-coil drive apparatus. In this case, even if an electromotive force due to mutual induction occurs only in one of the guidance-magnetic-field generating coils, since a closed circuit is formed by the guidance-coil drive apparatus, an electric current flows in the other guidance-magnetic-field generating coil too. Because of this, a magnetic field having a phase substantially opposite to that of the position-detection magnetic field is widely distributed in the induction space.

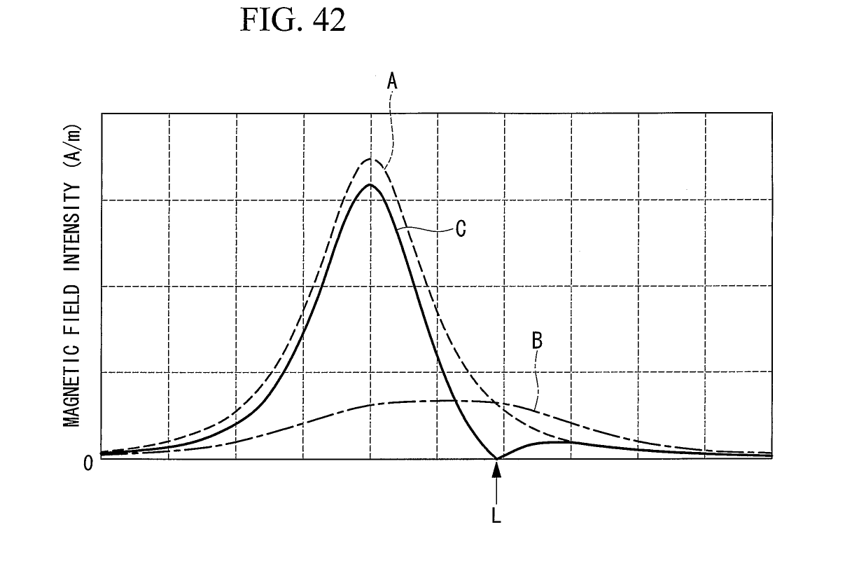

At this time, as shown in FIG. 42, a combined magnetic field (solid line C) of the position-detection magnetic field (broken line A) generated by the position-detection magnetic-field generating coil and the induced magnetic field (broken line B) generated by the induction-magnetic-field generating coil intersects the coil built into, for example, the capsule. In particular, depending on the relative positional relationship between the position-detection magnetic-field generating coil and the induction-magnetic-field generating coil, there is danger that some area (L) of the above-described position-detection magnetic field (broken line A) is nearly completely canceled out by the above-described mutual induction magnetic field (broken line B) even within the operating region of, for example, the medical capsule. As a result, there occurs a problem such that since no induction current flows as a result of no magnetic field intersecting the coil built into, for example, the capsule, no induced magnetic field is generated, and therefore, detection of the position of, for example, the medical capsule is impossible in that area.

In order to overcome the above-described problem, the following modifications can be employed to prevent the magnetic field intensity for position detection from decreasing within the operating region of the medical device.

First Modification

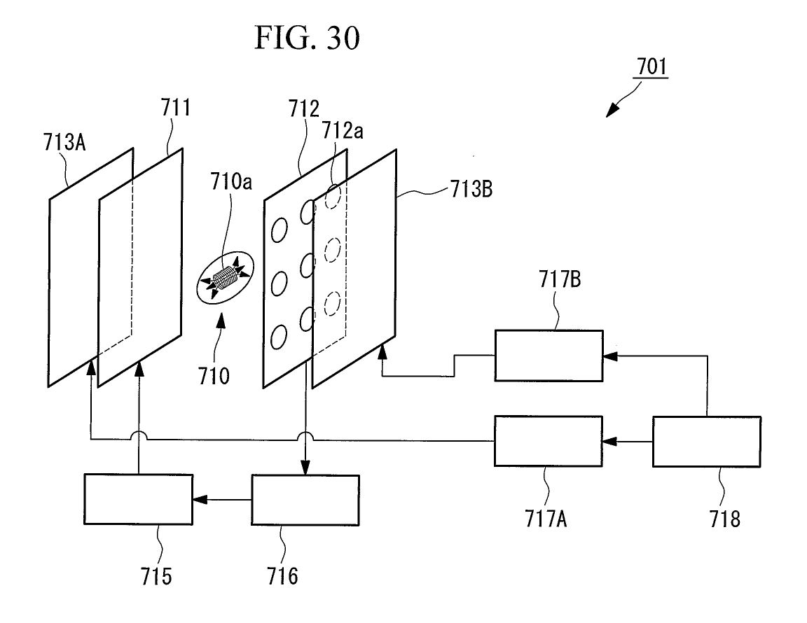

A first modification of the medical magnetic-induction and position-detection system according to the present invention will now be described with reference to FIGS. 30 through 33.

FIG. 30 is a schematic diagram showing the outline structure of the medical magnetic-induction and position-detection system according to this modification.

As shown in FIG. 30, a medical magnetic-induction and position-detection system 701 is mainly composed of a position-detection magnetic-field generating coil (first magnetic-field generating section, drive coil) 711 for generating a position-detection magnetic field (first magnetic field); a sense coil (magnetic field sensor, magnetic-field detection section) 712 for detecting an induced magnetic field generated by a magnetic induction coil (built-in coil) 710a installed in a capsule endoscope (medical device) 710; and guidance-magnetic-field generating coils (guidance-magnetic-field generating unit, electromagnets, opposed coils) 713A and 713B for generating guidance magnetic fields (second magnetic fields) for guiding the capsule endoscope to a predetermined position in a body cavity.

The capsule endoscope 710 is provided with a closed circuit including the magnetic induction coil 710a and a capacitor having predetermined capacitance; and a magnet (not shown in the figure) used to control the position and orientation of the capsule endoscope 710 in conjunction with the guidance magnetic field. The above-described closed circuit forms an LC resonant circuit that brings about resonance at a predetermined frequency. The above-described closed circuit can be constructed as an LC resonant circuit, or if a predetermined resonance frequency can be achieved with parasitic capacitance in the magnetic induction coil 710a, the magnetic induction coil 710a alone, with both ends open, can (equivalently) form the closed circuit.

As the capsule endoscope 710, various types of medical devices can be listed, including capsule endoscopes having an electronic imaging element, such as a CMOS device or a CCD, installed therein and devices for transporting a drug to a predetermined position in the body cavity of the subject and discharging the drug. The capsule endoscope 710 is not particularly limited.

The position-detection magnetic-field generating coil 711 is composed of a coil formed in a substantially planar shape and is electrically connected to a position-detection magnetic-field generating-coil drive section 715.

The sense coil 712 is composed of a plurality of detection coils 712a arranged in a substantially planar shape, and each detection coil 712a is electrically connected to the position-detection control section 716 so that the output of the detection coil 712a is input to the position-detection control section 716.

The position-detection control section 716 is electrically connected to the position-detection magnetic-field generating-coil drive section 715 so that a control signal generated by the position-detection control section 716 is input to the position-detection magnetic-field generating-coil drive section 715.

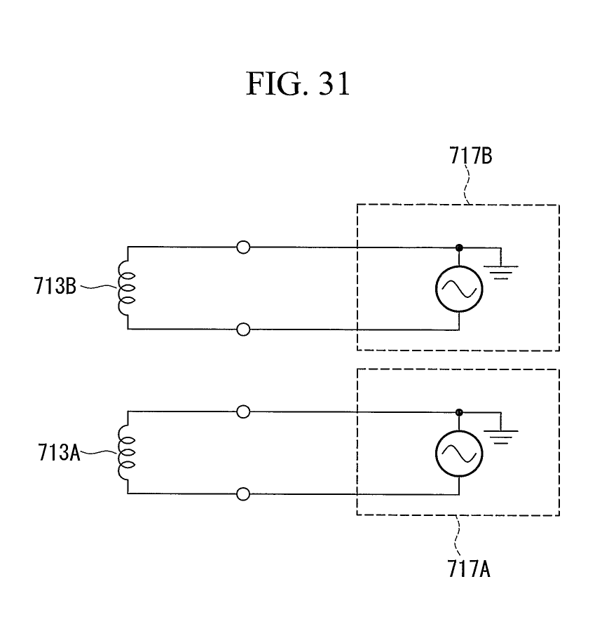

FIG. 31 is a connection diagram illustrating the structure of the guidance-magnetic-field generating coils shown in FIG. 30.

The guidance-magnetic-field generating coils 713A and 713B are composed of coils formed in a substantially planar shape and are electrically connected to guidance-magnetic-field-generating-coil drive sections 717A and 717B, respectively, as shown in FIGS. 30 and 31. The guidance-magnetic-field-generating-coil drive sections 717A and 717B are electrically connected to an induction control section 718, and a control signal generated by the induction control section 718 is input thereto.

The guidance-magnetic-field generating coil 713A is arranged so as to face the vicinity of the position-detection magnetic-field generating coil 711 and so as to be positioned at the opposite side of the position-detection magnetic-field generating coil 711 from the capsule endoscope 710. The guidance-magnetic-field generating coil 713B is arranged so as to face the vicinity of the sense coil 712 and so as to be positioned at the opposite side of the sense coil 712 from the capsule endoscope 710.

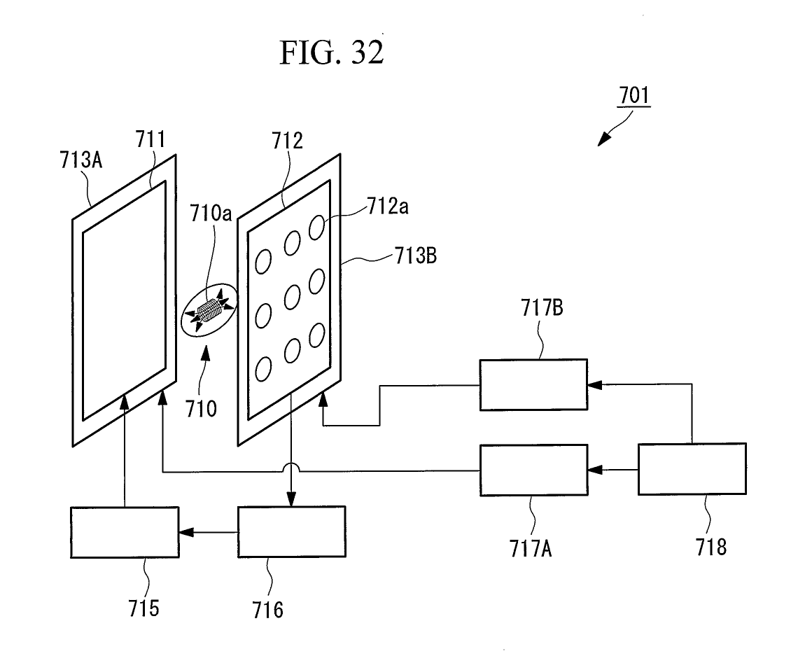

The positional relationship between the guidance-magnetic-field generating coil 713A and the position-detection magnetic-field generating coil 711 or the positional relationship between the guidance-magnetic-field generating coil 713B and the sense coil 712 can be switched. Furthermore, if the guidance-magnetic-field generating coil 713A has an air core and is shaped so as to accommodate therein the position-detection magnetic-field generating coil 711, then the guidance-magnetic-field generating coil 713A and the position-detection magnetic-field generating coil 711 may be arranged on substantially the same flat surface, as shown in FIG. 32. In addition, if the guidance-magnetic-field generating coil 713B has an air core and is shaped so as to accommodate therein the sense coil 712, then the guidance-magnetic-field generating coil 713B and the sense coil 712 may be arranged on substantially the same flat surface.

The operation of the medical magnetic-induction and position-detection system 701 with the above-described structure will now be described.

First, as shown in FIG. 30, a position-detection control signal, which is an AC signal having a predetermined frequency, is generated in the position-detection control section 716, and the position-detection control signal is output to the position-detection magnetic-field generating-coil drive section 715. The position-detection magnetic-field generating-coil drive section 715 amplifies the input position-detection control signal to a predetermined intensity and generates a drive current for driving the position-detection magnetic-field generating coil 711. The drive current is output to the position-detection magnetic-field generating coil 711, and the magnetic-field generating coil 11 forms a position-detection magnetic field therearound as a result of the drive current being supplied.

When the magnetic flux of the position-detection magnetic field intersects the capsule endoscope 710, a resonant current with a predetermined frequency is induced in the closed circuit having the magnetic induction coil 710a installed therein. When a resonant current is induced in the closed circuit, it causes the magnetic induction coil 710a to form therearound an induced magnetic field having a predetermined frequency.

Since the magnetic fluxes of the position-detection magnetic field and the induced magnetic field intersect the detection coils 712a of the sense coil 712, the detection coils 712a capture a magnetic flux generated by adding the magnetic fluxes of both the magnetic fields and generate an output signal that is an induction current based on a change in the intersecting magnetic fluxes. An output signal of each detection coil 712a is output to the position-detection control section 716.

The position-detection control section 716 controls the frequency of the position-detection magnetic field formed in the position-detection magnetic-field generating coil 711. More specifically, the frequency of the position-detection magnetic field is changed by changing the frequency of the above-described control signal generated in the position-detection control section 716. When the frequency of the position-detection magnetic field is changed, the relative relationship with the resonance frequency of the closed circuit in the capsule endoscope 710 is changed, and the intensity of the induced magnetic field formed in the magnetic induction coil 710a changes. In this example, a change in detection voltage in the vicinity of the resonance frequency is detected for the purpose of position calculation.

Furthermore, in the position-detection control section 716, the position of the magnetic induction coil 710a, namely the capsule endoscope 710, is estimated based on the output signal from the detection coil 712a using a known computation method.

As shown in FIGS. 30 and 31, the induction control section 718 generates a guidance control signal, which is an AC signal having a predetermined frequency, and the guidance control signal is output to the guidance-magnetic-field-generating-coil drive sections 717A and 717B.

The guidance-magnetic-field-generating-coil drive sections 717A and 717B amplify the input guidance control signals to a predetermined intensity and generate drive currents for driving the guidance-magnetic-field generating coils 713A and 713B. The drive currents are output to the guidance-magnetic-field generating coils 713A and 713B, and the guidance-magnetic-field generating coils 713A and 713B form therearound guidance magnetic fields as a result of the drive currents being supplied.

Since the guidance-magnetic-field generating coils are connected to the guidance-magnetic-field-generating-coil drive sections having significantly low output impedance, mutual induction occurs between both coils when the position-detection magnetic field intersects the guidance-magnetic-field generating coils. As a result, a generated electromotive force causes an electric current to flow in the closed circuit formed of the guidance-magnetic-field generating coils and the guidance-magnetic-field-generating-coil drive sections. Because of this, a magnetic field is generated in a direction canceling out the position-detection magnetic field by the guidance-magnetic-field generating coils.

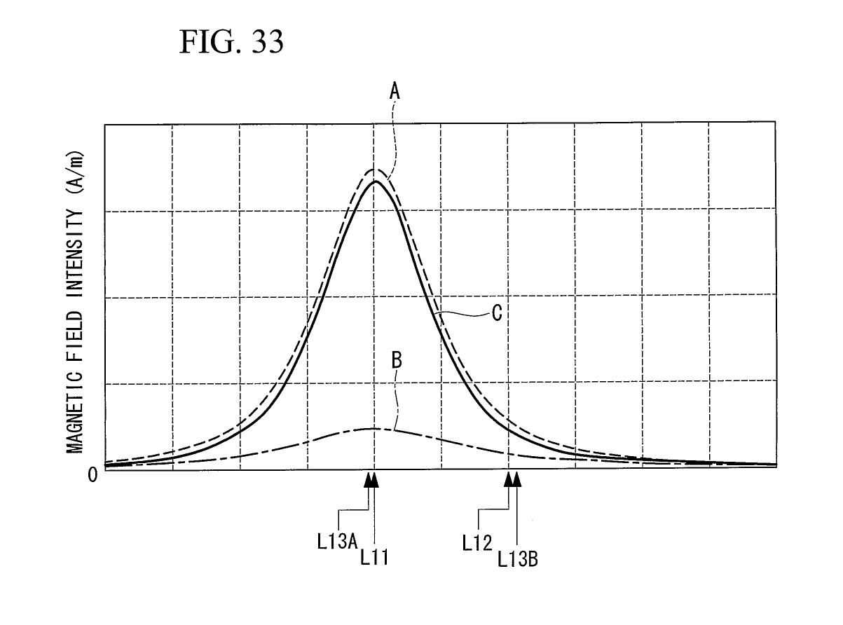

FIG. 33 is a diagram illustrating the magnetic field intensity formed in the medical magnetic-induction and position-detection system of FIG. 30.

The above-described position-detection magnetic-field generating coil 711 and the guidance-magnetic-field generating coils 713A and 713B form magnetic fields with the magnetic field intensity distributions shown in FIG. 33. The intensity distribution of the position-detection magnetic field formed by the position-detection magnetic-field generating coil 711 is indicated by broken line A in FIG. 33, the intensity distribution of the mutual induction magnetic field formed by the guidance-magnetic-field generating coil 713A is indicated by chain line B in FIG. 33, and the combined magnetic field of the position-detection magnetic field and the mutual induction magnetic field generated by the guidance-magnetic-field generating coil is indicated by solid line C in FIG. 33.

The intensity distribution of the position-detection magnetic field is such that the intensity is the maximum at a position L11 where the position-detection magnetic-field generating coil 711 is disposed, and the intensity decreases away from this position. The intensity distribution of the mutual induction magnetic field generated by the guidance-magnetic-field generating coil is such that the intensity is the maximum at a position L13A where the guidance-magnetic-field generating coil 713A is disposed, and the intensity decreases away from this position. Furthermore, the combined magnetic field of the position-detection magnetic field and the mutual induction magnetic field cancels out because the position-detection magnetic field and the mutual induction magnetic field have phases opposite to each other. Here, the position L13A at which the intensity of the mutual induction magnetic field becomes the maximum is near or at the position L11 at which the intensity of the position-detection magnetic field becomes the maximum, and the maximum intensity of the mutual induction magnetic field is lower than the maximum intensity of the position-detection magnetic field. For this reason, at least in the space interposed between the guidance-magnetic-field generating coils 713A and 713B, the intensity of the mutual induction magnetic field is substantially equal to or is less than the intensity of the position-detection magnetic field. Therefore, the combined magnetic field exhibits a magnetic field intensity distribution where the intensity is lower than that of the position-detection magnetic field. More specifically, the intensity becomes the maximum near the position L11 where the position-detection magnetic-field generating coil 711 is disposed and the position L13A where the guidance-magnetic-field generating coil 713A is disposed and decreases away from these positions.

With the above-described structure, as shown in FIG. 42, since an area where the combined magnetic field becomes substantially zero is prevented from occurring, an area where no induced magnetic field is generated is prevented from occurring in the magnetic induction coil 710a installed in the capsule endoscope 710. Accordingly, an area where the position of the capsule endoscope 710 cannot be detected is prevented from occurring.

Since the driving of the guidance-magnetic-field generating coils 713A and 713B is individually controlled by the guidance-magnetic-field-generating-coil drive sections 717A and 717B, respectively, an electric current originating from the electromotive force generated in the coil 713A does not flow in the guidance-magnetic-field generating coil 713B by controlling the driving of the guidance-magnetic-field generating coil 713B with the guidance-magnetic-field-generating-coil drive section 717B. Consequently, a magnetic field that substantially cancels out the position-detection magnetic field is prevented from occurring in the vicinity of the sense coil.

Furthermore, since the formation of the guidance magnetic field can be continued by controlling the driving of the guidance-magnetic-field generating coil 713A with the guidance-magnetic-field-generating-coil drive section 717A, guidance of the capsule endoscope 710 can be continued.

Second Modification

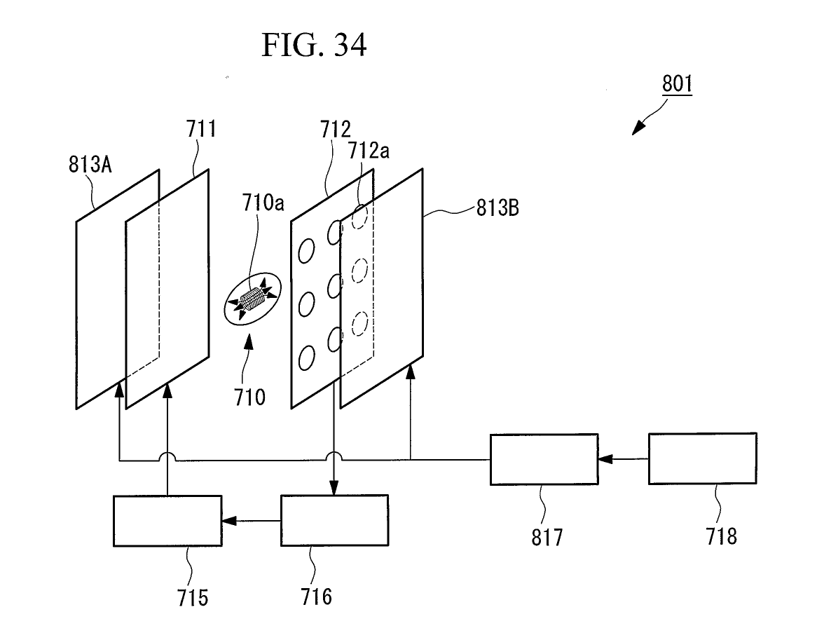

A second modification according to the present invention will now be described with reference to FIGS. 34 through 36.

The basic configuration of the medical magnetic-induction and position-detection system according to this modification is the same as that in the first modification; however, the structure of the induction-magnetic-field generating-coil drive section is different from that in the first modification. Therefore, in this modification, only the vicinity of the structure of the induction-magnetic-field generating-coil drive section will be described using FIGS. 34 through 36, and a description of the other components will be omitted.

FIG. 34 is a schematic diagram depicting the outline structure of a medical magnetic-induction and position-detection system according to this modification.

The same components as those in the first modification are denoted with the same reference numerals, and thus will not be described again here.

As shown in FIG. 34, a medical magnetic-induction and position-detection system 801 is mainly composed of a position-detection magnetic-field generating coil 711 for generating a position-detection magnetic field; a sense coil 712 for detecting an induced magnetic field generated by a magnetic induction coil 710a installed in a capsule endoscope 710; and guidance-magnetic-field generating coils (guidance-magnetic-field generating unit, electromagnets, opposed coils) 813A and 813B for generating guidance magnetic fields.

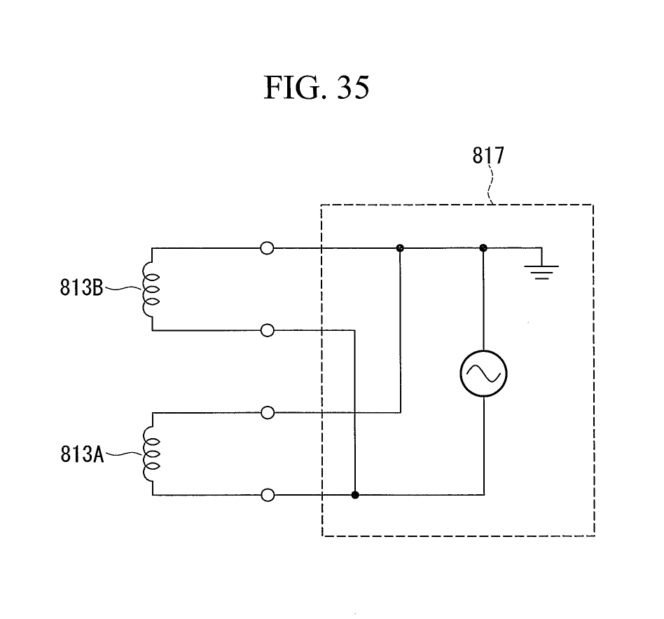

FIG. 35 is a connection diagram illustrating the structure of the guidance-magnetic-field generating coils of FIG. 34.

The guidance-magnetic-field generating coils 813A and 813B are composed of coils formed in a substantially planar shape and, as shown in FIGS. 34 and 35, are electrically connected to a guidance-magnetic-field-generating-coil drive section 817. The guidance-magnetic-field generating coils 813A and 813B are electrically connected in parallel to the guidance-magnetic-field-generating-coil drive section 817. The guidance-magnetic-field-generating-coil drive section 817 is electrically connected to an induction control section 718, and a control signal generated by the induction control section 718 is input thereto.

The guidance-magnetic-field generating coil 813A is arranged so as to face the vicinity of the position-detection magnetic-field generating coil 711 and so as to be positioned at the opposite side of the position-detection magnetic-field generating coil 711 from the capsule endoscope 710. The guidance-magnetic-field generating coil 813B is arranged so as to face the vicinity of the sense coil 712 and so as to be positioned at the opposite side of the sense coil 712 from the capsule endoscope 710.

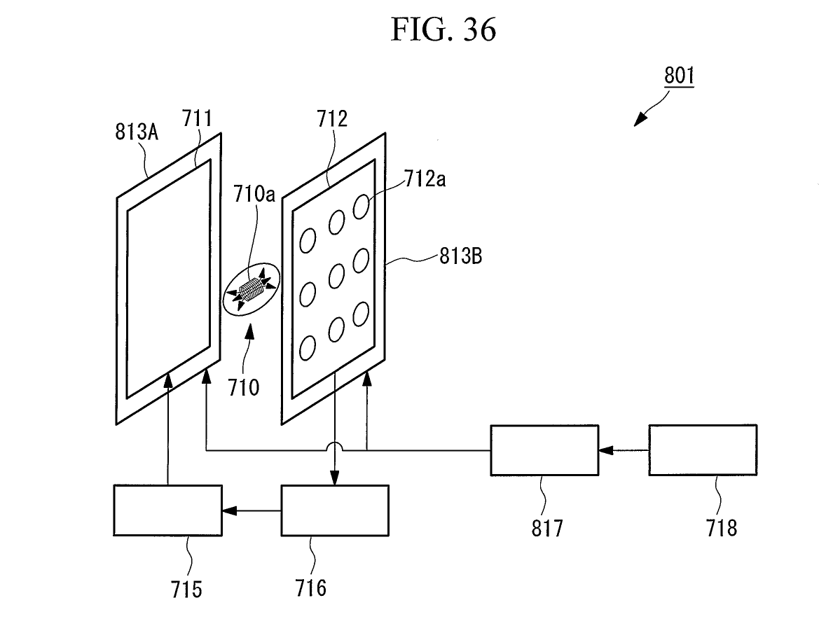

The positional relationship between the guidance-magnetic-field generating coil 813A and the position-detection magnetic-field generating coil 711 or the positional relationship between the guidance-magnetic-field generating coil 813B and the sense coil 712 can be switched. Furthermore, if the guidance-magnetic-field generating coil 813A has an air core and is shaped so as to accommodate therein the position-detection magnetic-field generating coil 711, then the guidance-magnetic-field generating coil 813A and the position-detection magnetic-field generating coil 711 may be arranged on substantially the same flat surface, as shown in FIG. 36. In addition, if the guidance-magnetic-field generating coil 813B has an air core and is shaped so as to accommodate therein the sense coil 712, then the guidance-magnetic-field generating coil 813B and the sense coil 712 may be arranged on substantially the same flat surface.

The operation of the medical magnetic-induction and position-detection system 801 with the above-described structure will now be described.

Operations related to detecting the position of the capsule endoscope 710, such as the formation of a position-detection magnetic field in the position-detection magnetic-field generating coil 711 and the formation of an induced magnetic field in the magnetic induction coil 710a, are the same as those in the first modification, and thus a description thereof shall be omitted here.

As shown in FIGS. 34 and 35, the induction control section 718 generates a guidance control signal, which is an AC signal having a predetermined frequency, and the guidance control signal is output to the guidance-magnetic-field-generating-coil drive section 817.

The guidance-magnetic-field-generating-coil drive section 817 amplifies the input guidance control signal to a predetermined intensity and generates a drive current for driving the guidance-magnetic-field generating coils 813A and 813B. The drive current is output to the guidance-magnetic-field generating coils 813A and 813B, and the guidance-magnetic-field generating coils 813A and 813B generate guidance magnetic fields therearound as a result of the drive current being supplied.

The position-detection magnetic field formed by the above-described position-detection magnetic-field generating coil 711 and the guidance-magnetic-field generating coils 813A and 813B, the mutual induction magnetic field issued from the guidance-magnetic-field generating coil, and the magnetic field intensity distribution of the combined magnetic field of these magnetic fields are the same as those in the first modification, and thus a description thereof shall be omitted here.

With the above-described structure, since an area where the combined magnetic field becomes substantially zero is prevented from occurring, an area where an induced magnetic field is not generated is prevented from occurring in the magnetic induction coil 710a installed in the capsule endoscope 710. Accordingly, an area where the position of the capsule endoscope 710 cannot be detected is prevented from occurring.

Since the guidance-magnetic-field generating coils 813A and 813B are electrically connected in parallel, the position-detection magnetic field is prevented from generating a mutual induction magnetic field in the guidance-magnetic-field generating coil 813B.

Furthermore, since the formation of the guidance magnetic field can be continued in the guidance-magnetic-field generating coil 813A, guidance of the capsule endoscope 710 can be continued.

Third Modification

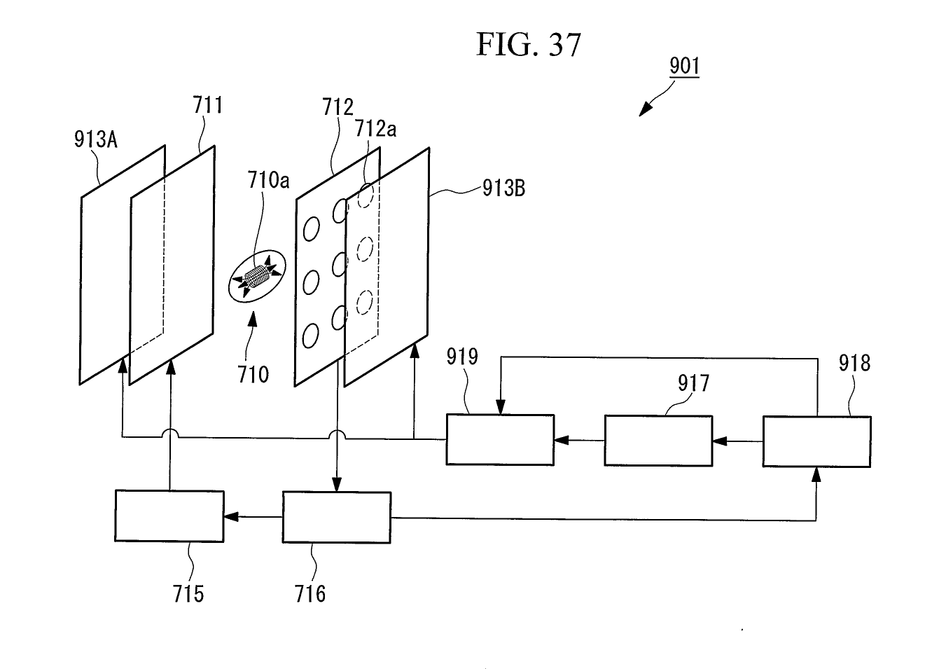

A third modification according to the present invention will now be described with reference to FIGS. 37 through 39.

The basic configuration of the medical magnetic-induction and position-detection system according to this modification is the same as that in the first modification; however, the structure of the induction-magnetic-field generating-coil drive section is different from that in the first modification. Therefore, in this modification, only the vicinity of the structure of the induction-magnetic-field generating-coil drive section will be described using FIGS. 37 through 39, and a description of the other components will be omitted.

FIG. 37 is a schematic diagram depicting the outline structure of a medical magnetic-induction and position-detection system according to this modification.

The same components as those in the first modification are denoted with the same reference numerals, and thus will not be described again here.

As shown in FIG. 37, a medical magnetic-induction and position-detection system 901 is mainly composed of a position-detection magnetic-field generating coil 711 for generating a position-detection magnetic field; a sense coil 712 for detecting an induced magnetic field generated by a magnetic induction coil 710a installed in a capsule endoscope 710; and guidance-magnetic-field generating coils (guidance-magnetic-field generating unit, electromagnets, opposed coils) 913A and 913B for generating guidance magnetic fields.

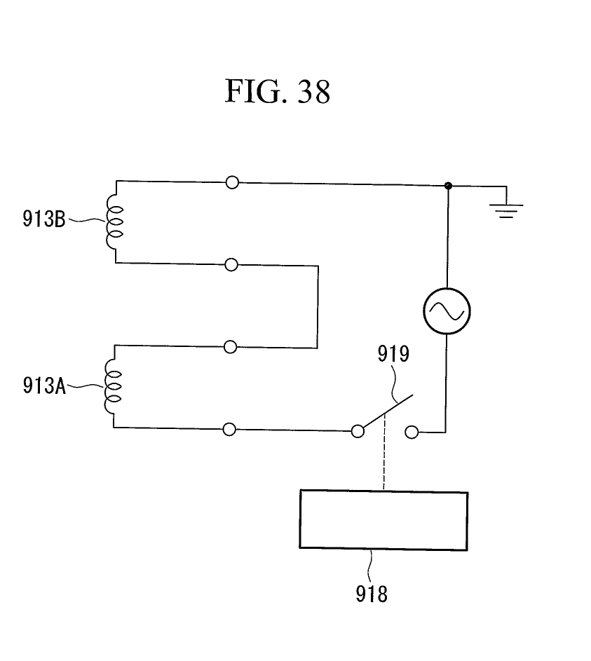

FIG. 38 is a connection diagram illustrating the structure of the guidance-magnetic-field generating coils of FIG. 37.

The guidance-magnetic-field generating coils 913A and 913B are composed of coils formed in a substantially planar shape and, as shown in FIGS. 37 and 38, are electrically connected to a guidance-magnetic-field-generating-coil drive section 917 via a switching section 919. The switching section 919 is provided in a closed circuit composed of the guidance-magnetic-field generating coils 913A and 913B and the guidance-magnetic-field-generating-coil drive section 917.

The guidance-magnetic-field generating coils 913A and 913B are electrically connected in series. The guidance-magnetic-field-generating-coil drive section 917 is electrically connected to the induction control section 918, and a control signal generated by the induction control section 918 is input thereto. The induction control section 918 is electrically connected to the switching section 919, and an ON/OFF signal generated by the induction control section 918 is input to the switching section 919. Furthermore, the induction control section 918 is also electrically connected to the position-detection control section 716 so that an operation signal output from the position-detection control section 716 is input to the induction control section 918.

The guidance-magnetic-field generating coil 913A is arranged so as to face the vicinity of the position-detection magnetic-field generating coil 711 and so as to be positioned at the opposite side of the position-detection magnetic-field generating coil 711 from the capsule endoscope 710. The guidance-magnetic-field generating coil 913B is arranged so as to face the vicinity of the sense coil 712 and so as to be positioned at the opposite side of the sense coil 712 from the capsule endoscope 710.

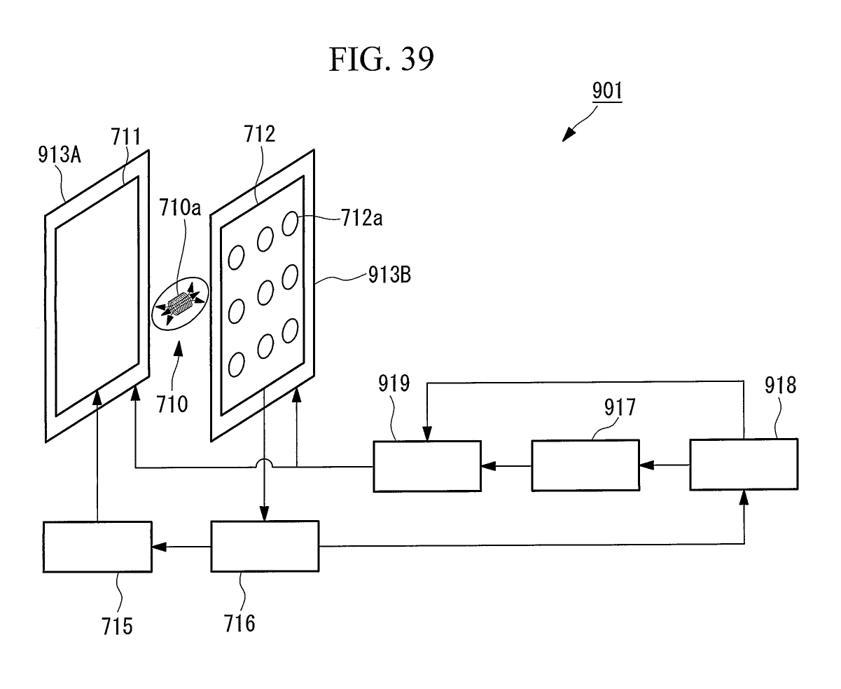

The positional relationship between the guidance-magnetic-field generating coil 913A and the position-detection magnetic-field generating coil 711 or the positional relationship between the guidance-magnetic-field generating coil 913B and the sense coil 712 can be switched. Furthermore, if the guidance-magnetic-field generating coil 913A has an air core and is shaped so as to accommodate therein the position-detection magnetic-field generating coil 711, then the guidance-magnetic-field generating coil 913A and the position-detection magnetic-field generating coil 711 may be arranged on substantially the same flat surface, as shown in FIG. 39. In addition, if the guidance-magnetic-field generating coil 913B has an air core and is shaped so as to accommodate therein the sense coil 712, then the guidance-magnetic-field generating coil 913B and the sense coil 712 may be arranged on substantially the same flat surface.

The operation of the medical magnetic-induction and position-detection system 901 with the above-described structure will now be described.

Operations related to detecting the position of the capsule endoscope 710, such as the formation of a position-detection magnetic field in the position-detection magnetic-field generating coil 711 and the formation of an induced magnetic field in the magnetic induction coil 710a, are the same as those in the first modification, and thus a description thereof shall be omitted here.

As shown in FIGS. 37 and 38, the induction control section 918 generates a guidance control signal, which is an AC signal having a predetermined frequency, and the guidance control signal is output to the guidance-magnetic-field-generating-coil drive section 917.

The guidance-magnetic-field-generating-coil drive section 917 amplifies the input guidance control signal to a predetermined intensity and generates a drive current for driving the guidance-magnetic-field generating coils 913A and 913B. The drive current is output to the guidance-magnetic-field generating coils 913A and 913B, and the guidance-magnetic-field generating coils 913A and 913B generate guidance magnetic fields therearound as a result of the drive current being supplied thereto.

An ON/OFF signal for controlling the switching section 919 based on an operation signal input from a position-detection control section 716 is output to the induction control section 918. The operation signal is generated based on a control signal output to a position-detection magnetic-field generating-coil drive section 715. More specifically, while the control signal for forming a position-detection magnetic field is output to the position-detection magnetic-field generating-coil drive section 715, an operation signal for turning off (opening) the switching section 919 is output.

On the other hand, while the control signal is not output, an operation signal for turning on (closing) the switching section 919 is output.

The induction control section 918 outputs an ON/OFF signal to the switching section 919 based on the control signal input as described above, and the ON/OFF state of the switching section 919 is controlled based on the ON/OFF signal.

When the switching section 919 is to be turned on/off, the ON/OFF state of the switching section 919 may be simply controlled as described above, or the induction control section 918 may gradually change the amplitude of the signal input to the induction-magnetic-field generating-coil drive section 917 based on the operation signal. By performing control as described above, a back-electromotive force due to self-induction of the guidance-magnetic-field generating coils 913A and 913B is prevented from damaging the guidance-magnetic-field-generating-coil drive section 917.

Alternatively, it is also acceptable that when the switching section 919 is to be turned off, the induction control section 918 gradually brings to zero the amplitude of the signal input to the guidance-magnetic-field-generating-coil drive section 917 based on the operation signal and turns off the switching section when the amplitude comes to zero.

With the above-described structure, the position-detection magnetic-field generating coil 711 and the guidance-magnetic-field generating coils 913A and 913B can be driven in a time-division manner. For this reason, mutual induction is prevented from occurring between the position-detection magnetic-field generating coil 711 and the guidance-magnetic-field generating coils 913A and 913B, and accordingly, an area where the intensity of a combined magnetic field of the position-detection magnetic field and the mutual induction magnetic fields generated by the guidance-magnetic-field generating coils becomes substantially zero is prevented from occurring. As a result, the intensity of the position-detection magnetic field is prevented from decreasing in the operating region of the capsule endoscope 710.

Fourth Modification

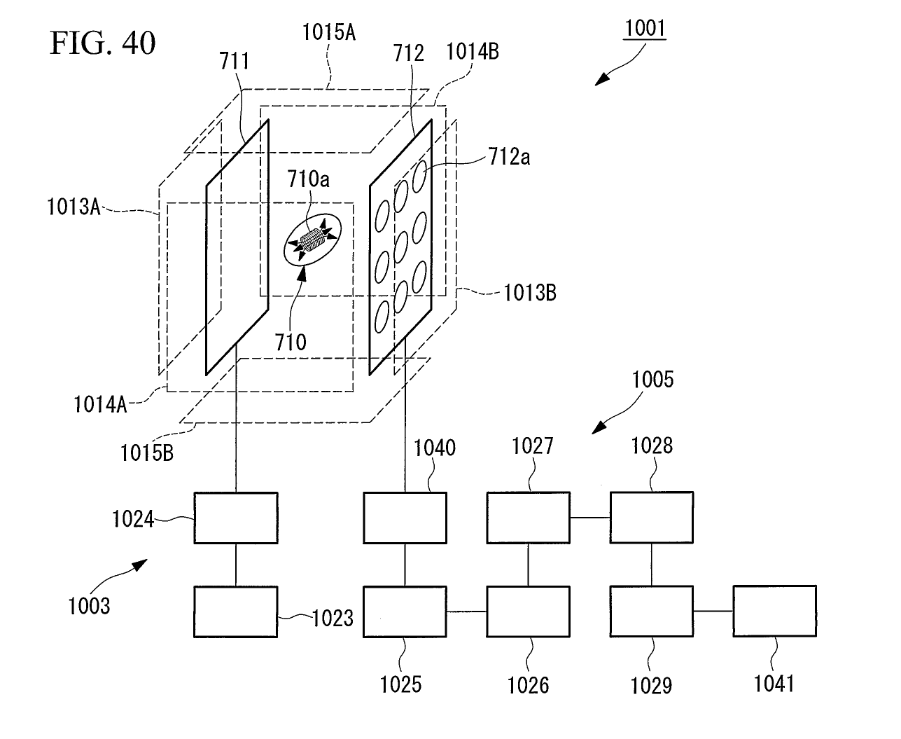

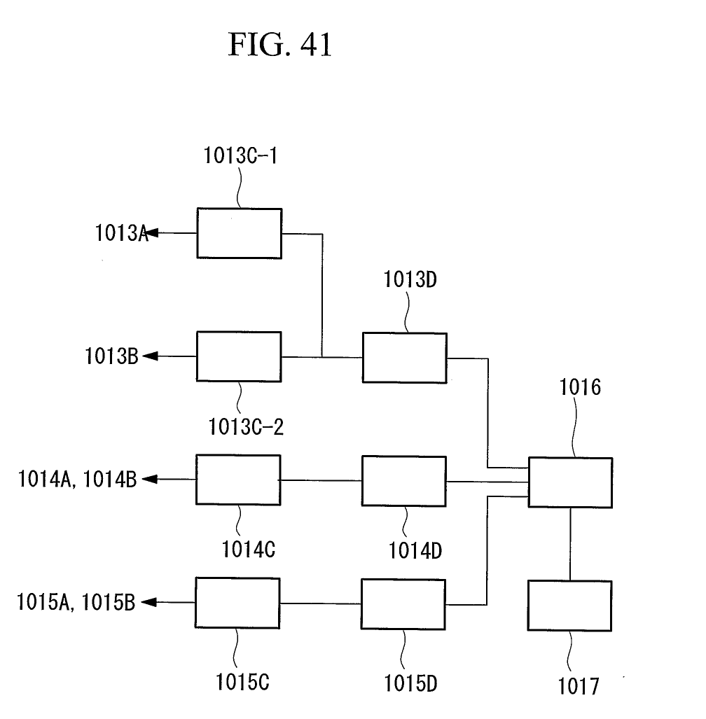

A fourth modification according to the present invention will now be described with reference to FIGS. 40 and 41.

The basic configuration of the medical magnetic-induction and position-detection system according to this modification is the same as that in the first modification; however, the structure in the vicinity of the induction-magnetic-field generating coil is different from that in the first modification. Therefore, in this modification, only the structure in the vicinity of the induction-magnetic-field generating coil will be described using FIGS. 40 and 41, and a description of the other components will be omitted.

FIG. 40 is a schematic diagram depicting the outline structure of a medical magnetic-induction and position-detection system according to this modification.

The same components as those in the first modification are denoted with the same reference numerals, and thus will not be described again here.

As shown in FIG. 40, a medical magnetic-induction and position-detection system 1001 is mainly composed of a position-detection magnetic-field generating coil 711 for generating a position-detection magnetic field; sense coils 712 for detecting an induced magnetic field generated by the magnetic induction coil 710a installed in the capsule endoscope 710; and guidance-magnetic-field generating coils (guidance-magnetic-field generating unit, electromagnets, opposed coils) 1013A, 1013B, 1014A, 1014B, 1015A, and 1015B for generating guidance magnetic fields for guiding the capsule endoscope to a predetermined position in a body cavity.

The position-detection magnetic-field generating coil 711 is provided with a drive section 1003 for controlling the driving of the position-detection magnetic-field generating coil 711, and the sense coils 712 are provided with a detection section 1005 for processing a signal output from the sense coils 712.

The drive section 1003 is mainly composed of a signal generating section 1023 for outputting an AC signal having the frequency of the alternating magnetic field generated in the position-detection magnetic-field generating coil 711 and a magnetic-field generating-coil drive section 1024 for amplifying the AC signal input from the signal generating section 1023 and driving the position-detection magnetic-field generating coil 711.

The detection section 1005 is mainly composed of a filter 1025 for cutting unwanted frequency components contained in an output signal from a detection coil 712a; an amplifier 1026 for amplifying the output signal from which unwanted components are cut; a DC converter 1027 for converting the amplified output signal from an AC signal to a DC signal; An A/D converter 1028 for converting the DC-converted output signal from an analog signal to a digital signal; a CPU 1029 for performing computational processing based on the output signal converted into a digital signal; and a sense coil selector (magnetic-field-sensor selecting unit) 1040 for selecting the output signal of a predetermined sense coil 712 from among the output signals of all sense coils 712.

A memory 1041 for saving an output signal acquired while the capsule endoscope 710 is not present is connected to the CPU 1029. By arranging the memory 1041, it is easier to subtract an output signal acquired while the capsule endoscope 710 is not present from an output signal acquired while the capsule endoscope 710 is present. For this reason, only an output signal associated with the induced magnetic field generated by the magnetic induction coil 710a of the capsule endoscope 710 can easily be detected.

Furthermore, an example of the DC converter 1027 is an RMS converter; it is not particularly limited, however. A known AC-DC converter can also be used.

The guidance-magnetic-field generating coils 1013A and 1013B, the guidance-magnetic-field generating coils 1014A and 1014B, and the guidance-magnetic-field generating coils 1015A and 1015B are arranged so as to face each other, with the distance therebetween satisfying Helmholtz conditions or a similar distance. For this reason, the spatial intensity gradients of magnetic fields generated by the guidance-magnetic-field generating coils 1013A and 1013B, the guidance-magnetic-field generating coils 1014A and 1014B, and the guidance-magnetic-field generating coils 1015A and 1015B are eliminated or negligibly small.

In addition, the central axes of the guidance-magnetic-field generating coils 1013A and 1013B, the guidance-magnetic-field generating coils 1014A and 1014B, and the guidance-magnetic-field generating coils 1015A and 1015B are arranged so as to be orthogonal to one another and also so as to form a rectangular space therein. The rectangular space serves as an operating space of the capsule endoscope 710, as shown in FIG. 40.

FIG. 41 is a block diagram illustrating the outline structure of the guidance-magnetic-field generating coils of FIG. 40.

The guidance-magnetic-field generating coils 1014A and 1014B are electrically connected in series, and the guidance-magnetic-field generating coils 1015A and 1015B are electrically connected in series. On the other hand, since the guidance-magnetic-field generating coils 1013A and 1013B are connected to different induction-magnetic-field generating-coil drive sections, they are not electrically connected in series, unlike the other coil pairs. More specifically, the guidance-magnetic-field generating coils 1013A and 1013B are individually electrically connected, so that outputs of different guidance-magnetic-field-generating-coil drive sections 1013C-1 and 1013C-2 are input to the respective guidance-magnetic-field generating coils 1013A and 1013B. In addition, the guidance-magnetic-field generating coils 1014A and 1014B are electrically series-connected to a guidance-magnetic-field-generating-coil drive section 1014C, and the guidance-magnetic-field generating coils 1015A and 1015B are electrically series-connected to a guidance-magnetic-field-generating-coil drive section 1015C. An electrical connection is provided so that the same control signal from a signal generator 1013D is input to the guidance-magnetic-field generating coils 1013C-1 and 1013C-2. Furthermore, an electrical connection is provided so that signals from signal generators 1014D and 1015D are input to the guidance-magnetic-field-generating-coil drive sections 1014C and 1015C, respectively. An electrical connection is provided so that a control signal from an induction control section 1016 is input to the signal generators 1013D, 1014D, and 1015D. An electrical connection is provided so that a signal from an input device 1017, to which an instruction as to the guidance direction of the capsule endoscope 710 is externally input, is input to the induction control section 1016.

The operation of the medical magnetic-induction and position-detection system 1001 with the above-described structure will now be described.

First, the operation of detecting the position of the capsule endoscope 710 in the medical magnetic-induction and position-detection system 1001 will be described.

As shown in FIG. 40, in the drive section 1003, the signal generating section 1023 generates an AC signal having a predetermined frequency, and the AC signal is output to the magnetic-field generating-coil drive section 1024. The magnetic-field generating-coil drive section 1024 amplifies the input AC signal to a predetermined intensity, and the amplified AC signal is output to the position-detection magnetic-field generating coil 711. The position-detection magnetic-field generating coil 711 forms an alternating magnetic field therearound as a result of the amplified AC signal being supplied.

When the magnetic flux of the above-described alternating magnetic field intersects the capsule endoscope 710, resonant current of a predetermined frequency is induced in the detector closed circuit having the magnetic induction coil 710a installed therein. When a resonant current is induced in the closed circuit of the capsule endoscope 71, the resonant current causes the magnetic induction coil 710a to form therearound an induced magnetic field having a predetermined frequency.

Since the magnetic fluxes of the alternating magnetic field and the induced magnetic field intersect the sense coils 712, the sense coils 712 capture a magnetic flux generated by adding the magnetic fluxes of both the magnetic fields and generate an output signal that is an induction current based on a change in the intersecting magnetic fluxes. The output signal of the sense coils 712 is output to the detection section 1005.

In the detection section 1005, the output signal that has been input is first input to the sense coil selector 1040. The sense coil selector 1040 passes only an output signal used for position detection of the capsule endoscope 710 therethrough and cuts out other output signals.

Examples of a method for selecting an output signal include selecting output signals with high signal intensity, output signals from sense coils 712 positioned near the capsule endoscope 710, or the like.

Only an output signal used for position detection may be selected by arranging the sense coil selector 1040 between the sense coils 712 and the filter 1025, as described above. Alternatively, by causing the sense coil selector 1040 to switch the connection from among a plurality of sense coils 712, the output signals from all sense coils 712 may be input to the detection section 1005 in an time-division manner. Furthermore, by connecting the line between the filter 1025 and the A/D converter 1028 to a plurality of sense coils 712, it is not necessary to use the sense coil selector 1040 or select an output signal. Thus, no particular restrictions are applied.

The selected output signal is input to the filter 1025, and frequency components in the output signal that are not used for position detection, for example, low-frequency components, are removed. The output signal from which unwanted components are removed is input to the amplifier 1026 and is then amplified so as to have an input level appropriate for the A/D converter 1028 downstream thereof.

The amplified output signal is input to the DC converter 1027, and the output signal, which is an AC signal, is converted into a DC signal. Thereafter, the output signal is input to the A/D converter 1028, and the output signal, which is an analog signal, is converted into a digital signal.

The output signal converted into a digital signal is input to the CPU 1029. On the other hand, the output signal acquired from the memory 1041 connected to the CPU 1029 while the capsule endoscope 710 is not present is input to the CPU 1029.

In the CPU 1029, an output signal associated with the induced magnetic field is obtained by calculating the difference between both the output signals that have been input, and computation for identifying the position of the magnetic induction coil 710a, namely the position of the capsule endoscope 710, is carried out based on the obtained output signal associated with the induced magnetic field. For the computation for identifying the position, a known computation method can be used, and no particular restrictions are applied.

The operation of guiding the capsule endoscope will now be described.

First, a movement that is to be applied to the capsule endoscope 710 for remote operation of the capsule endoscope 710 is input to the input device 1017. The input device 1017 outputs a signal to the induction control section 1016 based on the input information. Based on the input signal, the induction control section 1016 generates a control signal for generating a magnetic field for moving the capsule endoscope 710, and outputs it to the signal generators 1013D, 1014D, and 1015D.

In the signal generators 1013D, 1014D, and 1015D, signals output to the guidance-magnetic-field-generating-coil drive sections 1013C, 1014C, and 1015C are generated based on the input control signal. The guidance-magnetic-field-generating-coil drive sections 1013C, 1014C, and 1015C amplify the current of the input signals and cause the current to flow in the guidance-magnetic-field generating coils 1013A and 1013B, the guidance-magnetic-field generating coils 1014A and 1014B, and the guidance-magnetic-field generating coils 1015A and 1015B, respectively.

As described above, it is possible to generate a guidance magnetic field in an area near the capsule endoscope 710 by causing electric current to flow in the guidance-magnetic-field generating coils 1013A and 1013B, the guidance-magnetic-field generating coils 1014A and 1014B, and the guidance-magnetic-field generating coils 1015A and 1015B. With this generated magnetic field, the magnet in the capsule endoscope 710 can be moved, and accordingly, the capsule endoscope 710 can be moved by moving the magnet.

The operation when a mutual induction magnetic field is generated by the induction-magnetic-field generating coils 1013A and 1013B, the guidance-magnetic-field generating coils 1014A and 1014B, and the guidance-magnetic-field generating coils 1015A and 1015B will now be described.

The magnetic flux of the alternating magnetic field generated by the position-detection magnetic-field generating coil 711 intersects the guidance-magnetic-field generating coil 1013A arranged in the vicinity of the position-detection magnetic-field generating coil 711. At this time, as a result of the intersecting magnetic flux, the following induced electromotive force is generated in the guidance-magnetic-field generating coil 1013A, i.e., an electromotive force that forms a magnetic field having a direction in which variations in the magnetic field intensity are canceled out, namely, an inverse-phase magnetic field with a phase opposite to that of the above-described alternating magnetic field.

Since the guidance-magnetic-field generating coils 1013A and 1013B are driven by different induction-magnetic-field generating-coil drive sections 1013C-1 and 1013C-2, respectively, an induced electromotive force generated in 1013A causes electric current to flow in the closed circuit formed of the guidance-coil drive section 1013C-1 and the guidance-magnetic-field generating coil 1013A and form an inverse-phase magnetic field with a phase opposite to that of the position-detection magnetic field. On the other hand, because the electric current does not flow in the guidance-magnetic-field generating coil 1013B, no inverse-phase magnetic field with a phase opposite to that of the position-detection magnetic field is formed in the vicinity of the sense coils 712.

According to the above-described structure, the position-detection magnetic-field generating coil 711 generates a position-detection magnetic field for inducing an induced magnetic field in the magnetic induction coil 710a of the capsule endoscope 710. The induced magnetic field generated by the magnetic induction coil 710a is detected by the sense coils 712 and is used to detect the position or orientation of the capsule endoscope 710 having the magnetic induction coil 710a.

Furthermore, the guidance magnetic fields generated by the three sets of guidance-magnetic-field generating coils 1013A and 1013B, guidance-magnetic-field generating coils 1014A and 1014B, and guidance-magnetic-field generating coils 1015A and 1015B act on the magnet provided in the capsule endoscope 710 to control the position and orientation of the capsule endoscope 710. Here, since the three sets of guidance-magnetic-field generating coils 1013A and 1013B, guidance-magnetic-field generating coils 1014A and 1014B, and guidance-magnetic-field generating coils 1015A and 1015B are arranged such that the directions of their central axes are orthogonal to one another, the magnetic force lines of the guidance magnetic fields can be oriented in any three-dimensional direction. As a result, the position and orientation of the capsule endoscope 710 having the magnet can be controlled three-dimensionally.

In addition, since the two guidance-magnetic-field generating coils 1013A and 1013B are driven by the different guidance-magnetic-field-generating-coil drive sections 1013C-1 and 1013C-2, even if conditions whereby a position-detection magnetic field induces a mutual induction magnetic field in the guidance-magnetic-field generating coil 1013A are created, an electric current due to an electromotive force induced by the guidance-magnetic-field generating coil 1013A does not flow in the guidance-magnetic-field generating coil 1013B. Because of this, the guidance-magnetic-field generating coil 1013B does not generate a mutual induction magnetic field with a phase opposite to that of the position-detection magnetic field and generates only a guidance magnetic field. As a result, since a magnetic field that cancels out the position-detection magnetic field is prevented from occurring in the guidance-magnetic-field generating coil 1013B, an area where the position-detection magnetic field becomes substantially zero is prevented from occurring.

The technical field of the present invention is not limited to the modifications described above.

For example, although the above-described modifications are applied to a structure including one magnetic-field generating coil, one sense coil, one inverse-phase magnetic-field generating coil, and so forth that are arranged on substantially the same straight line, the modifications are not limited to this structure. The modifications may also be applied to a structure including a plurality of magnetic-field generating coils and so forth provided on a plurality of straight lines, where the number and positions of arranged components are not limited.

Furthermore, as the medical device, a description has been given of a device using a capsule endoscope that captures images of the interior of a body cavity of a subject; however, the invention is not limited to such a device using a capsule endoscope. It can be applied to various other types of medical device, such as a medical device that discharges a drug inside the body cavity of the subject; a medical device provided with a sensor for acquiring data on the interior of the body cavity; a medical device that can be left inside the body cavity for a long period of time; a medical device in which wiring lines for exchanging information and the like are connected to the exterior; and so forth.

### Sixth to Fifteenth Embodiments

In the above-described document 2, a technique for detecting the position of a capsule medical device by detecting electromagnetism issued from the capsule medical device provided with an LC resonant circuit using a plurality of external detection apparatuses is disclosed.

In document 2, however, there is danger that a magnet for induction driving or switching, for example, arranged in the capsule medical device adversely affects the LC resonant circuit and consequently changes the characteristic of the LC resonant circuit, or that the magnet shields the electromagnetic field (induced magnetic field) issued from the LC resonant circuit to decrease the position detection accuracy or even disable position detection. Furthermore, there is a problem that electrical power is consumed by the capsule medical device for position detection.

In the above-described document 3, a technique for detecting the position of a capsule medical device by means of a capsule endoscope having a magnetic induction coil installed therein, a drive coil for generating an induction current in the magnetic induction coil, and a detection apparatus for obtaining the relative position of the magnetic induction coil and the drive coil based on the induction current is disclosed.

In the above-described position detection technique, however, there is danger that a magnet for induction driving or switching, for example, arranged in the capsule medical device adversely affects the magnetic induction coil and consequently changes the characteristic of the magnetic induction coil or shields the induced magnetic field issued from the magnetic induction coil to decrease the position detection accuracy or even disable position detection. Furthermore, there is a problem that electrical power is consumed by the capsule medical device for position detection.

In the above-described document 4, a technique for driving a substantially cylindrical capsule medical device by forming a helical protrusion on the cylindrical surface of the capsule medical device and rotating the capsule medical device about the longitudinal axis is disclosed. The capsule medical device is rotationally driven by a magnet arranged in the capsule medical device and by an externally applied rotating magnetic field.

In the above-described document 1, however, a device for detecting the position of the capsule medical device is not described, and therefore, the capsule medical device cannot be driven and guided to a predetermined position.

Furthermore, it is easier to propose a method where the drive technique of the capsule medical device described in the above-described document 4 is combined with the position detection technique disclosed in the above-described document 2 or document 3, that is, a method where a magnetic position detection system using a magnetic induction coil is employed together with a capsule medical device having a guidance magnet built therein.

In this method, however, there is a danger that the guidance magnet interferes with the magnetic position detection system, which degrades the performance of the position detection system or disables position detection. Furthermore, a magnet used for purposes other than driving may also exhibit the same problem.

The above-described documents 1 and 5 disclose a motion control system for a movable micro-machine, including a magnetic-field generating section that generates a rotating magnetic field; a robot main body provided with a magnet that receives the rotating magnetic field that the magnetic-field generating section generates to generate propulsion by rotation; a position detector that detects the position of the robot main body; and a magnetic-field re-orienting unit that changes the orientation of the rotating magnetic field produced by the magnetic-field generating section based on the position of the robot main body detected by the position detector so as to be oriented in the direction in which the robot main body should move to reach the target. In the technology described above, the robot main body (capsule endoscope) is guided while controlling the orientation of the robot main body.

In the above-described position detection technique, however, since the polarization direction of the magnet arranged orthogonally to the rotation axis of the robot main body is detected, position detection needs to be carried out two or more times with different polarization directions of the magnet in order to identify the orientation, such as the rotation axis direction, of the robot main body. Furthermore, since the actual direction of the robot main body does not always follow the magnetic field that controls the position and direction of the robot main body, the guidance accuracy for the robot main body may decrease.

Furthermore, if a coil for carrying out, for example, information exchange with an external device via a magnetic field is arranged in the capsule medical device, since the magnet changes the coil characteristic or the magnet shields the magnetic field issued from the coil, there is danger of such information exchange and so forth being prevented.

In order to overcome the above-described problems, the following embodiments can be employed to provide a medical device and a medical magnetic-induction and position-detection system capable of effectively operating a magnetic position detection system in a medical device having a magnet built therein.

### Sixth Embodiment

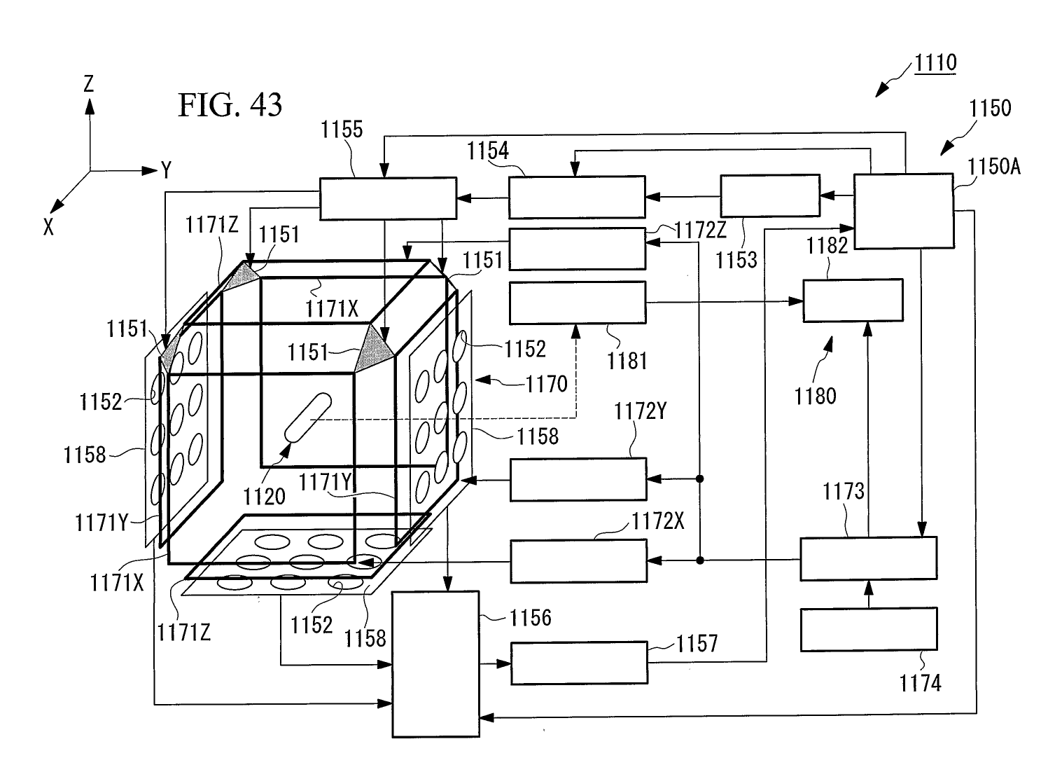

A sixth embodiment of a medical magnetic-induction and position-detection system according to the present invention will now be described with reference to FIGS. 43 to 73.

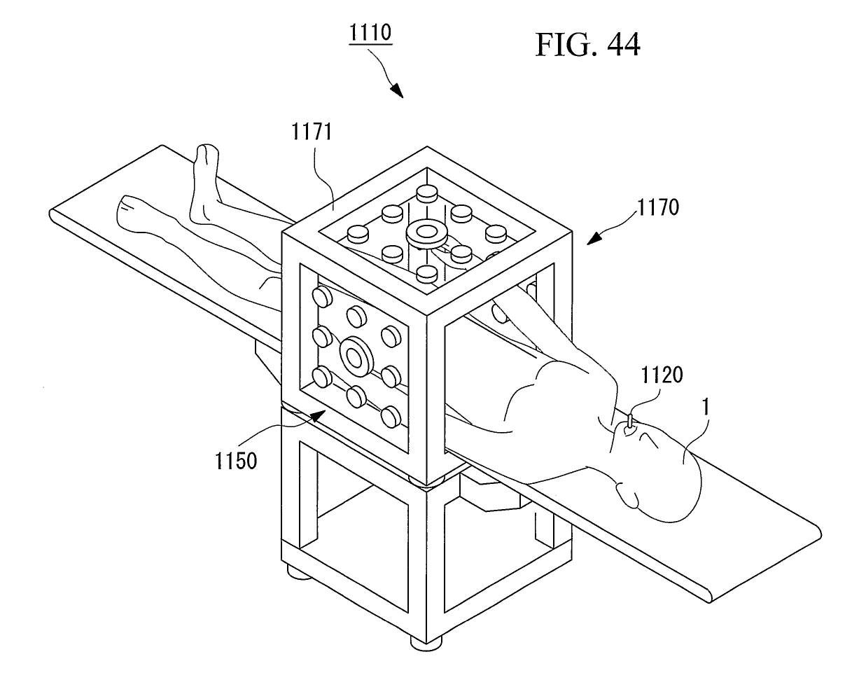

FIG. 43 is a diagram schematically showing a medical magnetic-induction and position-detection system according to this embodiment. FIG. 44 is a perspective view of the medical magnetic-induction and position-detection system.

As shown in FIGS. 43 and 44, a medical magnetic-induction and position-detection system 1110 is mainly formed of a capsule endoscope (medical device) 1120 that is introduced into a body cavity of a subject 1, per oral or per anus, to optically image an internal surface of a passage in the body cavity and wirelessly transmit an image signal; a position detection unit (position detection system, position detection apparatus, position detector, calculating apparatus) 1150 that detects the position of the capsule endoscope 1120; a magnetic induction apparatus 1170 that guides the capsule endoscope 1120 based on the detected position of the capsule endoscope 1120 and instructions from an operator; and an image display apparatus 1180 that displays the image signal transmitted from the capsule endoscope 1120.

As shown in FIG. 43, the magnetic induction apparatus 1170 is mainly formed of a three-axis guidance-magnetic-field generating unit (guidance-magnetic-field generating unit, electromagnet) 1171 that produces parallel magnetic fields for driving and guiding the capsule endoscope 1120; a Helmholtz-coil driver 1172 that controls the gain of currents supplied to the three-axis guidance-magnetic-field generating unit 1171; a rotation-magnetic-field control circuit (magnetic-field-orientation control unit) 1173 that controls the directions of the parallel magnetic fields for driving and guiding the capsule endoscope 1120; and an input device 1174 that outputs to the rotation-magnetic-field control circuit 1173 the direction of movement of the capsule endoscope 1120 that the operator inputs.

In this embodiment, the three-axis guidance-magnetic-field generating unit 1171 is described as applied to a coil unit where pairs of coils are opposed one another and electromagnets for generating parallel magnetic fields are arranged in the three axial directions. A preferable example of this coil may include a Helmholtz-coil unit having three Helmholtz coils arranged in the three axial directions.

Although in this embodiment a description is given assuming the coil is a Helmholtz-coil unit, the structure of the electromagnets is not limited to a Helmholtz-coil unit, and substantially rectangular opposing coils, such as those shown in FIG. 43, are also acceptable. In addition, the distance between the coils may be set freely rather than set to half the diameters of the coils, as long as a desired magnetic field can be obtained in the target space.

Furthermore, magnets of any structure are acceptable rather than opposing coils, as long as a desired magnetic field can be obtained.

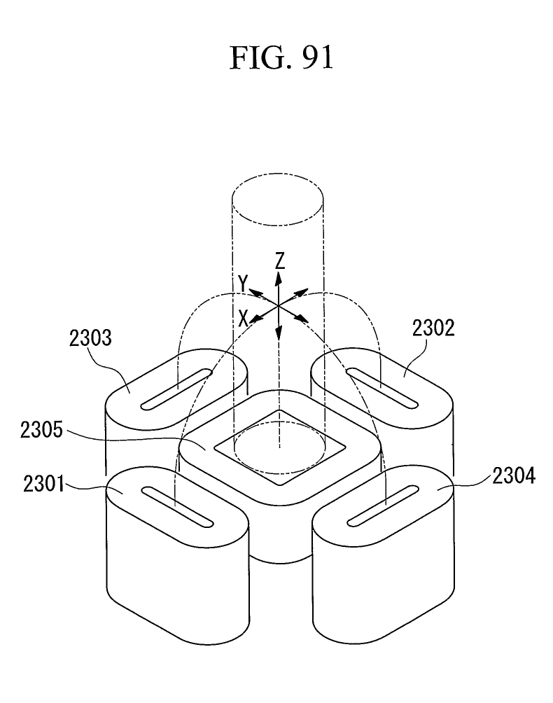

For example, as shown in FIG. 91, a magnetic field in the X-axis direction can be generated by arranging electromagnets 2301 to 2305 each on one side of the target area and then generating a magnetic field between the electromagnet 2301 and the electromagnet 2302. Similarly, a magnetic field in the Y-axis direction can be generated between the electromagnet 2303 and the electromagnet 2304, and a magnetic field in the Z-axis direction can be generated in the electromagnet 2305.

By using an electromagnet system with the above-described structure, similar advantages can be afforded.

As shown in FIGS. 43 and 44, the three-axis guidance-magnetic-field generating unit 1171 is formed in a substantially rectangular shape. The three-axis guidance-magnetic-field generating unit 1171 includes three-pairs of mutually opposing Helmholtz coils 1171X, 1171Y, and 1171Z, and each pair of Helmholtz coils 1171X, 1171Y, and 1171Z is disposed so as to be substantially orthogonal to the X, Y, and Z axes in FIG. 43. The Helmholtz coils disposed substantially orthogonally with respect to the X, Y, and Z axes are denoted as the Helmholtz coils 1171X, 1171Y, and 1171Z, respectively.

The Helmholtz coils 1171X, 1171Y, and 1171Z are disposed so as to form a rectangular space in the interior thereof. As shown in FIG. 43, the rectangular space serves as an operating space of the capsule endoscope 1120 and, as shown in FIG. 44, is the space in which the subject 1 is placed.

The Helmholtz-coil driver 1172 includes Helmholtz-coil drivers 1172X, 1172Y, and 1172Z for controlling the Helmholtz coils 1171X, 1171Y, and 1171Z, respectively.

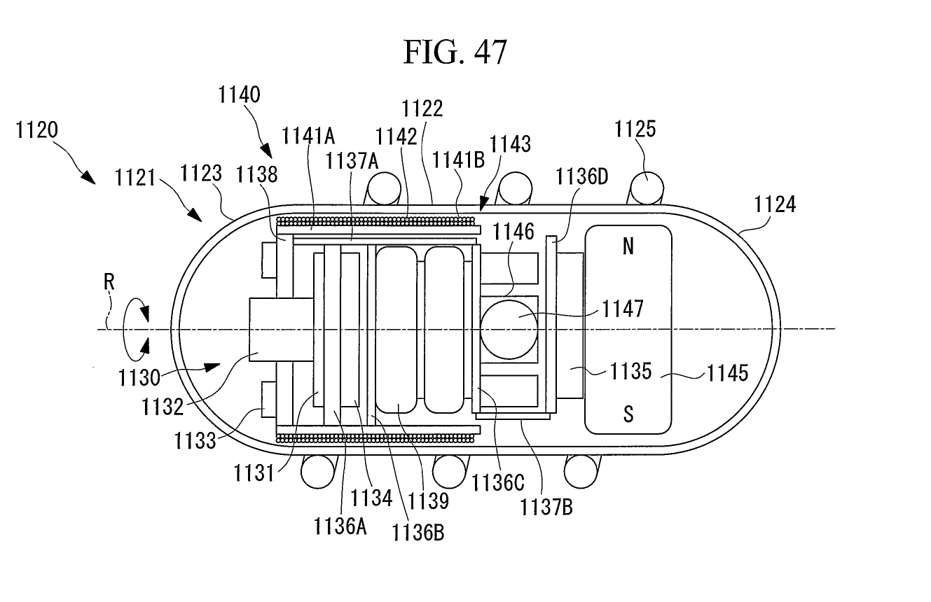

Direction-of-movement instructions for the capsule endoscope 1120, which the operator inputs from the input device 1174, are input to the rotation-magnetic-field control circuit 1173, together with data from the position detection unit 1150 indicating the direction in which the capsule endoscope 1120 is currently pointing (the direction of a rotation axis (central axis) R (refer to FIG. 47) of the capsule endoscope 1120). Then, signals for controlling the Helmholtz-coil drivers 1172X, 1172Y, and 1172Z are output from the rotation-magnetic-field control circuit 1173, and rotational phase data of the capsule endoscope 1120 is output to the image display apparatus 1180.

An input device for specifying the direction of movement of the capsule endoscope 1120 by moving a joystick is used as the input device 1174.

As mentioned above, the input device 1174 may use a joystick-type device, or another type of input device maybe used, such as an input device that specifies the direction of movement by pushing direction-of-movement buttons.

As shown in FIG. 43, the position detection unit 1150 is mainly formed of drive coils (drive section) 1151 that generate induced magnetic fields in a magnetic induction coil (described later) in the capsule endoscope 1120; sense coils (magnetic field sensors, magnetic-field detection sections) 1152 that detect the induced magnetic fields generated in the magnetic induction coil; and a position detection apparatus 1150A that computes the position of the capsule endoscope 1120 based on the induced magnetic fields that the sense coils 1152 detect and that controls the alternating magnetic fields formed by the drive coils 1151.

Between the position detection apparatus 1150A and the drive coils 1151 there are provided a sine-wave generating circuit 1153 that generates an AC current based on the output from the position detection apparatus 1150A; a drive-coil driver 1154 that amplifies the AC current input from the sine-wave generating circuit 1153 based on the output from the position detection apparatus 1150A; and a drive-coil selector 1155 that supplies the AC current to a drive coil 1151 selected on the basis of the output from the position detection apparatus 1150A.

Between the sense coils 1152 and the position detection apparatus 1150A there are provided a sense-coil selector (magnetic-field-sensor selecting unit) 1156 that selects from the sense coils 1152 AC current that includes position information of the capsule endoscope 1120 and so on, based on the output from the position detection apparatus 1150A; and a sense-coil receiving circuit 1157 that extracts an amplitude value from the AC current passing through the sense-coil selector 1156 and outputs it to the position detection apparatus 1150A.

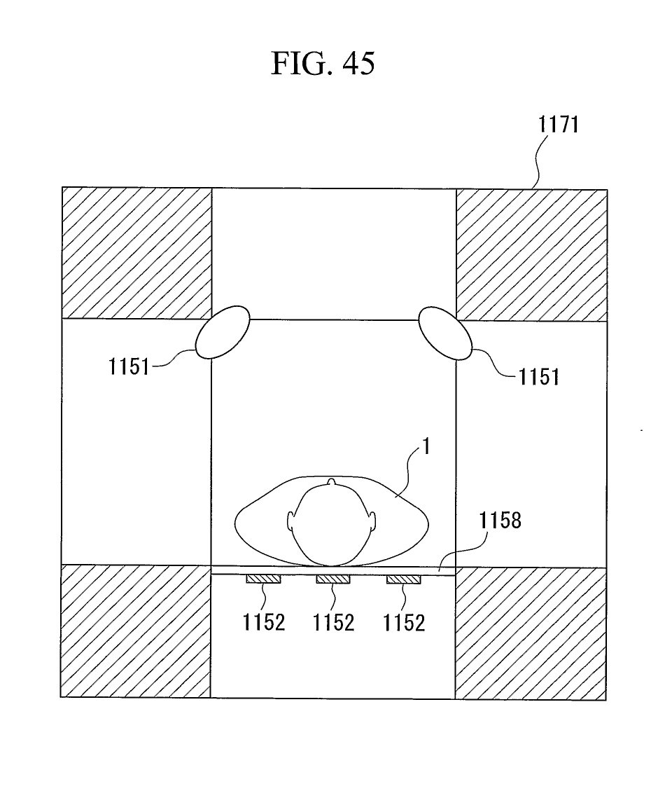

FIG. 45 is a schematic diagram showing a cross-section of the medical magnetic-induction and position-detection system.

Here, as shown in FIGS. 43 and 45, the drive coils 1151 are positioned at an angle at the four upper (in the positive direction of the Z-axis) corners of the substantially rectangular operating space formed by the Helmholtz coils 1171X, 1171Y, and 1171Z. The drive coils 1151 form substantially triangular coils that connect the corners of the square-shaped Helmholtz coils 1171X, 1171Y, and 1171Z. By disposing the drive coils 1151 at the top in this way, it is possible to prevent interference between the drive coils 1151 and the subject 1 (refer to FIG. 3).

The drive coils 1151 may be substantially triangular coils, as mentioned above, or it is possible to use coils of various shapes, such as circular coils, etc.

The sense coils 1152 are formed as air-core coils, and are supported, at the inner side of the Helmholtz coils 1171X, 1171Y, and 1171Z, by three planar coil-supporting parts 1158 that are disposed at positions facing the drive coils 1151 and at positions mutually opposing each other in the Y-axis direction, with the operating space of the capsule endoscope 1120 being disposed therebetween. Nine of the sense coils 1152 are arranged in the form of a matrix in each coil-supporting part 1158, and thus a total of 27 sense coils 1152 are provided in the position detection unit 1150.

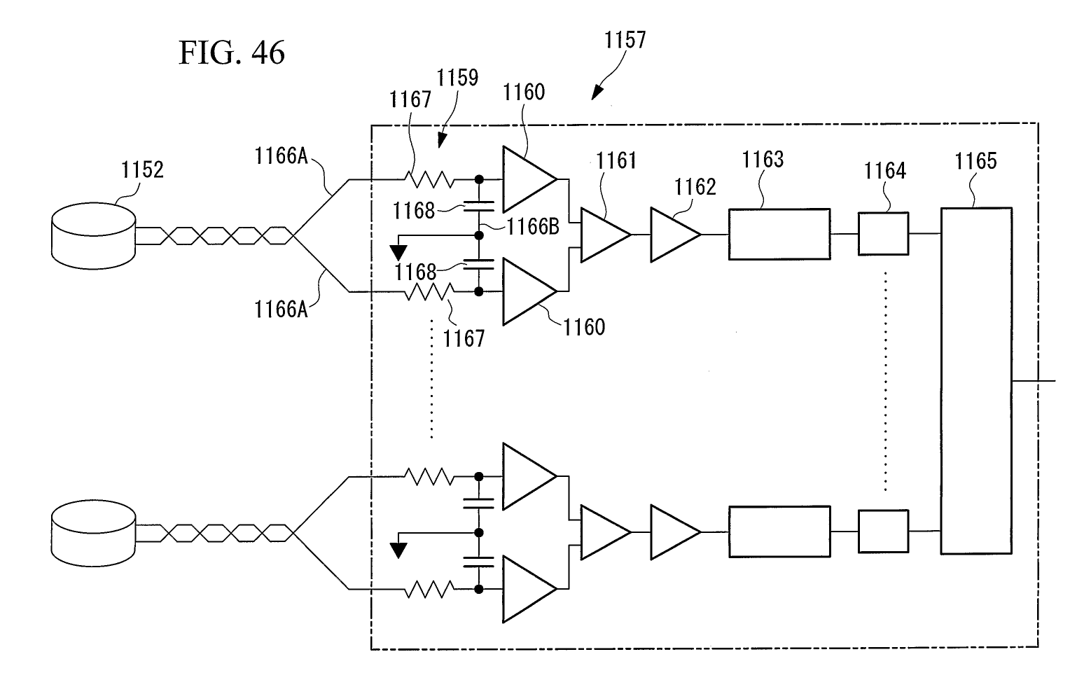

FIG. 46 is a schematic diagram showing the circuit configuration of the sense-coil receiving circuit 1157.

As shown in FIG. 46, the sense-coil receiving circuit 1157 is formed of a high-pass filter (HPF) 1159 that removes low-frequency components of input AC voltages including the position information of the capsule endoscope 1120; pre-amplifiers 1160 that amplify the AC voltages; a band-pass filter (BPF) 1161 that removes high frequencies included in the amplified AC voltages; an amplifier (AMP) 1162 that amplifies the AC voltage from which the high frequencies have been removed; a root-mean-square detection circuit (True RMS converter) 1163 that detects the amplitude of the AC voltage and that extracts and outputs an amplitude value; an A/D converter 1164 that converts the amplitude value to a digital signal; and a memory 1165 for temporarily storing the digitized amplitude value.

The high-pass filter 1159 is formed of resistors 1167 disposed in a pair of wires 1166A extending from the sense coil 1152; a wire 1166B that is connected to the pair of wires 1166A and that is grounded substantially at the center thereof; and a pair of capacitors 1168 disposed opposite each other in the wire 1166B, with the grounding point therebetween. The pre-amplifiers 1160 are disposed in the pair of wires 1166A, respectively, and the AC voltages output from the pre-amplifiers 1160 are input to the single band-pass filter 1161. The memory 1165 temporarily stores the amplitude values obtained from the nine sense coils 1152 and outputs the stored amplitude values to the position detection unit 1150.

The root-mean-square detection circuit 1163 may be used to extract the amplitude value of the AC voltage, as mentioned above, the amplitude value may be detected by smoothing the magnetic field information using a rectifying circuit and detecting the voltage, or the amplitude value may be detected using a peak detecting circuit that detects a peak in the AC voltage.

Regarding the waveform of the detected AC voltage, the phase with respect to a waveform applied to the drive coil 1151 changes depending on the presence and the position of a magnetic induction coil 1142, to be described later, in the capsule endoscope 1120. This phase change may be detected with a lock-in amplifier or the like.

As shown in FIG. 43, the image display apparatus 1180 is formed of an image receiving circuit 1181 that receives the image transmitted from the capsule endoscope 1120 and a display section 1182 that displays the image based on the received image signal and a signal from the rotation-magnetic-field control circuit 1173.

FIG. 47 is a schematic diagram showing the configuration of the capsule endoscope 1120.

As shown in FIG. 47, the capsule endoscope 1120 is mainly formed of an outer casing 1121 that accommodates various devices in the interior thereof; an imaging section (biological-information acquiring unit) 1130 that images an internal surface of a passage in the body cavity of the subject; a battery (power supply unit) 1139 for driving the imaging section 1130; an induced-magnetic-field generating section (induction-magnetic-field generating unit) 1140 that generates induced magnetic fields by means of the drive coils 1151 described above; and a guidance magnet (magnet) 1145 that drives and guides the capsule endoscope 1120.

The outer casing 1121 is formed of an infrared-transmitting cylindrical capsule main body (hereinafter abbreviated simply as main body) 1122 whose central axis defines a rotation axis (central axis) R of the capsule endoscope 1120, a transparent hemispherical front end portion 1123 that covers the front end of the main body 1122, and a hemispherical rear end portion 1124 that covers the rear end of the main body, to form a sealed capsule container with a watertight construction.

A helical part 1125 in which a wire having a circular cross-section is wound in the form of a helix about the rotation axis R is provided on the outer circumferential surface of the main body of the outer casing 1121.

The imaging section 1130 is mainly formed of a board 1136A positioned substantially orthogonal to the rotation axis R; an image sensor 1131 disposed on the surface at the front end portion 1123 side of the board 1136A; a lens group 1132 that forms an image of the internal surface of the passage inside the body cavity of the subject on the image sensor 1131; an LED (Light Emitting Diode) 1133 that illuminates the internal surface of the passage inside the body cavity; a signal processing section 1134 disposed on the surface at the rear end portion 1124 side of the board 1136A; and a radio device 1135 that transmits the image signal to the image display apparatus 1180.

The signal processing section 1134 is electrically connected to the battery 1139 via boards 1136A, 1136B, and 1136C and a flexible board 1137A, is electrically connected to the image sensor 1131 via the board 1136A, and is electrically connected to the LED 1133 via the board 1136A, the flexible board 1137A, and a support member 1138. Also, the signal processing section 1134 compresses the image signal that the image sensor 1131 acquires, temporarily stores it (memory), and transmits the compressed image signal to the exterior from the radio device 1135, and in addition, it controls the on/off state of the image sensor 1131 and the LED 1133 based on signals from a switch section 1146 to be described later.

The image sensor 1131 converts the image formed via the front end portion 1123 and the lens group 1132 to an electrical signal (image signal) and outputs it to the signal processing section 1134. CMOS (Complementary Metal Oxide Semiconductor) devices or CCDs (Charge Coupled Devices), for example, can be used as this image sensor 1131.

Moreover, a plurality of the LEDs 1133 are disposed on the support member 1138 positioned towards the front end portion 1123 from the board 1136A such that gaps are provided therebetween in the circumferential direction around the rotation axis R.

At the rear-end portion 1124 side of the signal processing section 1134, the battery 1139 is interposed between the boards 1136B and 1136C.

A switch section 1146, which is arranged on the board 1136C, is provided at the rear-end portion 1124 side of the battery 1139. The switch section 1146 has an infrared sensor 1147, is electrically connected to the signal processing section 1134 via the boards 1136A and 1136C and the flexible board 1137A, and is electrically connected to the battery 1139 via the boards 1136B and 1136C and the flexible board 1137A.

Also, a plurality of the switch sections 1146 are disposed in the circumferential direction about the rotation axis R at regular intervals, and the infrared sensor 1147 is disposed so as to face the outside in the diameter direction. In this embodiment, an example has been described in which four switch sections 1146 are disposed, but the number of switch sections 1146 is not limited to four; any number may be provided.

The radio device 1135 is disposed on the surface of the board 1136D at the rear end portion 1124 side. The radio device 1135 is electrically connected to the signal processing section 1134 via the boards 1136A, 1136C, and 1136D and the flexible boards 1137A and 1137B.

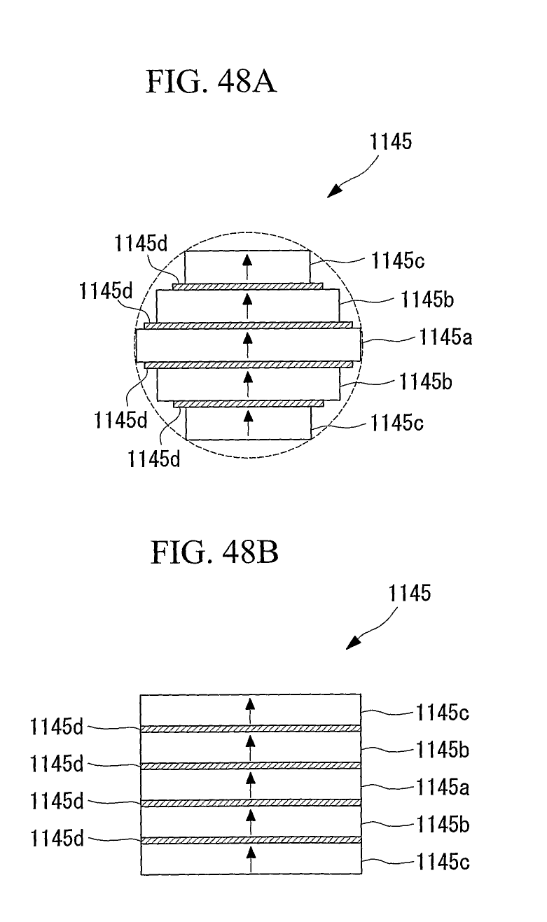

FIG. 48 is a diagram illustrating the structure of the guidance magnet 1145 provided in the capsule endoscope 1120. FIG. 48A is a diagram of the guidance magnet 1145 as viewed from the front-end portion 1123 side of the capsule endoscope 1120, whereas FIG. 48B is a diagram of the guidance magnet 1145 as viewed from the lateral surface.

As shown in FIG. 47, the guidance magnet 1145 is arranged at the rear-end portion 1124 side of the radio device 1135. The guidance magnet 1145 is arranged such that its center of gravity is positioned on the rotation axis R and that it is magnetized in a direction orthogonal to the rotation axis R (e.g., up and down direction in FIG. 47).

Therefore, a magnetic field formed by the guidance magnet 1145 at the position of a permalloy film, to be described later, is substantially orthogonal to the rotation axis R.

As shown in FIGS. 48A and 48B, the guidance magnet 1145 includes one large-size magnet piece (magnet piece) 1145a formed substantially in the shape of a plate, two medium-size magnet pieces (magnet pieces) 1145b, two small-size magnet pieces (magnet pieces) 1145c, and insulators (insulating materials) 1145d, such as vinyl sheets, interposed between the magnet pieces 1145a, 1145b, and 1145c, and is constructed so as to have a substantially cylindrical shape. In addition, the magnet pieces 1145a, 1145b, and 1145c are magnetized in the plate-thickness directions (up and down direction in the figure), and the direction indicated by the arrow in the figure represents the magnetization direction. More specifically, the side indicated by the arrow corresponds to the north pole, and the opposite side corresponds to the south pole.

Depending on the size of the capsule endoscope 1120, the typical shape and size of the guidance magnet 1145 are as follows: a cylinder diameter of about 6 mm to about 8 mm and a cylinder height of about 6 mm to about 8 mm. More specifically, a cylinder with a diameter of 8 mm and a height of 6 mm or a cylinder with a diameter of 6 mm and a height of 8 mm can be used for the guidance magnet 1145. In addition, the material of the magnet piece 1145a is, for example, neodymium-cobalt but is not limited to neodymium-cobalt.

The guidance magnet 1145 may be composed of the magnet pieces 1145a, 1145b, and 1145c and the insulators 1145d, as described above. Alternatively, the guidance magnet 1145 may be composed of only the magnet pieces 1145a, 1145b, and 1145c. Furthermore, the guidance magnet 1145 may be formed of a single cylindrical magnet.

As shown in FIG. 47, the induced-magnetic-field generating section 1140 is arranged in a cylindrical space between the main body 1122 and the battery 1139 and so forth.

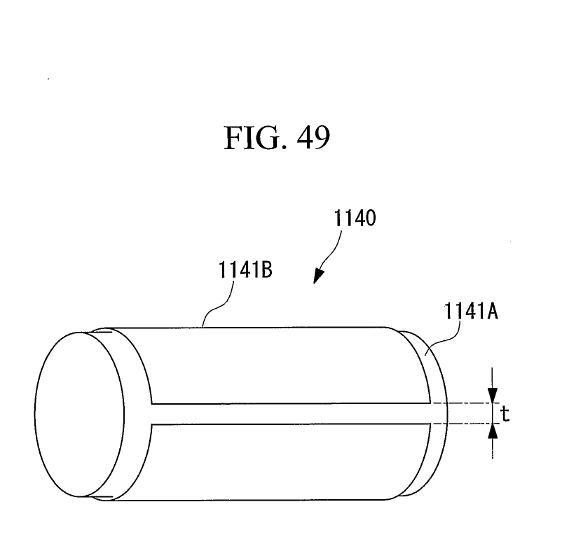

As shown in FIGS. 47 and 49, the induced-magnetic-field generating section 1140 is formed of a core member 1141A formed in the shape of a cylinder whose central axis is substantially coincident with the rotation axis R; a magnetic induction coil (built-in coil) 1142 disposed on the outer circumferential part of the core member 1141A; a permalloy film (core) 1141B disposed between the core member 1141A and the magnetic induction coil 1142; and a capacitor (not shown in the figure) that is electrically connected to the magnetic induction coil 1142 and that constitutes the LC resonant circuit (circuit) 1143.

The coil 1142 and the permalloy film 1141B are located at positions where the magnetic flux density in the permalloy film 1141B formed by the magnetic field of the guidance magnet 1145 is equal to or less than half the saturated flux density in the permalloy film 1141B. More specifically, the coil 1142 and the permalloy film 1141B are disposed at positions at least about 5 mm, preferably about 10 mm or more, away from the guidance magnet 1145. As shown in FIG. 49, the permalloy film 1141B is produced by forming permalloy, as a magnetic material, in a sheet membrane. Furthermore, when the permalloy film 1141B is wound around the core member 1141A, a gap t is produced.

As shown in FIG. 49, since the permalloy film 1141B is formed like a substantially cylindrical membrane with the rotation axis R as its central axis, the demagnetizing factor in the direction of the rotation axis R in the permalloy film 1141B is smaller than the demagnetizing factors in other directions.

The permalloy film 1141B may be formed of permalloy as described above, or may be formed of iron or nickel, which is also a magnetic material.

The LC resonant circuit 1143 may be formed of the magnetic induction coil 1142 and the capacitor as described above, or the LC resonant circuit 1143 may be a resonant circuit based on self resonance due to the magnetic induction coil 1142 rather than using a capacitor.

Next, the operation of the medical magnetic-induction and position-detection system 1110 having the above-described configuration will be described.

First, an overview of the operation of the medical magnetic-induction and position-detection system 1110 will be described.

As shown in FIGS. 43 and 44, the capsule endoscope 1120 is inserted, per oral or per anus, into a body cavity of a subject 1 who is lying down inside the position detection unit 1150 and the magnetic induction apparatus 1170. The position of the inserted capsule endoscope 1120 is detected by the position detection unit 1150, and it is guided to the vicinity of an affected area inside a passage in the body cavity of the subject 1 by the magnetic induction apparatus 1170. The capsule endoscope 1120 images the internal surface of the passage in the body cavity while being guided to the affected area and in the vicinity of the affected area. Then, data for the imaged internal surface of the passage inside the body cavity and data for the vicinity of the affected area are transmitted to the image display apparatus 1180. The image display apparatus 1180 displays the transmitted images on the display section 1182.

The operation of the position detection unit 1150 will now be described.

As shown in FIG. 43, in the position detection unit 1150, the sine-wave generator circuit 1153 generates an AC current based on the output from the position detection apparatus 1150A, and the AC current is output to the drive-coil driver 1154. The frequency of the generated AC current is in a frequency range from a few kHz to 100 kHz, and the frequency varies (sweeps) within the above-mentioned range over time, so as to include a resonance frequency, to be described later. The sweep range is not limited to the range mentioned above; it may be a narrower range or it may be a wider range, and is not particularly limited.

Instead of carrying out sweeping every time, a measurement frequency may be first determined by sweeping, and then the frequency may be fixed to the measurement frequency. By doing so, the measurement speed can be increased. Furthermore, sweeping may be carried out periodically to update the determined measurement frequency. This serves as a measure against temperature-dependent changes in resonance frequency.

The AC current is amplified in the drive-coil driver 1154 based on an instruction from the position detection apparatus 1150A and is output to the drive-coil selector 1155. The amplified AC current is supplied to the drive coil 1151 selected by the position detection apparatus 1150A in the drive-coil selector 1155. Then, the AC current supplied to the drive coil 1151 produces an alternating magnetic field in the operating space of the capsule endoscope 1120.

Due to the alternating magnetic field, an induced electromotive force is produced in the magnetic induction coil 1142 of the capsule endoscope 1120 disposed in the alternating magnetic field, and an induced current flows therein. When the induced current flows in the magnetic induction coil 1142, an induced magnetic field is produced by the induced current.

Since the magnetic induction coil 1142 forms the resonance circuit 1143 together with the capacitor, induced current flowing in the resonance circuit 1143 (magnetic induction coil 1142) increases and the induced magnetic field produced becomes intense when the period of the alternating magnetic field corresponds to the resonance frequency of the resonance circuit 1143. Furthermore, since the permalloy film 1141B is disposed at the inner side of the magnetic induction coil 1142, the induced magnetic field produced by the magnetic induction coil 1142 becomes even more intense.

The induced magnetic field described above produces an induced electromotive force in the sense coil 1152, and an AC voltage (magnetic field information) that includes position information of the capsule endoscope 1120 and so on is produced in the sense coil 1152. This AC voltage is input to the sense-coil receiving circuit 1157 via the sense-coil selector 1156, where an amplitude value (amplitude information) of the AC voltage is extracted.

As shown in FIG. 46, low frequency components included in the AC voltage input to the sense-coil receiving circuit 1157 are first removed by the high-pass filter 1159, and the AC voltage is then amplified by the pre-amplifiers 1160. Thereafter, high frequencies are removed by the band-pass filter 1161, and the AC voltage is amplified by the amplifier 1162. The amplitude value of the AC voltage from which unwanted components have been removed in this way is extracted by the root-mean-square detection circuit 1163. The extracted amplitude value is converted to a digital signal by the A/D converter 1164 and is stored in the memory 1165.

The memory 1165 stores, for example, an amplitude value corresponding to one period in which the sine-wave signal generated in the sine-wave generating circuit 1153 is swept close to the resonance frequency of the LC resonant circuit 1143 and outputs the amplitude value for one period at a time to the position detection apparatus 1150A.

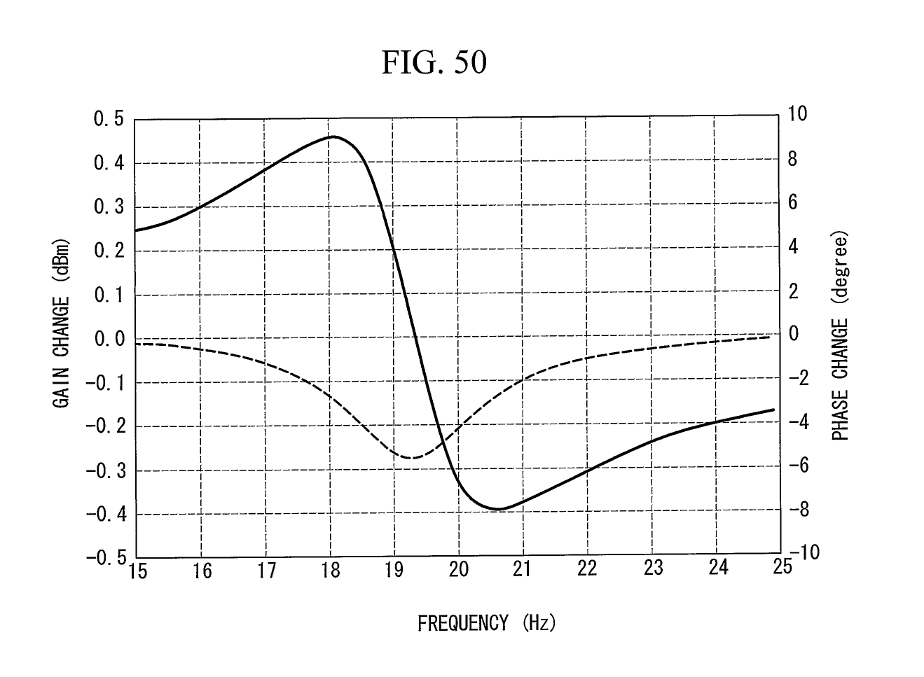

As shown in FIG. 50, the amplitude value of the AC voltage strongly varies depending on the relationship between the alternating magnetic field that the drive coil 1151 generates and the resonance frequency of the resonance circuit 1143. FIG. 50 shows the frequency of the alternating magnetic field on the horizontal axis and the variations in gain (dBm) and phase (degree) of the AC voltage flowing in the resonance circuit 1143 on the vertical axes. It is shown that the variation in gain, indicated by the solid line, exhibits a maximum value at a frequency smaller than the resonance frequency, is zero at the resonance frequency, and exhibits a minimum value at a frequency higher than the resonance frequency. Also, it is shown that the variation in phase, indicated by the broken line, drops most at the resonance frequency.

Depending on the measurement conditions, there may be cases where the gain exhibits a minimum value at a frequency lower than the resonance frequency and exhibits a maximum value at a frequency higher than the resonance frequency, and where the phase reaches a peak at the resonance frequency.

The extracted amplitude value is output to the position detection apparatus 1150A, and the position detection apparatus 1150A assumes the amplitude difference between the maximum value and the minimum value of the amplitude value in the vicinity of the resonance frequency as the output from the sense coil 1152. Then, the position detection apparatus 1150A obtains the position and so forth of the capsule endoscope 1120 by solving simultaneous equations involving the position, direction, and magnetic field strength of the capsule endoscope 1120 based on the amplitude difference obtained from the plurality of sense coils 1152.

Thus, by setting the output of the sense coils 1152 as the amplitude difference in this way, it is possible to cancel variations in amplitude that originate from variations in the magnetic field intensity due to environmental conditions (for example, temperature), and it is therefore possible to obtain the position of the capsule endoscope 1120 with a reliable degree of accuracy without being affected by environmental conditions.

The information on the position and so forth of the capsule endoscope 1120 includes six pieces of information, for example, X, Y, and Z positional coordinates, rotational phases φ and θ about axes that are orthogonal to each other and orthogonal to the central axis (rotation axis) of the capsule endoscope 1120, and the intensity of the induced magnetic field that the magnetic induction coil 1142 produces.

In order to estimate these six pieces of information by calculation, the outputs of at least six sense coils 1152 are necessary. Since the outputs of nine sense coils 1152 disposed in at least one plane are used to estimate the position of the capsule endoscope 1120, it is possible to obtain the six pieces of information mentioned above by calculation.

The position detection apparatus 1150A reports the amplification factor of the AC current supplied to the drive coil 1151 to the drive-coil driver 1154 based on the position of the capsule endoscope 1120 obtained by calculation. This amplification factor is set so that the induced magnetic field produced by the magnetic induction coil 1142 can be detected by the sense coil 1152.

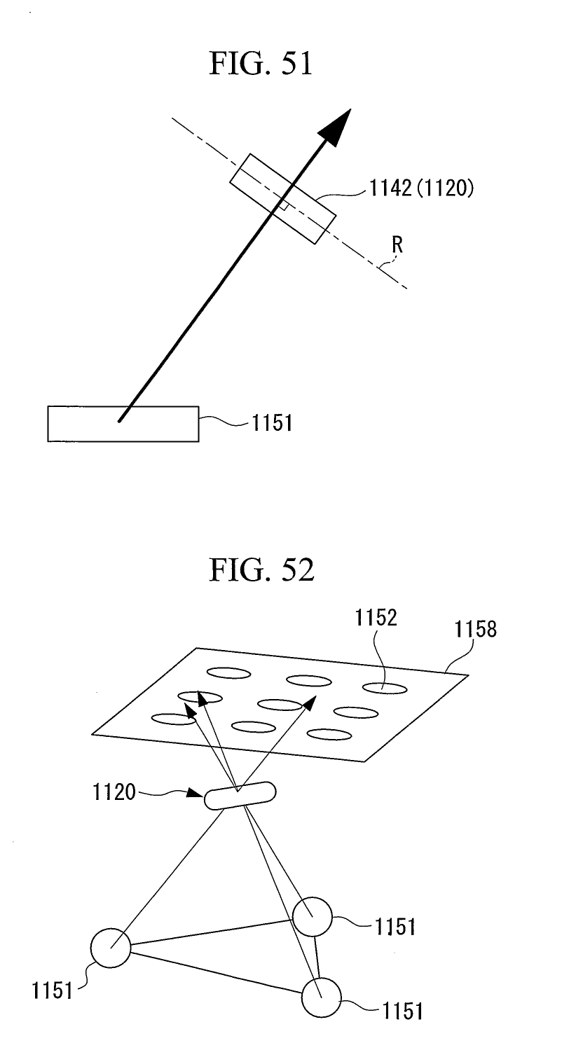

Also, the position detection apparatus 1150A selects drive coils 1151 for producing magnetic fields, and outputs to the drive coil selector 1155 an instruction for supplying the AC current to the selected drive coils 1151. As shown in FIG. 51, in the method of selecting the drive coils 1151, a drive coil 1151 for which a straight line (orientation of the drive coil 1151) connecting the drive coil 1151 and the magnetic induction coil 1142 and the central axis of the magnetic induction coil 1142 (the rotation axis R of the capsule endoscope 1120) are substantially orthogonal is excluded. In addition, as shown in FIG. 52, the drive coils 1151 are selected so as to supply the AC current to three of the drive coils 1151 in such a way that the orientations of the magnetic fields acting on the magnetic induction coil 4112 are linearly independent.

A more preferable method is a method in which a drive coil 1151 for which the orientation of the line of magnetic force produced by the drive coil 1151 and the central axis of the magnetic induction coil 1142 are substantially orthogonal is omitted.

The number of drive coils 1151 forming the alternating magnetic field may be limited using the drive-coil selector 1155, as described above, or the number of drive coils 1151 disposed may be initially set to three without using the drive-coil selector 1155.

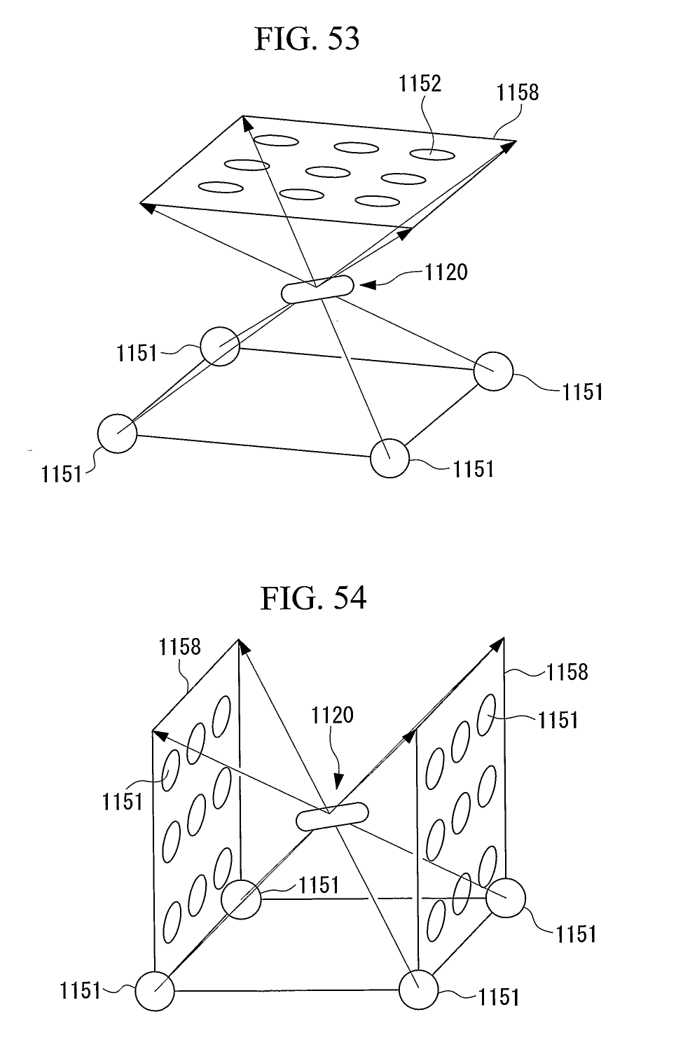

As described above, three drive coils 1151 may be selected to form the alternating magnetic field, or as shown in FIG. 53, the alternating magnetic field may be produced by all of the drive coils 1151.

Furthermore, the position detection apparatus 1150A selects sense coils 1152 whose detected amplitude differences are to be used to estimate the position of the capsule endoscope 1120 and outputs to the sense coil selector 1156 an instruction for inputting the AC currents from the selected sense coils 1152 to the sense-coil receiving circuit 1157.

The method of selecting the sense coils 1152 is not particularly limited. For example, as shown in FIG. 51, sense coils 1152 opposing the drive coils 1151 with the capsule endoscope 1120 disposed therebetween may be selected, or as shown in FIG. 54, sense coils 1152 that are disposed in mutually opposing planes adjacent to the plane in which the drive coils 1151 are disposed may be selected.

Furthermore, sense coils 1152 that are expected to induce large AC currents based on the acquired position and direction of the capsule endoscope 1120, such as sense coils 1152 disposed near the capsule endoscope 1120, may be selected.

AC currents that are induced in the sense coils 1152 disposed on the three coil-supporting parts 1158 may be selected by the sense-coil selector 1156, as described above, or, without using the sense-coil selector 1156, the number of coil-supporting parts 1158 provided may be set beforehand to either one or two, as shown in FIGS. 53 and 54.

Next, the operation of the magnetic induction apparatus 1170 will be described.

As shown in FIG. 43, in the magnetic induction apparatus 1170, first, the operator inputs a guidance direction for the capsule endoscope 1120 to the rotation-magnetic-field control circuit 1173 via the input device 1174. In the rotation-magnetic-field control circuit 1173, the orientation and rotation direction of a parallel magnetic field to be applied to the capsule endoscope 1120 are determined based on the input guidance direction and the orientation (rotation axis direction) of the capsule endoscope 1120 input from the position detection unit 1150.

Then, to produce the orientation of the parallel magnetic field, the required intensity of the magnetic fields produced by the Helmholtz coils 1171X, 1171Y, and 1171Z is calculated, and the electrical currents required to produce these magnetic fields are calculated.

The electric current data supplied to the individual Helmholtz coils 1171X, 1171Y, and 1171Z is output to the corresponding Helmholtz-coil drivers 1172X, 1172Y, and 1172Z, and the Helmholtz-coil drivers 1172X, 1172Y, and 1172Z carry out amplification control of the currents based on the input data and supply the currents to the corresponding Helmholtz coils 1171X, 1171Y, and 1171Z.

The Helmholtz coils 1171X, 1171Y, and 1171Z to which the currents are supplied produce magnetic fields according to the respective current values, and by combining these magnetic fields, a parallel magnetic field having the magnetic field orientation determined by the rotation-magnetic-field control circuit 1173 is produced.

The guidance magnet 1145 is provided in the capsule endoscope 1120 and, as described later, the orientation (rotation axis direction) of the capsule endoscope 1120 is controlled based on the force acting on the guidance magnet 1145 and the parallel magnetic field described above. Also, by controlling the rotation period of the parallel magnetic field to be about 0 Hz to a few Hz and controlling the rotation direction of the parallel magnetic field, the rotation direction about the rotation axis of the capsule endoscope 1120 is controlled, and the direction of movement and the moving speed of the capsule endoscope 1120 are also controlled.

Next, the operation of the capsule endoscope 1120 will be described.

As shown in FIG. 47, in the capsule endoscope 1120, first infrared light is irradiated onto the infrared sensor 1147 of the switch section 1146, and the switch section 1146 outputs a signal to the signal processing section 1134. When the signal processing section 1134 receives the signal from the switch section 1146, electrical current is supplied from the battery 1139 to the image sensor 1131, the LEDs 1133, the radio device 1135, and the signal processing section 1134 itself, which are built into the capsule endoscope 1120, and they are turned on.

The image sensor 1131 images a wall surface inside the passage in the body cavity of the subject 1, which is illuminated by the LEDs 1133, converts this image into an electrical signal, and outputs it to the signal processing section 1134. The signal processing section 1134 compresses the input image, temporarily stores it, and outputs it to the radio device 1135. The compressed image signal input to the radio device 1135 is transmitted to the image display apparatus 1180 as electromagnetic waves.

The capsule endoscope 1120 can move towards the front end portion 1123 or the rear end portion 1124 by rotating about the rotation axis R by means of the helical part 1125 provided on the outer circumference of the outer casing 1121. The direction of motion is determined by the rotation direction about the rotation axis R and the direction of rotation of the helical part 1125.

Next, the operation of the image display apparatus 1180 will be described.

As shown in FIG. 43, in the image display apparatus 1180, first the image receiving circuit 1181 receives the compressed image signal transmitted from the capsule endoscope 1120, and the image signal is output to the display section 1182. The compressed image signal is reconstructed in the image receiving circuit 1181 or the display section 1182, and is displayed by the display section 1182.

Also, the display section 1182 performs rotation processing on the image signal in the opposite direction to the rotation direction of the capsule endoscope 1120 based on the rotational phase data of the capsule endoscope 1120, which is input from the rotation-magnetic-field control circuit 1173, and displays it.

A test for a change in output of a magnetic induction coil depending on objects disposed in the magnetic induction coil will now be described.

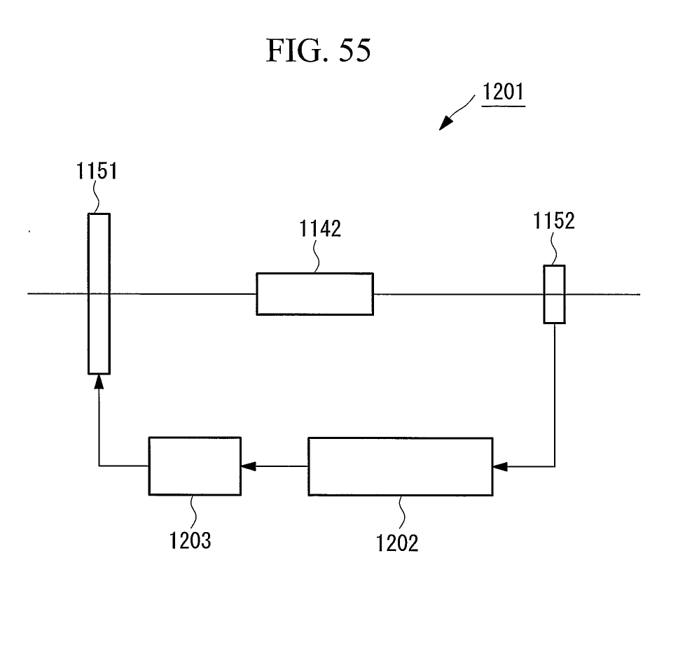

FIG. 55 is a diagram illustrating in outline an experimental apparatus used for the current test.

As shown in FIG. 55, an experimental apparatus 1201 includes a magnetic induction coil 1142 to be tested; a drive coil 1151 for applying a magnetic field to the magnetic induction coil 1142; a sense coil 1152 for detecting the induced magnetic field generated in the magnetic induction coil 1142; a network analyzer 1202 for analyzing the signal detected by the sense coil 1152; and an amplifier 1203 for amplifying the output of the network analyzer 1202 and outputting it to the drive coil 1151.

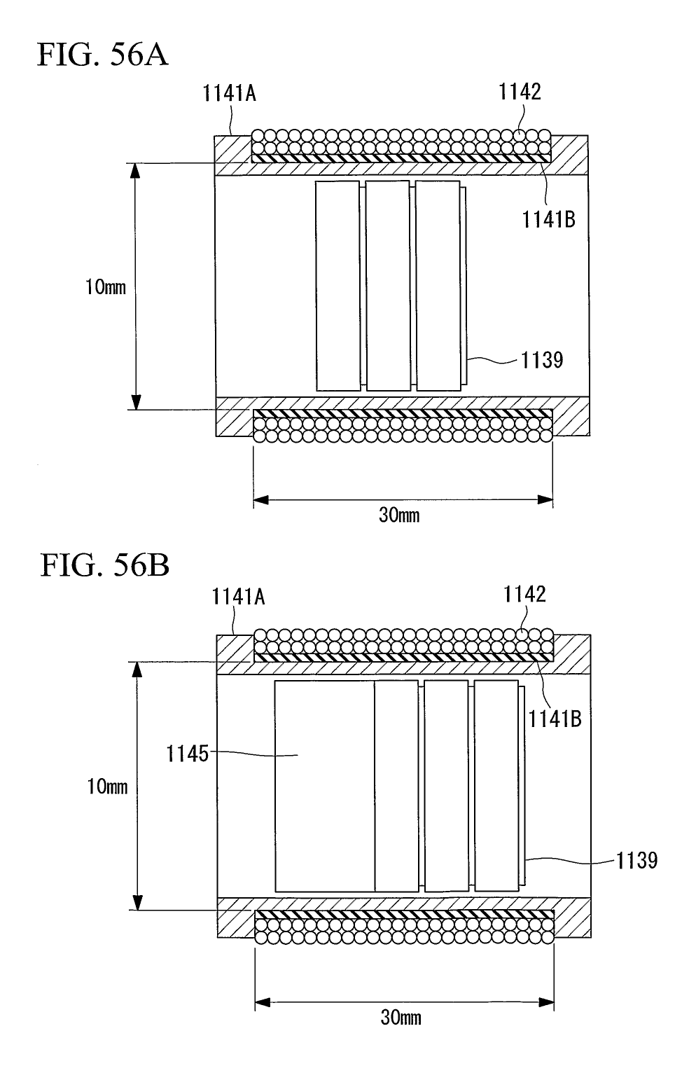

FIG. 56 is a diagram illustrating the magnetic induction coil 1142 and objects arranged in the magnetic induction coil 1142 for the current test. FIG. 56A is a diagram illustrating the magnetic induction coil 1142 and a battery 1139, and FIG. 56B is a diagram illustrating the magnetic induction coil 1142, the battery 1139, and a guidance magnet 1145.

As shown in FIGS. 56A and 56B, the magnetic induction coil 1142 is arranged on the circumferential surface of a cylindrical permalloy film 1141B with an inner diameter of about 10 mm and is formed to have a length of about 30 mm.

The battery 1139 used for the current test is formed of three button batteries arranged in series.

As shown in FIG. 56B, the guidance magnet 1145 used for the current test is a substantially cylindrical object with a diameter of about 8 mm and a length of about 6 mm and is formed of neodymium-cobalt.

In this test, the positional relationship between the magnetic induction coil 1142 and the battery 1139 and the positional relationship between the magnetic induction coil 1142, the battery 1139, and the guidance magnet 1145 are as shown in FIGS. 56A and 56B.

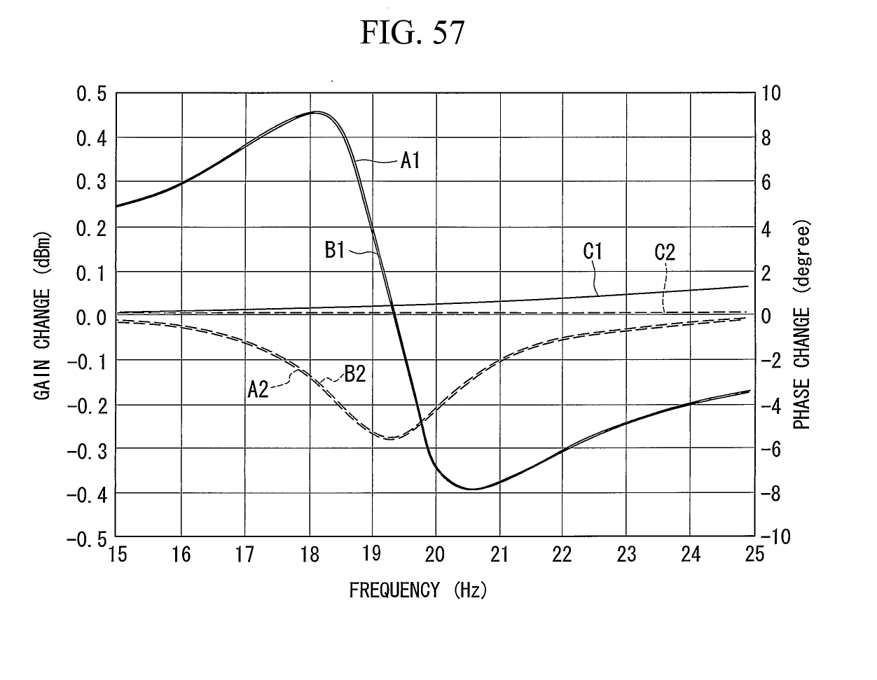

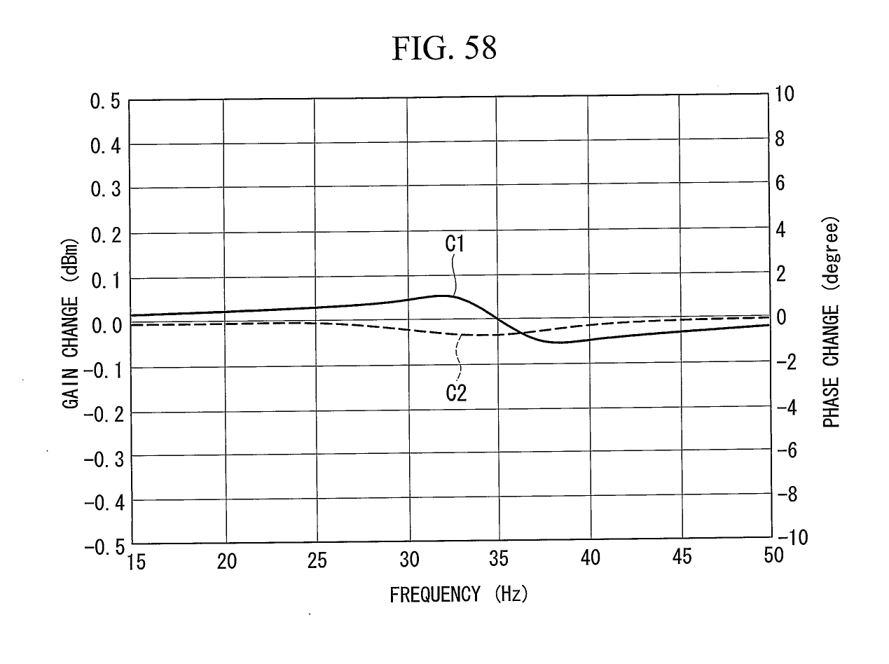

FIGS. 57 and 58 are diagrams depicting the relationship between the frequency of an alternating magnetic field formed by the drive coil 1151 and changes in gain and phase.

In FIGS. 57 and 58, A1 and A2 indicate a gain change and a phase change, respectively, when measured with only the magnetic induction coil 1142; B1 and B2 indicate a gain change and a phase change, respectively, measured when the battery 1139 is arranged in the magnetic induction coil 1142 (refer to FIG. 56A); and C1 and C2 indicate a gain change and a phase change, respectively, measured when the battery 1139 and the guidance magnet 1145 are arranged in the magnetic induction coil 1142 (refer to FIG. 56B).

As shown in FIGS. 57 and 58, no difference was found between the case of measurement with only the magnetic induction coil 1142 (A1, A2) and the case where the battery 1139 was arranged in the magnetic induction coil 1142 (B1, B2). On the other hand, in the case where the battery 1139 and the guidance magnet 1145 were arranged in the magnetic induction coil 1142 (C1, C2), the frequency at which a gain change and a phase change occur became closer to the high-frequency side and the range of gain change was smaller than in the other cases.

As a result, it was found that arranging the battery 1139 in the magnetic induction coil 1142 does not affect the characteristic of the magnetic induction coil 1142 and that arranging the guidance magnet 1145 tends to cause the output of the magnetic induction coil 1142 to become weak.

A test for a change in output of the magnetic induction coil depending on the distance to the guidance magnet will now be described.

As with the above-described test, the experimental apparatus 1201 shown in FIG. 55 is used for this test.

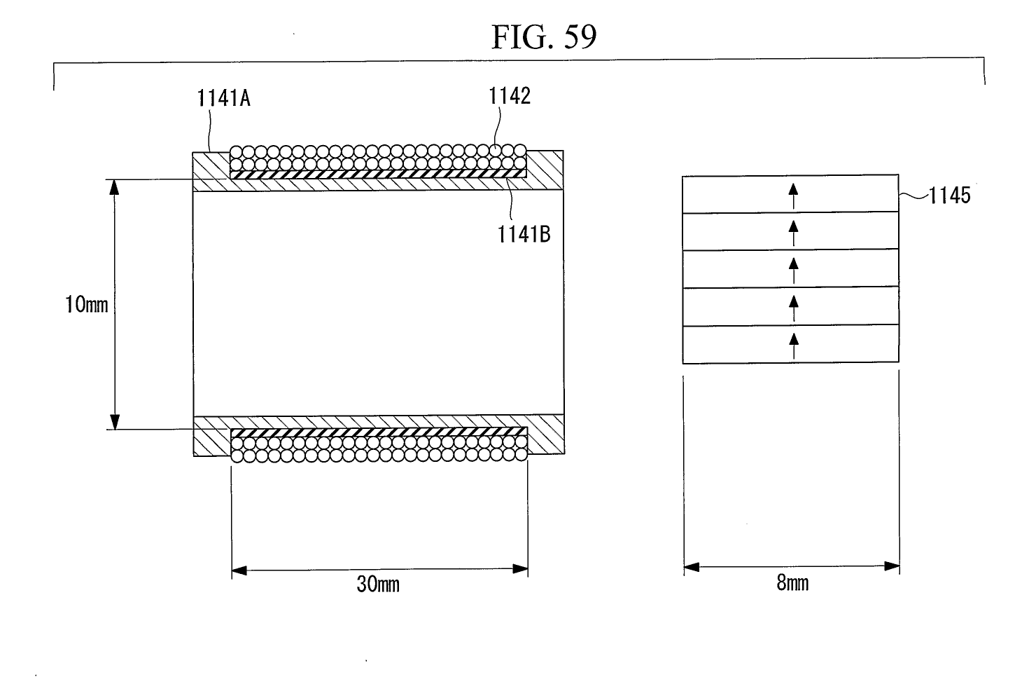

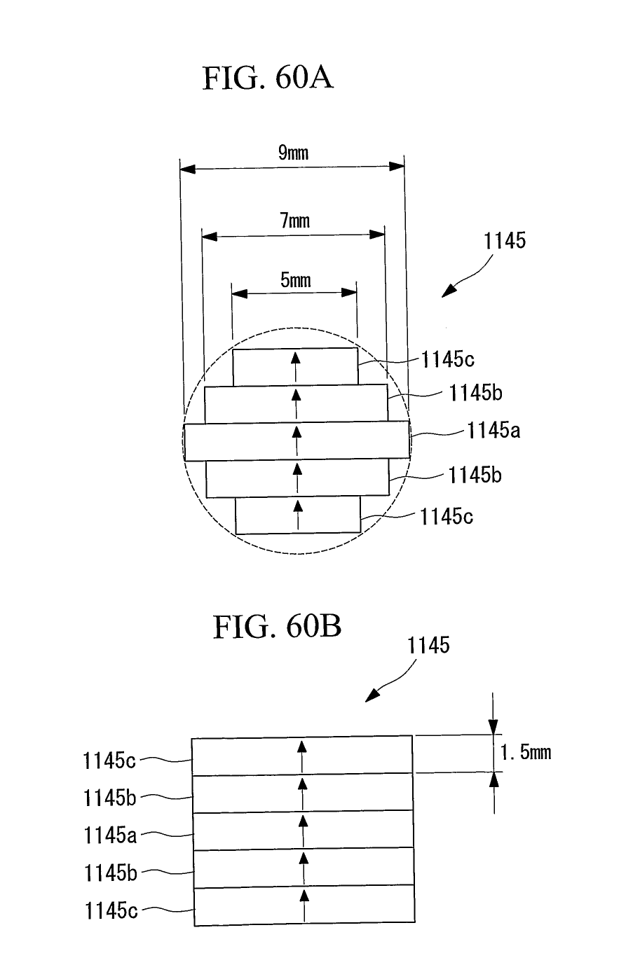

FIG. 59 is a diagram illustrating the positional relationship between the magnetic induction coil 1142 and the guidance magnet 1145 in the current test. FIG. 60 is a diagram illustrating the structure of the solid-core guidance magnet used for the current test. FIG. 60A is a front elevational view of the guidance magnet, and FIG. 60B is a side elevational view of the guidance magnet.

As shown in FIG. 59, the magnetic induction coil 1142 is arranged on the circumferential surface of the cylindrical permalloy film 1141B with an inner diameter of about 10 mm and is formed to have a length of about 30 mm.

As shown in FIGS. 60A and 60B, the solid-core guidance magnet 1145 is formed in a substantially cylindrical shape and is composed of one large-size magnet piece 1145a, two medium-size magnet pieces 1145b, and two small-size magnet pieces 1145c being substantially formed in the shape of a plate. The widths of the large-size magnet piece 1145a, the medium-size magnet pieces 1145b, and the small-size magnet pieces 1145c are about 9 mm, about 7 mm, and about 5 mm, respectively. The thicknesses and lengths of the magnet pieces are the same, more specifically, about 1.5 mm and about 8 mm, respectively. Furthermore, the magnet pieces are formed of neodymium-cobalt and magnetized in their thickness directions. The side indicated by the arrows in the figure corresponds to the north pole, and the opposite side corresponds to the south pole.

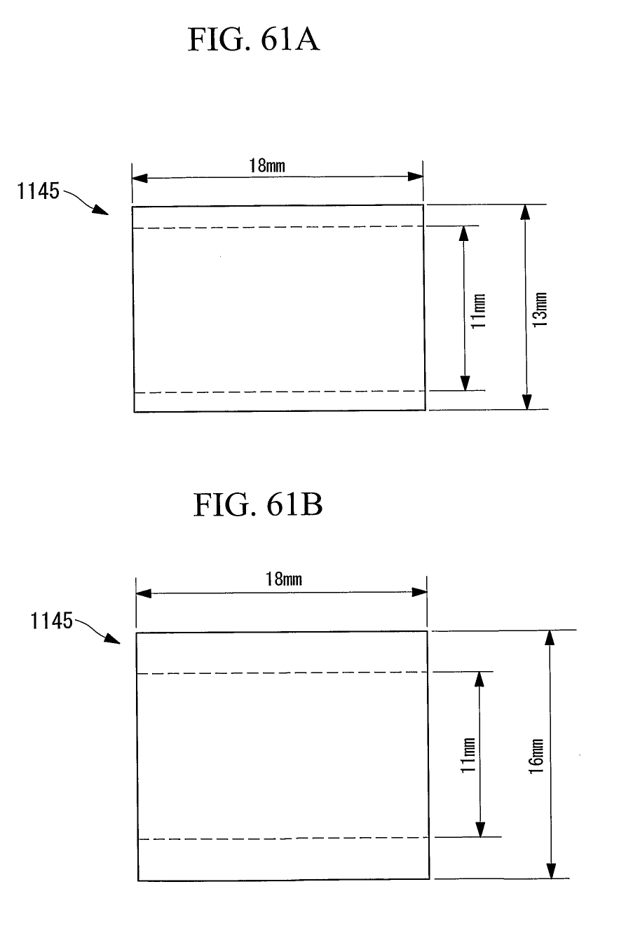

FIG. 61A is a side elevational view showing the structure of the hollow guidance magnet used for the current test. FIG. 61B is a side elevational view of the large-size hollow guidance magnet.

As shown in FIG. 61A, the hollow guidance magnet 1145 is formed like a cylinder with an outer diameter of about 13 mm, an inner diameter of about 11 mm, and a length of about 18 mm, and is formed of neodymium-cobalt. As shown in FIG. 61B, the large-size guidance magnet 1145 is formed like a cylinder with an outer diameter of about 16 mm, an inner diameter of about 11 mm, and a length of about 18 mm, and is formed of neodymium-cobalt.

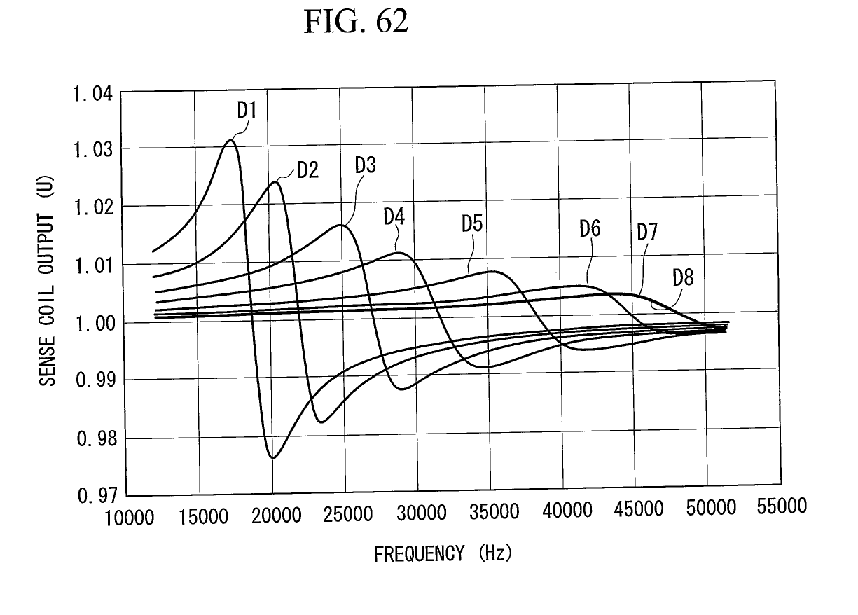

FIG. 62 is a diagram depicting the relationship between the frequency of an alternating magnetic field formed by the drive coil 1151 and the sense coil output in the guidance magnet 1145 composed of the five magnet pieces 1145a, 1145b, 1145b, 1145c, and 1145c.

In the figure, D1 is a graph showing a sense coil output when the guidance magnet 1145 is removed; D2 is a graph showing a sense coil output when the distance between the guidance magnet 1145 and the magnetic induction coil 1142 is 10 mm; D3 is a graph showing a sense coil output when the above-described distance is 5 mm; D4 is a graph showing a sense coil output when the above-described distance is 0 mm; D5 is a graph showing a sense coil output when the above-described distance is −5 mm (the guidance magnet 1145 is inside the magnetic induction coil 1142); D6 is a graph showing a sense coil output when the above-described distance is −10 mm; D7 is a graph showing a sense coil output when the above-described distance is −15 mm; and D8 is a graph showing a sense coil output when the above-described distance is −18 mm.

As shown in FIG. 62, as the distance between the guidance magnet 1145 and the magnetic induction coil 1142 becomes small, the output variation range becomes small and the frequency at which the output changes moves towards the high-frequency side.

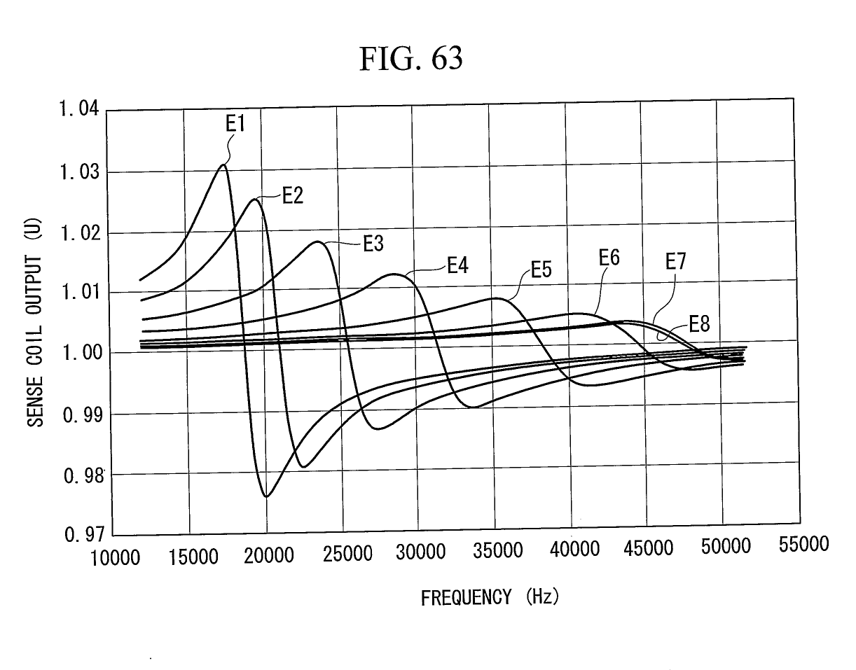

FIG. 63 is a diagram showing the relationship between a sense coil output and the frequency of an alternating magnetic field formed by the drive coil 1151 in a case where the guidance magnet 1145 is composed of the five magnet pieces 1145a, 1145b, 1145b, 1145c, and 1145c and vinyl sheets serving as insulators are interposed between the magnet pieces 1145a, 1145b, and 1145c.

In the figure, E1 is a graph showing a sense coil output when the guidance magnet 1145 is removed; E2 is a graph showing a sense coil output when the distance between the guidance magnet 1145 and the magnetic induction coil 1142 is 10 mm; E3 is a graph showing a sense coil output when the above-described distance is 5 mm; E4 is a graph showing a sense coil output when the above-described distance is 0 mm; E5 is a graph showing a sense coil output when the above-described distance is −5 mm (the guidance magnet 1145 is inside the magnetic induction coil 1142); E6 is a graph showing a sense coil output when the above-described distance is −10 mm; E7 is a graph showing a sense coil output when the above-described distance is −15 mm; and E8 is a graph showing a sense coil output when the above-described distance is −18 mm.

As shown in FIG. 63, with the insulators being interposed between the magnet pieces 1145a, 1145b, and 1145c, a decrease in output variation range when the distance is 10 mm (E2) becomes small and the displacement of the frequency at which the output changes towards the high-frequency side decreases.

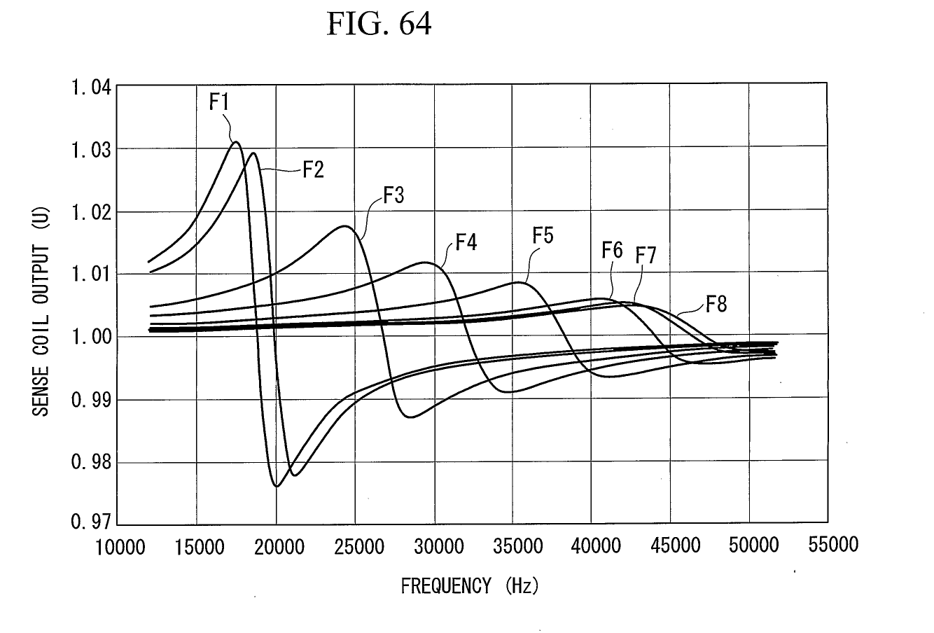

FIG. 64 is a diagram showing the relationship between a sense coil output and the frequency of an alternating magnetic field formed by the drive coil 1151 in a case where the guidance magnet 1145 is composed of one large-size magnet piece 1145a and two medium-size magnet pieces 1145b and 1145b and vinyl sheets serving as insulators are interposed between the magnet pieces 1145a and 1145b.

In the figure, F1 is a graph showing a sense coil output when the guidance magnet 1145 is removed; F2 is a graph showing a sense coil output when the distance between the guidance magnet 1145 and the magnetic induction coil 1142 is 10 mm; F3 is a graph showing a sense coil output when the above-described distance is 5 mm; F4 is a graph showing a sense coil output when the above-described distance is 0 mm; F5 is a graph showing a sense coil output when the above-described distance is −5 mm (the guidance magnet 1145 is inside the magnetic induction coil 1142); F6 is a graph showing a sense coil output when the above-described distance is −10 mm; F7 is a graph showing a sense coil output when the above-described distance is −15 mm; and F8 is a graph showing a sense coil output when the above-described distance is −18 mm.

As shown in FIG. 64, with a smaller volume of the guidance magnet 1145, a decrease in output variation range when the distance is 10 mm (F2) becomes smaller and the displacement of the frequency at which the output changes towards the high-frequency side decreases more.

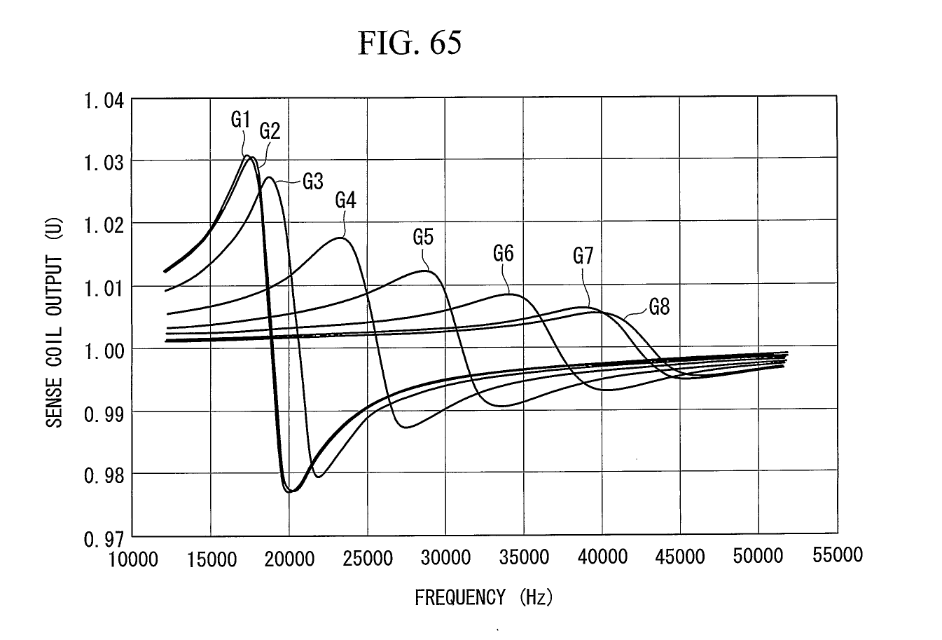

FIG. 65 is a diagram depicting the relationship between the frequency of an alternating magnetic field formed by the drive coil 1151 and the sense coil output in the guidance magnet 1145 composed of the one large-size magnet piece 1145a.

In the figure, G1 is a graph showing a sense coil output when the guidance magnet 1145 is removed; G2 is a graph showing a sense coil output when the distance between the guidance magnet 1145 and the magnetic induction coil 1142 is 10 mm; G3 is a graph showing a sense coil output when the above-described distance is 5 mm; G4 is a graph showing a sense coil output when the above-described distance is 0 mm; G5 is a graph showing a sense coil output when the above-described distance is −5 mm (the guidance magnet 1145 is inside the magnetic induction coil 1142); G6 is a graph showing a sense coil output when the above-described distance is −10 mm; G7 is a graph showing a sense coil output when the above-described distance is −15 mm; and G8 is a graph showing a sense coil output when the above-described distance is −18 mm.

As shown in FIG. 65, with an even smaller volume of the guidance magnet 1145, the graph in the case where the distance is 10 mm (G2) becomes nearly the same as the graph in the case where the guidance magnet 1145 is removed (G1), a decrease in output variation range under other conditions (e.g., G3) becomes small, and the displacement of the frequency at which the output changes towards the high-frequency side decreases.

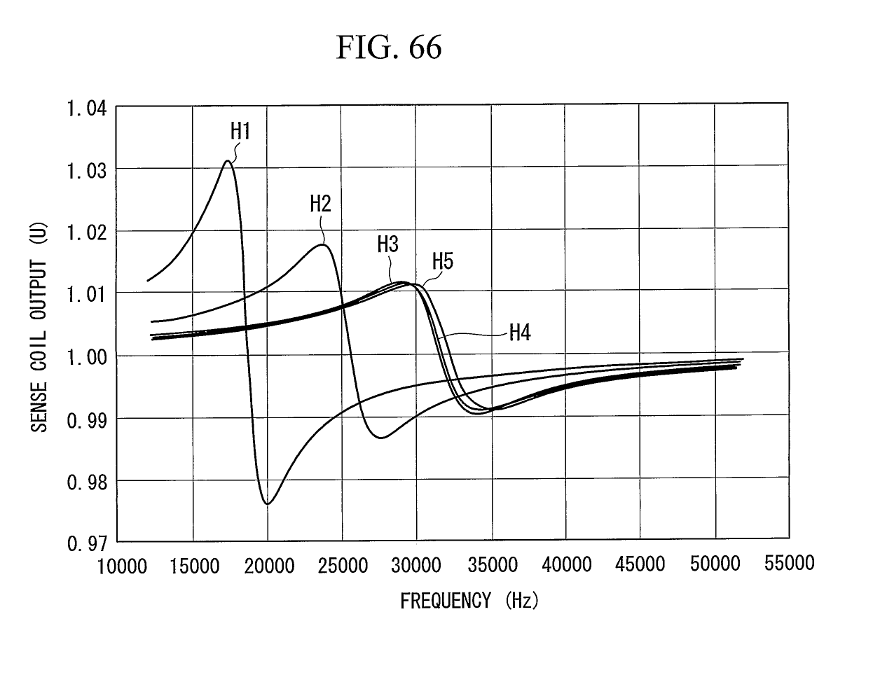

FIGS. 66 to 68 are diagrams showing the above-described results, classified by the distance between the guidance magnet 1145 and the magnetic induction coil 1142.

FIG. 66 is a diagram showing the results when the distance between the guidance magnet 1145 and the magnetic induction coil 1142 is 0 mm. In the figure, H1 is a graph showing the results when the guidance magnet 1145 is not present; H2 is a graph showing the results with the guidance magnet 1145 composed of the five magnet pieces 1145a, 1145b, 1145b, 1145c, and 1145c; H3 is a graph showing the results with the guidance magnet 1145 having insulators disposed between the five magnet pieces 1145a, 1145b, 1145b, 1145c, and 1145c; H4 is a graph showing the results with the guidance magnet 1145 composed of the three magnet pieces 1145a, 1145b, and 1145b having insulators disposed therebetween; and H5 is a graph showing the results with the guidance magnet 1145 composed of the one magnet piece 1145a.

As shown in FIG. 66, when the guidance magnet 1145 is present, the output variation range decreases and the frequency at which the output changes moves towards the high-frequency side.

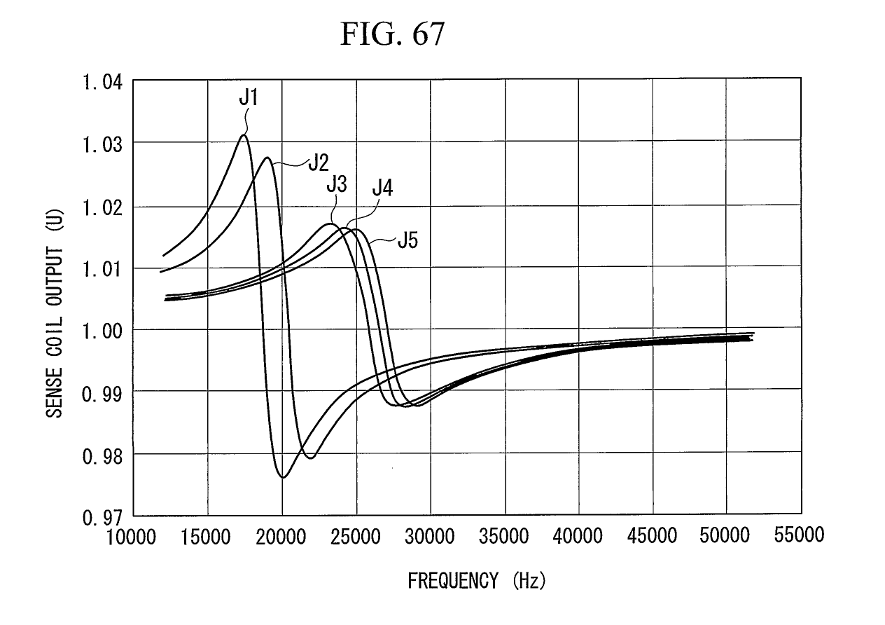

FIG. 67 is a diagram showing the results when the distance between the guidance magnet 1145 and the magnetic induction coil 1142 is 5 mm. In the figure, J1 is a graph showing the results when the guidance magnet 1145 is not present; J2 is a graph showing the results with the guidance magnet 1145 composed of the five magnet pieces 1145a, 1145b, 1145b, 1145c, and 1145c; J3 is a graph showing the results with the guidance magnet 1145 having insulators disposed between the five magnet pieces 1145a, 1145b, 1145b, 1145c, and 1145c; J4 is a graph showing the results with the guidance magnet 1145 composed of the three magnet pieces 1145a, 1145b, and 1145b having insulators disposed therebetween; and J5 is a graph showing the results with the guidance magnet 1145 composed of the one magnet piece 1145a.

As shown in FIG. 67, when the above-described distance becomes large, a decrease in output variation range becomes small and the displacement of the frequency at which the output changes towards the high-frequency side decreases.

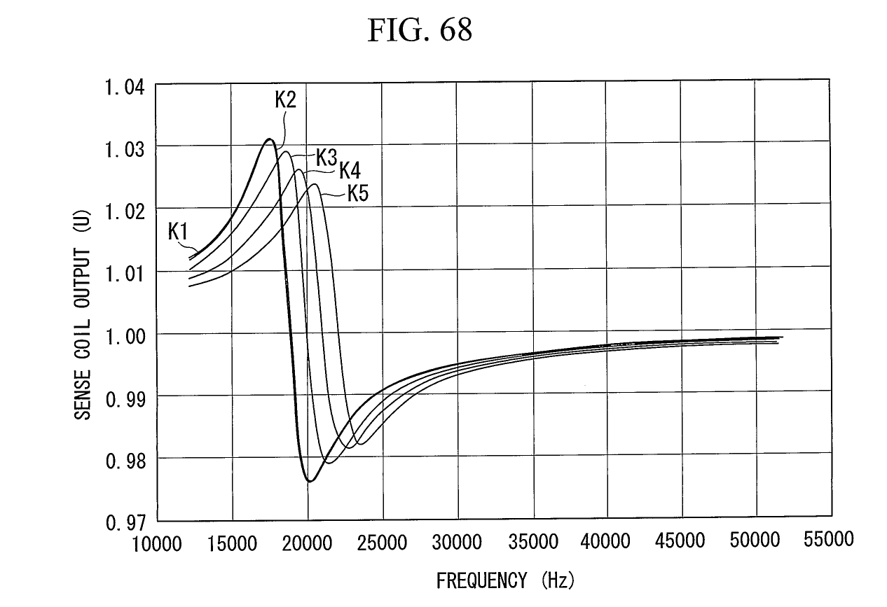

FIG. 68 is a diagram showing the results when the distance between the guidance magnet 1145 and the magnetic induction coil 1142 is 10 mm. In the figure, K1 is a graph showing the results when the guidance magnet 1145 is not present; K2 is a graph showing the results with the guidance magnet 1145 composed of the five magnet pieces 1145a, 1145b, 1145b, 1145c, and 1145c; K3 is a graph showing the results with the guidance magnet 1145 having insulators disposed between the five magnet pieces 1145a, 1145b, 1145b, 1145c, and 1145c; K4 is a graph showing the results with the guidance magnet 1145 composed of the three magnet pieces 1145a, 1145b, and 1145b having insulators disposed therebetween; and K5 is a graph showing the results with the guidance magnet 1145 composed of the one magnet piece 1145a.

As shown in FIG. 68, when the above-described distance becomes large, a decrease in output variation range becomes smaller and the displacement of frequency at which the output changes towards the high-frequency side decreases more.

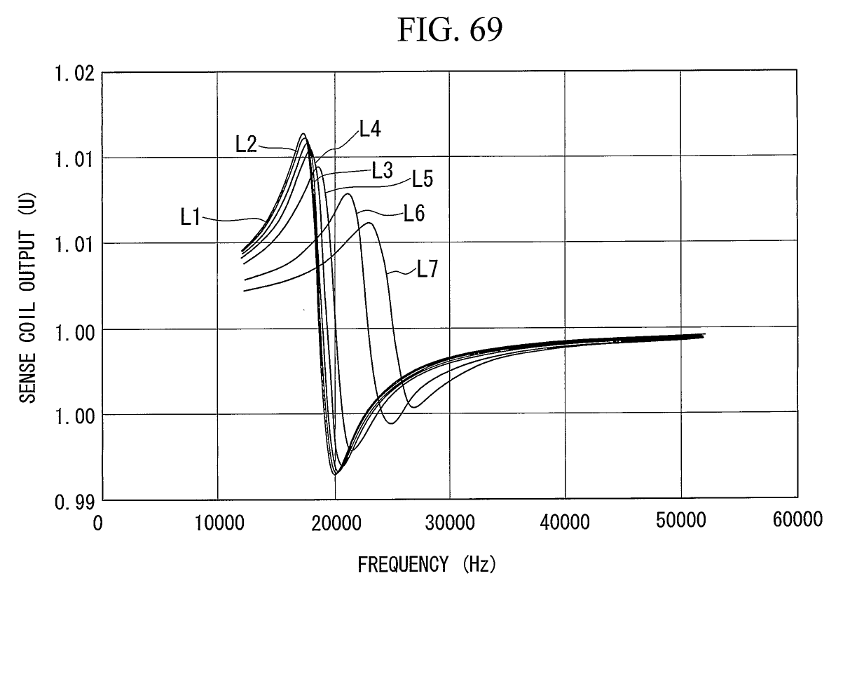

FIG. 69 is a diagram depicting the relationship between the frequency of an alternating magnetic field formed by the drive coil 1151 and the sense coil output in the hollow guidance magnet 1145 (refer to FIG. 61A).

In the figure, L1 is a graph showing a sense coil output when the guidance magnet 1145 is removed; L2 is a graph showing a sense coil output when the distance between the hollow guidance magnet 1145 and the magnetic induction coil 1142 is 15 mm; L3 is a graph showing a sense coil output when the above-described distance is 12 mm; L4 is a graph showing a sense coil output when the above-described distance is 10 mm; L5 is a graph showing a sense coil output when the above-described distance is 8 mm; L6 is a graph showing a sense coil output when the above-described distance is 5 mm; and L7 is a graph showing a sense coil output when the above-described distance is 2 mm.

As shown in FIG. 69, as the distance between the hollow guidance magnet 1145 and the magnetic induction coil 1142 becomes large, the output variation range becomes large and the frequency at which the output changes moves towards the low-frequency side.

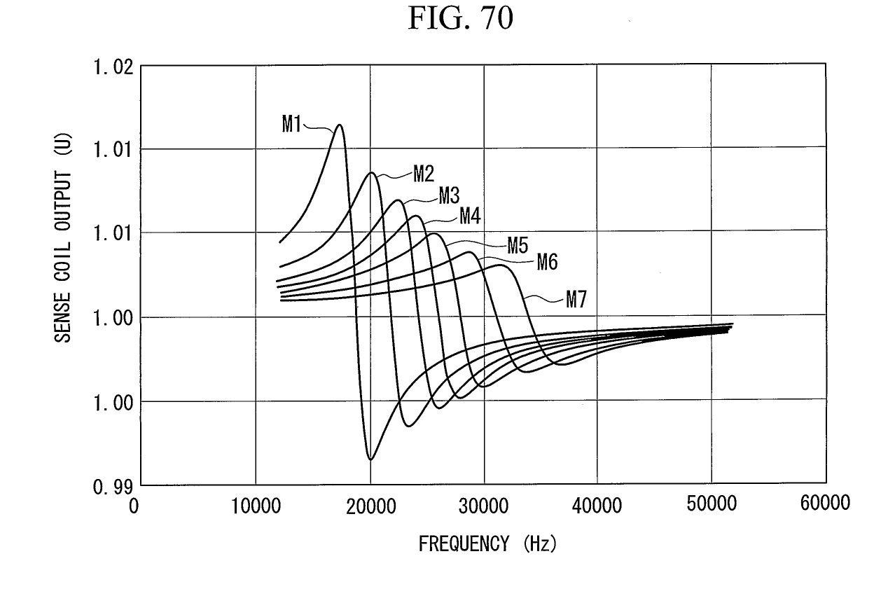

FIG. 70 is a diagram depicting the relationship between the frequency of an alternating magnetic field formed by the drive coil 1151 and the sense coil output in the large-size hollow guidance magnet 1145 (refer to FIG. 61B).

In the figure, M1 is a graph showing a sense coil output when the guidance magnet 1145 is removed; M2 is a graph showing a sense coil output when the distance between the large-size hollow guidance magnet 1145 and the magnetic induction coil 1142 is 15 mm; M3 is a graph showing a sense coil output when the above-described distance is 12 mm; M4 is a graph showing a sense coil output when the above-described distance is 10 mm; M5 is a graph showing a sense coil output when the above-described distance is 8 mm; M6 is a graph showing a sense coil output when the above-described distance is 5 mm; and M7 is a graph showing a sense coil output when the above-described distance is 2 mm.

As shown in FIG. 70, as the distance between the large-size hollow guidance magnet 1145 and the magnetic induction coil 1142 becomes large, the output variation range becomes large and the frequency at which the output changes moves towards the low-frequency side.

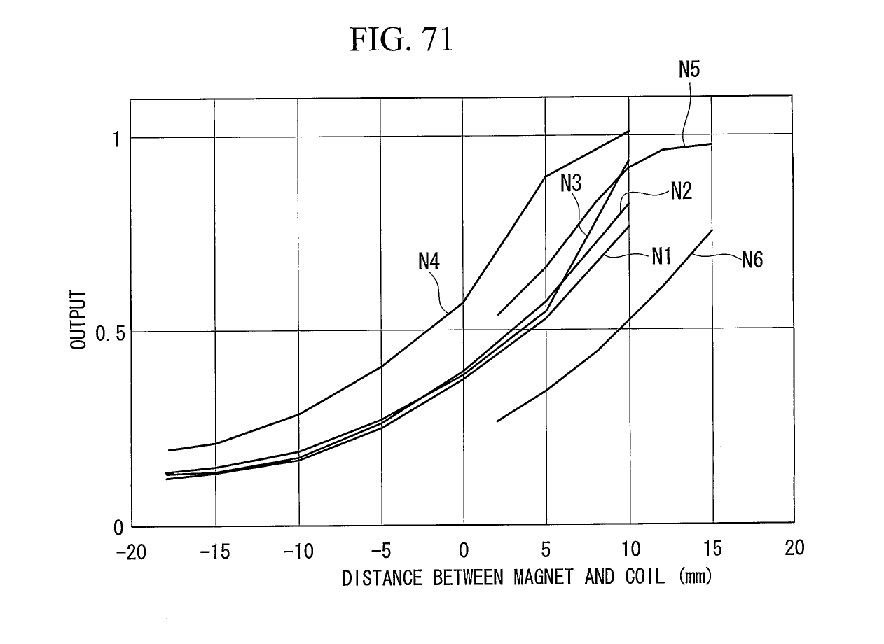

FIG. 71 is a diagram showing the above-described results, classified by the distance between the guidance magnet 1145 and the magnetic induction coil 1142 and by the magnitude of the output amplitude of the magnetic induction coil 1142. Here, the distance between the guidance magnet 1145 and the magnetic induction coil 1142 refers to the distance from the end surface of the guidance magnet 1145 to the center of the magnetic induction coil 1142. Furthermore, the magnitude of the output amplitude of the magnetic induction coil 1142 is represented relative to the output amplitude when the guidance magnet 1145 is not present.

In the figure, N1 is a graph showing the results with the guidance magnet 1145 composed of the five magnet pieces 1145a, 1145b, 1145b, 1145c, and 1145c; N2 is a graph showing the results with the guidance magnet 1145 composed of the five magnet pieces 1145a, 1145b, 1145b, 1145c, and 1145c having insulators disposed therebetween; N3 is a graph showing the results with the guidance magnet 1145 composed of the three magnet pieces 1145a, 1145b, and 1145b having insulators disposed therebetween; N4 is a graph showing the results with the guidance magnet 1145 composed of the one magnet piece 1145a; N5 is a graph showing the results with the hollow guidance magnet 1145; and N6 is a graph showing the results with the large-size hollow guidance magnet 1145.

As shown in FIG. 71, in all cases, as the above-described distance becomes large, the output amplitude of the magnetic induction coil 1142 becomes large. Furthermore, as the volume of the guidance magnet 1145 becomes small, the output amplitude of the magnetic induction coil 1142 becomes large.

In more detail, even with the guidance magnet 1145 composed of the five magnet pieces 1145a, 1145b, 1145b, 1145c, and 1145c, which are relatively large components built into the capsule endoscope 1120, or the large-size hollow guidance magnet 1145, a decrease in the output of the sense coil 1152 can be controlled to about 50% by setting the distance between the guidance magnet 1145 and the magnetic induction coil 1142 to 10 mm.

In addition, since the cylindrical guidance magnets (hollow guidance magnet, large-size hollow guidance magnet) cause the magnetic field in the magnetic induction coil 1142 to become less intense than the solid-core guidance magnet, the distance between the guidance magnet 1145 and the magnetic induction coil 1142 can be made smaller with the cylindrical guidance magnets. Alternatively, the volumes of the cylindrical guidance magnets can be increased.

Measurements of the intensity of the magnetic field formed by the guidance magnet 1145 at the center of the magnetic induction coil 1142 will be described in conjunction with the above-described results.

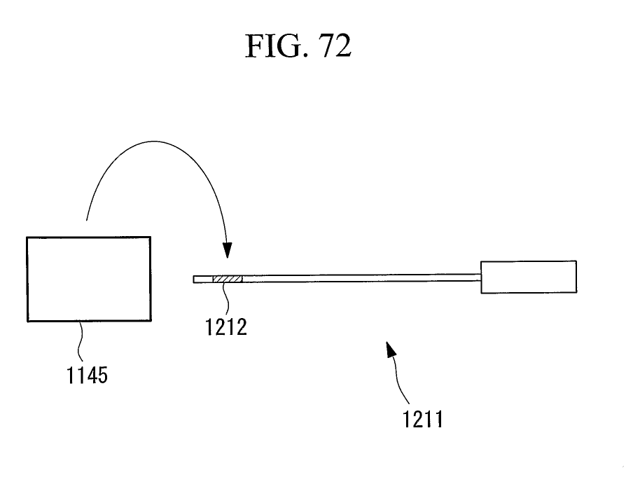

FIG. 72 is a diagram illustrating in outline an apparatus for measuring the magnetic field intensity formed by the guidance magnet 1145. As shown in FIG. 72, a gauss meter 1211 for measuring the magnetic field intensity of the guidance magnet 1145 is arranged such that a sensor section 1212 thereof substantially corresponds to the center of the guidance magnet 1145. For this reason, the magnetic field of the guidance magnet 1145 orthogonally intersects the sensor section 1212 of the gauss meter 1211.

Furthermore, the distance in the current measurement refers to the distance from the end surface of the guidance magnet 1145 to the center of the sensor section 1212.

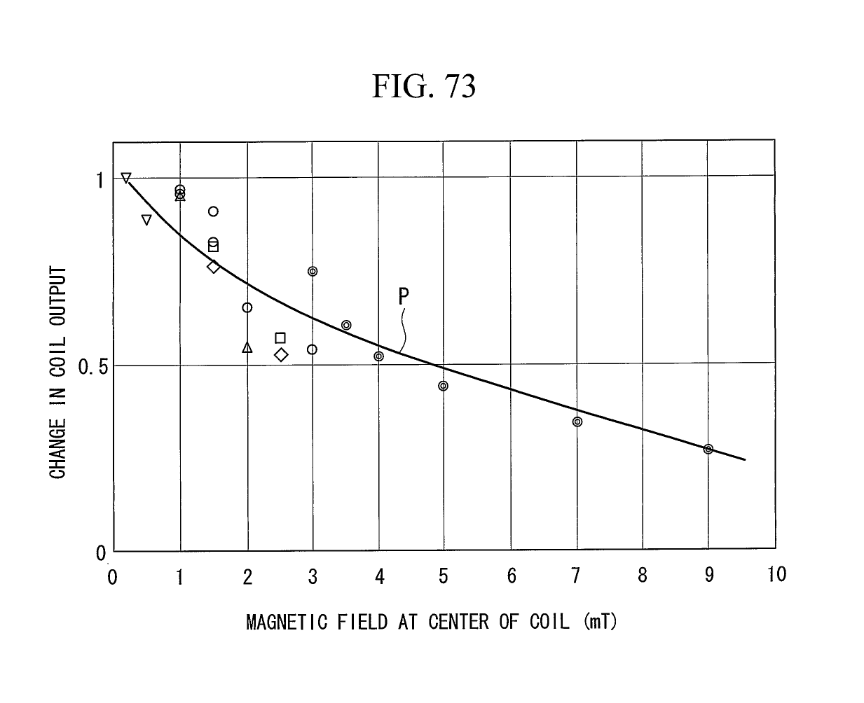

FIG. 73 is a diagram depicting the relationship between the intensity of a magnetic field formed by the guidance magnet at the center of the magnetic induction coil 1142 and the magnitude of the output amplitude of the magnetic induction coil 1142. The magnitude of the output amplitude is represented relative to the magnitude when the guidance magnet 1145 is not present.

In the figure, diamonds (⋄) indicate measurements with the guidance magnet 1145 composed of the five magnet pieces 1145a, 1145b, 1145b, 1145c, and 1145c; boxes (□) indicate measurements with the guidance magnet 1145 composed of the five magnet pieces 1145a, 1145b, 1145b, 1145c, and 1145c having insulators interposed therebetween; triangles (Δ) indicate measurements with the guidance magnet 1145 composed of the three magnet pieces 1145a, 1145b, and 1145b having insulators interposed therebetween; inverted triangles (∇) indicate measurements with the guidance magnet 1145 composed of the one magnet piece 1145a; circles (◯) indicate measurements with the hollow guidance magnet 1145; and double circles (⊚) indicate measurements with the large-size hollow guidance magnet 1145. P in the figure represents an approximate curve obtained from the above-described measurement points.

As shown in FIG. 73, regardless of the shape and volume of the guidance magnet 1145, the magnitude of the output amplitude of the magnetic induction coil 1142 decreases as the magnetic field intensity at the center of the magnetic induction coil 1142 increases. More specifically, if the intensity of the magnetic field produced at the center of the magnetic induction coil 1142 is about 5 mT, a decrease in output of the sense coil 1152 can be controlled to about 50%.

Therefore, by determining the arrangement distance between the guidance magnet 1145 and the magnetic induction coil 1142 according to the magnetic field intensity of the guidance magnet 1145 formed at the center of the magnetic induction coil 1142, the output amplitude of the magnetic induction coil 1142 can be prevented from decreasing, and therefore, problems can be prevented from occurring more reliably when the position of the magnetic induction coil 1142 is to be detected using the sense coil 1152.

A magnetic field and so forth formed in the permalloy film 1141B when a static magnetic field of the guidance magnet 1145 and an alternating magnetic field of the drive coil 1151 are formed at the position of the magnetic induction coil 1142 will now be described.

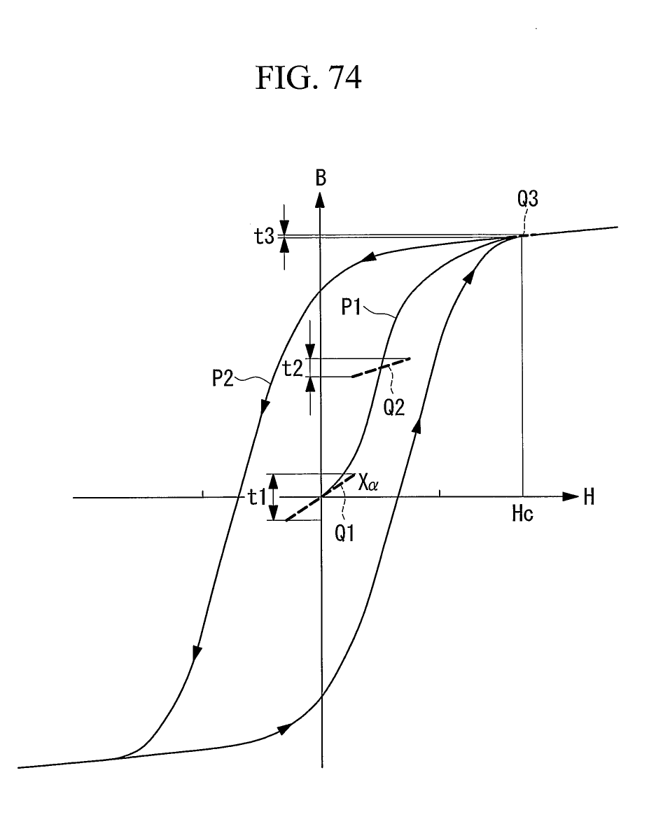

FIG. 74 is a diagram depicting a hysteresis loop and so forth in the permalloy film 1141B.

Magnetization curves indicating characteristics when a static magnetic field of the guidance magnet 1145 is formed at the position of the permalloy film 1141B are represented by solid-line curves P1 and P2 in FIG. 74.

The magnetization curve P1 is an initial magnetization curve P1, which represents the relationship between the static magnetic field and the magnetic flux density in the permalloy film 1141B when the guidance magnet 1145 is first brought near the permalloy film 1141B. The magnetization curve P2 represents a hysteresis loop.

In the hysteresis loop of FIG. 74, the horizontal axis represents the intensity of the magnetic field formed at the position of the permalloy film 1141B, and the vertical axis represents the magnetic flux density formed in the permalloy film 1141B.

Furthermore, magnetization curves indicating characteristics when an alternating magnetic field of the drive coil 1151 is formed at the position of the permalloy film 1141B are represented by broken straight lines Q1, Q2, and Q3 in FIG. 74.

The straight line Q1 represents a magnetization curve when the alternating magnetic field is formed without a static magnetic field formed at the position of the permalloy film 1141B. The straight line Q2 represents a magnetization curve when an alternating magnetic field is formed under a condition where a static magnetic field of about half the saturated magnetic field intensity (Hc) is formed at the position of the permalloy film 1141B. The straight line Q2 represents a magnetization curve when an alternating magnetic field is formed under a condition where a static magnetic field of the saturated magnetic field intensity (Hc) is formed at the position of the permalloy film 1141B. The slope of each of the straight lines Q1, Q2, and Q3 indicates the reversible magnetic susceptibility.

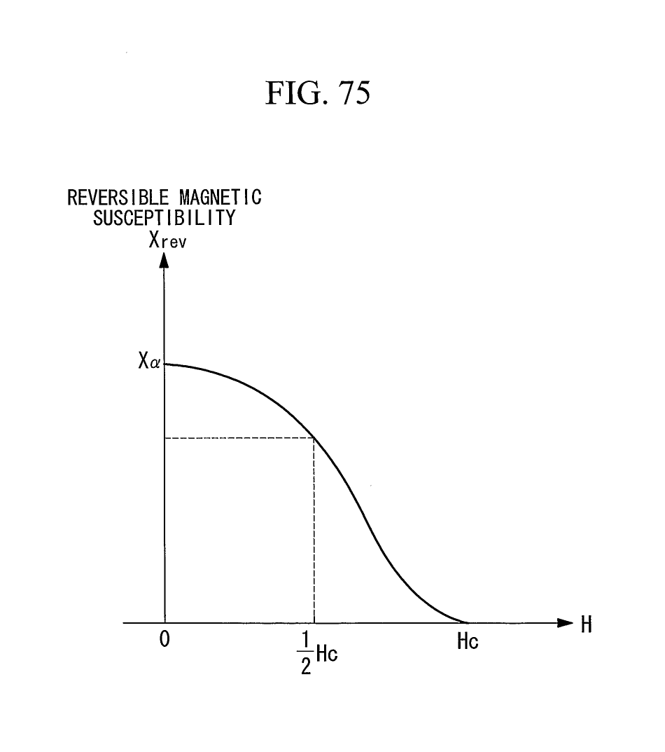

FIG. 75 is a graph showing the reversible magnetic susceptibility in the permalloy film 1141B. In FIG. 75, the horizontal axis represents the intensity of a magnetic field formed at the position of the permalloy film 1141B, and the vertical axis represents the reversible magnetic susceptibility with respect to the magnetic field formed at the position of the permalloy film 1141B.

As shown in FIG. 75, the reversible magnetic susceptibility exhibits the maximum value Xα in a state where no magnetic field is formed at the position of the permalloy film 1141B and decreases as the magnetic field intensity increases. The reversible magnetic susceptibility is 0 in a state where a magnetic field with the saturated magnetic field intensity (Hc) is formed at the position of the permalloy film 1141B.

Therefore, in FIG. 74, since the straight line Q1 corresponds to a case where no static magnetic field is formed at the position of the permalloy film 1141B, it is a straight line with a gradient equal to the reversible magnetic susceptibility Xα to the horizontal axis. A projected length t1 of the straight line Q1 onto the vertical axis represents a variation range of the magnetic flux density occurring due to the alternating magnetic field in the permalloy film 1141B.

As shown in FIGS. 74 and 75, the slopes of the straight lines Q2 and Q3 become small as the intensity of the magnetic field formed at the position of the permalloy film 1141B becomes high. Accordingly, projected lengths t2 and t3 of the straight lines Q2 and Q3 onto the vertical axis also become small, indicating that the variation range of the magnetic flux density occurring due to the alternating magnetic field in the permalloy film 1141B also become small.

The projected lengths t1, t2, and t3 of these straight lines Q1, Q2, and Q3 are related to the intensity of an induced magnetic field formed by the magnetic induction coil 1142 and are therefore related to the sense coil output. More specifically, by way of example of the sense coil output shown in FIG. 62, as the above-described projected lengths t1, t2, and t3 become small, the sense coil output changes from D1 to D8, indicating that the difference between the maximum value and the minimum value of the sense coil output becomes small.

When the magnetic field intensity at the position of the permalloy film 1141B is equal to the saturated magnetic field intensity, the permalloy film 1141B hardly functions, as shown by the above-described projected length t3 and the sense coil output D8, and the magnetic induction coil 1142 exhibits performance similar to that of an air-core coil.

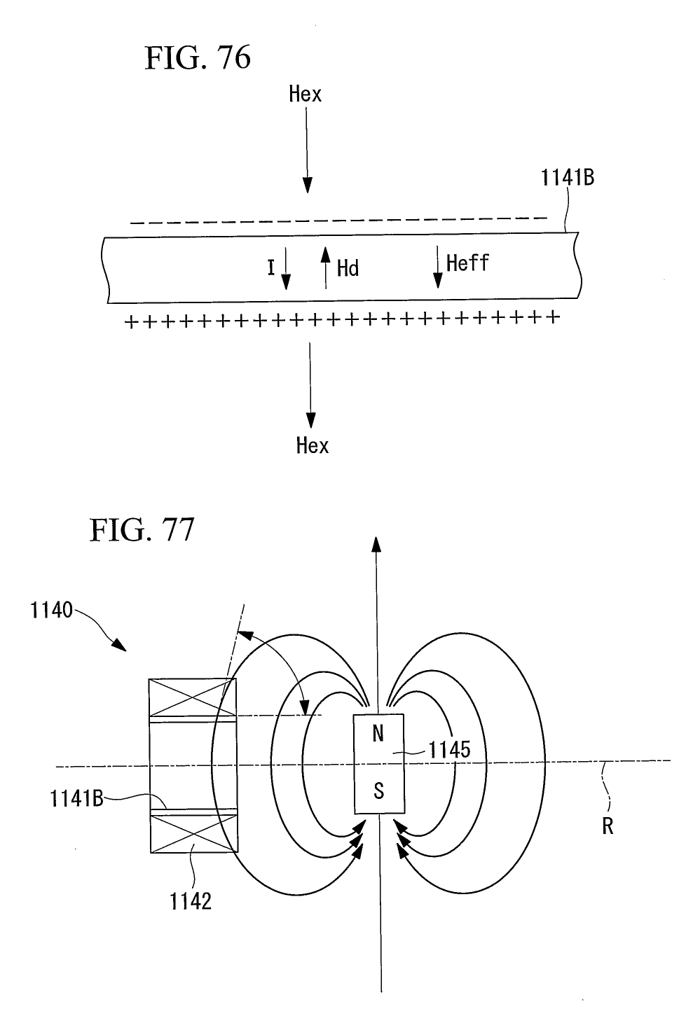

FIG. 76 is a schematic diagram illustrating the intensity of an effective magnetic field in the permalloy film 1141B.

As shown in FIG. 76, when an external static magnetic field (Hex) of the guidance magnet 1145 is formed at the position of the permalloy film 1141B, the permalloy film 1141B is magnetized (I) and exhibits an N (+) pole and an S (−) pole on the surface thereof.

At the same time, due to the N (+) pole and S (−) pole produced on the surface, a demagnetizing field (Hd) expressed by the equation below is formed in the permalloy film 1141B.
Hd=N(I/μ0) (1)

where N is a demagnetizing factor in the direction of the static magnetic field (Hex) in the permalloy film 1141B and μ0 is the magnetic permeability of a vacuum.

An effective magnetic field (Heff) operating effectively in the permalloy film 1141B is obtained by subtracting the demagnetizing field (Hd) from the static magnetic field (Hex) of the guidance magnet 1145, as expressed by the equation below.
Heff=Hex−N(I/μ0) (2)

As long as the above-described effective magnetic field (Heff) does not exceed the saturated magnetic field intensity (Hc), the permalloy film 1141B is not magnetically saturated.

FIG. 77 is a schematic diagram illustrating a demagnetizing factor in the permalloy film 1141B.

The demagnetizing factor (N) is a factor depending on the shape of a member formed of a magnetic material such as the permalloy film 1141B. More specifically, the demagnetizing factor in the thickness direction of a membranous member, such as permalloy film 1141B, is maximized, and the demagnetizing factor in the axial direction of a bar-shaped member is minimized.

In the case of the structure shown in FIG. 77, since the static magnetic field (Hex) of the guidance magnet 1145 is incident along the thickness direction of the permalloy film 1141B, the demagnetizing factor (N) is maximized. Therefore, the demagnetizing field (Hd) in the permalloy film 1141B is maximized, and the effective magnetic field (Heff) is minimized. Since the effective magnetic field (Heff) in the permalloy film 1141B becomes small, the permalloy film 1141B is used in an area with high reversible magnetic susceptibility in FIG. 75.

With the above-described structure, since the performance of the magnetic induction coil 1142 can be enhanced by employing the permalloy film 1141B composed of a magnetic material for the magnetic induction coil 1142, problems can be prevented from occurring when the position of the medical magnetic-induction and position-detection system 1110 is to be detected.

More specifically, when an alternating magnetic field of the drive coil 1151 is applied to the magnetic induction coil 1142, the intensity of the induced magnetic field formed by the magnetic induction coil 1142 becomes high compared with a case where the permalloy film 1141B is not used for the magnetic induction coil 1142. For this reason, the position detection unit 1150 can more easily detect the above-described induced magnetic field, and therefore, problems can be prevented from occurring when the position of the medical magnetic-induction and position-detection system 1110 is to be detected.

In addition, since the permalloy film 1141B is arranged at a position where the magnetic flux density in the permalloy film 1141B resulting from a static magnetic field of the guidance magnet 1145 is not magnetically saturated, a degradation in the performance of the magnetic induction coil 1142 can be prevented.

More specifically, when an alternating magnetic field of the drive coil 1151 and a static magnetic field of the guidance magnet 1145 are applied to the magnetic induction coil 1142, the variation range of the induced magnetic field intensity formed by the magnetic induction coil 1142 in response to a change in the intensity of the alternating magnetic field becomes large compared with a case where the permalloy film 1141B is arranged at a position that causes the magnetic flux density in the permalloy film 1141B to be magnetically saturated. Therefore, the position detection unit 1150 can more easily detect the variation range of the above-described induced magnetic field intensity, and therefore, problems can be prevented from occurring when the position of the medical magnetic-induction and position-detection system 1110 is to be detected.

Since the angle between the magnetic field orientation of the guidance magnet 1145 at the position of the magnetic induction coil 1142 and the direction in which the demagnetizing factor in the permalloy film 1141B is minimized is about 90 degree, the magnetic field of the guidance magnet 1145 is incident upon the permalloy film 1141B from a direction other than the direction in which the demagnetizing factor is minimized.

More specifically, since the permalloy film 1141B is shaped like a substantially cylindrical membrane, a magnetic field of the guidance magnet 1145 is incident upon the permalloy film 1141B from the direction in which the demagnetizing factor is maximized. Therefore, the demagnetizing field formed in the permalloy film 1141B can be maximized, and the effective magnetic field in the permalloy film 1141B can be minimized.

Since the magnetic induction coil 1142 is arranged at a position where the magnetic flux density formed by the magnetic field of the guidance magnet 1145 in the permalloy film 1141B is equal to or lower than half the saturated flux density of the permalloy film 1141B, a decrease in the reversible magnetic susceptibility in the permalloy film 1141B can be suppressed. Therefore, even if an alternating magnetic field of the drive coil 1151 is formed at the position of the permalloy film 1141B in addition to the magnetic field of the guidance magnet 1145, the magnetic flux density formed in the permalloy film 1141B is prevented from exceeding the saturated flux density, and a degradation in performance of the magnetic induction coil 1142 can be prevented.

Since the guidance magnet 1145 and the magnetic induction coil 1142 are arranged at a distance along the axial direction of the magnetic induction coil 1142, a problem can be prevented from occurring when the position of the magnetic induction coil 1142, namely, the position of the capsule endoscope 1120 is to be detected with the position detection unit 1150.

More specifically, when an electromotive force is induced in the magnetic induction coil by an alternating magnetic field formed by the drive coil 1151, the electromotive force induced in the magnetic induction coil 1142 is prevented from being weakened as a result of the guidance magnet 1145 shielding the above-described alternating magnetic field. Furthermore, the detection of the induced magnetic field by the sense coil 1152 is prevented from becoming degraded or disabled as a result of the magnetic field induced by the magnetic induction coil 1142 being shielded by the guidance magnet 1145. For this reason, the position of the capsule endoscope 1120 can be detected with improved accuracy, and problems such as the capsule endscope 1120 being undetectable are prevented from occurring.

Since the imaging section 1130 is provided in the capsule endoscope 1120, an image inside the subject 1 can be acquired as biological information. In addition, with the LED 1133, an image that is easy to visually recognize can be acquired by illuminating the inside of the subject 1.

Since the imaging section 1130, the battery 1139, and so forth are arranged in the hollow structure of the magnetic induction coil 1142, the size of the capsule endoscope 1120 can be reduced compared with a case where the imaging section 1130 and so forth are not arranged in the magnetic induction coil 1142. Therefore, the capsule endoscope 1120 can more easily be introduced into the body cavity of the subject 1.

The intensity of the induced magnetic field occurring in the induced-magnetic-field generating section 1140 can be enhanced by arranging the permalloy film 1141B, as a magnetic material, between the core member 1141A and the magnetic induction coil 1142.

Furthermore, by forming the permalloy film 1141B so as to have a substantially C-shaped cross-section, a shielding current flowing substantially in a circle is prevented from occurring in the cross-section of the permalloy film 1141B. Therefore, shielding of the magnetic field due to a shielding current can be prevented, and inhibition of the occurrence or the reception of a magnetic field in the magnetic induction coil 1142 can be prevented.

Since the plurality of magnet pieces 1145a, 1145b, and 1145c are formed in the shape of plates, they can easily be stacked one on another to construct the guidance magnet 1145. In addition, since the magnet pieces 1145a, 1145b, and 1145c are magnetized in their plate-thickness direction, they can more easily be stacked one on another, and therefore, the guidance magnet 1145 can more easily be manufactured.

Furthermore, the insulators 1145d can more easily be interposed between the magnet pieces. In addition, by interposing the insulators 1145d, a shielding current can be made more difficult to flow in the guidance magnet 1145, and therefore, a magnetic field generated or received by the magnetic induction coil 1142 is prevented from being shielded by such a shielding current flowing in the guidance magnet 1145.

By making the frequency of the alternating magnetic field formed by the drive coil 1151 the same as the resonance frequency (LC resonance frequency) of the LC resonant circuit 1143, it is possible to produce an induced magnetic field with an amplitude that is large compared to the case where another frequency is used. Therefore, the sense coil 1152 can easily detect the induced magnetic field, which makes it easy to detect the position of the capsule endoscope 1120.

Also, since the frequency of the alternating magnetic field varies over a frequency range in the vicinity of the LC resonance frequency, even if the resonance frequency of the LC resonant circuit 1143 changes due to variations in the environmental conditions (for example, the temperature conditions) or even if there is a shift in the resonance frequency due to individual differences in the LC resonant circuit 1143, it is possible to bring about resonance in the LC resonant circuit 1143.

Alternating magnetic fields are applied to the magnetic induction coil 1142 of the capsule endoscope 1120 from three or more different directions that are linearly independent. Therefore, it is possible to produce an induced magnetic field in the magnetic induction coil 1142 by alternating magnetic fields from at least one direction, irrespective of the orientation of the magnetic induction coil 1142.

As a result, it is always possible to produce an induced magnetic field in the magnetic induction coil 1142, irrespective of the orientation (axial direction of the rotation axis R) of the capsule endoscope 1120; therefore, an advantage is afforded in that it is possible to always detect the induced magnetic field by the sense coils 1152, which allows the position thereof to always be detected with accuracy.

Also, since the sense coils 1152 are disposed in three different directions with respect to the capsule endoscope 1120, an induced magnetic field of detectable intensity acts on the sense coils 1152 disposed in at least one direction of the sense coils 1152 disposed in the three directions, which allows the sense coils 1152 to always detect the induced magnetic field, irrespective of the position at which the capsule endoscope 1120 is disposed.

Furthermore, since the number of sense coils 1152 disposed in one direction is nine, as mentioned above, a sufficient number of inputs to acquire a total of six pieces of information by calculation is ensured, where the six pieces of information include the X, Y, and Z coordinates of the capsule endoscope 1120, the rotational phases φ and θ about two axes orthogonal to each other and orthogonal to the rotation axis R of the capsule endoscope 1120, and the intensity of the induced magnetic field.

By setting the frequency of the alternating magnetic field to the frequency at which the LC resonant circuit 1143 resonates (the resonance frequency), it is possible to produce an induced magnetic field with an amplitude that is large compared to a case where another frequency is used. Since the amplitude of the induced magnetic field is large, the sense coils 1152 can easily detect the induced magnetic field, which makes it easy to detect the position of the capsule endoscope 1120.

Also, since the frequency of the alternating magnetic field sweeps over a frequency range in the vicinity of the resonance frequency, even if the resonance frequency of the LC resonant circuit 1143 changes due to variations in the environmental conditions (for example, the temperature conditions) or even if there is a shift in the resonance frequency due to individual differences in the LC resonant circuit 1143, it is possible to bring about resonance in the LC resonant circuit 1143 so long as the changed resonance frequency or the shifted resonance frequency is included in the frequency range mentioned above.

Since the position detection unit 1150 selects the outputs of the sense coils 1152 that detect high-intensity induced magnetic fields by means of the sense-coil selector 1156, it is possible to reduce the volume of information that the position detection unit 1150 must calculate and to reduce the computational load. At the same time, since it is possible to simultaneously reduce the amount of computational processing, the time required for computation can be shortened.

Since the drive coils 1151 and the sense coils 1152 are located at positions opposing each other on either side of the operating region of the capsule endoscope 1120, the drive coils 1151 and the sense coils 1152 can be positioned so that they do not interfere with each other in terms of their construction.

By controlling the orientation of the parallel magnetic fields acting on the guidance magnet 1145 built into the capsule endoscope 1120, it is possible to control the orientation of the force acting on the guidance magnet 1145, which allows the direction of motion of the capsule endoscope 1120 to be controlled. Since it is possible to detect the position of the capsule endoscope 1120 at the same time, the capsule endoscope 1120 can be guided to a predetermined position, and therefore, an advantage is afforded in that it is possible to accurately guide the capsule endoscope based on the detected position of the capsule endoscope 1120.

By controlling the intensities of the magnetic fields produced by the three pairs of Helmholtz coils 1171X, 1171Y, and 1171Z that are disposed to face each other in mutually orthogonal directions, the orientations of the parallel magnetic fields produced inside the Helmholtz coils 1171X, 1171Y, and 1171Z can be controlled in a predetermined direction. Accordingly, a parallel magnetic field in a predetermined orientation can be applied to the capsule endoscope 1120, and it is possible to move the capsule endoscope 1120 in a predetermined direction.

Since the drive coils 1151 and the sense coils 1152 are disposed in the periphery of the space at the inner sides of the Helmholtz coils 1171X, 1171Y, and 1171Z, which is the space in which the subject 1 can be positioned, the capsule endoscope 1120 can be guided to a predetermined location in the body of the subject 1.

By rotating the capsule endoscope 1120 about the rotation axis R, the helical part 1125 produces a force that propels the capsule endoscope 1120 in the axial direction of the rotation axis. Since the helical part 1125 produces the propulsion force, it is possible to control the direction of the propulsion force acting on the capsule endoscope 1120 by controlling the direction of rotation about the rotation axis R of the capsule endoscope 1120.

Since the image display apparatus 1180 performs the processing for rotating a display image in the rotation direction opposite to that of the capsule endoscope 1120, based on information on the rotational phase about the rotational axis R of the capsule endoscope 1120, it is possible to display on the display section 1182 an image that is always fixed at a predetermined rotational phase, in other words, an image in which the capsule endoscope 1120 appears to travel along the rotation axis R without rotating about the rotation axis R, regardless of the rotational phase of the capsule endoscope 1120.

Accordingly, when the capsule endoscope 1120 is guided while the operator visually observes the image displayed on the display section 1182, showing the image displayed in the manner described above as a predetermined rotational phase image makes it easier for the operator to view and also makes it easier to guide the capsule endoscope 1120 to a predetermined location, compared to the case where the displayed image is an image that rotates along with the rotation of the capsule endoscope 1120.

### Seventh Embodiment

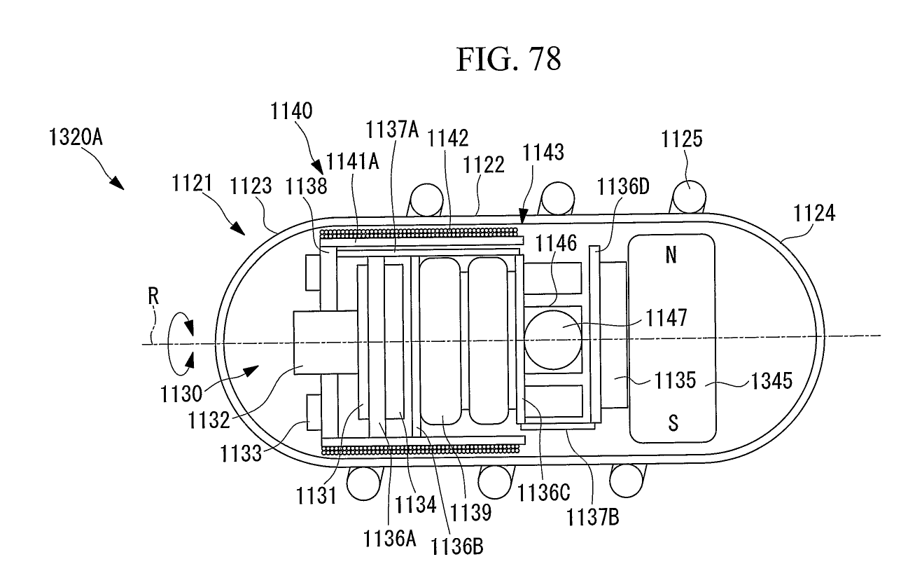

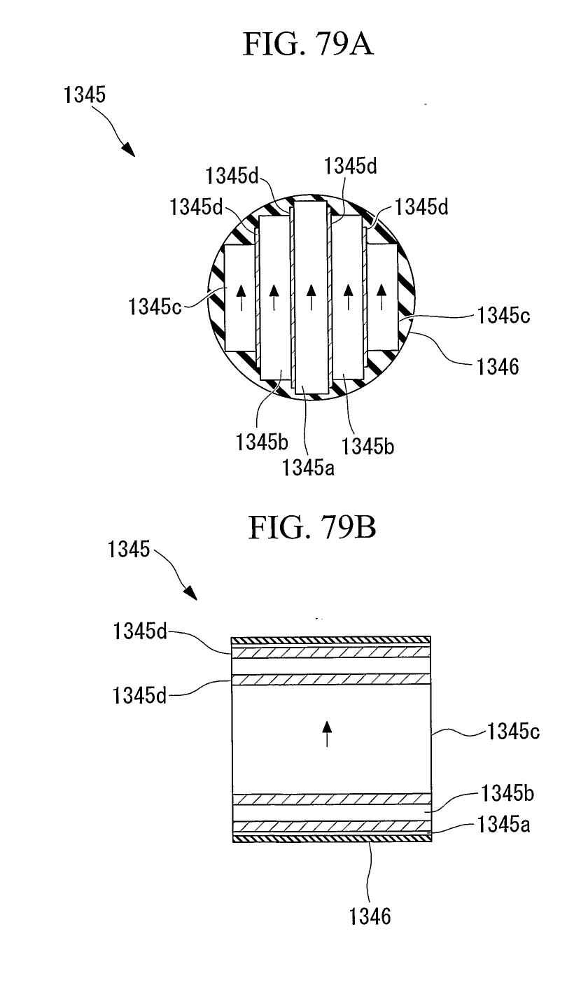

A seventh embodiment of the present invention will now be described with reference to FIGS. 78 and 79.

The basic configuration of the medical magnetic-induction and position-detection system according to this embodiment is the same as that in the sixth embodiment; however, the structure of the guidance magnet of the capsule endoscope is different from that in the sixth embodiment. Thus, in this embodiment, only the vicinity of the guidance magnet of the capsule endoscope shall be described with reference to FIGS. 78 and 79, and the description of the magnetic induction apparatus and so forth shall be omitted.

FIG. 78 is a diagram illustrating the structure of the capsule endoscope according to this embodiment.

The same components as those in the sixth embodiment are denoted with the same reference numerals, and thus will not be described.

As shown in FIG. 78, the capsule endoscope (medical device) 1320A is mainly formed of an outer casing 1121 that accommodates various devices in the interior thereof; an imaging section 1130 that images an internal surface of a passage in the body cavity of the subject; a battery 1139 for driving the imaging section 1130; an induced-magnetic-field generating section 1140 that generates induced magnetic fields by means of the drive coils 1151 described above; and a guidance magnet (magnet) 1345 that drives and guides the capsule endoscope 1320A.

FIG. 79A is a front elevational view illustrating the structure of the guidance magnet 1345 in the capsule endoscope 1320A shown in FIG. 78. FIG. 79B is a side elevational view of the guidance magnet 1345.

As shown in FIGS. 79A and 79B, the guidance magnet 1345 includes one large-size magnet piece (magnet piece) 1345a formed substantially in the shape of a plate; two medium-size magnet pieces (magnet pieces) 1345b; two small-size magnet pieces (magnet pieces) 1345c; and insulators (insulating materials) 1345d, such as vinyl sheets, interposed between the magnet pieces 1345a, 1145b, and 1345c, and is constructed so as to have a substantially cylindrical shape. In addition, the magnet pieces 1345a, 1345b, and 1345c are magnetized in a direction along their surfaces (up and down direction in the figure). More specifically, the side indicated by the arrow corresponds to the north pole, and the opposite side corresponds to the south pole.

The magnet pieces 1345a, 1345b, and 1345c are fixed by a fixing member 1346, such as adhesive or former, so that they are not separated from each other by their magnetic forces.

Since the operation of the medical magnetic-induction and position-detection system and the capsule endoscope with the above-described structure is the same as that in the sixth embodiment, a description thereof is omitted.

With the above-described structure, since the magnet pieces 1345a, 1345b, and 1345c are magnetized in the direction along the surfaces thereof, the magnetic force of the magnet pieces 1345a, 1345b, and 1345c can be increased compared with a case where they are magnetized in the thickness direction. Consequently, the magnetic force of the guidance magnet 1345, which is an aggregate of the magnet pieces 1345a, 1345b, and 1345c, can be increased.

### Eighth Embodiment

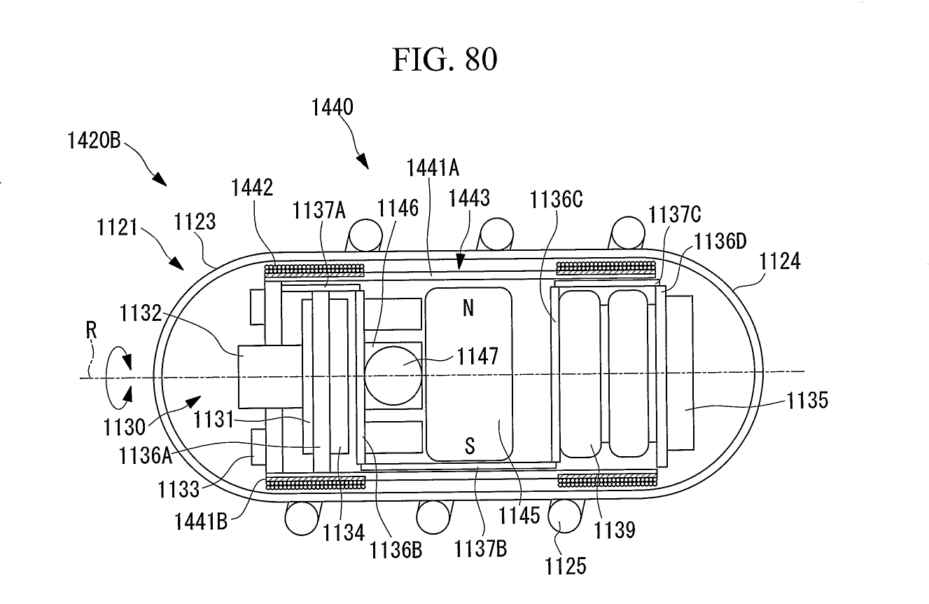

An eighth embodiment of the present invention will now be described with reference to FIG. 80.

The basic configuration of the medical magnetic-induction and position-detection system according to this embodiment is the same as that in the sixth embodiment; however, the structure of the induced-magnetic-field generating section of the capsule endoscope is different from that in the sixth embodiment. Thus, in this embodiment, only the vicinity of the induced-magnetic-field generating section of the capsule endoscope shall be described with reference to FIG. 80, and the description of the magnetic induction apparatus and so forth shall be omitted.

FIG. 80 is a diagram illustrating the structure of the capsule endoscope according to this embodiment.

A capsule endoscope (medical device) 1420B according to this embodiment has an induced-magnetic-field generating section (induction-magnetic-field generating unit) 1440 with a different structure and other devices have a different layout. Therefore, only these two points are described and a description of other devices is omitted.

Inside an outer casing 1121 of the capsule endoscope 1420B, a lens group 1132, an LED 1133, an image sensor 1131, a signal processing section 1134, a switch section 1146, a guidance magnet 1145, a battery 1139, and a radio device 1135 are disposed in sequence from a front end portion 1123. The guidance magnet 1145 is arranged near the center of gravity of the capsule endoscope 1420B.

The induced-magnetic-field generating section 1440 is arranged between the outer casing 1121 and the battery 1139 and so forth so as to cover the components from the support member 1138 of the LED 1133 to the battery 1139.

As shown in FIG. 80, the induced-magnetic-field generating section 1440 is formed of a core member 1441A formed in the shape of a cylinder whose central axis is substantially coincident with the rotation axis R; a magnetic induction coil (built-in coil) 1442 disposed on the outer circumferential part of the core member 1441A; a permalloy film (magnetic object) 1441B disposed between the core member 1441A and the magnetic induction coil 1442; and a capacitor (not shown in the figure) that is electrically connected to the magnetic induction coil 1442 and that constitutes the LC resonant circuit (circuit) 1443.

The magnetic induction coil 1442 is sparsely wound at the region where the guidance magnet 1145 is disposed and is densely wound at the front end portion 1123 side and at the rear end portion 1124 side.

Since the operation of the medical magnetic-induction and position-detection system and capsule endoscope with the above-described structure is the same as that in the sixth embodiment, a description thereof is omitted.

With the above-described structure, since the guidance magnet 1145 can be arranged near the center of gravity of the capsule endoscope 1420B, the capsule endoscope 1420B can easily be driven and guided compared with a case where the guidance magnet 1145 is arranged slightly towards the front-end portion 1123 side or the rear-end portion 1124 side of the capsule endoscope 1420B.

### Ninth Embodiment

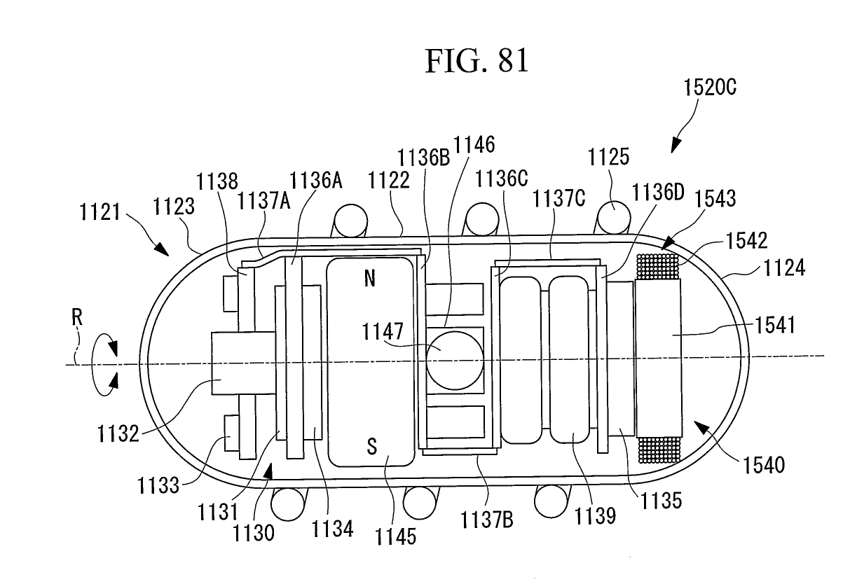

A ninth embodiment of the present invention will now be described with reference to FIG. 81.

The basic configuration of the medical magnetic-induction and position-detection system according to this embodiment is the same as that in the sixth embodiment; however, the structure of the induced-magnetic-field generating section of the capsule endoscope is different from that in the sixth embodiment. Thus, in this embodiment, only the vicinity of the induced-magnetic-field generating section of the capsule endoscope shall be described with reference to FIG. 81, and the description of the magnetic induction apparatus and so forth shall be omitted.

FIG. 81 is a diagram illustrating the structure of the capsule endoscope according to this embodiment.

The capsule endoscope (medical device) 1520C according to this embodiment has an induced-magnetic-field generating section (induction-magnetic-field generating unit) 1540 with a different structure and other devices have a different layout. Therefore, only these two points are described and a description of other devices is omitted.

As shown in FIG. 81, inside an outer casing 1121 of the capsule endoscope 1520C, a lens group 1132, an LED 1133, an image sensor 1131, a signal processing section 1134, a guidance magnet 1145, a switch section 1146, a battery 1139, a radio device 1135, and an induced-magnetic-field generating section 1540 are disposed in sequence from the front end portion 1123.

The induced-magnetic-field generating section 1540 is formed of a core member 1541 formed of ferrite in the shape of a cylinder whose central axis is substantially coincident with the rotation axis R; a magnetic induction coil (built-in coil) 1542 disposed on the outer circumferential part of the core member 1541; and a capacitor (not shown in the figure) that is electrically connected to the magnetic induction coil 1542 and that constitutes the LC resonant circuit (circuit) 1543.

The core member 1541 may be formed of a material such as iron, permalloy, or nickel instead of the above-described ferrite.

Since the operation of the medical magnetic-induction and position-detection system and capsule endoscope with the above-described structure is the same as that in the sixth embodiment, a description thereof is omitted.

With the above-described structure, since the core member 1541 formed of dielectric ferrite is disposed at the center of the magnetic induction coil 1542, the induced magnetic field is more easily concentrated in the core member 1541, and the induced magnetic field produced thus becomes even more intense.

### Tenth Embodiment

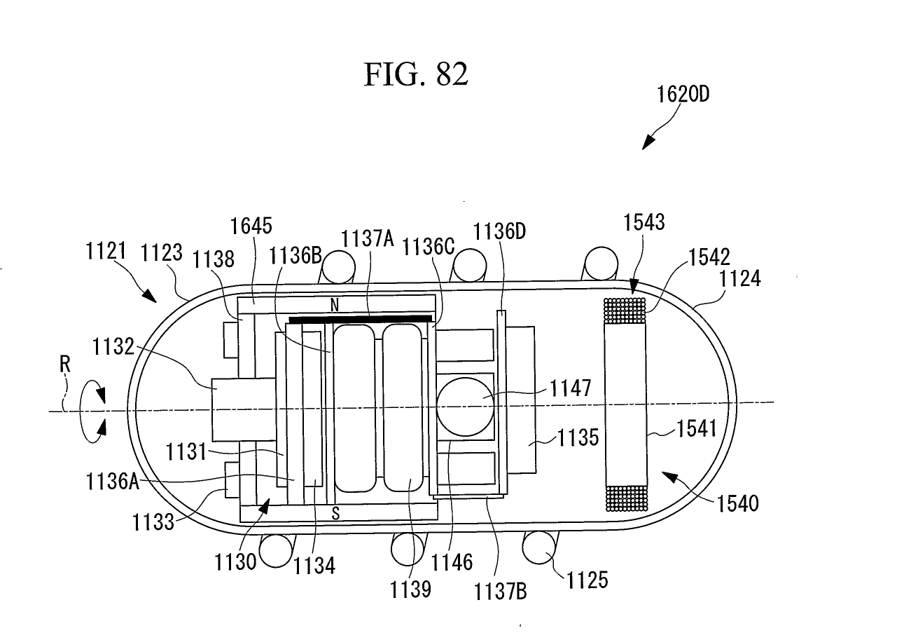

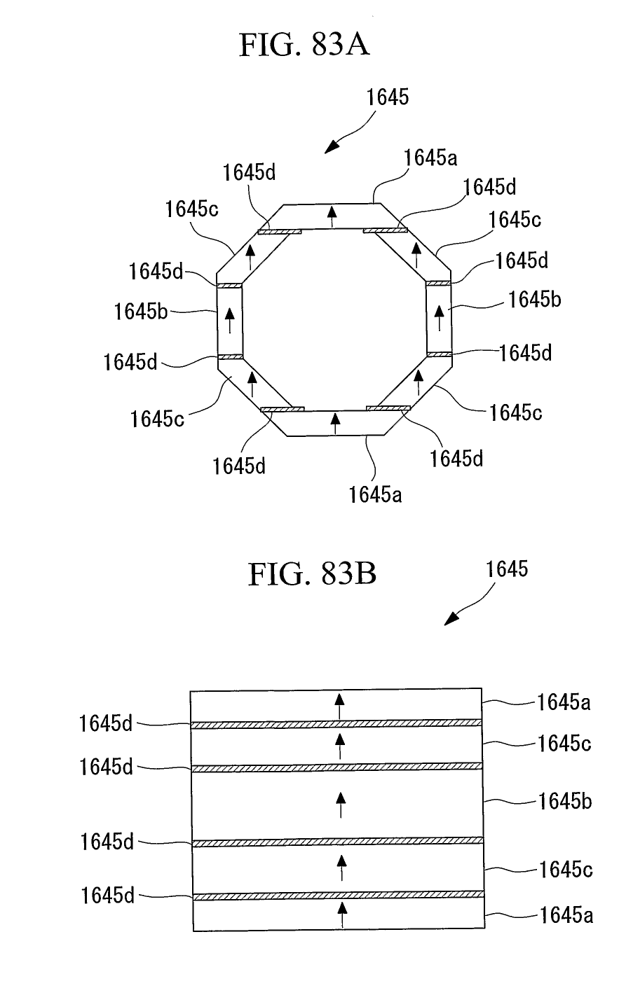

A tenth embodiment of the present invention will now be described with reference to FIGS. 82 and 83.

The basic configuration of the medical magnetic-induction and position-detection system according to this embodiment is the same as that in the ninth embodiment; however, the structure of the guidance magnet of the capsule endoscope is different from that in the ninth embodiment. Thus, in this embodiment, only the vicinity of the guidance magnet of the capsule endoscope shall be described with reference to FIGS. 82 and 83, and a description of the magnetic induction apparatus and so forth shall be omitted.

FIG. 82 is a diagram illustrating the structure of the capsule endoscope according to this embodiment.

The capsule endoscope (medical device) 1620D according to this embodiment has a guidance magnet (magnet) 1645 with a different structure and other devices have a different layout. Therefore, only these two points are described and a description of other devices is omitted

As shown in FIG. 82, inside an outer casing 1121 of the capsule endoscope 1620D, a lens group 1132, an LED 1133, an image sensor 1131, a signal processing section 1134, a battery 1139, a switch section 1146, a radio device 1135, and an induced-magnetic-field generating section 1540 are disposed in sequence from the front end portion 1123.

The guidance magnet 1645 is arranged between the outer casing 1121 and the battery 1139 and so forth so as to cover the components from the support member 1138 of the LED 1133 to the battery 1139.

FIG. 83A is a front elevational view illustrating the structure of the guidance magnet 1645 in the capsule endoscope 1620D shown in FIG. 82. FIG. 83B is a side elevational view of the guidance magnet 1645.

As shown in FIGS. 83A and 83B, the guidance magnet 1645 includes magnet pieces 1645a disposed in the upper and lower areas; magnet pieces 1645b disposed at the right and left sides; magnet pieces 1645c disposed in oblique areas; and insulators (insulating materials) 1645d disposed between the magnet pieces 1645a, 1645b, and 1645c, and is constructed to have a cylindrical shape.

The magnet pieces 1645a are magnetized in the plate-thickness direction, the magnet pieces 1645b are magnetized in the direction along their surfaces, and the magnet pieces 1645c are magnetized in oblique directions. In the figure, the side indicated by the arrow corresponds to the north pole, and the opposite side corresponds to the south pole.

Since the operation of the medical magnetic-induction and position-detection system and capsule endoscope with the above-described structure is the same as that in the ninth embodiment, a description thereof is omitted.

With the above-described structure, since the imaging section 1130, the battery 1139, and so forth are arranged in the hollow structure of the guidance magnet 1645, the size of the capsule endoscope 1620D can be reduced.

### Eleventh Embodiment

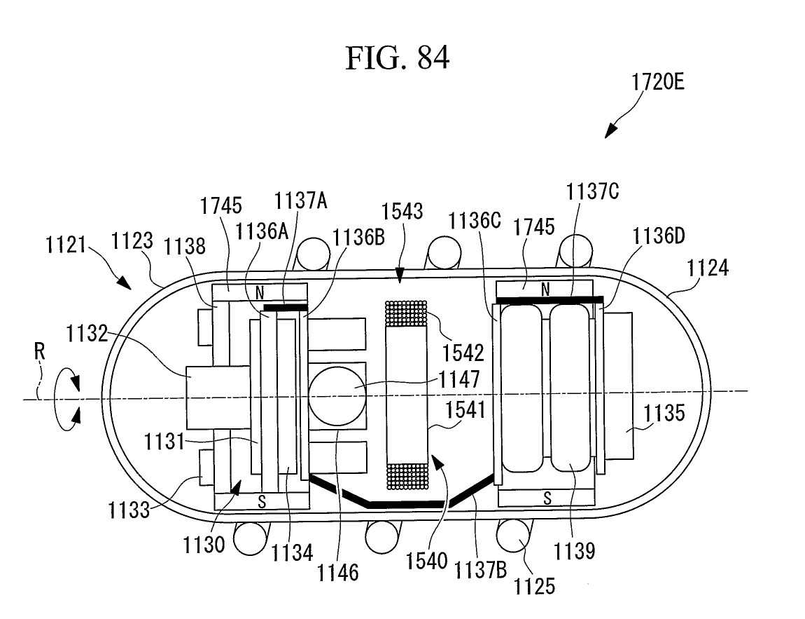

An eleventh embodiment of the present invention will now be described with reference to FIG. 84.

The basic configuration of the medical magnetic-induction and position-detection system according to this embodiment is the same as that in the tenth embodiment; however, the structure of the guidance magnet of the capsule endoscope is different from that in the tenth embodiment. Thus, in this embodiment, only the vicinity of the guidance magnet of the capsule endoscope shall be described with reference to FIG. 84, and a description of the magnetic induction apparatus and so forth shall be omitted.

FIG. 84 is a diagram illustrating the structure of the capsule endoscope according to this embodiment.

The capsule endoscope (medical device) 1720E according to this embodiment has a guidance magnet (magnet) 1745 with a different structure and other devices have a different layout. Therefore, only these two points are described and a description of other devices is omitted

As shown in FIG. 84, inside an outer casing 1121 of the capsule endoscope 1720E, a lens group 1132, an LED 1133, an image sensor 1131, a signal processing section 1134, a switch section 1146, a battery 1139, an induced-magnetic-field generating section 1540, and a radio device 1135 are disposed in sequence from the front end portion 1123. The induced-magnetic-field generating section 1540 is disposed substantially at the center of the capsule endoscope 1720E.

Guidance magnets 1745 are arranged at two locations between the outer casing 1121 and the battery 1139 and so forth, more specifically, so as to cover the components from the support member 1138 of the LED 1133 to the signal processing section 1134 and the battery 1139.

Since the operation of the medical magnetic-induction and position-detection system and capsule endoscope with the above-described structure is the same as that in the ninth embodiment, a description thereof is omitted.

With the above-described structure, since the induced-magnetic-field generating section 1540 can be disposed near the center of the capsule endoscope 1720E, the correct position of the capsule endoscope 1720E can be detected without correction, compared with a case where the induced-magnetic-field generating section 1540 is disposed slightly towards the front-end portion 1123 or the rear-end. portion 1124 of the capsule endoscope 1720E.

### Twelfth Embodiment

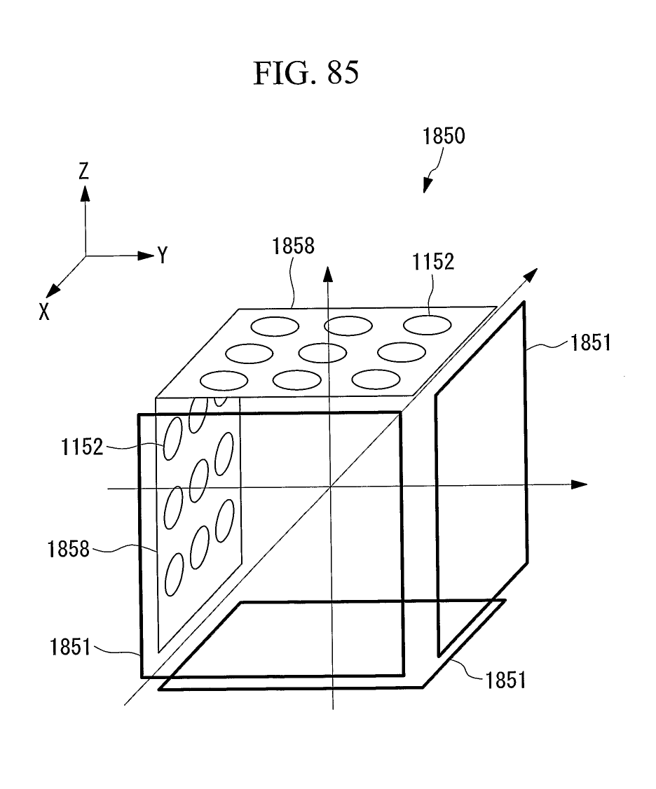

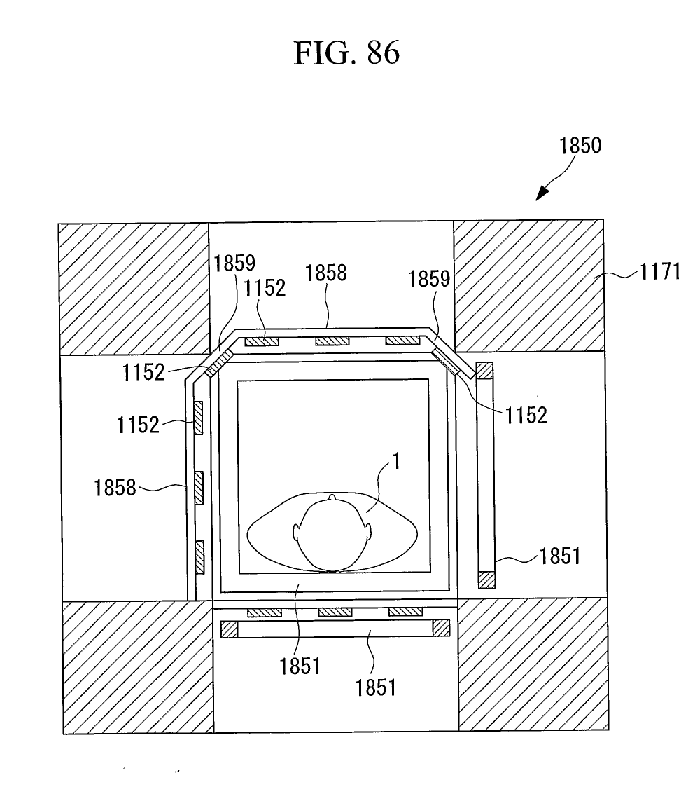

A twelfth embodiment of the present invention will now be described with reference to FIGS. 85 and 86.

The basic configuration of the medical magnetic-induction and position-detection system according to this embodiment is the same as that in the sixth embodiment; however, the structure of the position detection unit is different from that in the sixth embodiment. Thus, in this embodiment, only the vicinity of the position detection unit shall be described with reference to FIGS. 85 and 86, and a description of the magnetic induction apparatus and so forth shall be omitted.

FIG. 85 is a schematic diagram showing the arrangement of the drive coils and the sense coils in the position detection unit.

Since components other than the drive coils and sense coils of the position detection unit are the same as those in the sixth embodiment, a description thereof shall be omitted here.

As shown in FIG. 85, drive coils (drive section) 1851 and sense coils 1152 of the position detection unit (position detection system, position detection apparatus, position detector, calculating apparatus) 1850 are arranged such that three drive coils 1851 are orthogonal to the X, Y, and Z axes, respectively, and the sense coils 1152 are disposed on two planar coil-supporting parts 1858 orthogonal to the Y and Z axes, respectively.

Rectangular coils as shown in the figure, Helmholtz coils, or opposing coils may be used as the drive coils 1851.

As shown in FIG. 85, in the position detection unit 1850 having the configuration described above, the orientations of the alternating magnetic fields that the drive coils 1851 produce are parallel to the X, Y, and Z axial directions and are linearly independent, having a mutually orthogonal relationship.

With this configuration, it is possible to apply alternating magnetic fields to the magnetic induction coil 1142 in the capsule endoscope 1120 from linearly independent and mutually orthogonal directions. Therefore, an induced magnetic field is easier to generate in the magnetic induction coil 1142 compared to the sixth embodiment, regardless of the orientation of the magnetic induction coil 1142.

Also, since the drive coils 1851 are disposed so as to be substantially orthogonal to each other, selection of the drive coils by the drive-coil selector 1155 is simplified.

The sense coils 1152 may be disposed on the coil-support members 1858, which are perpendicular to the Y and Z axes, as described above, or, as shown in FIG. 86, sense coils 1152 may be provided on inclined coil-support members 1859 disposed in the upper part of the operating region of the capsule endoscope 1120.

By positioning them in this manner, the sense coils 1152 can be positioned without interfering with the subject 1.

### Thirteenth Embodiment

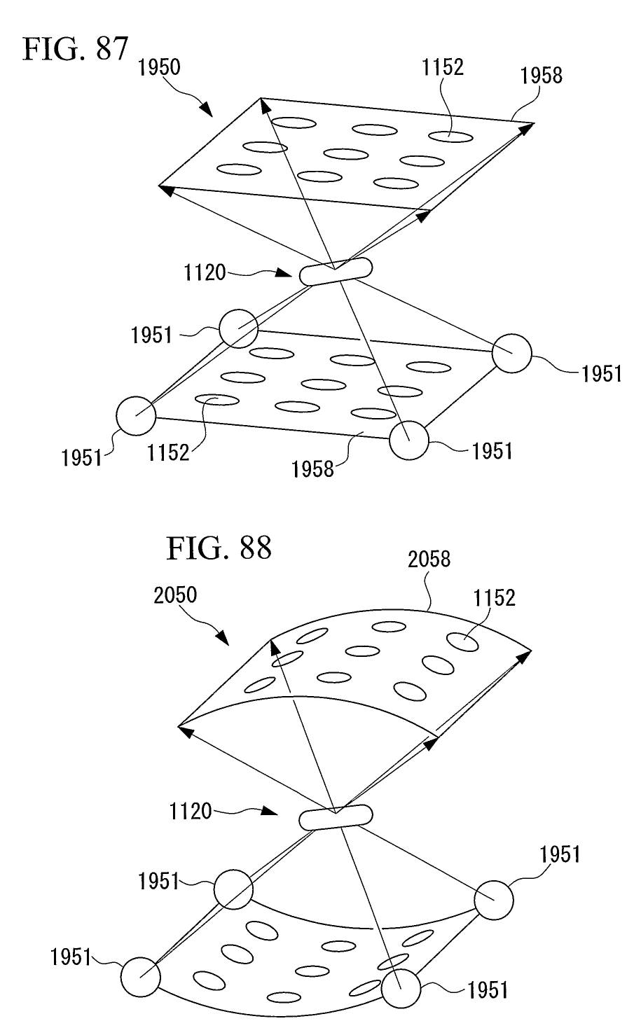

A thirteenth embodiment of the present invention will now be described with reference to FIG. 87.

The basic configuration of the medical magnetic-induction and position-detection system according to this embodiment is the same as that in the sixth embodiment; however, the structure of the position detection unit is different from that in the sixth embodiment. Thus, in this embodiment, only the vicinity of the position detection unit shall be described with reference to FIG. 87, and a description of the magnetic induction apparatus and so forth shall be omitted.

FIG. 87 is a schematic diagram showing the arrangement of the drive coils and the sense coils in the position detection unit.

Since components other than the drive coils and sense coils of the position detection unit are the same as those in the sixth embodiment, a description thereof shall be omitted here.

Regarding drive coils (drive section) 1951 and sense coils 1152 of the position detection unit (position detection system, position detection apparatus, position detector, calculating apparatus) 1950, as shown in FIG. 87, four drive coils 1951 are disposed in the same plane, and the sense coils 1152 are disposed on a planar coil-supporting member 1958, which is disposed at a position opposite the position where the drive coils 1951 are located, and on a planar coil-supporting member 1958, which is disposed at the same side where the drive coils 1951 are located, the operating region of the capsule endoscope 1120 being disposed therebetween.

The drive coils 1951 are arranged such that the orientations of the alternating magnetic fields that any three drive coils 1951 produce are linearly independent of each other, as indicated by the arrows in the figure.

According to this configuration, one of the two coil-supporting members 1958 is always located close with respect to the capsule endoscope 1120, regardless of whether the capsule endoscope 1120 is located in a nearby region or a distant region with respect to the drive coils 1951. Accordingly, a signal of sufficient intensity can be obtained from the sense coils 1152 when determining the position of the capsule endoscope 1120.

### Modification of Thirteenth Embodiment

Next, a modification of the thirteenth embodiment of the present invention will be described with reference to FIG. 88.

The basic configuration of the medical magnetic-induction and position-detection system of this modification is the same as that in the thirteenth embodiment; however, the configuration of the position detection unit is different from that in the thirteenth embodiment. Therefore, in this embodiment, only the vicinity of the position detection unit will be described using FIG. 88, and a description of the magnetic induction apparatus and the like will be omitted.

FIG. 88 is a schematic diagram showing the positioning of drive coils and sense coils of the position detection unit.

Since the components other than the drive coils and the sense coils of the position detection unit are the same as in the eighth embodiment, a description thereof is omitted here.

Regarding drive coils 1951 and sense coils 1152 of the position detection unit (position detection system, position detection apparatus, position detector, calculating apparatus) 2050, as shown in FIG. 88, four drive coils 1951 are disposed in the same plane, and the sense coils 1152 are disposed on a curved coil-supporting member 2058, which is disposed at a position opposite the position where the drive coils 1951 are located, and on a curved coil-supporting member 2058, which is disposed at the same side where the drive coils 1951 are located, the operating region of the capsule endoscope 1120 being disposed therebetween.

The coil-supporting members 2058 are formed in a curved shape that is convex towards the outer side relative to the operating region of the capsule endoscope 1120, and the sense coils 1152 are disposed over the curved surfaces.

The shape of the coil-supporting members 2058 may be curved surfaces that are convex towards the outer side with respect to the operating region, as described above, or they may be any other shape of curved surface and are not particularly limited.

With the configuration described above, since the degree of freedom of positioning the sense coils 1152 is improved, it is possible to prevent the sense coils 1152 from interfering with the subject 1.

### Fourteenth Embodiment

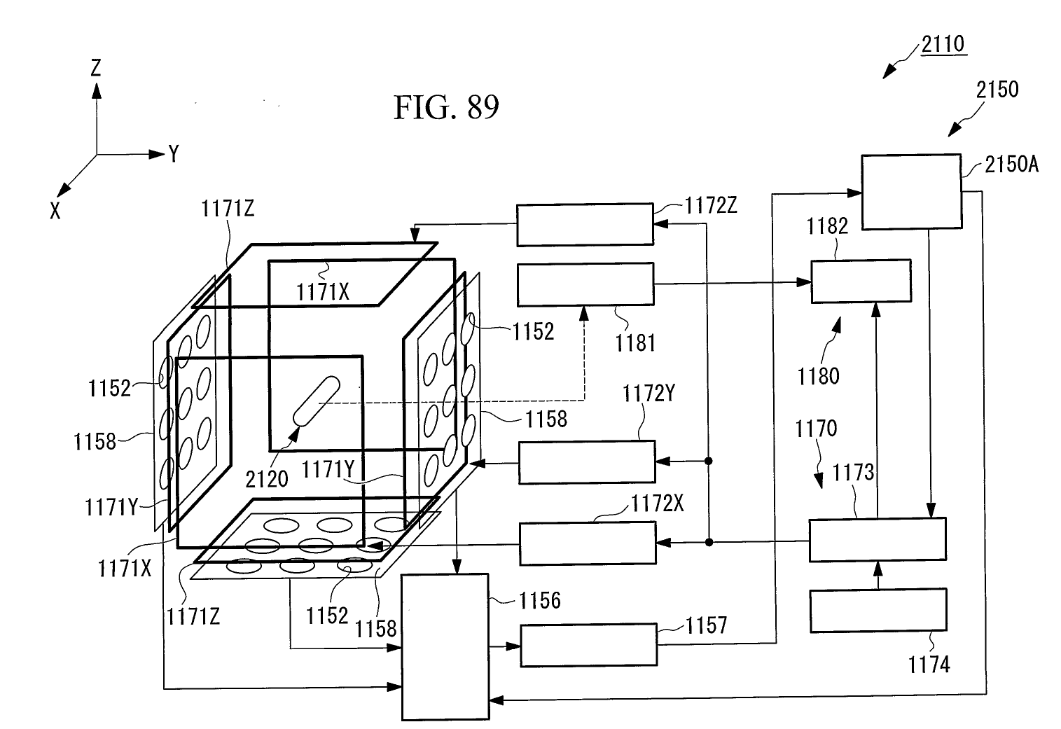

A fourteenth embodiment of the present invention will now be described with reference to FIG. 89.

The basic configuration of the medical magnetic-induction and position-detection system according to this embodiment is the same as that in the sixth embodiment; however, the structure of the position detection unit is different from that in the sixth embodiment. Thus, in this embodiment, only the vicinity of the position detection unit shall be described with reference to FIG. 89, and the description of the magnetic induction apparatus and so forth shall be omitted.

FIG. 89 is a diagram depicting in outline the medical magnetic-induction and position-detection system according to this embodiment.

Since components other than the drive coils and sense coils of the position detection unit are the same as those in the sixth embodiment, a description thereof shall be omitted here.

As shown in FIG. 89, a medical magnetic-induction and position-detection system 2110 is mainly formed of a capsule endoscope (medical device) 2120 that optically images an internal surface of a passage in the body cavity and wirelessly transmits an image signal; a position detection unit (position detection system, position detection apparatus, position detector, calculating apparatus) 2150 that detects the position of the capsule endoscope 2120; a magnetic induction apparatus 1170 that guides the capsule endoscope 2120 based on the detected position of the capsule endoscope 2120 and instructions from an operator; and an image display apparatus 1180 that displays the image signal transmitted from the capsule endoscope 2120.

As shown in FIG. 89, the position detection unit 2150 includes sense coils 1152 for detecting an induced magnetic field generated in the magnetic induction coil (internal magnetic field detector) of the capsule endoscope 2120.

Between the sense coils 1152 and the position detection apparatus 2150A, there are provided a sense coil selector 1156 that selects from the sense coils 1152 AC current that includes position information of the capsule endoscope 2120 and so on, based on the output from the position detection apparatus 2150A; and a sense-coil receiving circuit 1157 that extracts an amplitude value from the AC current passing through the sense coil selector 1156 and outputs it to the position detection apparatus 2150A.

An oscillating circuit is connected to the magnetic induction coil of the capsule endoscope 2120. By connecting the oscillating circuit to the magnetic induction coil, a magnetic field can be generated by the magnetic induction coil without using a drive coil and so forth, and the generated magnetic field can be detected with the sense coils 1152.

### Fifteenth Embodiment

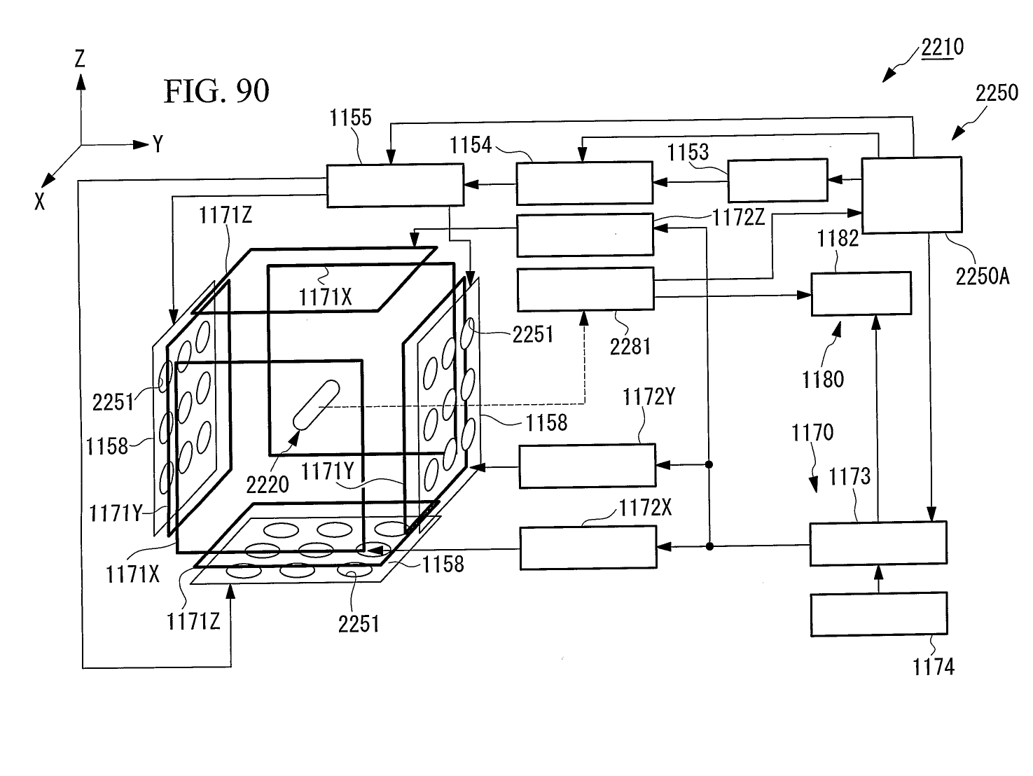

A fifteenth embodiment of the present invention will now be described with reference to FIG. 90.

The basic configuration of the medical magnetic-induction and position-detection system according to this embodiment is the same as that in the sixth embodiment; however, the structure of the position detection unit is different from that in the sixth embodiment. Thus, in this embodiment, only the vicinity of the position detection unit shall be described with reference to FIG. 90, and a description of the magnetic induction apparatus and so forth shall be omitted.

FIG. 90 is a schematic diagram showing the arrangement of the drive coils and the sense coils in the position detection unit.

Since components other than the drive coils and sense coils of the position detection unit are the same as those in the sixth embodiment, a description thereof shall be omitted here.

As shown in FIG. 90, a medical magnetic-induction and position-detection system 2210 is mainly formed of a (medical device) capsule endoscope 2220 that optically images an internal surface of a passage in the body cavity and wirelessly transmits an image signal; a position detection unit (position detection system, position detection apparatus, position detector, calculating apparatus) 2250 that detects the position of the capsule endoscope 2220; a magnetic induction apparatus 1170 that guides the capsule endoscope 2220 based on the detected position of the capsule endoscope 2220 and instructions from an operator; and an image display apparatus 1180 that displays the image signal transmitted from the capsule endoscope 2220.

As shown in FIG. 90, the position detection unit 2250 is mainly composed of drive coils (drive section) 2251 for generating an induced magnetic field in a magnetic induction coil, to be described later, inside the capsule endoscope 2220 and a drive-coil selector 1155 for calculating the position of the capsule endoscope 2220 based on induced electromotive force information, to be described later, and for controlling alternating magnetic fields generated by the drive coils 2251.

In addition, the drive coils 2251 are formed as air-core coils, and are supported by the three planar coil-supporting parts 1158 shown in the figure at the inner side of the Helmholtz coils 1171X, 1171Y, and 1171Z. Nine of the drive coils 2251 are arranged in the form of a matrix in each coil-supporting part 1158, and thus a total of 27 drive coils 2251 are provided in the position detection unit 2250.

As shown in FIG. 90, the image display apparatus 1180 is formed of an image receiving circuit 2281 that receives the image and induced electromotive force information, to be described later, transmitted from the capsule endoscope 2220 and a display section 1182 that displays the image based on the received image signal and a signal from the rotation-magnetic-field control circuit 1173.

An electromotive force detection circuit for detecting an induced electromotive force is connected to the magnetic induction coil of the capsule endoscope 2220.

The operation of the above-described medical magnetic-induction and the position-detection system 2210 will now be described.

The drive-coil selector 1155 generates an alternating magnetic field by time-sequentially switching among the drive coils 2251 based on a signal from the position detection unit 2250. The generated alternating magnetic field acts on the magnetic induction coil of the capsule endoscope 2220 to produce an induced electromotive force.

The electromotive-force detection circuit connected to the magnetic induction coil detects induced-electromotive-force information based on the above-described induced electromotive force.

When wirelessly transmitting acquired image data to the image reception circuit 2281, the capsule endoscope 2220 superimposes the detected induced electromotive force information on the image data. The image reception circuit 2281, which has received the image data and the induced electromotive force information, transmits the image data to the display section 1180 and transmits the induced electromotive force information to the position-detecting section 2250A. The position-detecting section 2250A calculates the position and orientation of the capsule endoscope based on the induced electromotive force information.

With the above-described structure, the position and direction of the capsule endoscope can be detected without providing a sense coil in the position detection unit 2250. Furthermore, by superimposing the induced electromotive force information on the image data to be transmitted, the position detection unit 2250 can be operated without providing a new transmitter in the capsule endoscope.

The technical field of the present invention is not limited to the aforementioned sixth to fifteenth embodiments, and various modifications may be applied within the scope thereof without departing from the gist of the invention.

For example, in the description of the aforementioned sixth to fifteenth embodiments, a capsule endoscope (medical device) provided with the imaging section 1130 is employed as a biological-information acquiring unit. Instead of the imaging section 1130, various devices can be employed as the biological-information acquiring unit, including a capsule medical device provided with a blood sensor to check for a bleeding site; a capsule medical device provided with a gene sensor to perform genetic diagnosis; a capsule medical device provided with a drug releasing unit to deliver a drug; a capsule medical device provided with a marking unit to place a mark in the body cavity; and a capsule medical device provided with a body-fluid-and-tissue collecting unit to collect body fluids and tissues in the body cavity.

Furthermore, although the sixth to fifteenth embodiments have been described by way of example of a capsule endoscope that is independent of the exterior, a capsule endoscope with a cord for connection to the exterior by the cord is also applicable.

1. A position detection system comprising:
a device equipped with a magnetic induction coil;
a drive coil for generating an alternating magnetic field;
a plurality of magnetic field sensors for detecting an induced magnetic field generated by the magnetic induction coil receiving the alternating magnetic field;
a frequency determining section for determining a position calculating frequency which is based on a resonance frequency of the magnetic induction coil; and
a position analyzing unit for calculating, at the position calculating frequency, at least one of the position and the orientation of the device based on the difference between outputs of the magnetic field sensors when only the alternating magnetic field is applied and outputs of the magnetic field sensors when the alternating magnetic field and the induced magnetic field are applied,
wherein, based on the position calculating frequency, at least one of a frequency range of the alternating magnetic field and an output frequency range of the magnetic field sensors is limited.

2. A position detection system according to claim 1, wherein the frequency determining section determines the position calculating frequency based on the outputs from the magnetic field sensors when the induced magnetic field is applied.

3. A position detection system according to claim 2, further comprising:
a magnetic-field-frequency varying section for time varying the frequency of the alternating magnetic field,
wherein the frequency determining section determines the position calculating frequency based on the outputs from the magnetic field sensors when applying the induced magnetic field generated by receiving the alternating magnetic field whose frequency is time varying.

4. A position detection system according to claim 2, further comprising:
an impulse-magnetic-field generating section for applying an impulse drive voltage to the drive coil to generate an impulse magnetic field,
wherein the frequency determining section determines the position calculating frequency based on the outputs from the magnetic field sensors when applying the induced magnetic field generated by receiving the impulse magnetic field.

5. A position detection system according to claim 1, further comprising:
a mixed-magnetic-field generating section for generating an alternating magnetic field in which a plurality of different frequencies are mixed; and
a variable band limiting section for limiting the output frequency range of the magnetic field sensors and for changing the range of limitation,
wherein the frequency determining section determines the position calculating frequency based on output which is acquired, through the variable band limiting section, the outputs of the magnetic field sensors when applying the induced magnetic field generated by receiving the alternating magnetic field in which a plurality of different frequencies are mixed.

6. A position detection system according to claim 1, further comprising:
a memory section for storing information concerning the resonance frequency of the magnetic induction coil,
wherein the frequency determining section receives the information and determines the position calculating frequency based on the information.

7. A position detection system according to claim 1, further comprising a band limiting section for limiting the output frequency band of the magnetic field sensors based on the position calculating frequency.

8. A position detection system according to claim 7, wherein the band limiting section uses a Fourier transform.

9. A position detection system according to claim 1, wherein the plurality of magnetic field sensors are disposed at a plurality of orientations facing an operating region of the device.

10. A position detection system according to claim 1, further comprising a magnetic-field-sensor selecting unit for selecting predeterminal number of magnetic field sensors whose signal outputs are strong from among the output signals of the plurality of magnetic field sensors.

11. A position detection system according to claim 1, wherein the drive coil and the magnetic field sensors are disposed at opposing positions on either side of the operating region of the device.

12. A position detection system according to claim 1, further comprising:
a relative-position measuring unit for measuring a relative position between the drive coil and the magnetic field sensors;
an information storing section for storing, in association with each other, a reference value, which is an output value from the magnetic field sensors when only the alternating magnetic field is applied, and an output from the relative-position measuring unit at that time; and
a present-reference-value generating section for generating, as a present reference value, a present output value of the magnetic field sensors when only the alternating magnetic field is applied, based on the output of the relative-position measuring unit and the information in the information storing section.

13. A position detection system according to claim 12, wherein the present-reference-value generating section generates, as the present reference value, the reference value which is associated with the relative position closest to the present output of the relative-position measuring unit.

14. A position detection system according to claim 12, wherein:
the present-reference-value generating section deter a predetermined approximate equation which relates the relative position and the reference value and generates the present reference value based on the predetermined approximate equation and the present output from the relative-position measuring unit.

15. A guidance system comprising:
a position detection system according to claim 1;
a guidance magnet installed in the device;
a guidance-magnetic-field generating unit for generating a guidance magnetic field to be applied to the guidance magnet; and
a guidance-magnetic-field-direction control unit for controlling the direction of the guidance magnetic field.

16. A guidance system according to claim 15, wherein:
the guidance-magnetic-field generating unit includes three pairs of frame shaped electromagnets disposed to oppose each other in mutually orthogonal directions;
a space in which a subject can be disposed is provided at the inner sides of the electromagnets; and
the drive coil and the magnetic field sensors are disposed around the space in which the subject can be disposed.

17. A guidance system according to claim 15, wherein a helical part for converting a rotary force around the longitudinal axis of the device into propulsion force in the direction of the longitudinal axis is provided on an outer surface of the device.

18. A position detection system according to claim 1, wherein the device is a capsule medical device.

19. A position detection method for a device, comprising:
a step of obtaining a characteristic of a magnetic induction coil installed in the device;
a step of determining a position calculating frequency from the characteristic;
a step of limiting at least one of a frequency range of an alternating magnetic field and a frequency range of a magnetic sensor based on the position calculating frequency;
a stop of generating the alternating magnetic field, which includes a position calculating frequency component;
a measuring step for obtaining an output from the magnetic field sensor; and
a position calculating step of determining at least one of the position and the orientation of the magnetic induction coil.

20. A position detection method according to claim 19, wherein the measuring step and the position calculating step are repeated.

21. A medical magnetic-induction and position-detection system comprising:
a medical device which is inserted inside a body of a subject and which includes at least one magnet and a circuit including a built-in coil;
a first magnetic-field generating section for generating a first magnetic field;
a magnetic-field detection section for detecting an induced magnetic field induced in the built-in coil by the first magnetic field; and
one or more sets of opposing coils for generating a second magnetic field to be applied to the magnet,
wherein the two coils constituting the opposing coils are driven separately.

22. A medical magnetic-induction and position-detection system according to claim 21, wherein at least three sets of the opposing coils are provided to surround a region where the magnet is disposed;
the first magnetic-field generating section includes a position-detecting magnetic-field generating coil disposed in the vicinity of one coil of at least one set of the opposing coils;
the position-detection unit includes a magnetic field sensor disposed in the vicinity of the other coil of the at least one set of opposing coils; and
of at least three sets of the opposing coils, the orientation of a central axis of at least one set of the opposing coils is disposed so as to intersect a plane formed by central axes of another two sets of the opposing coils.

23. A medical magnetic-induction and position-detection system comprising:
a medical device which is inserted inside a body of a subject and which includes at least one magnet and a circuit including a built-in coil;
a first magnetic-field generating section for generating a first magnetic field;
a magnetic-field detection section for detecting an induced magnetic field induced in the built-in coil by the first magnetic field;
one or more sets of opposing coils for generating a second magnetic field to be applied to the magnet; and
a switching section for electrically connecting to the opposing coils,
wherein the switching section is switched off only while the position-detection unit detects the position of the built-in coil.

24. A medical magnetic-induction and position-detection system according to claim 23, wherein at least three sets of the opposing coils are provided to surround a region where the magnet is disposed;
the first magnetic-field generating section includes a position-detecting magnetic-field generating coil disposed in the vicinity of one coil of at least one set of the opposing coils;
the position-detection unit includes a magnetic field sensor disposed in the vicinity of the other coil of the at least one set of opposing coils; and
of at least three sets of the opposing coils, the orientation of a central axis of at least one set of the opposing coils is disposed so as to intersect a plane formed by central axes of another two sets of the opposing coils.

25. A medical magnetic-induction and position-detection system comprising:
a medical device which is inserted inside a body of a subject and which includes at least one magnet and a circuit including a built-in coil;
a first magnetic-field generating section for generating a first magnetic field;
a magnetic-field detection section for detecting an induced magnetic field induced in the built-in coil by the first magnetic field; and
one or more sets of opposing coils for generating a second magnetic field to be applied to the magnet,
wherein the two coils constituting the opposing coils driven in parallel.

26. A medical magnetic-induction and position-detection system according to claim 25, wherein at least three sets of the opposing coils are provided to surround a region where the magnet is disposed;
the first magnetic-field generating section includes a position-detecting magnetic-field generating coil disposed in the vicinity of one coil of at least one set of the opposing coils;
the position-detection unit includes a magnetic field sensor disposed in the vicinity of the other coil of the at least one set of opposing coils; and
of at least three sets of the opposing coils, the orientation of a central axis of at least one set of the opposing coils is disposed so as to intersect a plane formed by central axes of another two sets of the opposing coils.

27. A medical device comprising at least one magnet and a circuit including a built-in coil having a core formed of magnetic material,
wherein the position of the built-in coil is detected by a magnetic position-detection unit disposed outside a body of a subject, and
wherein the core is disposed at a position where there is no magnetic saturation by the magnetic field that the magnet.

28. A medical device according to claim 27, wherein the core has a shape for which a demagnetizing factor in the core for the central axis direction of the built-in coil is smaller than a demagnetizing factor for other directions; and the direction of the magnetic field that the magnet produces at the core position is a direction intersecting the central axis direction.

29. A medical device according to claim 27, wherein the direction of the magnetic field that the magnet produces at the position of the built-in coil and the direction for which the demagnetizing factor in the core is minimized are different.

30. A medical device according to claim 29, wherein an angle formed between the direction of the magnetic field that the magnet produces at the position of the built-in coil and the direction for which the demagnetizing factor in the core is minimized is substantially 90 degrees.

31. A medical device according to claim 27, wherein the core is positioned so that a demagnetizing factor for the central axis direction is smaller than demagnetizing factors for other directions; and
the direction of the magnetic field that the magnet produces at the position of the built-in coil and the central axis direction are substantially orthogonal.

32. A medical device according to claim 31, wherein the magnet is disposed so that a center of gravity is located on the central axis; and
a magnetization direction of the magnet is substantially orthogonal to the central axis.

33. A medical device according to claim 27, wherein the built-in coil is disposed at a position where a magnetic flux density produced inside the core by the magnetic field of the magnet is ½ or less a saturated magnetic flux density in the core.

34. A medical device according to claim 27, wherein the circuit is a resonant circuit.

35. A medical device according to claim 27, wherein the built-in coil has a hollow structure;
the core is formed to be substantially C-shaped in cross-section perpendicular to the central axis direction; and
the core is disposed inside the hollow construction.

36. A medical device according to claim 33, further comprising a biological-information acquiring unit for acquiring information about the interior of the body of the subject;
wherein the magnet has a hollow structure, and
wherein at least a portion of the biological-information acquiring unit is disposed inside the hollow structure.

37. A medical device according to claim 34, further comprising a biological-information acquiring unit for acquiring information about the interior of the body of the subject;
wherein the magnet has a hollow structure, and
wherein at least a portion of the biological-information acquiring unit is disposed inside the hollow structure.

38. A medical device according to claim 27, wherein the magnet is formed of an assembly of plural magnet pieces, and insulators are disposed between the plural magnet pieces.

39. A medical device according to claim 38, wherein the plural magnet pieces are formed to be substantially plate-shaped.

40. A medical device according to claim 39, wherein the plural magnet pieces are polarized in thickness directions thereof.

41. A medical device according to claim 39, wherein the plural magnet pieces are polarized in directions along surfaces thereof.

42. A medical device according to claim 38, wherein the magnet which is an assembly of the plural magnet pieces is formed to be substantially cylindrical.

43. A capsule medical device, wherein a medical device according to claim 27, is inserted into the body of the subject and comprises a biological-information acquiring unit for acquiring information about the interior of the body of the subject.

44. A medical device according to claim 43, wherein the built-in coil has a hollow structure, and at least part of the biological-information acquiring unit is disposed inside the hollow structure.

45. A medical device according to claim 43, further comprising a power supply unit for driving the circuit and/or the biological-information acquiring unit, wherein the built-in coil has a hollow structure, and the power supply unit is disposed inside the hollow structure.

46. A medical device according to claim 43, further comprising a power supply unit for driving the circuit and/or the biological-information acquiring unit, wherein the magnet has a hollow structure, and the power supply unit is disposed inside the hollow structure.

47. A medical magnetic-induction and position-detection system comprising a medical device according to claim 27; and a position-detection unit including a driving section for generating an induced magnetic field in the built-in coil and a magnetic-field detecting section for detecting the induced magnetic field generated by the built-in coil, wherein the circuit is a magnetic-field generating unit for generating a magnetic field directed from the built-in coil to the position-detection unit.

48. A medical magnetic-induction and position-detection system according to claim 47, wherein the driving section of the position-detection unit forms a magnetic field in a region where the built-in coil is disposed, and the magnetic-field generating unit receives, by means of the built-in coil, the magnetic field that the position-detection unit produces to generate an induced magnetic field from the built-in coil.

49. A medical magnetic-induction and position-detection system according to claim 47, wherein the position-detection unit includes a plurality of the magnetic-field detecting sections and a calculating apparatus for calculating at least one of the position and orientation of the built-in coil based on the outputs of the plurality of magnetic-field detecting sections.

50. A medical magnetic-induction and position-detection system comprising: a medical device according to claim 27; and a position-detection unit including a driving section for forming magnetic fields from a plurality of directions to a region where the built-in coil is disposed, wherein the circuit includes an internal magnetic-field detecting section for receiving the plurality of magnetic fields that the position-detection unit forms and a position-information transmitting unit for transmitting information about the plurality of received magnetic fields to the position-detection unit.

51. A medical magnetic-induction and position-detection system according to claim 50, wherein the position-detection unit includes a calculating apparatus for calculating at least one of the position and orientation of the built-in coil based on the information about the plurality of magnetic fields detected at the internal magnetic-field detecting section.

52. A medical magnetic-induction and position-detection system according to claim 49, further comprising a guidance-magnetic-field generating unit, disposed outside an operating region of the medical device, for generating a driving magnetic field to be applied to the magnet; and a magnetic-field-direction control unit for controlling the direction of the driving magnetic field by controlling the guidance-magnetic-field generating unit.

53. A medical magnetic-induction and position-detection system according to claim 51, further comprising a guidance-magnetic-field generating unit, disposed outside an operating region of the medical device, for generating a driving magnetic field to be applied to the magnet; and a magnetic-field-direction control unit for controlling the direction of the driving magnetic field by controlling the guidance-magnetic-field generating unit.

54. A medical device in which the position of a built-in coil is detected by a magnetic position-detection unit disposed outside a body of a subject,
wherein two of the built-in coils are provided, and the two built-in coils are disposed so that central axes thereof are aligned with each other, as well as being disposed so as to be separated in the central axis direction.

55. A capsule medical device, wherein a medical device according to claim 54, is inserted into the body of the subject and comprises a biological-information acquiring unit for acquiring information about the interior of the body of the subject.

56. A medical device according to claim 55, wherein the built-in coil has a hollow structure, and at least part of the biological-information acquiring unit is disposed inside the hollow structure.

57. A medical device according to claim 55, further comprising a power supply unit for driving the circuit and/or the biological-information acquiring unit, wherein the built-in coil has a hollow structure, and the power supply unit is disposed inside the hollow structure.

58. A medical device according to claim 55, further comprising a power supply unit for driving the circuit and/or the biological-information acquiring unit, wherein the magnet has a hollow structure, and the power supply unit is disposed inside the hollow structure.

59. A medical magnetic-induction and position-detection system comprising a medical device according to claim 54; and a position-detection unit including a driving section for generating an induced magnetic field in the built-in coil and a magnetic field detecting section for detecting the induced magnetic field generated by the built-in coil, wherein the circuit is a magnetic-field generating unit for generating a magnetic field directed from the built-in coil to the position-detection unit.

60. A medical magnetic-induction and position-detection system according to claim 59, wherein the driving section of the position-detection unit forms a magnetic field in a region where the built-in coil is disposed, and the magnetic-field generating unit receives, by means of the built-in coil, the magnetic field that the position-detection unit produces to generate an induced magnetic field from the built-in coil.

61. A medical magnetic-induction and position-detection system according to claim 59, wherein the position-detection unit includes a plurality of the magnetic-field detecting sections and a calculating apparatus for calculating at least one of the position and orientation of the built-in coil based on the outputs of the plurality of magnetic-field detecting sections.

62. A medical magnetic-induction and position-detection system comprising: a medical device according to claim 54; and a position-detection unit including a driving section for forming magnetic fields from a plurality of directions to a region where the built-in coil is disposed, wherein the circuit includes an internal magnetic-field detecting section for receiving the plurality of magnetic fields that the position-detection unit forms and a position-information transmitting unit for transmitting information about the plurality of received magnetic fields to the position-detection unit.

63. A medical magnetic-induction and position-detection system according to claim 62, wherein the position-detection unit includes a calculating apparatus for calculating at least one of the position and orientation of the built-in coil based on the information about the plurality of magnetic fields detected at the internal magnetic-field detecting section.

64. A medical magnetic-induction and position-detection system according to claim 61, further comprising a guidance-magnetic-field generating unit, disposed outside an operating region of the medical device, for generating a driving magnetic field to be applied to the magnet; and a magnetic-field-direction control unit for controlling the direction of the driving magnetic field by controlling the guidance-magnetic-field generating unit.

65. A medical magnetic-induction and position-detection system according to claim 63, further comprising a guidance-magnetic-field generating unit, disposed outside an operating region of the medical device, for generating a driving magnetic field to be applied to the magnet, and a magnetic-field-direction control unit for controlling the direction of the driving magnetic field by controlling the guidance-magnetic-field generating unit.

66. A medical device which comprises two magnets and a circuit including a built-in coil and in which the position of the built-in coil is detected by a magnetic position-detection unit disposed outside a body of a subject,
wherein the built-in coil is disposed between the two magnets.

67. A capsule medical device, wherein a medical device according to claim 66, is inserted into the body of the subject and comprises a biological-information acquiring unit for acquiring information about the interior of the body of the subject.

68. A medical device according to claim 67, wherein the built-in coil has a hollow structure, and at least part of the biological-information acquiring unit is disposed inside the hollow structure.

69. A medical device according to claim 67, further comprising a power supply unit for driving the circuit and/or the biological-information acquiring unit, wherein the built-in coil has a hollow structure, and the power supply unit is disposed inside the hollow structure.

70. A medical device according to claim 67, further comprising a power supply unit for driving the circuit and/or the biological-information acquiring unit, wherein the magnet has a hollow structure, and the power supply unit is disposed inside the hollow structure.

71. A medical magnetic-induction and position detection system comprising a medical device according to claim 66; and a position-detection unit including a driving section for generating an induced magnetic field in the built-in coil and a magnetic field detecting section for detecting the induced magnetic field generated by the built-in coil, wherein the circuit is a magnetic-field generating unit for generating a magnetic field directed from the built-in coil to the position-detection unit.

72. A medical magnetic-induction and position-detection system according to claim 71, wherein the driving section of the position-detection unit forms a magnetic field in a region where the built-in coil is disposed, and the magnetic-field generating unit receives, by means of the built-in coil, the magnetic field that the position-detection unit produces to generate an induced magnetic field from the built-in coil.

73. A medical magnetic-induction and position-detection system according to claim 71, wherein the position-detection unit includes a plurality of the magnetic-field detecting sections and a calculating apparatus for calculating at least one of the position and orientation of the built-in coil based on the outputs of the plurality of magnetic-field detecting sections.

74. A medical magnetic-induction and position-detection system comprising: a medical device according to claim 66; and a position-detection unit including a driving section for forming magnetic fields from a plurality of directions to a region where the built-in coil is disposed, wherein the circuit includes an internal magnetic-field detecting section for receiving the plurality of magnetic fields that the position-detection unit forms and a position-information transmitting unit for transmitting information about the plurality of received magnetic fields to the position-detection unit.

75. A medical magnetic-induction and position-detection system according to claim 74, wherein the position-detection unit includes a calculating apparatus for calculating at least one of the position and orientation of the built-in coil based on the information about the plurality of magnetic fields detected at the internal magnetic-field detecting section.

76. A medical magnetic-induction and position-detection system according to claim 73, further comprising a guidance-magnetic-field generating unit, disposed outside an operating region of the medical device, for generating a driving magnetic field to be applied to the magnet; and a magnetic-field-direction control unit for controlling the direction of the driving magnetic field by controlling the guidance-magnetic-field generating unit.

77. A medical magnetic-induction and position-detection system according to claim 75, further comprising a guidance-magnetic-field generating unit, disposed outside an operating region of the medical device, for generating a driving magnetic field to be applied to the magnet; and a magnetic-field-direction control unit for controlling the direction of the driving magnetic field by controlling the guidance-magnetic-field generating unit.

78. A medical device which is inserted inside a body of a subject and which includes therein at least one magnet and a circuit including at least a built-in coil, the position of the built-in coil being detected by a magnetic position-detection unit disposed outside the body of the subject, wherein the magnet is disposed so as to be separated from the built-in coil in an axial direction of the built-in coil.

79. A medical device according to claim 78, wherein the position and orientation of the magnet are controlled by a magnetic field formed therearound.

80. A medical device according to claim 78, wherein a gap disposed between the magnet and the built-in coil is determined on the basis of a magnetic field intensity of the magnet, formed at the center of the built-in coil.

81. A medical device according to claim 78, wherein the circuit is a magnetic-field generating unit for generating a magnetic field directed from the built-in coil to the position-detection unit.

82. A medical device according to claim 78, wherein the position-detection unit forms magnetic fields, from a plurality of directions to a region where the built-in coil is disposed; and the circuit includes a mechanism for receiving with the built-in coil, the plurality of magnetic fields that the position-detection unit forms and a position-information transmitting unit for transmitting intensity information about the plurality of received magnetic fields towards the position-detection unit.

83. A medical device according to claim 78, wherein the position-detection unit forms a magnetic field in a region where the built-in coil is disposed; and
the circuit includes a mechanism for receiving, with the built-in coil, the magnetic field that the position-detection unit forms and an induced-magnetic-field generating unit for generating, by magnetic induction, an induced magnetic field directed from the built-in coil to the position-detection unit.

84. A medical device according to claim 78, further comprising a biological-information acquiring unit for acquiring biological information about the subject.

85. A medical device according to claim 84, wherein the biological-information acquiring unit includes an image-acquisition unit for imaging the biological information about the subject and an illumination unit for illuminating an image-acquisition region.

86. A medical device according to claim 84, wherein the built-in coil has a hollow structure, and at least a portion of the biological-information acquiring unit is disposed inside the hollow structure.

87. A medical device according to claim 84, further comprising a power supply unit for driving the circuit and/or the biological-information acquiring unit;
wherein the built-in coil has a hollow structure, and the power supply unit is disposed inside the hollow structure.

88. A medical device according to claim 86, wherein a magnetic body whose cross-section is formed substantially in the shape of a letter C is disposed inside the hollow structure of the built-in coil.

89. A medical device according to claim 86, wherein the magnetic body is formed of permalloy, iron, or nickel.

90. A medical device according to claim 84, wherein the magnet has a hollow structure, and at least a portion of the biological-information acquiring unit is disposed inside the hollow structure.

91. A medical device according to claim 84, further comprising a power supply unit for driving the circuit and/or the biological-information acquiring unit;
wherein the magnet has a hollow structure, and the power supply unit is disposed inside the hollow structure.

92. A medical device according to claim 78, having two of the built-in coils, wherein the two built-in coils are disposed so as to be separated from each other in the axial direction thereof, and the magnet is disposed between the two built-in coils.

93. A medical device according to claim 90, having two of the magnets, wherein the two magnets are disposed so as to be separated from each other in the axial direction of the built-in coil, and the built-in coil is disposed between the two magnets.

94. A medical device according to claim 78, wherein the magnet is formed of an assembly of plural magnet pieces, and insulators are disposed between the plural magnet pieces.

95. A medical device according to claim 94, wherein the plural magnet pieces are formed to be substantially plate-shaped.

96. A medical device according to claim 95, wherein the plural magnet pieces are polarized in the thickness direction thereof.

97. A medical device according to claim 95, wherein the plural magnet pieces are polarized in directions along surfaces thereof.

98. A medical device according to claim 94, wherein the magnet which is the assembly of the plural magnet pieces is formed to be substantially cylindrical.

99. A medical device according to claim 78, wherein the circuit forms a self-resonant circuit.

100. A medical device according to claim 78, wherein the circuit includes a capacitor, and the built-in coil and the capacitor are connected in parallel to form an LC resonant circuit.

101. A medical magnetic-induction and position-detection system for detecting the position of a medical device inserted inside a body of a subject, comprising:
a medical device according to claim 78; and
a position-detection unit including a drive coil, disposed outside an operating region of the medical device, for generating an induced magnetic field in the built-in coil and a magnetic field sensor, disposed outside the operating region of the medical device, for detecting the induced magnetic field generated by the built-in coil.

102. A medical magnetic-induction and position-detection system according to claim 101, wherein when the medical device is disposed at positions inside the operating region of the medical device,
the drive coil exerts magnetism, from three or more different directions, on the magnetic induction coil and is disposed so that, of the directions in which the magnetism of three or more directions is exerted, at least one direction intersects a plane formed from the other two directions.

103. A medical magnetic-induction and position-detection system according to claim 101, wherein a plurality of the magnetic field sensors are disposed in a plurality of orientations facing the operating region of the medical device.

104. A medical magnetic-induction and position-detection system according to claim 101, further comprising a magnetic-field-sensor selecting unit for selectively using an output signal whose signal output is strong, from among output signals of the plurality of magnetic field sensors.

105. A medical magnetic-induction and position-detection system according to claim 101, wherein the drive coil and the magnetic field sensor are disposed at opposing positions on either side of the operating region of the medical device.

106. A medical magnetic-induction and position-detection system comprising:
a position detection unit according to claim 101;
a guidance-magnetic-field generating unit, disposed outside the operating region of the medical device, for generating a guidance magnetic field acting on the magnet of the medical device; and
a magnetic-field-orientation control unit for controlling the orientation of the guidance magnetic field by controlling the guidance-magnetic-field generating unit.

107. A medical magnetic-induction and position-detection system according to claim 106, wherein the guidance-magnetic-field generating unit includes three pairs of frame-shaped electromagnets disposed in mutually orthogonal orientations;
a space in which the subject can be placed is provided at an inner side of the electromagnets, and the drive coil and the magnetic field sensor are disposed around the space.

108. A medical magnetic-induction and position-detection system according to claim 106, wherein a helical mechanism for converting rotary force about a longitudinal axis of the medical device to propulsive force in the longitudinal axis direction is provided on an outer surface of the medical device.

109. A medical magnetic-induction and position-detection system according to claim 108, wherein an image-acquisition unit having an optical axis parallel to the longitudinal axis of the medical device is provided in the medical device, a display unit for displaying an image acquired by the image-acquisition unit is provided, and an image control unit is provided for rotating the image acquired by the image-acquisition unit in an opposite direction, on the basis of rotation information about the longitudinal axis of the medical device due to the magnetic-field-orientation control unit, and for displaying the image on the display unit.

110. A medical magnetic-induction and position-detection system for detecting the position of a medical device that is inserted inside a body of a subject, comprising:
a medical device according to claim 99; and
a position-detection unit including a drive coil, disposed outside an operating region of the medical device, for generating an induced magnetic field in the built-in coil and a magnetic field sensor, disposed outside the operating region of the medical device, for detecting the induced magnetic field generated by the built-in coil,
wherein a frequency of an alternating magnetic field that the drive coil generates is close to a self-resonant frequency of the circuit.

111. A medical magnetic-induction and position-detection system according to claim 110, wherein when the medical device is disposed at positions inside the operating region of the medical device, the drive coil exerts magnetism, from three or more different directions, on the magnetic induction coil and is disposed so that, of the directions in which the magnetism of three or more directions is exerted, at least one direction intersects a plane formed from the other two directions.

112. A medical magnetic-induction and position-detection system according to claim 110, wherein a plurality of the magnetic field sensors are disposed in a plurality of orientations facing the operating region of the medical device.

113. A medical magnetic-induction and position-detection system according to claim 110, further comprising a magnetic-field-sensor selecting unit for selectively using an output signal whose signal output is strong, from among output signals of the plurality of magnetic field sensors.

114. A medical magnetic-induction and position-detection system according to claim 110, wherein the drive coil and the magnetic field sensor are disposed at opposing positions on either side of the operating region of the medical device.

115. A medical magnetic-induction and position-detection system comprising:
a position detection unit according to claim 110;
a guidance-magnetic-field generating unit, disposed outside the operating region of the medical device, for generating a guidance magnetic field acting on the magnet of the medical device; and
a magnetic-field-orientation control unit for controlling orientation of the guidance magnetic field by controlling the guidance-magnetic-field generating unit.

116. A medical magnetic-induction and position-detection system according to claim 115, wherein the guidance-magnetic-field generating unit includes three pairs of frame-shaped electromagnets disposed in mutually orthogonal orientations;
a space in which the subject can be placed is provided at an inner side of the electromagnets, and the drive coil and
the magnetic field sensor are disposed around the space.

117. A medical magnetic-induction and position-detection system according to claim 115, wherein a helical mechanism for converting rotary force about a longitudinal axis of the medical device to propulsive force in the longitudinal axis direction is provided on an outer surface of the medical device.

118. A medical magnetic-induction and position-detection system according to claim 117, wherein an image-acquisition unit having an optical axis parallel to the longitudinal axis of the medical device is provided in the medical device, a display unit for displaying an image acquired by the image-acquisition unit is provided, and an image control unit is provided for rotating the image acquired by the image-acquisition unit in an opposite direction, on the basis of rotation information about the longitudinal axis of the medical device due to the magnetic-field-orientation control unit, and for displaying the image on the display unit.

119. A medical magnetic-induction and position-detection system for detecting the position of a medical device that is inserted inside a body of a subject, comprising:
a medical device according to claim 100; and
a position-detection unit including a drive coil, disposed outside an operating region of the medical device, for generating an induced magnetic field in the built-in coil and a magnetic field sensor, disposed outside the operating region of the medical device, for detecting the induced magnetic field generated by the built-in coil,
wherein a frequency of an alternating magnetic field that the drive coil generates is close to an LC resonance frequency of the LC resonant circuit.

120. A medical magnetic-induction and position-detection system according to claim 119, wherein when the medical device is disposed at positions inside the operating region of the medical device, the drive coil exerts magnetism, from three or more different directions, on the magnetic induction coil and is disposed so that, of the directions in which the magnetism of three or more directions is exerted, at least one direction intersects a plane formed from the other two directions.

121. A medical magnetic-induction and position-detection system according to claim 119, wherein a plurality of the magnetic field sensors are disposed in a plurality of orientations facing the operating region of the medical device.

122. A medical magnetic-induction and position-detection system according to claim 119, further comprising a magnetic-field-sensor selecting unit for selectively using an output signal whose signal output is strong, from among output signals of the plurality of magnetic field sensors.

123. A medical magnetic-induction and position-detection system according to claim 119, wherein the drive coil and the magnetic field sensor are disposed at opposing positions on either side of the operating region of the medical device.

124. A medical magnetic-induction and position-detection system comprising:
a position detection unit according to claim 119;
a guidance-magnetic-field generating unit, disposed outside the operating region of the medical device, for
generating a guidance magnetic field acting on the magnet of the medical device; and
a magnetic-field-orientation control unit for controlling the orientation of the guidance magnetic field by controlling the guidance-magnetic-field generating unit.

125. A medical magnetic-induction and position-detection system according to claim 124, wherein the guidance-magnetic-field generating unit includes three pairs of frame shaped electromagnets disposed in mutually orthogonal orientations;
a space in which the subject can be placed is provided at an inner side of the electromagnets, and the drive coil and the magnetic field sensor are disposed around the space.

126. A medical magnetic-induction and position-detection system according to claim 124, wherein a helical mechanism for converting rotary force about a longitudinal axis of the medical device to propulsive force in the longitudinal axis direction is provided on an outer surface of the medical device.

127. A medical magnetic-induction and position detection system according to claim 126, wherein an image-acquisition unit having an optical axis parallel to the longitudinal axis of the medical device is provided in the medical device, a display unit for displaying an image acquired by the image-acquisition unit is provided, and an image control unit is provided for rotating the image acquired by the image-acquisition unit in an opposite direction, on the basis of rotation information about the longitudinal axis of the medical device due to the magnetic-field-orientation control unit and for displaying the image on the display unit.
